(19) United States
(12) Patent Application Publication
(10) Pub. No.: US 2022/0331377 A1
(43) Pub. Date: Oct. 20, 2022

(54) DELIVERY VEHICLE FOR IN SITU DELIVERING OF PHARMACEUTICAL AGENTS

(71) Applicant: RIVER STONE BIOTECH ISG APS, Copenhagen (DK)

(72) Inventors: Susanne Manuela GERMANN, Copenhagen (DK); Swee Chuang Lim HALLWYL, Copenhagen (DK); Jørgen HANSEN, Copenhagen (DK); Javier Porcayo LOZA, Copenhagen (DK)

(73) Assignee: RIVER STONE BIOTECH ISG APS, Copenhagen (DK)

(21) Appl. No.: 17/640,307

(22) PCT Filed: Sep. 9, 2020
(86) PCT No.: PCT/EP2020/075152

§ 371 Date: Mar. 3, 2022

(30) Foreign Application Priority Data
Sep. 9, 2019 (EP) 19196147.3
Apr. 15, 2020 (EP) 20169733.1

(51) Int. Cl. A61K 35/742; A61K 36/064; C12N 15/81; A61K 9/16; A61K 9/00

(52) U.S. Cl. CPC A61K 35/742; A61K 36/064; C12N 15/81; A61K 9/16; A61K 9/0053

(57) ABSTRACT

The present invention relates to delivery vehicles comprising a system producing one or more active pharmaceutical ingredient (API) capable of preventing, treating and/or relieving one or more diseases in a mammal, wherein the vehicle comprises genetically modified microbial host cells suitable for administering to the mammal and wherein the vehicle is capable of delivering the produced API in situ of the location in the mammal body in need of preventing, treating and/or relieving the disease.

### FIELD OF THE INVENTION

The present disclosure pertains to vehicles capable of producing and delivering active pharmaceutical compounds in situ for preventing, treating and/or relieving a disease.

### BACKGROUND OF THE INVENTION

The background art in the field of the present invention includes WO2010124387 disclosing highly BSH active bacteria. Further US2017360850 discloses certain probiotic strains of the species Saccharomyces boulardii, and Castagliuolo et al. (1999) discloses that a certain Saccharomyces boulardii protease inhibits the effects of Clostridioides difficile toxins A and B in human colonic mucosa. Other art including WO2015168534 pertains to therapeutic treatment of gastrointestinal microbial imbalances through competitive microbe replacement. Further art including CN106754843, CN106591272, CN106399284 and WO2017123592 pertains to bacteria expressing bile salt hydrolases, while others including CN106754445, CN106399284, and CN106754844 pertain to Pichia pastoris yeast producing a bile salt hydrolase enzyme. Hudson et al., 2014 discloses a genetically engineered S. boulardii heterologously expressing a protein in the gastrointestinal tract of mice. Hudson et al., 2014 discloses a genetically engineered S. boulardii heterologously expressing a simple 27 kDa green flourescent protein (frequently used as a reporter of expression since this has previously been achieved in many species, including bacteria, yeasts, fungi, fish and mammals) in the gastrointestinal tract of mice. Subsequently, Bagherpour et al., 2018 engineered S. boulardii to heterologously express a fusion protein comprising the highly immunogenic ovalbumin and a claudin-targeting sequence from Clostridium perfringens enterotoxin to assit rapid transportation out of the gut across the intestinal barrier. When administered orally to mice, these recombinant S. boulardii significantly increased anti-OVA IgG in serum of the mice, i.e. triggered an immune response. Neither of these publications teach that an active pharmaceutical ingredient, specifically an enzyme, may be expressed using recombinant S. boulardii. Neither demonstrated enzyme activity within the gut (i.e. in situ of a disease location), nor evidence beneficial effects attributable to heterologously expressed enzyme activity within the gut, nor do any of these publications present a method of treating a disease using recombinant S. boulardii heterologously expressing an enzyme.

The term “BSH” as used herein refers to Bile Salt Hydrolase, which is an enzyme natively produced by intestinal microflora and is a selection criteria for probiotics (Begley et al., 2006), that catalyzes the deconjugation of a conjugated bile acid into an unconjugated bile acid. Examples of this reaction include deconjugating glycocholic acid (GCA) or taurocholic acid (TCA) into cholic acid (CA); glycodeoxycholic acid (GDCA) or taurodeoxycholic acid (TDCA) into deoxycholic acid (DCA), and glycochenodeoxycholic acid (GCDCA) or taurochenodeoxycholic acid (TCDCA) into chenodeoxycholic acid (CDCA). In the gut, where BSH is active, the bile salts will be in the form of bile acids. Use of probiotics for alleviation of a vast range of maladies have been alleged or reported. For example, WO2019053218 pertains to enhancing probiotic potency of the yeast Saccharomyces boulardii.

### SUMMARY OF THE INVENTION

Providing certain technical improvement, advantages and/or advancements over the background art the present invention provides in a first aspect a delivery vehicle comprising a genetically modified microbial host cell comprising one or more heterologous polynucleotides encoding and producing a one or more enzyme active pharmaceutical ingredient (API), wherein the vehicle is suitable for administering to the mammal and wherein the modified microbial host cell is capable of producing and delivering the one or more enzyme API in situ of the location in the body of a mammal in need of preventing, treating and/or relieving a disease.

In a further aspect the invention provides a polynucleotide construct comprising a polynucleotide sequence encoding the API, operably linked to one or more control sequences.

In a further aspect the invention provides a cell culture, comprising the microbial cell of the invention. and a growth medium.

In a further aspect the invention provides a method for producing the cell culture of the invention comprising
a) culturing the microbial cell of any preceding claim at conditions allowing growth of the microbial cell; and
b) optionally recovering and/or isolating the cell culture.

In a further aspect the invention provides a fermentation liquid or composition comprising cell culture of the invention.

In a further aspect the invention provides a composition comprising the vehicle, and/or cell culture of the invention and one or more carriers, agents, additives and/or excipients.

In a further aspect the invention provides a pharmaceutical composition comprising the vehicle, microbial cell and/or cell culture of the invention and one or more pharmaceutical grade excipient, additives and/or adjuvants.

In a further aspect the invention provides a method for preparing a pharmaceutical preparation comprising mixing the vehicle and/or cell culture of the invention with one or more pharmaceutical grade excipient, additives and/or adjuvants.

In a further aspect the invention provides a method for treating a disease comprising administering the pharmaceutical preparation of the invention in an amount for the vehicle to produce and deliver in situ a therapeutically effective amount of the API.

### FIGURES

FIG. 1. Deconjugation of six conjugated bile acids into their respective three deconjugated bile acids by S. boulardii yeast strains expressing selected codon-optimized BSH enzymes (Sb^{BSH}, listed in Table 1) under aerob conditions in mineral media. The respective BSH enzymes were expressed in a genetic background where the yeast gene combination Sc_KEX2/Sc_BiP was overexpressed. Strains made in Example 1 were analyzed under screening conditions as described in Example 2 in Delft media, aerob, and 24 h of incubation with 10 mM BAM (bile acid mix, for details see Example 2). Commercial Chologlycine hydrolase (CGH) from Clostridium perfingens (Sigma) was used as positive control for deconjugation. Error bars represent standard deviations between technical triplicates. Values above 100% deconjugation reflect artifacts due to normalization to the media control.

FIG. 2. Deconjugation of six conjugated bile acids by Sb^{BSH} strains expressing codon-optimized B. ovatus BSH (Bo_BSH_co) with and without His-tag under aerob conditions in mineral media. Bo_BSH_co was expressed in different genetic backgrounds where the yeast gene combinations Sc_KEX2/Sc_BiP/Sc_PDI, Sc_KEX2/Sc_BiP or Sc_KEX2/Sc_PDI were overexpressed, and where Bo_BSH_co was either non-tagged or His-tagged (+His-tag). Strains made in Example 3 and listed in Table 1 were analyzed under screening conditions as described in Example 2 in Delft media, aerob, and 24 h of incubation with 10 mM BAM. Commercial Chologlycine hydrolase (CGH) from Clostridium perfingens (Sigma) was used as positive control for deconjugation. Error bars represent standard deviations between four independent biological clones of the initial screening.

FIG. 3. Deconjugation of six conjugated bile acids by Sb^{BSH} strains expressing codon-optimized B. ovatus BSH (Bo_BSH_co) under anaerob conditions in FeSSCoF media mimicking gastrointestinal conditions. Bo_BSH_co was expressed in different genetic backgrounds where the yeast gene combinations Sc_KEX2/Sc_BiP/Sc_PDI, Sc_KEX2/Sc_BiP or Sc_KEX2/Sc_PDI were overexpressed. Strains made in Example 1 and listed in Table 1 were analyzed under screening conditions as described in Example 2 in falcon tubes, FeSSCoF media, anaerob, and 72 h of incubation with 10 mM BAM. Time points were taken at 24 h, 48 h and 72 h after addition of BAM.

FIG. 4. Deconjugation of six conjugated bile acids by Sb^{BSH} strains listed in Table 1 expressing selected codon-optimized BSH enzymes in different media and with different conjugated bile acid substrates. The respective BSH enzymes were expressed in a genetic background where the yeast gene combination Sc_KEX2/Sc_BiP was overexpressed. A) Strains made in Example 1 were analyzed under screening conditions as described in Example 5a in BHIS media, semi-anaerob, and 24 h of incubation with 10 mM BAM. Commercial Chologlycine hydrolase (CGH) from Clostridium perfingens (Sigma) was used as positive control for deconjugation. Error bars represent standard deviations between technical triplicates. Values above 100% deconjugation reflect artifacts due to normalization to the media control. B) Strains made in Example 1 were analyzed under screening conditions as described in Example 5a in FeSSCoF media, semi-anaerob, and 24 h of incubation with 10 mM BAM. Commercial Chologlycine hydrolase (CGH) from Clostridium perfingens (Sigma) was used as positive control for deconjugation. Error bars represent standard deviations between technical triplicates. C) Strains made in Example 1 were analyzed under screening conditions as described in Example 5a in FeSSCoF media, semi-anaerob, and 24 h of incubation with 2% Ox Bile (Sigma Aldrich). Commercial Chologlycine hydrolase (CGH) from Clostridium perfingens (Sigma) was used as positive control for deconjugation. Error bars represent standard deviations between technical triplicates.

FIG. 5. Aerobic growth curve of yeast strains. Panel A shows the growth of the wild type and five Sb^{BSH} strains, and panel B shows the wild type and four other Sb^{BSH} strains, all listed in Table 1. Strains were subcultured (1/100) from an overnight culture and grown in BHIS (37° C., 250 rpm) for 25 h with OD₆₀₀ measurmeents being recorded hourly.

FIG. 6. Anaerobic growth curve of yeast strains. Panel A shows the growth of the wild type and five Sb^{BSH} strains, and panel B shows the wild type and four other Sb^{BSH} strains. Strains were subcultured (1/100) from an overnight, aerobic culture and grown in pre-reduced BHIS (37° C., 250 rpm) anaerobically for 72 h with OD₆₀₀ measurements being recorded periodically.

FIG. 7A. Inhibition of CD630 growth by Sb^{BSH}strains. The amount of growth inhibition (%) against CD630 after 24 h by Sb^{BSH}strains grown in 0, 5 mM or 10 mM BAM is displayed. Growth inhibition was calculated using the OD₆₀₀ measurements of the sample and the media only control. Each bar represents the mean of a reaction performed in duplicate and the errors bars represent standard deviation. Statistical analysis: Unpaired t-test results are indicated as follows: ns—not significant, *p≤0.05, **p≤0.01, ***p≤0.001.

FIG. 7B. Inhibition of R20291 growth by Sb^{BSH} strains. The amount of growth inhibition (%) against R20291 after 24 h by Sb^{BSH} strains grown in 0, 5 mM or 10 mM BAM is displayed. Growth inhibition was calculated using the OD₆₀₀ measurements of the sample and the media only control. Each bar represents the mean of a reaction performed in duplicate and the errors bars represent standard deviation. Statistical analysis: Unpaired t-test results are indicated as follows: ns—not significant, *p≤0.05, **p≤0.01, ***p≤0.001.

FIG. 8. Effect of cholestyramine on the activity of 0% Ox bile Sb^{BSH} supernatants against C. difficile. Non-supplemented Sb^{BSH}growth supernatants were either treated or not with cholestyramine (50 mg/ml) and tested for their inhibitory activity against CD630 (Panel A) or against R20291 (Panel B). Growth inhibtion was calculated using the OD₆₀₀ measurements of the sample and the media only control. Each bar represents the mean of a reaction performed in duplicate and the errors bars represent standard deviation.

FIG. 9. Effect of cholestyramine on the activity of 1% Ox bile Sb^{BSH} supernatants against C. difficile. Supernatants from Sb strains supplemented with 1% Ox bile were either treated or not with cholestyramine (50 mg/ml) and tested for their inhibitory activity against CD630 (Panel A) or against R20291 (Panel B). Growth inhibition was calculated using the OD₆₀₀ measurements of the sample and the media only control. Each bar represents the mean of a reaction in duplicate and the errors bars represent standard deviation. Unpaired t-test, *p≤0.05, p≤0.01, ***p≤0.001.

FIG. 10. Inhibition of C. difficile spore germination by Sb^{BSH} Supernatants from Sb^{BSH} strains grown in FeSSCoF+/−10 mM BAM were incubated for 15 min with 10⁷ CD630 (Panel A) or R20291 (Panel B) spores in the presence of 1% TCA and plated on BHIS with/without 0.1% NaTa. Germination (%) is shown.

FIG. 11. Growth profile of Sb^{BSH}strains. Evaluation of the growth of strains sSMT88 (Sb^{WT}), sSMT91 (Sb^{BSH1}) and sSMT95 (Sb^{BSH2}) in different complex medias in order to grade their performance. Top panel shows growth profile in YPD and YPD 2xGlu. Bottom panel shows the specific growth rates of the individual strains in YPD and YPD 2xGlu.

FIG. 12A. Symptoms Group 1 (PBS, placebo). Percentage of animals displaying mild, moderate or severe symptoms in group 1. Deceased hamsters included. On day −3 animals were administered the appropriate Sb strain or PBS sham treatment as specified in Table 4, once per day (0.5 ml/dose). This was repeated daily until the end of the experiment on day 2 (50 hours). On day −1 clindamycin 2-phosphate was given to hamsters by single i.g. gavage at 30 mg/kg. Adverse effects and/or CDI symptoms were recorded daily from day −3. On day 0 all hamsters were challenged with 1×10² spores of C. difficile strain 630 (200 μl oral gavage) 20 h after clindamycin treatment. The study endpoint was reached at 50 h when all hamsters had succumbed to infection. The CDI symptom grading of animals is subjective and the following symptom details were used as a guideline. CDI symptoms were scored and grouped into three distrinct categories. Mild diarrhoea and a wet tail (diameter4<2 cm) were considered to be mild symptoms (+). Heavy diarrhoea, a wet tail (diameter 4<2 cm) and lethargy were considered to be moderate symptoms (++). A wet tail (diameter 4>2 cm), extreme lethargy and visible weakness were considered to be severe symptoms (+++). Hamsters were observed closely after symptoms had developed and moribundity and death often rapidly followed the onset of severe symptoms.

FIG. 12B. Symptoms Group 2 (sSMT88, Sb^{wild type}). Percentage of animals displaying mild, moderate or severe symptoms in group 2. Deceased hamsters included. CDI symptom grading was performed as described in FIG. 11A.

FIG. 12C. Symptoms Group 3 (sSMT91, Sb^{BSH1}). Percentage of animals displaying mild, moderate or severe symptoms in group 3. Deceased hamsters included. CDI symptom grading was performed as described in FIG. 11A.

FIG. 12D. Symptoms Group 4 (sSMT95, Sb^{BSH2}). Percentage of animals displaying mild, moderate or severe symptoms in group 4. Deceased hamsters included. CDI symptom grading was performed as described in FIG. 11A.

FIG. 13. Time from challenge to colonization. Colonization is defined by the presence of symptoms. Graphic representation of data shown in Table 5.

FIG. 14. Time from challenge to death. Graphic representation of data shown in Table 5.

FIG. 15. Kaplan-Meier survival analysis. Kaplan-Meier survival estimates after oral challenge of these animals with 100 spores of C. difficile 630.

FIG. 16. C. difficile spores CFU in cecum (individual values). Upon death, hamsters were immediately frozen at −20° C. On the day of analysis, hamsters were thawed, cecum removed and used for immediate C. difficile CFU analysis.

FIG. 17. Toxin A levels in cecum (individual values). Upon death, hamsters were immediately frozen at −20° C. On the day of analysis, hamsters were thawed, cecum removed and used for immediate toxin analysis by ELISA.

FIG. 18. S. boulardii CFU in feces (individual values). Once a hamster had succumbed to infection, feces were collected from the cage and analyzed for S. boulardii CFU.

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION OF THE INVENTION

### Definitions

Any EC numbers used herein refers to Enzyme Nomenclature 1992 from NC-IUBMB, Academic Press, San Diego, Calif. including 30 supplements 1-5 published in Eur. J. Bio-chem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see e.g. http://enzyme.expasy.org/.

The term “API” or “Active Pharmaceutical Ingredient” as used interchangeable herein refers to any substance or mixture of substances intended to be used in the manufacture of a drug (medicinal) product and that, when used in the production of a drug, becomes an active ingredient of the drug product. Such substances are intended to furnish pharmacological activity or other direct and/or indirect effect in the diagnosis, cure, mitigation, treatment, or prevention of disease or to affect the structure or function of the body of humans or other animals. In this context and “active Ingredient” refers to any component of a drug product intended to furnish pharmacological activity or other direct and/or indirect effect in the diagnosis, cure, mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect. Specifically, the term API includes substances or mixtures of substances having an indirect pharmacological activity or other effect, by causing and/or activating another substance or mixture of substances to have a direct pharmacological activity or effect. Examples of such substances having indirect pharmacological activity or other effect include enzymes or enzyme co-factors promoting conversion of a substance or mixture of substances having less pharmacological activity into a substance or mixture of substances having more pharmacological activity. A non-limiting example of an API considered herein is BSH.

The term “BSH” as used herein refers to Bile Salt Hydrolase, which is an enzyme catalyzing the deconjugation of a conjugated bile acid into an unconjugated bile acid. As used herein, the term “deconjugation” may be used interchangeably with the “bioconversion” of conjugated into unconjugated bile acid(s). Examples of this reaction include deconjugating glycocholic acid (GCA) into cholic acid (CA); taurocholic acid (TCA) into cholic acid (CA); glycodeoxycholic acid (GDCA) into deoxycholic acid (DCA), taurodeoxycholic acid (TDCA) into deoxycholic acid (DCA), taurochenodeoxycholic acid (TCDCA) into chenodeoxycholic acid (CDCA), and glycochenodeoxycholic acid (GCDCA) into chenodeoxycholic acid (CDCA).

The terms “heterologous” or “recombinant” or “genetically modified” and their grammatical equivalents as used herein interchangeably refer to entities “derived from a different species or cell”. For example, a heterologous or recombinant polynucleotide gene is a gene in a host cell not naturally containing that gene, i.e. the gene is from a different species or cell type than the host cell. The terms as used herein about microbial host cells refers to microbial host cells comprising and expressing heterologous or recombinant polynucleotide genes.

The term “in vivo”, as used herein refers to within a living cell or organism, including, for example an animal, a plant or a microorganism.

The term “in vitro”, as used herein refers to outside a living cell or organism, including, without limitation, for example, in a microwell plate, a tube, a flask, a beaker, a tank, a reactor and the like.

The term “in situ”, as used herein refers to an event that takes place locally, for example inside a living organism or cell, where the biological context is intact, for example where the cause or a symptom of a disease presents itself.

The term “substrate” or “precursor”, as used herein refers to any compound that can be converted into a different compound. For example, a conjugated bile acid can be a substrate for BSH and can be converted into a deconjugated bile acid. For clarity, substrates and/or precursors include both compounds generated in situ by an enzymatic reaction in a cell or exogenously provided compounds, such as exogenously provided organic molecules that the host cell can metabolize into a desired compound.

Term “endogenous” or “native” as used herein refers to a gene or a polypepetide in a host cell which originates from the same host cell.

The term “deletion” as used herein refers to manipulation of a gene so that it is no longer expressed in a host cell.

The term “disruption” as used herein refers to manipulation of a gene or any of the machinery participating in the expression the gene, so that it is no longer expressed in a host cell.

The term “attenuation” as used herein refers to manipulation of a gene or any of the machinery participating in the expression the gene, so that it the expression of the gene is reduced as compared to expression without the manipulation.

The terms “substantially” or “approximately” or “about”, as used herein refers to a reasonable deviation around a value or parameter such that the value or parameter is not significantly changed. These terms of deviation from a value should be construed as including a deviation of the value where the deviation would not negate the meaning of the value deviated from. For example, in relation to a reference numerical value the terms of degree can include a range of values plus or minus 10% from that value. For example, deviation from a value can include a specified value plus or minus a certain percentage from that value, such as plus or minus 9%, 8%, 7%, 6%, 5%, 4%, 3%, 2%, or 1% from the specified value.

The term “and/or” as used herein is intended to represent an inclusive “or”. The wording X and/or Y is meant to mean both X or Y and X and Y. Further the wording X, Y and/or Z is intended to mean X, Y and Z alone or any combination of X, Y, and Z.

The term “isolated” as used herein about a compound, refers to any compound, which by means of human intervention, has been put in a form or environment that differs from the form or environment in which it is found in nature. Isolated compounds include but are not limited to compounds of the invention for which the ratio of the compounds relative to other constituents with which they are associated in nature is increased or decreased. In an important embodiment the amount of compound is increased relative to other constituents with which the compound is associated in nature. In an embodiment the compound of the invention may be isolated into a pure or substantially pure form. In this context a substantially pure compound means that the compound is separated from other extraneous or unwanted material present from the onset of producing the compound or generated in the manufacturing process. Such a substantially pure compound preparation contains less than 10%, such as less than 8%, such as less than 6%, such as less than 5%, such as less than 4%, such as less than 3%, such as less than 2%, such as less than 1%, such as less than 0.5% by weight of other extraneous or unwanted material usually associated with the compound when expressed natively or recombinantly. In an embodiment the isolated compound is at least 90% pure, such as at least 91% pure, such as at least 92% pure, such as at least 93% pure, such as at least 94% pure, such as at least 95% pure, such as at least 96% pure, such as at least 97% pure, such as at least 98% pure, such as at least 99% pure, such as at least 99.5% pure, such as 100% pure by weight.

The term “non-naturally occurring” as used herein about a substance, refers to any substance that is not normally found in nature or natural biological systems. In this context the term “found in nature or in natural biological systems” does not include the finding of a substance in nature resulting from releasing the substance to nature by deliberate or accidental human intervention. Non-naturally occurring substances may include substances completely or partially synthetized by human intervention and/or substances prepared by human modification of a natural substance.

The term “% identity” as used herein about amino acid sequences refers to the degree of identity in percent between two amino acid sequences obtained when using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

$$\frac{identical amino acid residues}{Length of alignment - total number of gaps in alignment} \times 100$$

The protein sequences of the present invention can further be used as a “query sequence” to perform a search against sequence databases, for example to identify other family members or related sequences. Such searches can be performed using the BLAST programs. Software for performing BLAST analyses is publicly available through the National Center for Biotechnology Information (http://www.ncbi.nlm.nih.gov). BLASTP is used for amino acid sequences and BLASTN for nucleotide sequences. The BLAST program uses as defaults:
Cost to open gap: default=5 for nucleotides/11 for proteins
Cost to extend gap: default=2 for nucleotides/1 for proteins
Penalty for nucleotide mismatch: default=-3
Reward for nucleotide match: default=1
Expect value: default=10
Wordsize: default=11 for nucleotides/28 for megablast/3 for proteins.

Furthermore, the degree of local identity between the amino acid sequence query or nucleic acid sequence query and the retrieved homologous sequences is determined by the BLAST program. However only those sequence segments are compared that give a match above a certain threshold. Accordingly, the program calculates the identity only for these matching segments. Therefore, the identity calculated in this way is referred to as local identity.

The term “mature polypeptide” or “mature enzyme” as used herein refers to a polypeptide in its final active form following translation and any post-translational modifications, such as N-terminal processing, endo- and exoproteolytic cleavage, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide.

The term “cDNA” refers to a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, 3′-end processing and polyadenylation, before appearing as mature spliced mRNA.

The term “coding sequence” refers to a nucleotide sequence, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequence” as used herein refers to a nucleotide sequence necessary for expression of a polynucleotide encoding a polypeptide. A control sequence may be native (i.e., from the same gene) or heterologous or foreign (i.e., from a different gene) to the polynucleotide encoding the polypeptide. Control sequences include, but are not limited to promoter sequences, intronic splicing elements, and transcription terminator (stop) sequences on the DNA level, and leader sequences/5′ untranslated regions (5′UTR) and poly(A) signal sequences on the mRNA level. To be operational, control sequences usually must include promoter sequences and terminator sequences. Control sequences may be provided with linkers for the purpose of introducing specific restriction sites facilitating ligation of the control sequences with the region of a polynucleotide encoding a polypeptide.

The term “signal peptide” as used herein refers to a polypeptide sequences that in some embodiments prompts a cell to translocate a protein to a location within or outside the cell. Examples are pre-peptides or pre-pro-peptides for protein secretion, and other signal peptides such as nuclear or mitochondrial location signals.

The term “pre-protein” refers to a protein that has a signal sequence. As used herein, the term “pro-protein” refers to an inactive polypeptide that may be converted by the host into an active protein by proteolysis of an inhibitory sequence. A pre-pro-protein has both sequences still present.

In order for a polypeptide to enter the secretory pathway in S. cerevisiae, N-terminal signal peptides such as pre-, pro-, or pre-pro sequences are required.

The term “expression” includes any step involved in the production of a polypeptide including, but not limited to, transcription, post-transcriptional modification, translation, post- translational modification, and secretion.

The term “expression vector” refers to a DNA molecule, double stranded, either linear or circular, which comprises a polynucleotide encoding a polypeptide and is operably linked to control sequences that provide for its expression. Expression vectors include expression cassettes for the integration of genes into a host cell as well as plasmids and/or chromosomes comprising such genes.

The term “microbial host cell” refers to any cell type that is susceptible to transformation, transfection, transduction, or the like with a nucleic acid construct or expression vector comprising a polynucleotide of the present invention. Microbial host cell encompasses any progeny of a parent cell that is not identical to the parent cell due to mutations that occur during replication.

The term “polynucleotide construct” refers to a polynucleotide, either single- or double stranded, which is isolated from a naturally occurring gene or is modified to contain segments of nucleic acids in a manner that would not otherwise exist in nature or which is synthetic, and which comprises a polynucleotide encoding a polypeptide and one or more control sequences.

The term “operably linked” refers to a configuration in which a control sequence is placed at an appropriate position relative to the coding polynucleotide such that the control sequence directs expression of the coding polynucleotide.

The terms “nucleotide sequence and “polynucleotide” are used herein interchangeably.

The term “comprise” and “include” as used throughout the specification and the accompanying claims as well as variations such as “comprises”, “comprising”, “includes” and “including” are to be interpreted inclusively. These words are intended to convey the possible inclusion of other elements or integers not specifically recited, where the context allows.

The articles “a” and “an” are used herein refers to one or to more than one (i.e. to one or at least one) of the grammatical object of the article. By way of example, “an element” may mean one element or more than one element.

Terms like “preferably”, “commonly”, “particularly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

The term “cell culture” as used herein refers to a culture medium comprising a plurality of host cells of the invention. A cell culture may comprise a single strain of host cells or may comprise two or more distinct host cell strains. The culture medium may be any medium that may comprise a recombinant host, e.g., a liquid medium (i.e., a culture broth) or a semi-solid medium, and may comprise additional components, e.g., a carbon source such as dextrose, sucrose, glycerol, or acetate; a nitrogen source such as ammonium sulfate, urea, or monosodium glutamate; a phosphate source; vitamins; trace elements; salts; amino acids; nucleobases; yeast extract; aminoglycoside antibiotics such as G418 and hygromycin B.

### Delivery Vehicles for In Situ API Delivery

As used herein, the term “vehicle” and “delivery vehicle” are used interchangeably a system capable of delivering an active pharmaceutical ingredient. The first aspect of the invention relates to a delivery vehicle comprising a system producing an active pharmaceutically ingredient (API) capable of preventing, treating and/or relieving one or more diseases in a mammal, wherein the vehicle is suitable for administering to the mammal and wherein the vehicle is capable of delivering the produced API in situ of the location in the mammal body in need of preventing, treating and/or relieving the disease.

The delivery vehicle of the invention can be any vehicle capable of hosting the system producing the API. The system can include polynucleotides encoding the API and means for expressing such polynucleotides into the API. Further to this embodiment, the vehicle can comprise one or more microbial host cells comprising the one or more polynucleotides encoding the API. Particularly, the vehicle can be one or more microbial host cell, which optionally have been subjected to a protective encapsulation as described below. In a preferred embodiment such microbial host cell is genetically modified and particularly the one or more polynucleotides encoding the API is heterologous to the genetically modified cell. In another embodiment, the delivery vehicle comprises an extract of one or more microbial host cell cultures, said one or more microbial host cells comprising polynucleotides encoding the API and means for expressing such polynucleotides into the API.

### API

The API of the invention can be an organic molecule particularly an organic molecule having a molecular weight of more than 200 g/mol, such as more than 500 g/mol, such as more than 1000 g/mol, such as more than 1500 g/mol; such as more than 2000 g/mol, such as more than 5000 g/mol. Particularly, the API can be a polypeptide, more particularly an enzyme. In one embodiment the API is capable of in situ converting a substance or mixture of substances which is inactive in the preventing, treating and/or relieving one or more diseases into a compound which is active in the preventing, treating and/or relieving the one or more diseases. In a preferred embodiment, the API is an enzyme that is secreted from a host cell. In one preferred embodiment, the API is a BSH enzyme that is secreted out of a host cell and capable of performing a bile acid deconjugation reaction in the gastrointestinal tract of a subject to be treated. In one embodiment, the API is BSH active in the prevention, treatment and/or relief of infection by a pathogenic strain of Clostridioides difficile.

Pathogenic strains of C. difficile produce cytotoxins that strongly correlate to the occurrence to the occurrence of pseudomembranous colitis (Meyers et al., 1981). The two major toxins, TcdA and TcdB (also called toxin A and toxin B), have been studied intensively since their reognition as major C. difficle virulence factors, and are the primary markers for diagnosis of CDI.

In one embodiment, the API is a Bile Salt Hydrolase (EC 3.5.1.24) (BSH) comprising a polypeptide selected from:
i) a polypeptide which is at least 50%, such as at least 60%, such as at least 70%, such as at least 80%, such as at least 90%, such as at least 95%, such as at least 96%, such as at least 97%, such as at least 98%, such as at least 99%, such as 100% identical to the mature BSH enzyme of anyone of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, or 111;
ii) a polypeptide encoded by a polynucleotide which is at least 50%, such as at least 60%, such as at least 70%, such as at least 80%, such as at least 90%, such as at least 95%, such as at least 96%, such as at least 97%, such as at least 98%, such as at least 99%, such as 100% identical to the polynucleotide comprised in anyone of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, or 112 encoding the mature polypeptide of anyone of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, or 111; or genomic DNA thereof; and
iii) a functional variant of the mature polypeptide of (i) or (ii) having BSH activity.

### Polynucleotides

In another embodiment the vehicle of the invention comprises one or more polynucleotides encoding the API, wherein the one or more polynucleotides are at least 50%, such as at least 60%, such as at least 70%, such as at least 80%, such as at least 90%, such as at least 95%, such as at least 96%, such as at least 97%, such as at least 98%, such as at least 99%, such as 100% identical to any one of the polynucleotides comprised in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110 or 112, or genomic DNA thereof encoding the mature polypeptide of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, or 111.

In SEQ ID NO 1 the mature enzyme comprises the amino acids in positions 22 to 356, whereas the amino acids in positions 1 to 21 comprise a pre-signal sequence. In SEQ ID NO: 3 the mature enzyme comprises the amino acids in positions 22 to 365, whereas the amino acids in positions 1 to 21 comprise a pre-signal sequence. In SEQ ID NO: 5 the mature enzyme comprises the amino acids in positions 22 to 344, whereas the amino acids in positions 1 to 21 comprise a pre-signal sequence. In SEQ ID NO: 7 the mature enzyme comprises the amino acids in positions 22 to 345, whereas the amino acids in positions 1 to 21 comprise a pre-signal sequence. In SEQ ID NO: 9 the mature enzyme comprises the amino acids in positions 28 to 351, whereas the amino acids in positions 1 to 27 comprise a pre-signal sequence. In SEQ ID NO: 11 the mature enzyme comprises the amino acids in positions 22 to 344, whereas the amino acids in positions 1 to 21 comprise a pre-signal sequence. In SEQ ID NO: 13 the mature enzyme comprises the amino acids in positions 22 to 344, whereas the amino acids in positions 1 to 21 comprise a pre-signal sequence. In SEQ ID NO: 15 the mature enzyme comprises the amino acids in positions 22 to 345, whereas the amino acids in positions 1 to 21 comprise a pre-signal sequence. In SEQ ID NO: 17 the mature enzyme comprises the amino acids in positions 22 to 338, whereas the amino acids in positions 1 to 21 comprise a pre-signal sequence. In SEQ ID NO: 19 the mature enzyme comprises the amino acids in positions 22 to 345, whereas the amino acids in positions 1 to 21 comprise a pre-signal sequence. In SEQ ID NO: 21 the mature enzyme comprises the amino acids in positions 22 to 345, whereas the amino acids in positions 1 to 21 comprise a pre-signal sequence. In SEQ ID NO: 23 the mature enzyme comprises the amino acids in positions 22 to 345, whereas the amino acids in positions 1 to 21 comprise a pre-signal sequence. In SEQ ID NO: 25 the mature enzyme comprises the amino acids in positions 22 to 257, whereas the amino acids in positions 1 to 21 comprise a pre-signal sequence. In SEQ ID NO: 27 the mature enzyme comprises the amino acids in positions 22 to 336, whereas the amino acids in positions 1 to 21 comprise a pre-signal sequence. In SEQ ID NO: 29 the mature enzyme comprises the amino acids in positions 22 to 358, whereas the amino acids in positions 1 to 21 comprise a pre- signal sequence. In SEQ ID NO: 31 the mature enzyme comprises the amino acids in positions 22 to 348, whereas the amino acids in positions 1 to 21 comprise a pre-signal sequence. In SEQ ID NO: 33 the mature enzyme comprises the amino acids in positions 22 to 337, whereas the amino acids in positions 1 to 21 comprise a pre-signal sequence. In SEQ ID NO: 35 the mature enzyme comprises the amino acids in positions 22 to 345, whereas the amino acids in positions 1 to 21 comprise a pre-signal sequence. In SEQ ID NO: 37 the mature enzyme comprises the amino acids in positions 22 to 358, whereas the amino acids in positions 1 to 21 comprise a pre-signal sequence. In SEQ ID NO: 39 the mature enzyme comprises the amino acids in positions 22 to 334, whereas the amino acids in positions 1 to 21 comprise a pre-signal sequence. In SEQ ID NO: 41 the mature enzyme comprises the amino acids in positions 22 to 337, whereas the amino acids in positions 1 to 21 comprise a pre-signal sequence. In SEQ ID NO: 43 the mature enzyme comprises the amino acids in positions 22 to 337, whereas the amino acids in positions 1 to 21 comprise a pre-signal sequence. In SEQ ID NO: 45 the mature enzyme comprises the amino acids in positions 22 to 337, whereas the amino acids in positions 1 to 21 comprise a pre-signal sequence. In SEQ ID NO: 47 the mature enzyme comprises the amino acids in positions 22 to 349, whereas the amino acids in positions 1 to 21 comprise a pre-signal sequence. In SEQ ID NO: 49 the mature enzyme comprises the amino acids in positions 22 to 346, whereas the amino acids in positions 1 to 21 comprise a pre-signal sequence. In SEQ ID NO: 51 the mature enzyme comprises the amino acids in positions 22 to 345, whereas the amino acids in positions 1 to 21 comprise a pre-signal sequence. In SEQ ID NO: 53 the mature enzyme comprises the amino acids in positions 22 to 336, whereas the amino acids in positions 1 to 21 comprise a pre-signal sequence. In SEQ ID NO: 55 the mature enzyme comprises the amino acids in positions 38 to 363, whereas the amino acids in positions 1 to 37 comprise a pre-signal sequence. In SEQ ID NO: 57 the mature enzyme comprises the amino acids in positions 22 to 355, whereas the amino acids in positions 1 to 21 comprise a pre-signal sequence. In SEQ ID NO: 59 the mature enzyme comprises the amino acids in positions 22 to 349, whereas the amino acids in positions 1 to 21 comprise a pre-signal sequence. In SEQ ID NO: 61 the mature enzyme comprises the amino acids in positions 22 to 336, whereas the amino acids in positions 1 to 21 comprise a pre-signal sequence. In SEQ ID NO: 63 the mature enzyme comprises the amino acids in positions 22 to 336, whereas the amino acids in positions 1 to 21 comprise a pre-signal sequence. In SEQ ID NO: 65 the mature enzyme comprises the amino acids in positions 26 to 351, whereas the amino acids in positions 1 to 25 comprise a pre-signal sequence. In SEQ ID NO: 67 the mature enzyme comprises the amino acids in positions 22 to 341, whereas the amino acids in positions 1 to 21 comprise a pre-signal sequence. In SEQ ID NO: 69 the mature enzyme comprises the amino acids in positions 22 to 344, whereas the amino acids in positions 1 to 21 comprise a pre-signal sequence. In SEQ ID NO: 71 the mature enzyme comprises the amino acids in positions 22 to 345, whereas the amino acids in positions 1 to comprise a pre-signal sequence. In SEQ ID NO: 73 the mature enzyme comprises the amino acids in positions 27 to 349, whereas the amino acids in positions 1 to 26 comprise a pre-signal sequence. In SEQ ID NO: 75 the mature enzyme comprises the amino acids in positions 22 to344, whereas the amino acids in positions 1 to 21 comprise a pre-signal sequence. In SEQ ID NO: 77 the mature enzyme comprises the amino acids in positions 25 to 346, whereas the amino acids in positions 1 to 24 comprise a pre-signal sequence. In SEQ ID NO: 79 the mature enzyme comprises the amino acids in positions 25 to 261, whereas the amino acids in positions 1 to 24 comprise a pre-signal sequence. In SEQ ID NO: 81 the mature enzyme comprises the amino acids in positions 22 to 349, whereas the amino acids in positions 1 to 21 comprise a pre-signal sequence. In SEQ ID NO: 83 the mature enzyme comprises the amino acids in positions 22 to 349, whereas the amino acids in positions 1 to 21 comprise a pre-signal sequence. In SEQ ID NO: 85 the mature enzyme comprises the amino acids in positions 22 to 345, whereas the amino acids in positions 1 to 21 comprise a pre-signal sequence. In SEQ ID NO: 87 the mature enzyme comprises the amino acids in positions 22 to 328, whereas the amino acids in positions 1 to 21 comprise a pre-signal sequence. In SEQ ID NO: 89 the mature enzyme comprises the amino acids in positions 22 to 367, whereas the amino acids in positions 1 to 21 comprise a pre-signal sequence. In SEQ ID NO: 91 the mature enzyme comprises the amino acids in positions 26 to 352, whereas the amino acids in positions 1 to 25 comprise a pre-signal sequence. In SEQ ID NO: 93 the mature enzyme comprises the amino acids in positions 25 to 360, whereas the amino acids in positions 1 to 24 comprise a pre-signal sequence. In SEQ ID NO: 95 the mature enzyme comprises the amino acids in positions 22 to 355, whereas the amino acids in positions 1 to 21 comprise a pre-signal sequence. In SEQ ID NO: 97 the mature enzyme comprises the amino acids in positions 19 to 357, whereas the amino acids in positions 1 to 18 comprise a pre-signal sequence. In SEQ ID NO: 99 the mature enzyme comprises the amino acids in positions 27 to 359, whereas the amino acids in positions 1 to 26 comprise a pre-signal sequence. In SEQ ID NO: 101 the mature enzyme comprises the amino acids in positions 26 to 363, whereas the amino acids in positions 1 to 25 comprise a pre-signal sequence. In SEQ ID NO: 103 the mature enzyme comprises the amino acids in positions 23 to 359, whereas the amino acids in positions 1 to 22 comprise a pre-signal sequence. In SEQ ID NO: 105 the mature enzyme comprises the amino acids in positions 22 to 345, whereas the amino acids in positions 1 to 21 comprise a pre-signal sequence. In SEQ ID NO: 107 the mature enzyme comprises the amino acids in positions 22 to 356, whereas the amino acids in positions 1 to 21 comprise a pre-signal sequence. In SEQ ID NO: 109 the mature enzyme comprises the amino acids in positions 22 to 366, whereas the amino acids in positions 1 to 21 comprise a pre-signal sequence. In SEQ ID NO: 111 the mature enzyme comprises the amino acids in positions 22 to 350, whereas the amino acids in positions 1 to 21 comprise a pre-signal sequence.

### Microbial Host Cells.

In the embodiment where the vehicle of the invention comprises microbial host cell, such host cell can be any suitable host cell such as a fungus or a bacterium. In particular, fungi may be selected from the phylas consisting of Ascomycota, Basidiomycota, Neocallimastigomycota, Glomeromycota, Blastocladiomycota, Chytridiomycota, Zygomycota, Oomycota and Microsporidia. More particularly fungus is a yeast selected from the genera consisting of Saccharomyces, Kluveromyces, Candida, Pichia, Debaromyces, Hansenula, Yarrowia, Zygosaccharomyces, and Schizosaccharomyces. Even more particularly the yeast may be selected from the species consisting of Kluyveromyces lactis, Saccharomyces boulardii, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica. In one special embodiment the yeast is a

Saccharomyces boulardii. In some embodiments, the Saccharomyces boulardii host cell may be a strain, variant or sub-type of the species Saccharomyces boulardii, referred to individually and collectively herein as an Sb strain. Preferably, the vehicle is a genetically modified yeast cell of the species S. boulardii secreting BSH, examples of which have been created for the first time and referred to herein as Sb^{BSH} strain(s).

In some embodiments, fungal and in particular yeast cells are particularly preferred where the disease to be treated or prevented is a bacterial infection resistant to treatment with broad spectrum antibiotics, and the API is affecting these pathogenic bacteria in an inhibitory fashion. Since antibiotics do not have any effect on fungi, for example S. boulardii will be able to colonize the colon during broad spectrum antibiotic treatment and thereby be effective by colonization competition and by production of the API in situ.

Among bacteria, bacterial host cells may be selected from the genera consisting of Lactobacillus, Leuconostoc, Streptomyces, Pediococcus, Lactococcus, Bifidobacterium, Weissella, Streptococcus, Komagataeibacter, Acetobacter, Escherichia, and Gluconacetobacter. Particularly, bacterial host cells may be selected from the species consisting of Escherichia coli, Lactobacillus acidophilus, Lactobacillus casei, Lactobacillus bulgaricus, Lactobacillus reuteri, Lactobacillus plantarum, Lactobacillus brevis, Lactobacillus paraplantarum, Lactobacillus coryniformis, Lactobacillus pentosus, Lactobacillus fermentum, Lactobacillus delbrueckii subsp. bulgaricus, Lactobacillus helveticus, Lactobacillus kefiranofaciens, Lactobacillus paracasei, Lactococcus lactis, Bifidobacterium bifidum, Leuconostoc mesenteroides, Leuconostoc citreum, Leuconostoc argentinum, Pediococcus pentosaceus, Weissella spp., Streptococcus thermophilus, Streptomycess spp., Gluconacetobacter xylinus, Acetobacter pasteurianus, Acetobacter aceti and Gluconobacter oxydans.

In another embodiment the microbial host cell of the invention further comprises at least one transporter molecule facilitating transport of the API and/ or any substrate or precursor required to produce the API in the vehicle. Further, the microbial host cell of the invention may also comprise one or more native or native codon-optimized genes which has been overexpressed, attenuated, disrupted and/or deleted, for example to enhance the production of the API and/or to stabilize the host cell. Still further, the microbial host cell of the invention may comprise at least 2 copies or more of a polynucleotide encoding the API, or encoding one or more polypeptides comprising the operative protein production and/or secretion pathway.

As disclosed herein the vehicle may comprise a microbial cell comprising the system producing the API and in particular, the vehicle is a microbial cell comprising the said system. Accordingly, in an important embodiment, the vehicle is a genetically modified yeast strain, particularly of the genus Saccharomyces, more particularly of the species S. boulardii comprising and expressing a heterologous gene encoding a BSH enzyme and thereby producing and secreting said BSH enzyme.

In some embodiments, the vehicle may comprise one or more recombinant microbial host cells capable of delivering one or more API to the location of a C. difficile infection or C. difficile-associated colitis. In further embodiments, the recombinant microbial host comprises more than one system for producing API and is capable of in situ production of a selection of APIs (such as a selection of BSH) considered to be most efficacious in the prevention or treatment of a certain condition (Jia et al., 2020—Metagenomic analysis of the human microbiome reveals the association between the abundance of gut bile salt hydrolases and host health).

### Formulations

It is well understood to those skilled in the art that for API's to be delivered for example to the gastrointestinal tract, it is desirable to protect such API's from degradation in storage or in the body due to gastric juices, thermal and pH stresses, osmotic shock, and oxidative stress. General drug delivery technologies for API's often include encapsulation in enteric polymer coatings or capsules which can be triggered to release upon specific pH or osmotic conditions, in the presence of enzymes, or through time-release technologies. See for example https://www.capsugel.com/biopharmaceutical-technologies/enteric-drug-delivery-technologies); S. Amidon et al;. AAPS PharmSciTech. 2015 August; 16(4): 731-741; J. Li, D. J. Mooney; Nat. Rev. Mater. 2016 Dec; 1(12): 16071; H. Wen et al.; AAPS J. 2015 November; 17(6): 1327-1340; and L. Liu et al.; “pH-Responsive carriers for oral drug delivery: challenges and opportunities of current platforms,” Drug Delivery Volume 24, 2017, Issue 1. Such enterically coated capsules and other gastro-resistant formulations are part of a well-established field of drug delivery technology. Moreover, delivery of live cells such as bacteria and fungi can be achieved through conventional pharmaceutical formulations or through formulation into functional foods. When delivering therapeutic probiotic microorganisms in food, one must also consider the effect of the water activity and other food ingredients on the formulation. Some exemplary review articles on delivery of microbial formulations include M. Govender et al.; AAPS PharmSciTech. 2014 February; 15(1): 29-43 and J. Kim et al.; Journal of Pharmaceutical Investigation Online ISSN 2093-6214. A common technique for microbial cells such as probiotics is encapsulation with for example gums, proteins, or alginate, and sometimes multi-layer coatings are used and can include materials such as starches, dextran sulfate and chitosan. Further, viability of Saccharomyces boulardii can be enhanced with encapsulation of layers of chitosan-dextran sulfate polyelectrolytes that are responsive to pH (Ben Thomas M et al.; J. Food Eng.; 2014; 136:1-8.

Accordingly, in one embodiment the vehicle of the invention is coated by a protective coating. In another embodiment the vehicle of the invention is encapsulated by a membrane, in a capsule, microcapsule, sphere and/or microsphere. The coating, membrane, capsule, microcapsule, sphere and/or microsphere may in some embodiments be enteric, which is optionally triggered to release the vehicle and/or the API by pH, by osmotic pressure, by enzymatic digestion and/or by time-release.

The coating, capsule, microcapsule, sphere and/or microsphere of the invention may comprise one or more materials selected from gums, proteins, waxes, polyols, alginates, starches, dextrans and chitosans. In a particular embodiment the coating, capsule, microcapsule, sphere and/or microsphere is insoluble in mammal gastro-intestinal juices. In another embodiment the coating, capsule, microcapsule, sphere and/or microsphere is permeable to the API. In a further embodiment where the vehicle comprises a microbail cell, the coating, capsule, microcapsule, sphere and/or microsphere is impermeable to the cell. Particularly, the coating, capsule, microcapsule, sphere and/or microsphere can be made of alginate-poly-L-lysine-alginate (APA). More particularly the coating, capsule, microcapsule, sphere and/or microsphere can be made of materials selected from alginate/poly-L-lysine/pectin/poly-L-lysine/alginate (APPPA), alginate/poly-L-lysine/pectin/poly-L-lysine/pectin (APPPP), and alginate/poly-L-lysine/chitosan/poly-L-lysine/alginate (APCPA).

### Diseases

The disease, which is prevented, treated and/or relieved, by the API produced from the system in the vehicle of the invention can be any disease towards which the API has a prophylactic, treatment and/or relieving pharmaceutical effect. Where the API is BSH, the disease can be infections by pathogenic microorganisms such as bacteria of the genus of Clostridioides. It has been shown that broth of the BSH secreting bacteria Bacteroides ovatus inhibits proliferation of vegetative cells of Clostridioides difficile only in the presence of bile salts (Yoon et al., 2017), so, in another embodiment the pathogenic microorganism is Clostridioides difficile and the disease C. difficile infection (CDI) and/or C. difficile-induced colitis (CDAD, C. difficile-associated disease) as disclosed for example in: John S. Fordtran; Proc (Bayl Univ Med Cent); 2006 January; 19(1): 3-12. As a non-limiting example, the infection to be treated may be by a pathogenic C. difficile strain with ribotype 027 as described by Tijerina-Rodriguez et al.; 2019, or any other pathogenic C. difficile strain with a different ribotype. The infection can be non-recurrent C. difficile infection (NR-CDI) or recurrent C. difficile infection (R-CDI).

Furthermore, it has previously been noted that high-fat diets increase bile acid levels in the gut. High-level expression of BSH in recombinant mice has been seen to result in a significant reduction of conjugated bile acids, plasma cholesterol, liver triglycerides, and host weight gain. BSH enzymes native to Bacteroidetes may ameliorate obesity (Jia et al., 2020). Indeed, the known correlation of more abundant gut bile salt hydrolases with improved specific aspecs of human health (summarized in Jia et al., 2020) is such that in some aspects diseases suitable for treatment by embodiments of the current invention comprising administration of a vehicle compring genetically modified microbial host cells expressing BSH include, but are not limited to, obesity, type 2 diabetes, cardiovascular disease, colon cancer, polycystic ovary syndrome, neurological diseases, diseases of the liver including nonalcoholic steatohepatitis, cirrhosis and/or liver cancer.

Accordingly, in some embodiments the API is BSH active in the prevention, treatment and/or relief of infection by a pathogenic strain of Clostridioides, such as C. difficile.

In another embodiment BSH as API may prevent, treat and/or relieve dysbiosis and/or diarrhea.

In a further embodiment, the API (such as BSH) may prevent, treat and/or relieve one or more diseases selected from a Clostridioides infection, Clostridioides infection induced colitis, obesity, type 2 diabetes, cardiovascular disease, colon cancer, polycystic ovary syndrome, a neurological disease, diseases of the liver including nonalcoholic steatohepatitis, cirrhosis and/or liver cancer.

In a special embodiment the vehicle of the invention is a genetically modified S. boulardii host cell secreting BSH, active in the treatment of one or more diseases, particularly a C. difficile gut infection in a mammal, wherein the host cell is suitable for administration to the mammal and wherein the host cell is capable of producing and delivering the produced API(s) in situ at the specific location within the mammal's body in need of preventing, treating and/or relieving the disease.

### Polynucleotide Constructs and Expression Vectors

In a separate aspect, the invention also provides a polynucleotide construct comprising a polynucleotide sequence of the invention encoding a polypeptide of the operative metabolic pathway, operably linked to one or more control sequences, which direct the expression of the pathway polypeptide in a microbial host cell harboring the polynucleotide construct. Conditions for the expression should be compatible with the control sequences. In some embodiments, one or more control sequences may be of a different source organism to the one or more polynucleotides whose expression they regulate. In some embodiments the one or more polynucleotide sequence encoding the pathway polypeptide, or the control sequence, or both, may be heterologous to the microbial host cell comprising the construct. In some embodiments, one or more polynucleotide sequences encoding a pathway polypeptide may be heterologous to the host organism, whilst one or more other polynucleotide sequences encoding other pathway polypeptides may be endogenous, homologous, or variants to polynucleotide sequences native to the host organism. In one embodiment the polynucleotide construct is comprised in an expression vector.

Polynucleotides may be manipulated in a variety of ways that allow expression of an API. Manipulation of the polynucleotide prior to its insertion into an expression vector may be desirable or necessary depending on the expression vector. The techniques for modifying polynucleotides utilizing recombinant DNA methods are well known in the art.

The control sequence may be a promoter, which is a polynucleotide that is recognized by the host cell for expression of a gene. The promoter contains transcriptional control sequences that mediate the expression of the gene. The promoter may be any polynucleotide that shows transcriptional activity in the host cell including mutant, truncated, synthetic, and hybrid promoters, and may be obtained from genes encoding extracellular or intracellular polypeptides either homologous or heterologous to the host cell. The promoter may also be an inducible promoter.

Examples of suitable promoters for directing transcription of the nucleic acid construct of the invention in fungal host cell are promoters obtained from the genes for Aspergillus nidulans acetamidase, Aspergillus niger neutral α-amylase, Aspergillus niger acid stable α-amylase, Aspergillus niger or Aspergillus awamori glucoamylase (glaA), Aspergillus nidulans gpdA, Aspergillus oryzae TAKA amylase, Aspergillus oryzae alkaline protease, Aspergillus oryzae triose phosphate isomerase, A. niger or A. awamori endoxylanase (xlnA) or β-xylosidase (xln D), Fusarium oxysporum trypsin-like protease (WO 96/00787), Fusarium venenatum amyloglucosidase (WO2000/56900), Fusarium venenatum Dania (WO 00/56900), Fusarium venenatum Quinn (WO 00/56900), Rhizomucor miehei lipase, Rhizomucor miehei aspartic proteinase, Trichoderma reesie β-glucosidase, Trichoderma reesie cellobiohydrolase I, Trichoderma reesie cellobiohydrolase II, Trichoderma reesie endoglucanase I, Trichoderma reesie endoglucanase II, Trichoderma reesie endoglucanase III, Trichoderma reesei endoglucanase IV, Trichoderma reesie endoglucanase V, Trichoderma reesie xylanase I, Trichoderma reesei xylanase II, Trichoderma reesie I3-xylosidase, as well as the NA2-tpi promoter and mutant, truncated, and hybrid promoters thereof. NA2-tpi promoter is a modified promoter from an Aspergillus neutral α-amylase gene in which the untranslated leader has been replaced by an untranslated leader from an Aspergillus triose phosphate isomerase gene. Examples of such promoters include modified promoters from an Aspergillus niger neutral α-amylase gene in which the untranslated leader has been replaced by an untranslated leader from an Aspergillus nidulans or Aspergillus oryzae triose phosphate isomerase gene. Other examples of promoters are the promoters described in WO2006/092396, WO2005/100573 and WO2008/098933, incorporated herein by reference.

Examples of suitable promoters for directing transcription of the nucleic acid construct of the invention in a yeast host include promoters obtained from the genes for Saccharomyces cerevisiae translational elongation factor EF-1 alpha (TEF1, TEF2), S. cerevisiae fructose 1,6-bisphosphate aldolase (FBA1), S. cerevisiae glyceraldehyde-3-phosphate dehydrogenases (TDH1, TDH2, TDH3), S. cerevisiae enolase (EN01), S. cerevisiae galactokinase (GAL1), S. cerevisiae alcohol dehydrogenase/glyceraldehyde-3-phosphate dehydrogenase (ADH1, or fusion promoter ADH2/GAP), S. cerevisiae triose phosphate isomerase (TPI1), S. cerevisiae metallothionein (CUP1), and S. cerevisiae 3-phosphoglycerate kinase (PGK1). Other useful promoters for yeast host cells are described by Romanos et al., 1992, Yeast 8: 423-488. Selecting a suitable promoter for expression in yeast is well known and is well understood by persons skilled in the art.

The control sequence may also be a transcription terminator, which is recognized by a host cell to terminate transcription. The terminator is operably linked to the 3′-terminus of the gene encoding the polypeptide. Any terminator that is functional in the host cell may be used.

Useful terminators for fungal host cells can be obtained from the genes encoding Aspergillus nidulans anthranilate synthase, Aspergillus niger glucoamylase, Aspergillus niger α-glucosidase, Aspergillus oryzae TAKA amylase, and Fusarium oxysporum trypsin-like protease; while useful terminators for yeast host cells can be obtained from the genes for S. cerevisiae enolase (ENO1), S. cerevisiae cytochrome C (CYC1), S. cerevisiae alcohol dehydrogenase (ADH1), and S. cerevisiae glyceraldehyde-3-phosphate dehydrogenase (TDH3). Other useful terminators for yeast host cells are described by Romanos et al., 1992, supra.

The control sequence may also be an mRNA stabilizer region downstream of a promoter and upstream of the coding sequence of a gene which increases expression of the gene.

The control sequence may also be a 5′ untranslated region (5′ UTR, or leader sequence), a non-translated region of an mRNA that is important for translation by the host cell. The leader sequence is operably linked to the 5′-terminus of the mRNA encoding the polypeptide. Any leader that is functional in the host cell may be used.

Useful leader sequences for fungal host cells can be obtained from the promoters of the genes for Aspergillus oryzae TAKA amylase and Aspergillus nidulans triose phosphate isomerase, while useful leaders for yeast host cells can be obtained from the promoters of genes for S. cerevisiae enolase (EN01), S. cerevisiae 3-phosphoglycerate kinase (PGK1), and S. cerevisiae alcohol dehydrogenase/glyceraldehyde-3-phosphate dehydrogenase (ADH2/GAPDH).

The control sequence may also be a poly(A) signal sequence, which is a sequence operably linked to the 3′-terminus of the polynucleotide and that, during transcription, is recognized by the host cell RNA polymerase as a signal to add polyadenosine residues to the mRNA. Any poly(A) signal sequence that is functional in the host cell may be used. Useful poly(A) signal sequences for fungal host cells can be obtained from the genes for Aspergillus nidulans anthranilate synthase, Aspergillus niger glucoamylase, Aspergillus niger α-glucosidase, Aspergillus oryzae TAKA amylase, and Fusarium oxysporum trypsin-like protease; while useful polyadenylation sequences for yeast host cells are described by Guo and Sherman, 1995, Mol. Cellular Biol. 15: 5983-5990.

In some embodiments, signal sequences may be present in the polynucleotides encoding polypetides so that the latter may undergo protein maturation and/or secretion. In order to enter the secretory pathway, polypeptides contain pre-, pro-, or pre-pro-peptide sequences that include a cleavage site that is recognized by signal peptidase in the ER lumen (reviewed in Barlowe et al., 2013).

After entry into the ER, a polypeptide folds with or without the help of chaperones, and may undergo additional modifications such as disulfide bond formation, glycosylation, and oligomerization. Subsequently, the protein is transported from the ER to the Golgi apparatus and then to the extracellular space. One type of secretion signal can be the “pre-peptide” that terminates in a signal peptidase cleavage site. In some cases, this pre-peptide is followed by a “pro-peptide”, which is removed in the late Golgi by the Kex2 endoprotease, as is the case in the MF(alpha)1 pre-pro-peptide (Brake et al., 1984).

In some embodiments, one or more polynucleotides encoding a polypeptide may comprise a suitable pre-, or pre-pro-sequence recognizable by the host cell. For example, in some embodiments, bacterial pre-peptides are recognized in eukaryotes, this can be analyzed using the algorithm SignalP-5.0 (http://www.cbs.dtu.dk/services/SignalP/). BSH polypeptides derived from Gram-negative bacteria carry pre-peptides that possibly can be recognized in S. boulardii, whereas BSH polypeptides derived from Gram-positive bacteria only carry a Methionine as start amino acid that is cleaved during protein processing in these bacteria, but has to be replaced with a pre-, or pre-pro-peptide that can be recognized in eukaryotes in order to enable protein secretion in S. boulardii.

It may also be desirable to add regulatory sequences that regulate expression of the polypeptide relative to the growth of the host cell. Examples of regulatory systems are those that cause expression of the gene to be turned on or off in response to a chemical or physical stimulus, including the presence of a regulatory compound. In fungi, the Aspergillus niger glucoamylase promoter, Aspergillus oryzae TAKA α-amylase promoter, and Aspergillus oryzae glucoamylase promoter may be used; while in yeast, the ADH2 system or GAL 1 system may be used.

Various nucleotide sequences in addition to the polynucleotide construct of the invention may be joined together to produce a recombinant expression vector, which may include one or more convenient restriction sites to allow for insertion or substitution of the polynucleotide sequence encoding the pathway polypeptide of the invention at such sites. The recombinant expression vector may be any vector (e.g., a plasmid or virus or chromosomal) that can be conveniently subjected to recombinant DNA procedures and can cause expression of the gene encoding the pathway polypeptide. The choice of the vector will typically depend on the compatibility of the vector with the host cell into which the vector is to be introduced. The vector may be an autonomously replicating vector, i.e., a vector that exists as an extrachromosomal entity, the replication of which is independent of chromosomal replication, e.g., a plasmid (linear or closed circular plasmid), an extrachromosomal element, a minichromosome, or an artificial chromosome. The vector may contain any means for assuring self-replication. Alternatively, the vector may, when introduced into the host cell, integrate into the host genome and replicate together with the chromosome(s) into which it has been integrated. Furthermore, a single vector or plasmid or two or more vectors or plasmids that together contain the total DNA to be introduced into the genome of the host cell, or a transposon, may be used.

The vector may contain one or more selectable markers that permit convenient selection of transformed, transfected, transduced, or the like cells. A selectable marker is a gene from which the product provides for biocide or viral resistance, resistance to heavy metals, prototrophy to auxotrophies, and the like. Useful selectable markers for fungal host cells include amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Useful selectable auxotrophic markers for yeast host cells include, but are not limited to, ADE2, HIS3, LEU2, LYS2, MET3, TRP1, and URA3. Useful drug resistance markers for yeast host cells include, but are not limited to, hph (hygromycin B phosphotransferase from Escherichia coli), ble (phleomycin resistance gene from Streptoalloteichus hindustanus), nat (nourseothricin N-acetyl transferase from Streptomyces noursei), and neo/kan (aminoglycoside 3′-phosphotransferase from transposon Tn903 giving resistance to G418). The vector may further contain element(s) that permits integration of the vector into the genome of the host cell or permits autonomous replication of the vector in the cell independently of the genome. For integration into the host cell genome, the vector may rely on the polynucleotide encoding the pathway polypeptide or any other element of the vector for integration into the genome by homologous or non-homologous recombination. When yeast is the host, integration into the host genome is preferred by homologous recombination. Alternatively, in some embodiments, one or more additional “helper” vectors may be introduced into the host which contain additional polynucleotides encoding gene products for directing integration by homologous recombination into the genome of the host cell at precise location(s) in the chromosome(s). To increase the likelihood of integration at a precise location, the integrational elements should contain a sufficient number of nucleic acids, such as approximately 45 to approximately 2,000 base pairs, such as approximately 500 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides.

For autonomous replication, the vector may further comprise an origin of replication enabling the vector to replicate autonomously in the host cell in question. The origin of replication may be any sequence mediating autonomous replication that functions in a cell. The term “origin of replication” or “plasmid replicator” refers to a DNA sequence that enables a plasmid or vector to replicate in vivo. Useful origins of replication for fungal cells include AMA1 and ANS1 (Gems et al., 1991, Gene 98: 61-67; Cullen et al., 1987, Nucleic Acids Res. 15: 9163-9175; WO 00/24883). Isolation of the AMA1 sequence and construction of a plasmid comprising AMA1 can be accomplished using the methods disclosed in WO2000/24883. Useful origins of replication for yeast host cells are the 2-micron origin of replication, ARS1, ARS4, the combination of ARS1 and CEN3, and the combination of ARS4 and CEN6.

As mentioned, supra, more than one copy of a polynucleotide encoding the pathway polypeptide of the invention may be inserted into a host cell to increase production of the API. An increase in the copy number can be obtained by integrating one or more additional copies of the enzyme coding sequence into the host cell genome or by including a less efficient selectable marker gene on the plasmid, so that only cells containing multiple copies of the selectable marker gene—and thereby additional copies of the polynucleotide—can be selected by cultivating the cells in the presence of the appropriate selectable agent or media lacking the specific amino acid for auxotrophic growth. The procedures used to ligate the elements described above to construct the recombinant expression vectors of the present invention are well known to one skilled in the art (see, e.g., Sambrook et al., 1989; Mikkelsen et al., 2012; supra).

In alignment with the above the vehicles of this disclose also include those comprising a microbial host cell comprising the polynucleotide construct as described, supra.

### Cultures

The invention also provides a cell culture, comprising a host cell of the invention and a growth medium. Suitable growth mediums for host cells such as fungi and/or yeasts are known in the art.

### Methods of Producing Vehicles or Cultures of the Invention.

The invention also provides a method for producing the cell culture of the invention comprising
a) culturing the microbial host cell of the invention at conditions allowing growth of the microbial host cell; and
b) optionally recovering and/or isolating the cell culture.

The cell culture can be cultivated in a nutrient medium suitable for production of the compound of the invention and/or propagating cell count using methods known in the art. For example, the culture may be cultivated by shake flask cultivation, or small-scale or large-scale fermentation (including continuous, batch, fed-batch, or solid-state fermentations) in laboratory or industrial fermenters in a suitable medium and under conditions allowing the host cells to grow and/or propagate, optionally to be recovered and/or isolated.

The cultivation can take place in a suitable nutrient medium comprising carbon and nitrogen sources and inorganic salts, using procedures known in the art. Suitable media are available from commercial suppliers or may be prepared according to published recipes (e.g. from catalogues of the American Type Culture Collection). The selection of the appropriate medium may be based on the choice of host cell and/or based on the regulatory requirements for the host cell. Such media are available in the art. The medium may, if desired, contain additional components favoring the transformed expression hosts over other potentially contaminating microorganisms. Accordingly, in an embodiment a suitable nutrient medium comprise a carbon source (e.g. glucose, maltose, molasses, starch, cellulose, xylan, pectin, lignocellolytic biomass hydrolysate, etc.), a nitrogen source (e. g. ammonium sulfate, ammonium nitrate, ammonium chloride, mono sodium glutamate etc.), an organic nitrogen source (e.g. yeast extract, malt extract, peptone, etc.) and inorganic nutrient sources (e.g. phosphate, magnesium, potassium, zinc, iron, etc.).

The cultivating of the host cell may be performed over a period of from about 0.5 to about 30 days. The cultivation process may be a batch process, continuous or fed-batch process, suitably performed at a temperature in the range of 0-100° C. or 0-80° C., for example, from about 0° C. to about 50° C. and/or at a pH, for example, from about 2 to about 10. Preferred fermentation conditions for yeasts and filamentous fungi are a temperature in the range of from about 25° C. to about 55° C. and at a pH of from about 2 to about 9. The appropriate conditions are usually selected based on the choice of host cell. Accordingly, in an embodiment the method of the invention further comprises one or more elements selected from:
a) culturing the cell culture in a nutrient medium;
b) culturing the cell culture under aerobic or anaerobic conditions
c) culturing the cell culture under agitation;
d) culturing the cell culture at a temperature of between 25 to 50° C.;
e) culturing the cell culture at a pH of between 3-9; and
f) culturing the cell culture for between 10 hours to 30 days.

The cell culture of the invention may be recovered and or isolated using methods known in the art. For example, the compound(s) may be recovered from the nutrient medium by conventional procedures including, but not limited to, centrifugation, filtration, spray-drying, or lyophilization.

### Fermentation Composition

The invention further provides a fermentation composition comprising the cell culture of the invention. In one embodiment the fermentation composition comprises an API produced by the culture and one or more compounds selected from trace metals, vitamins, salts, yeast nitrogen base (YNB), carbon source, and/or amino acids of the fermentation; wherein the concentration of the API is at least 1 mg/l composition.

Preferably, the API concentration in the fermentation composition is at least 100 μg/L, 500 μg/L, 1 mg/L, 5 mg/L, such as at least 10 mg/L, such as at least 20 mg/l, such as at least 50 mg/L, such as at least 100 mg/L, such as at least 500 mg/L, such as at least 1000 mg/L, such as at least 5000 mg/L, such as at least 10000 mg/L, such as at least 50000 mg/L. Further, the fermentation composition of the invention may have a cell density of at least 10⁷ CFU/ml, such as at least 10⁸ CFU/ml, such as at least 10⁹ CFU/ml, such as at least 10¹⁰ CFU/ml, such as at least 10¹¹ CFU/ml, such as at least 10¹⁷ CFU/ml.

Compositions and Use

In a further aspect the invention provides a composition comprising the vehicle, cell culture and/or fermentation composition of the invention and one or more carriers, agents, additives and/or excipients. Carriers, agents, additives and/or excipients includes formulation additives, stabilising agent, fillers and the like. The composition may be formulated into a dry solid form by using methods known in the art, such as spray drying, spray cooling, lyophilization, flash freezing, granulation, microgranulation, encapsulation or microencapsulation. The composition may also be formulated into liquid stabilized form using methods known in the art, such as formulation into a stabilized liquid comprising one or more stabilizers such as sugars and/or polyols (e.g. sugar alcohols) and/or organic acids (e.g. lactic acid).

In particular, the composition is a pharmaceutical composition comprising the vehicle, cell culture and/or fermentation composition of the invention and one or more pharmaceutical grade excipient, additives and/or adjuvants. The pharmaceutical composition can be in form of a powder, tablet or capsule, or it can be liquid in the form of a pharmaceutical solution, suspension, lotion or ointment.

The invention further provides a method for preparing a pharmaceutical composition comprising mixing the vehicle, cell culture and/or fermentation composition of the invention with one or more pharmaceutical grade excipient, additives and/or adjuvants. The pharmaceutical composition is suitably used as a medicament in a method for treating a disease, in particular for preventing, treating and/or relieving CDI and/or CDI induced colitis. In such use the prevention, treatment and/or relief is in one embodiment inhibiting proliferation of a pathogenic strain of the genus Clostridioides, particularly by inhibition of Clostridioides spore germination as well as vegetative growth, the API is BSH and the treatment comprises contacting the pathogenic strain in the presence of a conjugated bile acids with the pharmaceutical composition at conditions allowing the vehicle, cell culture and/or fermentation composition to produce and deliver BSH in amounts converting the conjugated bile acids into therapeutically effective amounts of deconjugated bile acids inhibiting germination or proliferation of the pathogenic strain.

In a further embodiment the pharmaceutical preparation and the treatment is performed in situ of the pathogenic strain in and/or on the body of a mammal. The use and/or method of treatment of the invention can comprise administering the pharmaceutical composition of the invention in an amount for the vehicle, the cell culture and/or the fermentation composition of the invention to produce and deliver a therapeutically effective amount of the API, in situ, of the cause of the disease. The use and/or method of treatment suitably includes administering the vehicle and or cell culture in an amount of at least 3 mg lyophilized cells, such as at least 10 mg, such as at least 25 mg such as at least 50 mg, such as at least 100 mg, such as at least 500 mg, per kg body mass per day to the mammal to be treated. Additionally or alternatively the treatment suitably includes administering the vehicle and or cell culture in the form of 1 or more dosages of an amount of at least 10⁶ CFU, such as at least 10⁷ CFU, such as at least 10⁸ CFU, such as at least 10⁹ CFU, such as at least 10¹⁰ CFU, such as at least 10¹¹ CFU, such as at least 10¹² CFU per dose to the mammal to be treated. The dosage regimen may have a frequency of doses of at least once, such as at least twice, such as least three times, such as at least four times, such as at least five times, such as at least six times daily. The pharmaceutical preparation can be administered orally (preferably), or parenterally, such as topically, epicutaneously, sublingually, buccally, nasally, intradermally, intralesionally, (intra)ocularly, intramuscular, intrapulmonary and/or intravaginally. The pharmaceutical composition can also be administered enterally to the gastrointestinal tract.

In one embodiment, a composition suitable for oral administration comprises approximately 3-5×10⁹CFU/capsule in a plant cellulose capsule. In another embodiment, a composition suitable for oral administration comprises approximately 500 mg of lypholized host cells per capsule, said capsule comprising a body of gelatin and titanium dioxide, encapsulated by a coating comprising gelatin, titanium dioxide, red iron oxide and indigotin.

In some embodiments, compositions suitable for oral administration comprising a cell culture, isolated recombinant host cells and/or fermentation composition of the invention may be administered after or simultaneously with one or more antibiotics. Non-limiting examples of suitable antibiotic treatments include vancomycin orally 4 times a day or fidaxomicin twice daily, both for a total of 10 days. In circumstances when antibiotic treatment has contributed to disease onset (such as when treatment with broad spectrum antibiotics depletes the gut biome of the patient and is followed by C. difficile bloom), or when antibiotic treatment is administered as part of the therapy for the disease or for treatment or co-treatment of other conditions, the recombinant host cells and/or fermentation composition of the invention are preferably yeast cells, more preferably Saccharomyces cells, most preferably Saccharomyces boulardii cells. In further embodiments, a suitable course of combined antibiotic and recombinant host cell treatment may be followed by the administration of one or more prebiotic polysaccharide(s) optionally in combination with supplementary doses of a cell culture, isolated recombinant host cells and/or fermentation composition of the invention. Examples of suitable prebiotics include but are not limited to fructooligosaccharides (FOS), inulins, galactooligosaccharides (GOS), resistant starch, pectin, beta-glucans and/or xylooligosaccharides. Such treatment regimes are beneficial in post-antibiotic re-colonisation and sustained maintenance of a population of recombinant host cells of the current invention in the gut.

### Sequence Listings

The present application contains a Sequence Listing prepared in Patentln included below but also submitted electronically in ST25 format which is hereby incorporated by reference in its entirety.

| | | | | |
|---|---|---|---|---|
| SEQ ID NO: 1 | protein sequence of | Bo_bsh_co | From | Bacteroides ovatus |

MTKNLLLGIAAVCGSTFQAVACTGISLTSRDGSYVQARTIEWARGVLQSEYVIIPRGQQLTSFTPTGVNGLTFTAKYGVVGLAVVQKEF
IAEGINEAGLSAGLFFFPHYGGYKTYDAAQNQRTLADLQVTEWLLSQFSTIDEVKAALSSVRVVGLEKTAVVHWRIGEPSGRQVVLEIV
GGVPHFYENEVGVLTNAPGFEWQLTNLNNYVNLHPGDASVQKLSGITLQPTGGNSGFLGIPGDATPPSRFVRAAFYRGTAPQRATGFDT
VQQCFHLLNNFDIPIGIEHSQGDIPDIPSATQWTSAIDLTNRKVYYKTAYNNSIRCIDLKAIDFSKVKYQSQPLDKIQEQPVEMVKI
PR*

| | | | | |
|---|---|---|---|---|
| SEQ ID NO: 2 | DNA coding sequence of | Bo_bsh_co | From | Bacteroides ovatus |

ATGACCAAGAACTTGTTGTTGGGTATTGCTGCTGTTTGTGGTTCTACTTTTCAAGCTGTTGCTTGTACCGGTATTTCTTTGACTTCTAG
AGATGGTTCTTACGTTCAAGCCAGAACTATTGAATGGGCTAGAGGTGTTTTACAGTCCGAATACGTTATTATCCCAAGAGGTCAACAAT
TGACTTCTTTCACTCCAACTGGTGTTAACGGTTTGACTTTTACTGCTAAGTATGGTGTTGTTGGTTTGGCCGTTGTTCAGAAAGAATTC
ATTGCTGAAGGTATTAACGAAGCTGGTTTGTCTGCTGGTTTATTCTTTTTTCCACATTACGGTGGTTACAAGACTTACGATGCTGCTCA
AAATCAAAGAACTTTGGCCGACTTACAAGTTACCGAATGGTTGTTGTCTCAATTCTCCACTATCGATGAAGTTAAGGCTGCTTTGTCAT
CCGTTAGAGTTGTAGGTTTGGAAAAGACTGCTGTTGTTCATTGGAGAATAGGTGAACCATCTGGTAGACAAGTTGTTTTGGAAATAGTT
GGTGGTGTTCCACACTTCTACGAAAATGAAGTTGGTGTTTTGACTAACGCTCCAGGTTTTGAATGGCAATTGACTAACTTGAACAACTA
CGTTAACTTGCATCCAGGTGATGCCTCTGTTCAAAAATTGTCTGGTATTACCTTGCAACCTACTGGTGGTAATTCTGGTTTTTTGGGTA
TTCCTGGTGATGCTACTCCACCATCTAGATTTGTTAGAGCTGCTTTTTACAGAGGTACTGCTCCACAAAGAGCTACTGGTTTTGATACT
GTTCAACAATGCTTCCACCTGTTGAACAATTTCGATATTCCAATCGGTATCGAACACTCCCAAGGTGATATTCCAGATATTCCTTCTGC
TACTCAATGGACCTCTGCTATTGATTTGACCAACAGAAAGGTTTACTACAAGACCGCTTACAACAACTCCATTAGATGCATCGATTTGA
AGGCCATCGATTTCTCTAAGGTCAAGTATCAATCTCAGCCATTGGACAAGATTCAAGAACAACCAGTTGAAATGGTCAAGATCCCAAGA
TAA

| | | | | |
|---|---|---|---|---|
| SEQ ID NO: 3 | protein sequence of | Bo_bsh_co_6xHis | From | Bacteroides ovatus |

MTKNLLLGIAAVCGSTFQAVACTGISLTSRDGSYVQARTIEWARGVLQSEYVIIPRGQQLTSFTPTGVNGLTFTAKYGVVGLAVVQKEF
IAEGINEAGLSAGLFFFPHYGGYKTYDAAQNQRTLADLQVTEWLLSQFSTIDEVKAALSSVRVVGLEKTAVVHWRIGEPSGRQVVLEIV
GGVPHFYENEVGVLTNAPGFEWQLTNLNNYVNLHPGDASVQKLSGITLQPTGGNSGFLGIPGDATPPSRFVRAAFYRGTAPQRATGFDT
VQQCFHLLNNFDIPIGIEHSQGDIPDIPSATQWTSAIDLTNRKVYYKTAYNNSIRCIDLKAIDFSKVKYQSQPLDKIQEQPVEMVKIPR
CATHHHHHH*

| | | | | |
|---|---|---|---|---|
| SEQ ID NO: 4 | DNA coding sequence of | Bo_bsh_co_6xHis | from | Bacteroides ovatus |

ATGACCAAGAACTTGTTGTTGGGTATTGCTGCTGTTTGTGGTTCTACTTTTCAAGCTGTTGCTTGTACCGGTATTTCTTTGACTTCTAG
AGATGGTTCTTACGTTCAAGCCAGAACTATTGAATGGGCTAGAGGTGTTTTACAGTCCGAATACGTTATTATCCCAAGAGGTCAACAAT
TGACTTCTTTCACTCCAACTGGTGTTAACGGTTTGACTTTTACTGCTAAGTATGGTGTTGTTGGTTTGGCCGTTGTTCAGAAAGAATTC
ATTGCTGAAGGTATTAACGAAGCTGGTTTGTCTGCTGGTTTATTCTTTTTTCCACATTACGGTGGTTACAAGACTTACGATGCTGCTCA
AAATCAAAGAACTTTGGCCGACTTACAAGTTACCGAATGGTTGTTGTCTCAATTCTCCACTATCGATGAAGTTAAGGCTGCTTTGTCAT
CCGTTAGAGTTGTAGGTTTGGAAAAGACTGCTGTTGTTCATTGGAGAATAGGTGAACCATCTGGTAGACAAGTTGTTTTGGAAATAGTT
GGTGGTGTTCCACACTTCTACGAAAATGAAGTTGGTGTTTTGACTAACGCTCCAGGTTTTGAATGGCAATTGACTAACTTGAACAACTA
CGTTAACTTGCATCCAGGTGATGCCTCTGTTCAAAAATTGTCTGGTATTACCTTGCAACCTACTGGTGGTAATTCTGGTTTTTTGGGTA
TTCCTGGTGATGCTACTCCACCATCTAGATTTGTTAGAGCTGCTTTTTACAGAGGTACTGCTCCACAAAGAGCTACTGGTTTTGATACT
GTTCAACAATGCTTCCACCTGTTGAACAATTTCGATATTCCAATCGGTATCGAACACTCCCAAGGTGATATTCCAGATATTCCTTCTGC
TACTCAATGGACCTCTGCTATTGATTTGACCAACAGAAAGGTTTACTACAAGACCGCTTACAACAACTCCATTAGATGCATCGATTTGA
AGGCCATCGATTTCTCTAAGGTCAAGTATCAATCTCAGCCATTGGACAAGATTCAAGAACAACCAGTTGAAATGGTCAAGATTCCAAGA
TGTGCTACTCATCATCACCACCATCATTAA

| | | | | |
|---|---|---|---|---|
| SEQ ID NO: 5 | protein sequence of | Ef_bsh1_co_SP-Bo | from | Enterococcus |
| | | | | faecium |

MTKNLLLGIAAVCGSTFQAVACTSITYVTSDHYFGRNFDYEISYNEVVTITPRNYKLNFRKVNDLDTHYAMIGIAAGIADYPLYYDATN
EKGLSMAGLNFSGYADYKEIQEGKDNVSPFEFIPWILGQCSTVGEAKKLLKNINLANINYSDELPLSPLHWLLADKEKSIVIESMKDGL
HIYDNPVGVLTNNPSFDYQLFNLNNYRVLSSETPKNNFSNQISLNAYSRGMGGIGLPGDLSSVSRFVKATFTKLNSVSGDSESESISQF
FHILGSVEQQKGLCDVGDGKYEYTIYSSCCNVDKGIYYYRTYEDSQITAIDMNKEDLDSHKLISYPIIEKQQIKYIN*

| | | | | |
|---|---|---|---|---|
| SEQ ID NO: 6 | DNA coding sequence of | Ef_bsh1_co_SP-Bo | from | Enterococcus |
| | | | | faecium |

ATGACCAAGAACTTGTTGTTGGGTATTGCTGCTGTTTGTGGTTCTACTTTTCAAGCTGTTGCTTGTACCTCTATTACCTACGTTACTTC
CGATCATTACTTCGGTAGAAACTTCGACTACGAGATCTCTTACAACGAAGTTGTTACTATCACCCCTAGGAACTACAAGTTGAACTTCA
GAAAGGTTAACGATTTGGATACCCATTACGCCATGATTGGTATTGCAGCTGGTATAGCTGATTATCCCTTGTATTATGATGCCACCAAC
GAAAAGGGTTTGTCTATGGCTGGTTTGAATTTTTCTGGTTACGCCGACTACAAAGAAATCCAAGAAGGTAAGGATAACGTCAGCCCATT
TGAATTCATTCCATGGATTTTGGGTCAATGCTCTACTGTTGGTGAAGCTAAAAAGCTGTTGAAGAACATTAACCTGGCCAACATCAACT
ACTCTGATGAATTGCCATTGTCTCCATTGCATTGGTTGTTGGCTGACAAAGAAAAGTCCATCGTTATCGAATCTATGAAGGATGGCTTG
CACATCTACGATAATCCAGTTGGTGTTTTGACTAACAACCCATCTTTTGACTACCAGCTGTTCAACCTGAACAACTACAGAGTTTTGTC
ATCTGAAACCCCAAAGAACAACTTCTCCAATCAGATCTCTTTGAACGCTTACTCTAGAGGTATGGGTGGTATTGGTTTGCCAGGTGATT
TGTCATCAGTTTCCAGATTTGTTAAGGCTACCTTCACCAAGTTGAATTCCGTTTCTGGTGATTCCGAATCCGAATCCATTTCTCAATTC
TTCCATATCTTGGGTTCCGTCGAACAACAAAAAGGTTTGTGTGATGTTGGTGACGGTAAATACGAGTACACTATCTACTCCTCTTGTTG
CAATGTTGACAAGGGTATCTACTACTACAGAACCTACGAAGATTCTCAAATTACCGCCATCGACATGAACAAAGAAGATTTGGATTCTC
ACAAGCTGATCTCGTACCCAATTATTGAAAAGCAGCAGATCAAGTACATCAACTGA

| | | | | |
|---|---|---|---|---|
| SEQ ID NO: 7 | protein sequence of | La_bshA_co_SP-Bo | from | Lactobacillus |
| | | | | acidophilus |
| | | | | strain LA11 |

MTKNLLLGIAAVCGSTFQAVACTSIIFSPKDHYFGRNLDLEITFGQQVVITPRNYTFKFRKMPSLKKHYAMIGISLDMDDYPLYFDATN
EKGLGMAGLNYPGNATYYEEKENKDNIASFEFIPWILGQCSTISEVKDLLSRINIADLNFSEKMQASSLHWLIADKTGTSLVVETDKDG
MHIYDNPVGCLTNNPQFPKQLFNLNNYADVSPKMPKNNFSDKVNMAGYSRGLGSHNLPGGMDSESRFVRVAFNKFNAPIAETEEENIDT
YFHILHSVEQQKGLDEVGPNSFEYTIYSDGTNLDKGIFYYTTYSNKQINVVDMNKEDLDSSNLITYDMLDKTKFNHQN*

| | | | | |
|---|---|---|---|---|
| SEQ ID NO: 8 | DNA coding sequence of | La_bshA_co_SP-Bo | from | Lactobacillus |
| | | | | acidophilus |
| | | | | strain LA11 |

ATGACCAAGAACTTGTTGTTGGGTATTGCTGCTGTTTGTGGTTCTACTTTTCAAGCTGTTGCTTGCACCTCCATTATTTTCTCACCAAA
GGATCATTACTTCGGCAGAAACTTGGATTTGGAAATTACCTTCGGTCAACAAGTTGTTATCACCCCAAGAAACTACACCTTCAAGTTTA
GAAAGATGCCCTCCTTGAAGAAACATTACGCCATGATTGGTATCTCATTGGATATGGATGACTACCCCTTGTACTTTGATGCTACAAAC
GAAAAAGGTTTAGGTATGGCTGGTTTGAATTACCCAGGTAATGCTACTTACTACGAAGAGAAAGAAAACAAGGACAATATCGCCTCCTT
CGAATTCATTCCATGGATTTTGGGTCAATGCTCCACTATCTCTGAAGTTAAGGATTTGTTGTCCAGGATCAACATTGCCGATTTGAACT
TCTCTGAAAAGATGCAAGCTTCCTCATTGCATTGGTTGATTGCTGATAAGACTGGTACTTCCTTGGTTGTTGAAACTGATAAGGATGGT
ATGCACATCTACGATAATCCAGTTGGTTGTTTGACTAACAACCCACAATTTCCTAAGCAGCTGTTCAACTTAAACAACTACGCTGATGT
TTCTCCCAAGATGCCAAAGAACAATTTCTCCGATAAGGTTAATATGGCCGGTTACTCTAGAGGTTTGGGTTCTCATAATTTGCCAGGTG
GTATGGATTCTGAATCTAGGTTTGTTAGAGTTGCCTTTAACAAGTTCAACGCTCCAATTGCTGAAACCGAAGAAGAAAACATTGATACC
TACTTCCACATCTTGCACTCCGTCGAACAACAAAAGGGTTTAGATGAAGTTGGTCCAAACTCTTTCGAGTACACTATCTATTCTGACGG
TACAAATTTGGACAAGGGTATCTTCTACTACACCACCTATTCTAACAAGCAAATCAACGTTGTCGACATGAACAAAGAGGATTTGGACT
CCTCTAACTTGATCACCTATGATATGTTGGACAAGACGAAGTTCAACCACCAAAACTGA

| | | | | |
|---|---|---|---|---|
| SEQ ID NO: 9 | protein sequence of | Bf_bsh_co | from | Bacteroides |
| | | | | fragilis |

MRRKIMMATLIVIAVNFIWSGQSVKACTRAVYIGPDNMVITGRTMDWKEDIQSNLYLFPRGIKRAGYNKGNTVEWISKYGSIVATGYDI
GTCDGMNEKGLVASLLFLPESIYVRRNDTRPVMGISIWTQYVLDNFATVSEAVEELKKQTFRIDAPDMPNGSASTLHMAITDETGNSAV
LEYIDGNLIIHEGKEYQVMTNSPRYDLQLAVNDYWKEVGGLNMLPGTNRSSDRFVRASFYIHAIPQTSDAKIAVPSVLSVMRNVSVPFG
ITTPDKPYISSTRWRSVSDQKNRVYYFESTLTPNLFWIDLHKVDFSPNASIKKLSLTHGEVYAGDVVKDFKDSRSFTFMFELPQ*

| | | | | |
|---|---|---|---|---|
| SEQ ID NO: 10 | DNA coding sequence of | Bf_bsh_co | from | Bacteroides |
| | | | | fragilis |

ATGAGGCGTAAGATTATGATGGCTACCTTGATCGTTATTGCCGTTAACTTTATTTGGTCCGGTCAATCTGTTAAGGCTTGTACTAGAGC
TGTTTACATTGGTCCAGATAACATGGTTATTACTGGTAGAACCATGGATTGGAAAGAGGACATTCAATCCAACTTGTACTTGTTCCCAA
GAGGTATTAAGAGAGCTGGTTACAACAAGGGTAATACCGTTGAATGGATTTCCAAGTACGGTTCTATAGTTGCTACCGGTTATGATATC
GGTACTTGTGATGGTATGAACGAGAAAGGTTTGGTTGCTTCTTTGTTGTTCTTGCCAGAATCCATCTACGTTAGAAGAAATGATACCAG
ACCAGTCATGGGTATTTCTATTTGGACTCAATACGTCTTGGATAACTTCGCTACTGTTTCTGAAGCTGTCGAAGAATTGAAGAAGCAAA
CCTTCAGAATTGATGCTCCAGATATGCCAAATGGTTCTGCTTCTACATTGCATATGGCTATTACTGACGAAACTGGTAACTCTGCTGTT
TTGGAGTACATTGATGGTAACTTGATCATCCACGAGGGTAAAGAATACCAAGTTATGACTAACTCTCCAAGGTACGACTTGCAATTGGC
TGTTAATGATTACTGGAAAGAAGTTGGCGGTTTGAATATGTTGCCAGGTACTAATAGATCCTCCGATAGATTTGTTAGAGCCTCCTTTT
ACATTCACGCTATTCCACAAACATCCGATGCTAAAATTGCTGTTCCATCCGTTTTGTCCGTTATGAGAAATGTTTCTGTTCCATTCGGT
ATTACTACCCCAGATAAGCCATATATCTCTTCTACTAGATGGCGTTCTGTCTCCGATCAAAAGAATAGAGTTTACTACTTCGAGTCCAC
TTTGACCCCAAATTTGTTTTGGATTGACTTGCACAAGGTTGACTTTTCTCCAAACGCTTCCATCAAAAAGTTGTCTTTGACTCACGGTG
AAGTTTACGCTGGTGATGTTGTTAAGGATTTCAAGGACTCTAGAAGCTTCACCTTCATGTTTGAATTGCCACAGTGA

| | | | | |
|---|---|---|---|---|
| SEQ ID NO: 11 | protein sequence of | Ed_bsh_co_SP-Bo | from | Enterococcus |
| | | | | durans |

MTKNLLLGIAAVCGSTFQAVACTSITYVTSDHYFGRNFDYEISYNEVVTVTPRNYKLNFRKVNDLDTHYAMIGIAAGIADYPLYYDATN
EKGLSMAGLNFSGYADYKEIQEGKDNVSPFEFIPWILGQCSTVGEAKKLLKNINLANINYSDELPLSPLHWLLADKEKSIVIESMKDGL
HIYDNPVGVLTNNPSFDYQLFNLNNYRVLSSETPKNNFSNQISLNAYSRGMGGIGLPGDLSSVSRFVKATFTKLNSVSGDSESESISQF
FHILGSVEQQKGLCDVGDGKYEYTIYSSCCNVDKGIYYYRTYEDSQITAIDMNKEDLDSHKLISYPIIEKQQIKYIN*

| | | | | |
|---|---|---|---|---|
| SEQ ID NO: 12 | DNA coding sequence of | Ed_bsh_co_SP-Bo | from | Enterococcus |
| | | | | durans |

ATGACCAAGAACTTGTTGTTGGGTATTGCTGCTGTTTGTGGTTCTACTTTTCAAGCTGTTGCTTGTACCTCTATTACCTACGTTACTTC
CGATCATTACTTCGGTAGAAACTTCGACTACGAGATCTCTTACAACGAAGTTGTTACTGTTACCCCTAGGAACTACAAGTTGAACTTCA
GAAAGGTTAACGATTTGGATACCCATTACGCCATGATTGGTATTGCAGCTGGTATAGCTGATTATCCCTTGTATTATGATGCCACCAAC
GAAAAGGGTTTGTCTATGGCTGGTTTGAATTTTTCTGGTTACGCCGACTACAAAGAAATCCAAGAAGGTAAGGATAACGTCAGCCCATT
TGAATTCATTCCATGGATTTTGGGTCAATGCTCTACTGTTGGTGAAGCTAAAAAGCTGTTGAAGAACATTAACCrGGCCAACATCAACT
ACTCTGATGAATTGCCATTGTCTCCATTGCATTGGTTGTTGGCTGACAAAGAAAAGTCCATCGTTATCGAATCTATGAAGGATGGCTTG
CACATCTACGATAATCCAGTTGGTGTTTTGACTAACAACCCATCTTTTGACTACCAGCTGTTCAACCTGAACAACTACAGAGTTTTGTC
ATCTGAAACCCCAAAGAACAACTTCTCCAATCAGATCTCTTTGAACGCTTACTCTAGAGGTATGGGTGGTATTGGTTTGCCAGGTGATT
TGTCATCAGTTTCCAGATTTGTTAAGGCTACCTTCACCAAGTTGAATTCCGTTTCTGGTGATTCCGAATCCGAATCCATTTCTCAATTC
TTCCATATCTTGGGTTCCGTCGAACAACAAAAAGGTTTGTGTGATGTTGGTGACGGTAAATACGAGTACACTATCTACTCCTCTTGTTG
CAATGTTGACAAGGGTATCTACTACTACAGAACCTACGAAGATTCTCAAATTACCGCCATCGACATGAACAAAGAAGATTTGGATTCTC
ACAAGCTGATCTCGTACCCAATTATTGAAAAGCAGCAGATCAAGTACATCAACTGA

| | | | | |
|---|---|---|---|---|
| SEQ ID NO: 13 | protein sequence of | Ed_bsh_co_SP-Bo | from | Enterococcus |
| | | | | faecalis |

MTKNLLLGIAAVCGSTFQAVACTAITYVSKDHYFGRNFDYEISYNEVVTITPRNYKFSFREVGNLDHHFAIIGIAAGIADYPLYYDAIN
EKGLGMAGLNFSGYADYKKIEEGKENVSPFEFIPWVLGQCSTVDEAKKLLKNLNLVNINFSDELPLSPLHWLLADKEQSIVVESTKEGL
RVFDNPVGVLTNNPTFDYQLFNLNNYRVLSTRTPKNNFSDQIELDIYSRGMGGIGLPGDLSSVSRFVKATFTKLNSVSRSSEYESISQF
FHILSSVEQQKGLCDVGDEKYEYTIYSSCCNLEKGIYYYRTYDNSQITAVDMNKENLEKDSLIVYPMVETQQINYAN*

| | | | | |
|---|---|---|---|---|
| SEQ ID NO: 14 | DNA coding sequence of | Ed_bsh_co_SP-Bo | from | Enterococcus |
| | | | | faecalis |

ATGACCAAGAACTTGTTGTTGGGTATTGCTGCTGTTTGTGGTTCTACTTTTCAAGCTGTTGCTTGTACTGCTATCACCTACGTTTCTAA
GGATCATTACTTCGGTAGAAACTTCGACTACGAGATCTCTTACAACGAAGTTGTTACTATCACCCCTAGGAACTACAAGTTCTCATTCA
GAGAAGTTGGTAACTTGGATCATCATTTCGCCATTATTGGTATTGCAGCTGGTATAGCTGATTACCCCTTGTATTATGATGCCATCAAC
GAAAAAGGTTTAGGTATGGCTGGTTTGAACTTTTCTGGTTACGCTGACTACAAGAAGATCGAAGAAGGTAAAGAAAACGTCAGCCCATT
CGAATTCATTCCATGGGTTTTGGGTCAATGTTCTACTGTTGATGAAGCCAAGAAGCTGTTGAAGAACTTGAACTTGGTTAACATCAACT
TCTCCGACGAATTGCCATTGTCTCCATTGCATTGGTTGTTGGCTGACAAAGAACAATCCATCGTTGTCGAATCTACCAAAGAAGGTTTG
AGAGTTTTCGATAACCCAGTTGGTGTTTTGACTAACAACCCAACTTTTGACTACCAGCTGTTCAACCTGAACAACTACAGAGTTTTGTC
TACTAGAACCCCTAAGAACAACTTCTCAGACCAAATCGAATTGGACATCTACTCTAGAGGTATGGGTGGTATTGGTTTGCCAGGTGATT
TGTCATCTGTTTCCAGATTTGTTAAGGCTACCTTCACCAAGTTGAACTCCGTTTCTAGATCTTCCGAATACGAATCCATCTCTCAATTC
TTCCACATCTTGTCCTCTGTCGAACAACAAAAGGGTTTGTGTGATGTTGGTGACGAAAAGTACGAGTACACTATCTACTCCTCTTGTTG
CAATTTGGAGAAGGGTATCTACTACTACAGAACCTACGATAACTCCCAAATTACCGCTGTTGACATGAACAAAGAAAACCTGGAAAAGG
ACTCCTTGATCGTTTATCCAATGGTTGAAACCCAACAAATCAACTACGCTAACTAA

| | | | | |
|---|---|---|---|---|
| SEQ ID NO: 15 | protein sequence of | La_bshB_co_SP-Bo | from | Lactobacillus |
| | | | | acidophilus |
| | | | | strain LA11 |

MTKNLLLGIAAVCGSTFQAVACTSICYNPNDHYFGRNLDYEIAYGQKVVIVPRNYEFKYREMPSQKMHYAFIGVSVVNDDYPLLCDAIN
EKGLGIAGLNFQGPNHYFPKIEGKKNIASFELMPYLLSNCENTDDVKEILDNANILNISFSANYPAADLHWILSDKAGKSIAVESTNSG
LHIYDNPVNVLTNNPEFPDQLIKLSDYADVTPHNPKNTLVPNVDLNLYSRGLGTHHLPGGMDSSSRFVKVAFVLAHTPQGKNEVENVTN
YFHILHSVEQPDGLDEVEDNRYEYTMYTDCMNLDKGILYFTTYDNNRINAVDMHKADLGSEDLICYDLFKKQDIEYMN*

| | | | | |
|---|---|---|---|---|
| SEQ ID NO: 16 | DNA coding sequence of | La_bshB_co_SP-Bo | from | Lactobacillus |
| | | | | acidophilus |
| | | | | strain LA11 |

ATGACCAAGAACTTGTTGTTGGGTATTGCTGCTGTTTGTGGTTCTACTTTTCAAGCTGTTGCTTGTACCTCTATTTGCTACAATCCAAA
CGATCACTACTTCGGTAGAAACTTGGATTACGAAATTGCCTACGGTCAAAAGGTTGTTATCGTTCCAAGAAACTACGAGTTCAAGTACA
GAGAAATGCCCTCTCAAAAAATGCATTACGCCTTCATTGGTGTTTCCGTTGTTAATGATGATTACCCTTTGTTGTGTGACGCCATCAAC
GAAAAAGGTTTAGGTATTGCCGGTTTGAATTTCCAAGGTCCAAATCATTACTTCCCAAAGATCGAAGGCAAGAAGAACATTGCTTCTTT
CGAATTGATGCCCTACTTGTTGTCTAACTGTGAAAACACCGATGACGTCAAAGAAATTTTGGATAACGCCAACATCCTGAACATTTCTT
TCTCTGCTAATTATCCAGCTGCTGACTTGCATTGGATTTTGTCTGATAAGGCTGGTAAGTCCATTGCTGTTGAATCTACTAATTCTGGC
TTGCACATCTACGATAACCCAGTTAATGTTCTGACTAACAACCCAGAATTCCCAGACCAATTGATTAAGTTGTCTGATTACGCTGATGT
TACCCCACATAATCCAAAGAATACTTTGGTTCCAAACGTCGACTTGAACTTGTATTCTAGAGGTTTGGGTACACATCATTTGCCAGGTG
GTATGGATTCTTCATCTAGATTTGTTAAGGTCGCTTTCGTTTTGGCTCATACTCCACAAGGTAAGAACGAAGTTGAAAACGTTACCAAC
TACTTCCACATCTTGCACTCTGTTGAACAACCAGATGGTTTGGATGAAGTCGAAGATAATAGATACGAGTACACTATGTACACCGACTG
TATGAATTTGGACAAGGGTATCTTGTACTTCACTACCTATGACAACAACAGAATCAACGCTGTTGATATGCATAAGGCTGATTTGGGTT
CCGAAGATTTGATTTGCTACGACTTGTTCAAGAAGCAAGACATCGAGTACATGAACTAA

| | | | | |
|---|---|---|---|---|
| SEQ ID NO: 17 | protein sequence of | Lb_bsh_co_SP-Bo | from | Lactobacillus |
| | | | | brevis |

MTKNLLLGIAAVCGSTFQAVACTSLTYTSATGHHFFARTMDFPTTTPWRPVFLPRHYHWETALHVTRTTQYAILGGGRIAADFSAYLLA
DGFNESGISCAELYLPGVVDYAPAPVAGQLNLTPQDFINWALGEHATLAAIAADLPHVTLVDQPWGADRYVYPFHWILSDRSGQTLVIE
PTGGPLTAQLNPAGVLTNTPILATHLRNLNDFLNVDAPDFSPATVAAARSYLVSGQPLPSGPVPTHRFIHTAIRRWGNPPATTTTAAVT
HLFQWLAEVQLPYQPKRRHDRNHNYTHYRGICDLDTKSYLFIPRTTGRLQQITLTPEMAACRHFPHAFPAN*

| | | | | |
|---|---|---|---|---|
| SEQ ID NO: 18 | DNA coding sequence of | Lb_bsh_co_SP-Bo | from | Lactobacillus |
| | | | | brevis |

ATGACCAAGAACTTGTTGTTGGGTATTGCTGCTGTTTGTGGTTCTACTTTTCAAGCTGTTGCTTGTACTTCTTTGACTTACACTTCTGC
TACTGGTCATCATTTCTTCGCTAGAACTATGGATTTCCCAACTACTACTCCTTGGAGGCCAGTTTTTTTGCCAAGACATTATCATTGGG
AAACCGCCTTGCATGTTACTAGAACTACTCAATACGCTATCTTAGGTGGTGGTAGAATTGCTGCAGATTTTTCTGCTTATTTGTTGGCC
GATGGTTTCAACGAATCTGGTATTTCTTGTGCTGAGTTGTATTTGCCAGGTGTTGTTGATTATGCTCCAGCTCCAGTTGCTGGTCAATT
GAATTTGACTCCACAAGATTTCATTAACTGGGCCTTGGGTGAACATGCTACTTTGGCTGCTATTGCAGCTGATTTGCCACATGTTACTT
TAGTTGACCAACCATGGGGTGCTGATAGATATGTTTATCCATTCCATTGGATCCTGTCCGATAGATCTGGTCAAACTTTGGTTATTGAA
CCTACTGGTGGTCCATTGACTGCACAATTGAATCCAGCTGGTGTTTTGACTAACACTCCAATTTTGGCTACCCACTTGAGAAACTTGAA
CGATTTCTTGAATGTTGATGCCCCAGATTTCTCTCCAGCTACTGTTGCAGCTGCTAGATCTTATTTGGTTTCTGGTCAACCATTGCCAT
CTGGTCCAGTTCCAACTCATAGATTCATTCATACCGCTATTAGAAGATGGGGTAATCCACCAGCTACTACTACAACAGCTGCTGTTACT
CATTTGTTTCAATGGTTGGCTGAAGTCCAATTGCCATATCAACCTAAAAGAAGGCATGACAGAAACCATAACTACACCCATTACAGAGG
TATTTGCGATTTGGATACCAAGTCCTACTTGTTCATTCCAAGAACTACTGGTAGATTGCAACAGATTACTTTGACACCAGAAATGGCTG
CTTGTAGACATTTTCCACATGCTTTTCCAGCTAACTAA

| | | | | |
|---|---|---|---|---|
| SEQ ID NO: 19 | protein sequence of | Lf_bsh_co_SP-Bo | from | Lactobacillus |
| | | | | fermentum |

MTKNLLLGIAAVCGSTFQAVACTSINVIAQDGYHVLGRTMDWDDLLVSPIFTPRHYQLASVFDHRVHENPYAIIGGGSITERRTDVSDG
VNEFGLMAQKLTFKNGARLVDERHPDKVQLAAFELIFYLLGHFKSVADVAAHLDQIELMNDVNADVPFGYSEQHFVLSDPTGRCVVIEP
SEHPLKLIDNPLGIMTNMPKFDHQLERLQDYLDFTPDFLNGTLAPNTFHVTTGKLSGKKTPPGAYTPKGRYVRAAYMKELADQPASKDE
ALATTWHLLDSVTVPKSKAHRPTFSVYRAATVAEDRTYYFQSYHQAQVTSVKLTDDLLKRATPLVFDTADVWAPVKLN*

| | | | | |
|---|---|---|---|---|
| SEQ ID NO: 20 | DNA coding sequence of | Lf_bsh_co_SP-Bo | from | Lactobacillus |
| | | | | fermentum |

ATGACCAAGAACTTGTTGTTGGGTATTGCTGCTGTTTGTGGTTCTACTTTTCAAGCTGTTGCTTGCACCTCCATTAACGTTATTGCTCA
AGATGGTTACCACGTTTTGGGTAGAACTATGGATTGGGATGATTTGTTGGTTTCCCCAATTTTCACTCCAAGACATTACCAATTGGCCT
CTGTTTTCGATCATAGAGTTCACGAAAATCCATACGCCATTATTGGTGGTGGTTCTATTACTGAAAGAAGGACTGACGTTTCTGATGGT
GTTAATGAATTTGGTTTGATGGCCCAAAAGCTGACTTTTAAGAATGGTGCTAGATTGGTTGACGAAAGACATCCAGATAAGGTTCAATT
GGCTGCTTTCGAATTGATCTTCTATTTGTTGGGTCACTTCAAGTCCGTTGCTGATGTTGCTGCTCATTTGGATCAAATTGAATTGATGA
ACGATGTCAACGCCGATGTTCCATTTGGTTATTCTGAACAACACTTCGTTTTGTCTGATCCAACTGGTAGATGCGTTGTTATTGAACCA
TCTGAACATCCCTTGAAGTTGATCGATAATCCATTGGGTATTATGACCAACATGCCAAAGTTCGATCACCAATTGGAAAGATTGCAAGA
CTACTTGGATTTCACCCCAGATTTTTTGAATGGTACTTTGGCTCCAAACACCTTCCATGTTACTACTGGTAAATTGTCCGGTAAAAAAA
CTCCACCAGGTGCTTATACTCCAAAGGGTAGATATGTTAGAGCTGCCTATATGAAGGAATTGGCTGATCAACCAGCTTCTAAAGATGAA
GCTTTGGCTACTACATGGCACTTGTTGGATTCTGTTACTGTTCCAAAATCCAAGGCTCATAGACCAACTTTCTCTGTTTACAGAGCTGC
TACTGTTGCTGAAGATAGAACTTACTACTTCCAATCCTACCATCAAGCTCAAGTTACCTCTGTTAAGTTGACTGATGACTTGTTGAAAA
GAGCTACCCCATTGGTTTTTGATACAGCTGATGTTTGGGCTCCAGTCAAGTTGAACTAA

| | | | | |
|---|---|---|---|---|
| SEQ ID NO: 21 | protein sequence of | Lg_bsh_co_SP-Bo | from | Lactobacillus |
| | | | | gallinarum |

MTKNLLLGIAAVCGSTFQAVACTSIIFSPRDHYFGRNLDLEVTFGQQVVITPRDYVFKFRDMPKIDHHYAMVGIALNADSYPLYFDAAN
EKGLGMVGLNYPDNAIYYDVKEGKDNIASFEFIPWILGQAATVAEAKKLLSTINITKKNFSDKMQASPLHWIIADKTGVSIVVETDADG
MHVYDNPVGCLTNNPQFPKQLFNLNNYADVSAAMPKNNFSDQVKMDGYSRGLGSRNLPGGMDSESRFVRVAFNKFNAPTSDSEEENVDT
YFHILHSVEQQKGLDQVGPDAFEYTIYSDGINLDKGIFYYTTYTNKQINVVDMNKEDLDSSDLITYDMLTKGKFNHQN*

| | | | | |
|---|---|---|---|---|
| SEQ ID NO: 22 | DNA coding sequence of | Lg_bsh_co_SP-Bo | from | Lactobacillus |
| | | | | gallinarum |

ATGACCAAGAACTTGTTGTTGGGTATTGCTGCTGTTTGTGGTTCTACTTTTCAAGCTGTTGCTTGCACCTCCATTATTTTCTCACCAAG
AGATCATTACTTCGGCAGAAACTTGGATTTGGAAGTTACTTTCGGTCAACAAGTTGTTATCACCCCAAGAGATTACGTGTTCAAGTTTA
GAGATATGCCAAAGATCGATCATCACTACGCTATGGTTGGTATTGCTTTGAATGCTGATTCTTACCCCTTGTACTTTGATGCTGCTAAC
GAAAAAGGTTTAGGTATGGTCGGTTTGAACTACCCAGATAATGCTATCTACTACGACGTCAAAGAAGGCAAGGATAACATTGCTTCCTT
CGAATTCATTCCATGGATTTTGGGTCAAGCTGCTACTGTTGCTGAAGCTAAAAAGTTGTTGTCCACCATTAACATCACCAAGAAGAACT
TCTCCGATAAGATGCAAGCTTCTCCATTGCATTGGATTATTGCTGATAAGACCGGTGTTTCCATCGTTGTTGAAACTGATGCTGATGGT
ATGCACGTTTACGATAATCCAGTTGGTTGTTTGACTAACAACCCACAATTTCCAAAGCAGCTGTTCAACTTAAACAACTACGCTGATGT
TTCTGCTGCCATGCCAAAAAACAATTTCTCCGATCAAGTTAAGATGGACGGTTACTCTAGAGGTTTGGGTTCTAGAAATTTGCCAGGTG
GTATGGATTCTGAATCTAGGTTTGTTAGAGTTGCCTTTAACAAGTTCAACGCTCCAACTTCTGATTCCGAAGAAGAAAACGTTGATACC
TACTTCCATATCTTGCACTCCGTCGAACAACAAAAGGGTTTAGATCAAGTTGGTCCAGATGCTTTTGAGTACACTATCTATTCCGATGG
TATCAACTTGGACAAGGGTATTTTCTACTACACTACCTACACCAACAAGCAAATCAACGTTGTCGACATGAACAAAGAGGATTTGGATT
CCTCTGATTTGATCACCTACGATATGTTGACCAAGGGTAAATTCAACCACCAGAACTGA

| | | | | |
|---|---|---|---|---|
| SEQ ID NO: 23 | protein sequence of | LgAM1_bsh_co_SP- | from | Lactobacillus |
| | | Bo | | gasseri Am1 |

MTKNLLLGIAAVCGSTFQAVACTSILYSPKDHYFGRNLDYEIAYGQKVVITPRNYEFEFTDLPVEKSHYAMIGVAAVADNTPLYCDAIN
EKGLGVAGLSFAGQGKYFPNAVNKKNIASFEFISYLLATYETVDQVKESLTNANISNVSFAKNTPASELHWLVGDKTGKSIVVESDEKG
LHVYNNPVNALTNAPLFPEQLTNLVNFASVVPGEPDNNFLPGVNLKLYSRSLGTHHLPGGMDSESRFVKVCFALNHAPKDSDEVENVTN
FFHILESVEQAKGMDQVGPNSFEYTMYTSCMNLEKGILYFNCYDDSRISAVDMNKEDLDSSDLVVYDLFKKQDISFIN*

| | | | | |
|---|---|---|---|---|
| SEQ ID NO: 24 | DNA coding sequence of | LgAM1_bsh_co_SP- | from | Lactobacillus |
| | | Bo | | gasseri Am1 |

ATGACCAAGAACTTGTTGTTGGGTATTGCTGCTGTTTGTGGTTCTACTTTTCAAGCTGTTGCTTGTACCTCTATCTTGTACTCTCCAAA
GGATCATTACTTCGGTAGAAACTTGGATTACGAAATTGCCTACGGTCAAAAGGTTGTTATTACCCCAAGAAACTACGAGTTCGAATTCA
CTGATTTGCCAGTTGAAAAGTCCCATTACGCTATGATTGGTGTTGCTGCAGTTGCTGATAATACCCCATTATATTGTGATGCCATCAAC
GAGAAAGGTTTGGGTGTTGCAGGTTTGTCTTTTGCTGGTCAAGGTAAATACTTTCCAAACGCCGTTAACAAGAAGAATATTGCCTCCTT
CGAGTTCATCTCTTACTTGTTGGCTACTTACGAAACCGTTGACCAAGTCAAAGAATCCTTGACTAACGCCAACATCTCTAACGTTTCTT
TCGCTAAGAATACCCCAGCTTCTGAATTGCATTGGTTGGTTGGTGATAAGACTGGTAAGTCTATCGTTGTTGAATCTGACGAAAAGGGC
TTGCATGTTTACAACAATCCAGTTAACGCTTTGACCAATGCTCCTTTGTTTCCAGAACAATTGACCAACTTGGTTAACTTCGCTTCTGT
TGTTCCAGGTGAACCAGATAACAATTTTTTGCCAGGTGTCAACCTGAAGTTGTACTCTAGATCTTTGGGTACTCATCATTTGCCTGGTG
GTATGGATTCTGAATCAAGATTCGTTAAGGTTTGCTTCGCTTTGAATCATGCCCCAAAAGATTCTGATGAAGTTGAAAACGTTACCAAC
TTCTTCCACATCTTGGAATCTGTTGAACAAGCCAAAGGTATGGATCAAGTAGGTCCAAATTCTTTCGAGTACACTATGTACACCTCTTG
CATGAATTTGGAGAAAGGCATCTTGTATTTCAACTGCTACGATGACTCTAGAATCTCCGCTGTTGATATGAACAAAGAGGATTTGGACT
CCTCCGATTTGGTTGTTTACGATTTGTTCAAGAAGCAGGACATCTCCTTCATCAACTGA

| | | | | |
|---|---|---|---|---|
| SEQ ID NO: 25 | protein sequence of | Lh_bsh_co_SP-Bo | from | Lactobacillus |
| | | | | helveticus |

MTKNLLLGIAAVCGSTFQAVACSSIIFSPKDHYFGRNLDLEITFGQQVIITPRDYVFKFRDMPEIDHHYAMVGIALNAGGYPLYFDAAN
EKGLGMGGLNYPDNAIYYDVKEGKDNIASFEFIPWILSQAATVEEAKKLLAKINITKKNFSDKMHVSPLHWIIADKTGASIVVETDADG
MHVYDNPVGCLTNNPQFPKQLFNLNNYQDVSPAMPKNNFSSKINMDGYSRGLGSRNLPGGMDAESRFVRVAFNKRAWIK*

| | | | | |
|---|---|---|---|---|
| SEQ ID NO: 26 | DNA coding sequence of | Lh_bsh_co_SP-Bo | from | Lactobacillus |
| | | | | helveticus |

ATGACCAAGAACTTGTTGTTGGGTATTGCTGCTGTTTGTGGTTCTACTTTTCAAGCTGTTGCTTGCTCCTCCATTATTTTCTCACCAAA
GGATCATTACTTCGGCAGAAACTTGGATTTGGAAATTACCTTTGGTCAGCAAGTTATCATCACCCCAAGAGATTACGTTTTCAAGTTCA
GAGATATGCCAGAAATCGATCATCATTACGCCATGGTTGGTATTGCTTTGAATGCTGGTGGTTACCCATTATACTTTGATGCTGCTAAC
GAAAAAGGTCTAGGTATGGGTGGTTTGAATTACCCAGATAATGCTATCTACTACGACGTCAAAGAAGGCAAGGATAACATTGCTTCCTT
CGAATTCATTCCCTGGATTTTGTCTCAAGCTGCTACTGTTGAAGAAGCCAAAAAGTTGTTGGCCAAGATTAACATCACCAAGAAGAACT
TCTCCGACAAGATGCATGTTTCTCCATTGCATTGGATTATTGCTGATAAGACTGGTGCCTCTATCGTTGTTGAAACTGATGCTGATGGT
ATGCACGTTTACGATAATCCAGTTGGTTGTTTGACTAACAACCCACAATTTCCTAAGCAGCTGTTCAACCTGAACAACTACCAAGATGT
TTCACCAGCTATGCCAAAGAACAACTTCAGCTCTAAGATCAACATGGACGGTTATTCTAGAGGTTTGGGTTCTAGAAATTTGCCAGGTG
GTATGGATGCTGAATCCAGATTTGTTAGAGTTGCCTTTAACAAGAGAGCCTGGATCAAGTAA

| | | | | |
|---|---|---|---|---|
| SEQ ID NO: 27 | protein sequence of | Lj100_cbshbeta_co_SP- | from | Lactobacillus |
| | | Bo | | johnsonii 100-100 |

MTKNLLLGIAAVCGSTFQAVACTGLRFTDDQGNLYFGRNLDVGQDYGEGVIITPRNYPLPYKFLDNTTTKKAVIGMGIVVDGYPSYFDC
YNEDGLGIAGLNFPHFAKFSDGPIDGKINLASYEIMLWVTQNFTHVSEVKEALKNVNLVNEAINTSFAVAPLHWIISDSDEAIIVEVSK
QYGMKVFDDKVGVLTNSPDFNWHLTNLGNYTGLNPHDATAQSWNGQKVAPWGVGTGSLGLPGDSIPADRFVKAAYLNANYPTVKGEKAN
VAKFFNILKSVAMIKGSVVNDQGSDEYTVYTACYSSGSKTYYCNFEDDFELKTYKLDDHTMNSTSLVTY*

| | | | | |
|---|---|---|---|---|
| SEQ ID NO: 28 | DNA coding sequence of | Lj100_cbshbeta_co_SP- | from | Lactobacillus |
| | | Bo | | johnsonii 100-100 |

ATGACCAAGAACTTGTTGTTGGGTATTGCTGCTGTTTGTGGTTCTACTTTTCAAGCTGTTGCTTGTACTGGTTTGAGATTCACTGATGA
TCAAGGCAACTTGTACTTCGGTAGAAATTTGGATGTTGGTCAAGATTATGGTGAGGGTGTTATTATCACTCCAAGAAATTATCCCTTGC
CATACAAGTTTTTGGATAACACTACTACCAAAAAGGCCGTTATTGGTATGGGTATAGTTGTTGATGGTTACCCATCTTACTTCGACTGT
TACAATGAAGATGGTTTGGGTATCGCTGGTTTGAATTTTCCACATTTCGCTAAGTTTTCCGATGGTCCAATTGATGGTAAGATTAACTT
GGCCTCCTACGAAATTATGTTGTGGGTTACTCAAAACTTCACCCACGTTTCTGAAGTCAAAGAAGCTTTGAAGAACGTCAACTTGGTTA
ACGAAGCTATCAACACTTTCTTTTGCTGTTGCTCCATTGCATTGGATCATCTCTGATTCTGATGAAGCCATCATCGTTGAAGTCTCTAA
ACAATACGGTATGAAGGTTTTCGATGATAAGGTTGGTGTTTTGACTAACTCCCCAGATTTTAACTGGCATTTGACCAATTTGGGTAACT
ACACAGGTTTGAATCCACATGATGCTACTGCTCAATCTTGGAATGGTCAAAAAGTTGCTCCTTGGGGTGTTGGTACTGGTTCTTTGGGT
TTGCCAGGTGATTCTATTCCAGCTGATAGATTTGTTAAGGCTGCTTACTTGAATGCTAACTACCCAACTGTTAAGGGTGAAAAGGCTAA
TGTTGCTAAGTTCTTCAACATCTTGAAGTCCGTTGCTATGATTAAGGGTTCCGTTGTTAACGATCAAGGTTCTGATGAGTACACTGTCT
ATACTGCTTGTTACTCTTCTGGTTCTAAGACCTACTACTGCAACTTCGAAGATGACTTCGAACTGAAAACCTACAAGTTGGATGATCAC
ACCATGAACTCTACATCTTTGGTTACCTACTAA

| | | | | |
|---|---|---|---|---|
| SEQ ID NO: 29 | protein sequence of | Lp_bsh2_co_SP-Bo | from | Lactobacillus |
| | | | | plantarum |

MTKNLLLGIAAVCGSTFQAVACTSLTYTNSHGGHFLARTMDFNVDFETRIMFMPRHYRVTGDLGDFTTTYGFIGAGRQLNHEIFTDGVN
ECGVSIAALYFPNHAIYQPHSNQDKIDLAPHDFVAWVLGKITSVADLRERVKDVQLISSTAELINEIPPLHFIISDQTGETAVLEPTSG
ELRLINNPVGVLTNSPNLKWQLQNLSKYGTLTNTERPLNKFINYQPGSQGPGTGALGLPGDYTSMSRFARTVFLKHYAQVPATTTDTVN
LLQHILNAVTIPKGAKVAANGQATYTEYRSYMDLNHQTYALELYENPGVIQQVNLTDHLLEKQTVPLEYALSRTPHVQLLTPDIATLPA
AH*

| | | | | |
|---|---|---|---|---|
| SEQ ID NO: 30 | DNA coding sequence of | Lp_bsh2_co_SP-Bo | from | Lactobacillus |
| | | | | plantarum |

ATGACCAAGAACTTGTTGTTGGGTATTGCTGCTGTTTGTGGTTCTACTTTTCAAGCTGTTGCTTGTACTTCTTTGACCTACACTAATTC
TCATGGTGGTCATTTCTTGGCTAGAACCATGGATTTTAACGTTGACTTCGAAACCAGGATCATGTTCATGCCAAGACATTATAGAGTTA
CCGGTGATTTGGGTGATTTCACTACTACTTACGGTTTTATTGGTGCCGGTAGACAATTGAACCACGAAATTTTCACTGATGGTGTTAAC
GAATGCGGTGTTTCTATTGCTGCATTATACTTTCCAAACCACGCTATCTATCAGCCACATTCCAATCAAGATAAGATTGATTTGGCCCC
ACATGATTTTGTTGCTTGGGTTTTGGGTAAGATTACCTCTGTTGCTGATTTGAGAGAAAGAGTTAAGGACGTCCAATTGATTTCTTCTA
CCGCCGAATTGATTAACGAAATCCCACCATTGCATTTCATCATCTCTGATCAAACTGGTGAAACTGCTGTTTTGGAACCTACTTCTGGT
GAATTGAGATTGATCAACAATCCAGTTGGTGTCTTGACTAACTCTCCAAATTTGAAGTGGCAACTGCAGAACTTGTCTAAGTATGGTAC
TTTGACTAACACCGAAAGGCCATTGAACAAGTTCATTAACTATCAACCAGGTTCTCAAGGTCCAGGTACTGGTGCTTTGGGTTTGCCAG
GTGATTATACTTCTATGTCTAGATTCGCTAGGACCGTTTTTTTGAAACATTACGCTCAAGTTCCAGCTACTACTACTGATACTGTTAAC
TTGTTGCAGCATATCTTGAACGCTGTTACTATTCCAAAAGGTGCTAAGGTTGCTGCTAATGGTCAAGCTACTTATACTGAGTACAGATC
CTACATGGATTTGAACCATCAAACTTACGCTTTGGAGTTGTACGAAAACCCAGGTGTTATTCAACAAGTCAACTTGACCGATCACTTGT
TGGAAAAACAAACCGTTCCATTGGAATACGCTTTGTCTAGAACTCCACACGTTCAATTATTGACCCCAGATATTGCTACTTTGCCAGCT
GCTCATTAA

| | | | | |
|---|---|---|---|---|
| SEQ ID NO: 31 | protein sequence of | Lp_bsh3_co_SP-Bo | from | Lactobacillus |
| | | | | plantarum |

MTKNLLLGIAAVCGSTFQAVACTSLTIQTTAGDQFLARTMDFAFELGGRPVAIPRNHHFDSVTNADGFDSPYSFVGTGRDLNGYIFVDG
VNEHGVSAAALYFSGQAHFTQQTKAGKVNLAPHEVLMWILGNVKSTAELGERIADLNVMEAAAPLLNIVVPLHWIISDKSGSTYVLELE
NDGVHYMKNPVGVMTNTPDFEWHLKNLSNYVNLQPGPHPSRQYGDMTVNPFGPGTGASGMPGDYTSVARFVRTVFMREHTDAVTTDAEA
VNALSHMLNSVEIPKGVKMQDNGTPDYTQYRAYMSMDEPAFYMQPYADQTITRVELTPALMTAAQPTEFELKTTQQFRLAN*

| | | | | |
|---|---|---|---|---|
| SEQ ID NO: 32 | DNA coding sequence of | Lp_bsh3_co_SP-Bo | from | Lactobacillus |
| | | | | plantarum |

ATGACCAAGAACTTGTTGTTGGGTATTGCTGCTGTTTGTGGTTCTACTTTTCAAGCTGTTGCTTGTACCTCTTTGACTATTCAAACTAC
TGCTGGTGATCAATTCTTGGCTAGAACTATGGATTTCGCCTTTGAATTAGGTGGTAGACCAGTTGCTATTCCAAGAAACCATCATTTCG
ATTCTGTTACCAACGCCGATGGTTTTGATTCTCCATATTCTTTTGTTGGCACCGGTAGAGATTTGAACGGTTACATTTTTGTTGACGGT
GTTAACGAACATGGTGTTTCTGCTGCTGCATTATACTTTTCTGGTCAAGCTCATTTCACTCAACAAACTAAGGCTGGTAAGGTTAATTT
GGCTCCACATGAAGTTTTGATGTGGATTTTGGGTAACGTTAAGTCTACTGCTGAATTGGGTGAAAGAATTGCTGACTTGAATGTTATGG
AAGCTGCTGCTCCTTTGTTGAATATCGTTGTTCCATTGCATTGGATCATCTCCGATAAGTCTGGTTCTACCTATGTCTTGGAATTGGAA
AACGATGGTGTTCACTACATGAAGAATCCAGTTGGTGTTATGACTAACACCCCAGATTTTGAATGGCACTTGAAGAACTTGAGCAACTA
CGTTAACTTGCAACCAGGTCCACATCCATCTAGACAATATGGTGATATGACTGTTAATCCATTCGGTCCAGGTACTGGTGCTTCTGGTA
TGCCAGGTGATTATACTTCTGTTGCTAGATTCGTTAGGACCGTGTTTATGAGAGAACATACTGATGCTGTTACTACCGATGCTGAAGCT
GTTAATGCTTTGTCTCATATGTTGAACTCCGTCGAAATTCCAAAGGGTGTTAAGATGCAAGATAACGGTACTCCAGATTACACTCAGTA
TAGAGCTTACATGTCTATGGATGAACCAGCCTTTTACATGCAACCATATGCTGATCAAACCATCACCAGAGTTGAATTGACTCCAGCTT
TGATGACTGCTGCTCAACCTACTGAATTTGAGTTGAAAACAACCCAGCAATTCAGATTGGCTAACTAA

| | | | | |
|---|---|---|---|---|
| SEQ ID NO: 33 | protein sequence of | Lp_bsh4_co_SP-Bo | from | Lactobacillus |
| | | | | plantarum |

MTKNLLLGIAAVCGSTFQAVACTSLTYLDTDNHRYFARTMDFPTTTPWRPIFLPRRYPWPTGLATTRMTQYAILGGGRLPDHFKACLMA
DGINEAGLVCAELYLPHAVEYATQPQVNQINLTPQAFINWALGEHQSVAAVIADLPSVNLVGASWGDDTGEVYPFHWYLSDAHTSVVIE
PTGGPLTAQPNPAGVLTNTPVLSDHQRRLNLYLAVSGNQITTATRQAAQHVIQTKQPLPSGPIPTDRFIHMALRRLGTPQLAPQQVPTT
LFRWLQEVSLPYHADRHHLISHNYTHYRCLITLATRTYRFIPRTTGHEQRLTLTPEMAATWRTPYLFPAD*

| | | | | |
|---|---|---|---|---|
| SEQ ID NO: 34 | DNA coding sequence of | Lp_bsh4_co_SP-Bo | from | Lactobacillus |
| | | | | plantarum |

ATGACCAAGAACTTGTTGTTGGGTATTGCTGCTGTTTGTGGTTCTACTTTTCAAGCTGTTGCTTGTACTTCTTTGACCTACTTGGATAC
CGATAACCATAGATACTTCGCTAGAACTATGGATTTCCCAACTACTACTCCTTGGAGGCCAATTTTCTTGCCAAGAAGATATCCTTGGC
CAACTGGTTTGGCTACTACTAGAATGACTCAATACGCTATTTTAGGTGGTGGTAGATTGCCAGATCATTTCAAAGCTTGTTTGATGGCC
GATGGTATTAACGAAGCTGGTTTGGTTTGTGCCGAATTATACTTGCCACATGCTGTTGAATATGCTACTCAACCACAAGTTAACCAGAT
TAACTTGACTCCACAAGCCTTTATTAACTGGGCTTTGGGTGAACATCAATCTGTTGCTGCAGTTATTGCTGATTTGCCATCCGTTAATT
TGGTTGGTGCTTCTTGGGGTGATGATACTGGTGAAGTTTATCCATTTCACTGGTACTTGTCTGATGCTCATACCTCrGTTGTTATTGAA
CCTACTGGTGGTCCATTGACTGCTCAACCTAATCCAGCTGGTGTTTTGACTAATACTCCAGTTTTGTCCGATCACCAGAGAAGATTGAA
CTTGTATTTGGCCGTTTCCGGTAATCAAATTACTACTGCTACTAGACAAGCTGCCCAACATGTTATTCAAACTAAGCAACCATTGCCAT
CTGGTCCAATTCCAACTGATAGATTCATTCATATGGCCTTGAGAAGGTTGGGTACTCCACAATTGGCTCCACAACAAGTGCCAACTACT
TTGTTTAGATGGTTGCAAGAAGTCTCCTTGCCATATCATGCTGATAGACATCATTTGATCTCCCATAACTACACCCATTACAGATGCTT
GATTACTTTGGCTACAAGAACCTACAGATTCATCCCAAGAACTACTGGTCATGAACAAAGATTGACTTTGACACCAGAAATGGCTGCTA
CTTGGAGAACTCCATATTTGTTTCCAGCTGACTGA

| | | | | |
|---|---|---|---|---|
| SEQ ID NO: 35 | protein sequence of | Lr_bsh_co_SP-Bo | from | Lactobacillus |
| | | | | reuteri |

MTKNLLLGIAAVCGSTFQAVACTSVIYTAGDYYFGRNLDLEVNLGQEVVITPRNKTLEFREMPNLEHHYAIIGMSIVRDDYPLYFDGVN
EKGVGMAGLNFDGPAHYFPVQEGKDNIASFELVPYILVTASSVAEAKKLLSNANIANINFSDKLQAAPLHWIIADKTGASVTVESTAKG
LNVYDNPVGVLTNNPEFPRQLLNLSNYRSVAPANPANVFAPDVDLPVYSRGLGTHFLPGGMDSESRFVKATFTKMHAPVGNSEVENITD
YFHILQSVEQQKGLDEVAPDTFEYTIYSDGSNLKKGIFYYKTYENNQINAVDMHKEDLEASELITYPVQNKQIINQQN*

| | | | | |
|---|---|---|---|---|
| SEQ ID NO: 36 | DNA coding sequence of | Lr_bsh_co_SP-Bo | from | Lactobacillus |
| | | | | reuteri |

ATGACCAAGAACTTGTTGTTGGGTATTGCTGCTGTTTGTGGTTCTACTTTTCAAGCTGTTGCrTGTACCTCTGTTATCTATACTGCTGG
TGATTACTACTTCGGTAGAAACTTGGATTTGGAAGTCAACTTGGGTCAAGAAGTTGTTATTACCCCAAGAAACAAGACCTTGGAATTCA
GAGAAATGCCAAACTTGGAACATCATTACGCCATTATCGGTATGTCCATCGTTAGAGATGATTACCCCTTGTACTTTGATGGTGTCAAC
GAAAAAGGTGTTGGTATGGCTGGTTTGAATTTTGATGGTCCAGCTCATTACTTCCCAGTTCAAGAAGGTAAGGATAATATCGCCTCTTT
TGAATTGGTCCCCTATATTTTGGTTACCGCTTCTTCTGTTGCTGAGGCTAAAAAGTTGTTGTCCAATGCTAATATTGCCAACATCAACT
TCTCCGATAAGTTGCAAGCTGCTCCATTGCATTGGATTATTGCTGATAAGACTGGTGCTTCTGTTACCGTTGAATCTACTGCTAAAGGT
TTGAACGTTTACGATAACCCAGTTGGTGTTTTGACTAACAATCCAGAATTTCCAAGGCAGCTGTTGAACTTGTCTAACTATAGATCTGT
TGCTCCAGCTAACCCAGCTAATGTTTTTGCTCCAGATGTTGATTTGCCAGTCTATTCTAGAGGTTTGGGTACTCATTTTTTGCCAGGTG
GTATGGATTCTGAATCTAGGTTTGTTAAGGCTACCTTCACTAAGATGCATGCTCCAGTTGGTAATTCCGAAGTTGAAAACATTACCGAC
TACTTCCACATCTTGCAATCCGTCGAACAACAAAAGGGTTTAGATGAAGTTGCCCCAGATACTTTTGAGTACACTATCTATTCTGACGG
CTCCAATTTGAAGAAGGGTATCTTTTACTACAAGACCTACGAGAACAATCAAATCAACGCTGTTGACATGCACAAAGAAGATCTTGAAG
CCTCTGAATTGATCACATACCCTGTTCAAAACAAGCAGATCATCAACCAGCAAAACTAA

| | | | | |
|---|---|---|---|---|
| SEQ ID NO: 37 | protein sequence of | Lr_bsh_co_SP-Bo | from | Lactobacillus |
| | | | | rhamnosus |

MTKNLLLGIAAVCGSTFQAVACSSMTIKSLQGDIFWGRTMDYNTSFFHESPAGGVPGKIVSLPANQTLPAQTATWKTKYAAVGVGVDQS
VALFDGVNSEGLAGDLQVLVECSWASAESLAKRNLKPIKGEEFVTLALTTCKNVAEVRALASQYGLLDEPFQFGGQGVKIPLHYTFVDP
SGAGLVIELTGHGAFKLYDSVGAMTNSPEYGWHTTNLRNYVSLNDRDYPEGADLGDQHLEPIELGTGYGMFGLPGDYTSPSRFVRAMFV
SRNLDPFNSNEGIRVLYNAFKTVLIPQGLGRDPKHSILTDYTQYWSGYDLSKRAIFVQDANTLTMTTKTLDPNLTEVTYDDLVKTEQFN
QL*

| | | | | |
|---|---|---|---|---|
| SEQ ID NO: 38 | DNA coding sequence of | Lr_bsh_co_SP-Bo | from | Lactobacillus |
| | | | | rhamnosus |

ATGACCAAGAACTTGTTGTTGGGTATTGCTGCTGTTTGTGGTTCTACTTTTCAAGCTGTTGCTTGTTCTTCCATGACCATCAAATCTTT
ACAAGGTGATATCTTCTGGGGTCGTACTATGGATTACAACACTTCATTTTTCCATGAATCTCCAGCTGGTGGTGTTCCAGGTAAAATCG
TTTCTTTGCCAGCTAATCAAACATTGCCAGCTCAAACTGCTACTTGGAAAACTAAGTACGCTGCTGTTGGTGTTGGTGTAGATCAATCA
GTTGCTTTGTTTGATGGTGTCAACTCTGAAGGTTTGGCTGGTGACTTACAAGTTTTGGTTGAATGTTCTTGGGCTTCCGCTGAATCTTT
GGCTAAAAGAAATTTGAAGCCAATCAAGGGTGAAGAGTTCGTTACTTTGGCTTTGACTACTTGTAAGAACGTTGCTGAAGTTAGAGCTT
TGGCTTCTCAATATGGTTTGTTGGATGAACCATTTCAATTCGGTGGTCAAGGTGTTAAGATTCCATTGCATTACACTTTCGTTGATCCA
TCTGGTGCTGGTTTGGTTATTGAATTGACAGGTCATGGTGCTTTCAAGTTGTATGATTCTGTTGGTGCTATGACCAACTCTCCAGAATA
TGGTTGGCATACCACTAACTTGAGAAACTACGTTTCCTTGAACGATAGAGATTATCCTGAAGGTGCTGATTTGGGTGATCAACATTTGG
AACCTATCGAATTAGGTACTGGTTACGGTATGTTTGGTTTGCCAGGTGATTATACTTCTCCATCCAGATTTGTTAGAGCCATGTTTGTC
TCTAGAAACTTGGATCCATTCAACTCCAACGAAGGTATTAGAGTCTTGTACAACGCTTTCAAGACCGTTTTGATTCCACAAGGTTTGGG
TAGAGATCCAAAGCACTCTATTTTGACTGATTACACCCAATACTGGTCCGGTTACGATTTGTCTAAGAGAGCTATTTTCGTTCAAGATG
CCAACACTTTGACTATGACTACCAAGACTTTGGACCCAAACTTGACTGAAGTTACCTATGATGATTTGGTCAAGACCGAACAGTTTAAC
CAGTTGTGA

| | | | | |
|---|---|---|---|---|
| SEQ ID NO: 39 | protein sequence of | Ba_bsh_co_SP-Bo | from | Bifidobacterium |
| | | | | animalis |

MTKNLLLGIAAVCGSTFQAVACTAVRFDDGQNNMYFGRNLDWSEDYGEKIVFAPHDYHYAPAFNAEDKNHPVIGIGIIVEDTPLYFDCM
NDAGLAVAGLNFAKYCKYATEAVNFTTNVAAYEFPLWVTRNFTSVDDVQEALKNVTIVGKPINDRFPVATLHW/IIADNTRSIVVECTE
DGMHVYDDDVDVLTNQPPFPQQIEHLDNYAYVSPRTGKSVKWGSSELETNQDSNSSQGLPGGYGSMARFVRAAYNNTHYPTQSGENANV
NRLFKTLSTAAVIEGTAISANAEFEKTLFSDCYSTATQTVYLKKYDDMAVHSYAVKDFDASSNQLQSK*

| | | | | |
|---|---|---|---|---|
| SEQ ID NO: 40 | DNA coding sequence of | Ba_bsh_co_SP-Bo | from | Bifidobacterium |
| | | | | animalis |

ATGACCAAGAACTTGTTGTTGGGTATTGCTGCTGTTTGTGGTTCTACTTTTCAAGCTGTTGCTTGTACTGCTGTTAGATTTGATGATGG
TCAGAACAACATGTACTTCGGTAGAAATTTGGACTGGTCTGAAGATTACGGTGAGAAGATAGTTTTTGCCCCACATGATTATCATTACG
CTCCAGCTTTTAACGCCGAAGATAAGAATCATCCAGTTATTGGTATCGGTATCATCGTTGAAGATACTCCCTTGTACTTTGATTGCATG
AATGATGCTGGTTTGGCTGTTGCAGGTTTGAATTTTGCTAAATACTGCAAGTACGCTACCGAAGCTGTTAATTTCACTACTAATGTTGC
TGCCTACGAATTCCCATTGTGGGTTACTAGAAATTTCACCTCCGTTGATGATGTTCAAGAAGCCTTGAAAAACGTTACCATCGTTGGTA
AGCCAATCAACGATAGATTTCCAGTTGCTACCTTGCATTGGATTATTGCTGATAACACCAGATCCATCGTTGTTGAATGTACTGAAGAT
GGTATGCACGTTTACGATGATGATGTCGATGTTTTGACTAACCAGCCACCATTTCCACAACAAATCGAACATTTGGATAACTACGCTTA
CGTTTCTCCAAGAACTGGTAAATCTGTTAAGTGGGGTTCTTCCGAATTGGAAACTAATCAAGACTCCAACTCTTCTCAAGGTTTGCCAG
GTGGTTATGGTTCTATGGCTAGATTTGTTAGAGCTGCTTACAACAACACTCACTACCCAACTCAATCTGGTGAAAATGCTAATGTCAAC
AGGTTGTTCAAGACTTTGTCTACCGCTGCTGTTATTGAAGGTACTGCTATTTCTGCTAACGCTGAATTCGAAAAGACCTTGTTTTCTGA
TTGCTACTCTACTGCTACTCAAACCGTCTACTTGAAAAAGTACGATGATATGGCCGTTCATTCCTACGCTGTTAAGGATTTTGATGCTT
CTTCTAACCAGCTGCAGTCTAAGTAA

| | | | | |
|---|---|---|---|---|
| SEQ ID NO: 41 | protein sequence of | Ba_bsh_co_SP-Bo | from | Bifidobacterium |
| | | | | breve |

MTKNLLLGIAAVCGSTFQAVACTGFRFSDDEGNTYFGRNLDWSFSYGETILVTPRGYHYDTVFGAGGKAKPNAVIGVGVVMADRPMYFD
CANEHGLAIAGLNFPGYASFVHEPVEGTENVATFEYPLWVARNFDSVDEVEEALRNVTLVSQIVPGQQESLLHWFIGDGKRSIVVEQMA
DGMHVHHDDVDVLTNQPTFDFHMENLRNYMCVSNEMAEPTSWGKASLTAWGAGVGMHGIPGDVSSPSRSVRVAYTNAHYPQQNDEAANV
SRLFHTLGSVQMVDGMAKMGNGQFERTLYTSGYSSKTNTYYMNTYDDPAIRSYAMADYDMDSSELISVAR*

| | | | | |
|---|---|---|---|---|
| SEQ ID NO: 42 | DNA coding sequence of | Ba_bsh_co_SP-Bo | from | Bifidobacterium |
| | | | | breve |

ATGACCAAGAACTTGTTGTTGGGTATTGCTGCTGTTTGTGGTTCTACTTTTCAAGCTGTTGCCGATATTATGAACGTCCAAGCTAATCA
ATCTGACGGTAGAAAGTCTGTTATGTGTACTGGTTTCAGATTCTCCGATGATGAAGGTAATACTTACTTCGGTAGAAACTTGGACTGGT
CTTTTTCTTACGGTGAAACCATTTTGGTTACCCCAAGAGGTTATCATTACGATACTGTTTTTGGTGCTGGTGGTAAAGCTAAACCTAAT
GCTGTTATAGGTGTTGGTGTTGTTATGGCTGATAGACCAATGTACTTTGATTGCGCTAATGAACATGGTTTGGCTATTGCTGGTTTGAA
TTTTCCAGGTTACGCTTCCTTTCTTCACGAACCAGTTGAAGGTACTGAAAACGTTGCTACTTTTGAATACCCATTGTGGGTTGCTAGAA
ACTTCGATTCTGTTGATGAAGTTGAAGAAGCCTTGAGAAACGTTACCTTGGTTTCTCAAATAGTCCCAGGTCAACAAGAATCTTTGTTG
CATTGGTTTATCGGTGACGGTAAGAGATCTATCGTTGTTGAACAAATGGCTGATGGTATGCATGTTCATCACGATGATGTTGATGTTTT
GACTAACCAGCCAACCTTCGATTTTCACATGGAAAACTTGAGGAACTACATGTGCGTTTCTAACGAAATGGCAGAACCTACAAGTTGGG
GTAAAGCTTCTTTGACTGCTTGGGGTGCCGGTGTTGGTATGCACGGTATTCCAGGTGATGTTTCTTCACCATCTAGATCTGTTAGAGTT
GCTTACACTAATGCTCACTACCCACAACAAAATGATGAAGCTGCTAATGTCTCTAGGTTGTTCCATACTTTGGGTTCCGTTCAAATGGT
AGACGGTATGGCTAAAATGGGTAATGGTCAATTTGAAAGGACCTTGTACACTTCCGGTTACTCTTCTAAAACTAACACCTACTACATGA
ACACCTACGATGATCCAGCTATTAGATCTTATGCTATGGCCGATTACGATATGGACTCCTCTGAATTGATTTCCGTTGCTAGATGA

| | | | | |
|---|---|---|---|---|
| SEQ ID NO: 43 | protein sequence of | Bi_bshC_co_SP-Bo | from | Bifidobacterium |
| | | | | longum subsp. |
| | | | | Infantis |

MTKNLLLGIAAVCGSTFQAVACTGVRFSDDEGNTYFGRNLDWSFSYGETILVTPRGYHYDTVFGAGGKAKPNAVIGVGVVMADRPMYFD
CANEHGLAIAGLNFPGYASFVHEPVEGTENVATFEFPLWVARNFDSVDEVEEALRNVTLVSQIVPGQQESLLHWFIGDGKRSIVVEQMA
DGMHVHHDDVDVLTNQPTFDFHMENLRNYMCVSNEMAEPTSWGKASLTAWGAGVGMHGIPGDVSSPSRFVRVAYTNAHYSQQNDEAANV
SRLFHTLGAVQMVDGMAKMGNGQFERTLFTSGYSSKTNTYYMNTYDDPAIRSYAMADYDMDSSELISVAR*

| | | | | |
|---|---|---|---|---|
| SEQ ID NO: 44 | DNA coding sequence of | Bi_bshC_co_SP-Bo | from | Bifidobacterium |
| | | | | longum subsp. |
| | | | | Infantis |

ATGACCAAGAACTTGTTGTTGGGTATTGCTGCTGTTTGTGGTTCTACTTTTCAAGCTGTTGCTTGTACTGGTGTTAGATTCTCTGATGA
TGAAGGTAACACTTACTTCGGTAGAAACTTGGATTGGTCTTTCTCTTACGGTGAAACCATTTTGGTTACTCCAAGAGGTTACCATTACG
ATACTGTTTTTGGTGCTGGTGGTAAAGCTAAACCTAATGCTGTTATAGGTGTTGGTGTTGTTATGGCTGATAGACCAATGTACTTTGAT
TGCGCTAATGAACATGGTTTGGCTATTGCTGGTTTGAATTTTCCAGGTTACGCTTCCTTTCTTCACGAACCAGTTGAAGGTACTGAAAA
CGTTGCTACTTTTGAATTCCCATTGTGGGTTGCTAGAAACTTCGATAGTGTTGATGAAGTTGAAGAAGCCTTGAGAAACGTTACCTTGG
TTTCTCAAATAGTCCCAGGTCAACAAGAATCTTTGTTGCATTGGTTTATCGGTGACGGTAAGAGATCTATCGTTGTTGAACAAATGGCT
GATGGTATGCATGTTCATCACGATGATGTTGATGTTTTGACTAACCAGCCAACCTTCGATTTTCACATGGAAAACTTGAGGAACTACAT
GTGCGTTTCTAACGAAATGGCAGAACCTACTTCTTGGGGTAAAGCTTCTTTGACTGCTTGGGGTGCCGGTGTTGGTATGCACGGTATTC
CAGGTGATGTTTCTTCACCATCTAGATTCGTTAGAGTTGCTTACACTAACGCCCATTACTCTCAACAAAATGATGAAGCTGCTAACGTG
TCTAGGTTGTTTCATACTTTGGGTGCTGTTCAAATGGTAGACGGTATGGCTAAAATGGGTAATGGTCAATTCGAAAGAACCTTGTTCAC
TTCCGGTTACTCTTCTAAGACTAACACCTACTACATGAACACCTATGATGATCCAGCCATTAGATCTTATGCTATGGCAGATTACGATA
TGGACTCCTCCGAATTGATTTCTGTTGCTAGATGA

| | | | | |
|---|---|---|---|---|
| SEQ ID NO: 45 | protein sequence of | Bl_bsh_co_SP-Bo | from | Bifidobacterium |
| | | | | longum |

MTKNLLLGIAAVCGSTFQAVACTGVRFSDDEGNTYFGRNLDWSFSYGETILVTPRGYHYDTVFGAGGKAKPNAVIGVGVVMADRPMYFD
CANEHGLAIAGLNFPGYASFVHEPVEGTENVATFEFPLWVARNFDSVDEVEETLRNVTLVSQIVPGQQESLLHWFIGDGKRSIVVEQMA
DGMHVHHDDVDVLTNQPTFDFHMENLRNYMCVSNEMAEPTSWGKASLTAWGAGVGMHGIPGDVSSPSRFVRVAYTNAHYPQQNDEAANV
SRLFHTLGSVQMVDGMAKMGDGQFERTLFTSGYSSKTNTYYMNTYDDPAIRSYAMADYDMDSSELISVAR*

| | | | | |
|---|---|---|---|---|
| SEQ ID NO: 46 | DNA coding sequence of | Bl_bsh_co_SP-Bo | from | Bifidobacterium |
| | | | | longum |

ATGACCAAGAACTTGTTGTTGGGTATTGCTGCTGTTTGTGGTTCTACTTTTCAAGCTGTTGCTTGTACFGGTGTTAGATTCFCrGATGA
TGAAGGTAACACTTACTTCGGTAGAAACTTGGATTGGTCTTTCFCTTACGGTGAAACCATTTTGGTTACTCCAAGAGGTTACCATTACG
ATACTGTTTTTGGTGCTGGTGGTAAAGCTAAACCTAATGCTGTTATAGGTGTTGGTGTTGTTATGGCTGATAGACCAATGTACTTTGAT
TGCGCTAATGAACATGGTTTGGCTATTGCTGGTTTGAATTTTCCAGGTTACGCTTCCTTTCTTCACGAACCAGTTGAAGGTACTGAAAA
CGTTGCTACTTTTGAATTCCCATTGTGGGTTGCTAGAAACTTCGATAGTGTTGATGAAGTCGAAGAAACCTTGAGAAACGTTACCTTGG
TTTCTCAAATAGTCCCAGGTCAACAAGAATCTTTGTTGCATTGGTTTATCGGTGACGGTAAGAGATCTATCGTTGTTGAACAAATGGCT
GATGGTATGCATGTTCATCACGATGATGTTGATGTTTTGACTAACCAGCCAACCTTCGATTTTCACATGGAAAACTTGAGGAACTACAT
GTGCGTTTCTAACGAAATGGCAGAACCFACTTCTTGGGGTAAAGCTTCTTTGACTGCTTGGGGTGCCGGTGTTGGTATGCACGGTATTC
CAGGTGATGTTTCTTCACCATCTAGATTCGTTAGAGTTGCTTACACTAATGCTCACFACCCACAACAAAATGATGAAGCTGCTAATGTC
TCTAGGTTGTTCCATACTTTGGGTTCCGTTCAAATGGTAGACGGTATGGCTAAGATGGGTGATGGTCAATTTGAAAGAACCTTGTTCAC
TTCCGGTTACTCTTCTAAGACTAACACCTACTACATGAACACCTATGATGATCCAGCCATTAGATCTTATGCTATGGCAGATTACGATA
TGGACTCCTCCGAATTGATTTCTGTTGCTAGATGA

| | | | | |
|---|---|---|---|---|
| SEQ ID NO: 47 | protein sequence of | Cp_bsh_co_SP-Bo | from | Clostridium |
| | | | | perfringens |

MTKNLLLGIAAVCGSTFQAVACTGLALETKDGLHLFGRNMDIEYSFNQSIIFIPRNFKCVNKSNKKELTTKYAVLGMGTIFDDYPTFAD
GMNEKGLGCAGLNFPVYVSYSKEDIEGKTNIPVYNFLLWVLANFSSVEEVKEALKNANIVDIPISENIPNTTLHWMISDITGKSIVVEQ
TKEKLNVFDNNIGVLTNSPTFDWHVANLNQYVGLRYNQVPEFKLGDQSLTALGQGTGLVGLPGDFTPASRFIRVAFLRDAMIKNDKDSI
DLIEFFHILNNVAMVRGSTRTVEEKSDLTQYTSCMCLEKGIYYYNTYENNQINAIDMNKENLDGNEIKTYKYNKTLSINHVN*

| | | | | |
|---|---|---|---|---|
| SEQ ID NO: 48 | DNA coding sequence of | Cp_bsh_co_SP-Bo | from | Clostridium |
| | | | | perfringens |

ATGACCAAGAACTTGTTGTTGGGTATTGCTGCTGTTTGTGGTTCTACTTTTCAAGCTGTTGCTTGTACTGGTTTGGCTTTGGAAACAAA
GGATGGCTTGCATTTGTTTGGTAGAAACATGGACATCGAGTACTCATTCAACCAGTCCATTATTTTCATCCCCAGAAACTTCAAGTGCG
TCAACAAGTCTAACAAGAAAGAATTGACTACCAAGTACGCCGTTTTAGGTATGGGTACTATTTTCGATGATTACCCAACTTTCGCTGAC
GGTATGAACGAAAAAGGTTTGGGTTGTGCTGGTTTGAATTTCCCAGTTTACGTGTCCTACTCCAAAGAAGATATTGAAGGCAAGACTAA
CATCCCCGTCTACAATTTTTTGTTGTGGGTTTTAGCCAACTTCAGCTCTGTTGAAGAAGTCAAAGAAGCTTTGAAGAACGCCAACATCG
TTGATATTCCAATCTCCGAAAACATTCCAAACACrACCTTGCATTGGATGATCTCTGATATCACTGGTAAGTCCATCGTTGTTGAACAG
ACCAAAGAAAAGTTGAACGTGTTCGATAACAACATCGGTGTTTTGACTAACTCTCCAACCTTTGATTGGCATGTTGCCAACTTGAATCA
ATACGTTGGTTTGAGATACAACCAGGTGCCAGAATTCAAATTGGGTGATCAATCTTTGACCGCTTTAGGTCAAGGTACAGGTTTGGTTG
GTTTGCCAGGTGATTTTACTCCAGCTTCTAGATTCATTAGAGTCGCTTTTTTGAGAGATGCCATGATCAAGAACGATAAGGATTCCATC
GATCTGATCGAATTCTTCCACATCTTGAACAACGTTGCTATGGTTAGAGGTTCTACCAGAACCGTTGAAGAAAAGTCTGATTTGACTCA
GTACACCTCTTGTATGTGCTTGGAAAAAGGTATCTACTACTACAACACCTACGAGAACAATCAAATCAACGCCATCGACATGAACAAAG
AAAACTTGGATGGTAACGAGATCAAGACCTACAAGTACAACAAGACCTTGTCCATCAACCACGTTAACTGA

| | | | | |
|---|---|---|---|---|
| SEQ ID NO: 49 | protein sequence of | Lj_La1_bsh12_co_SP- | from | Lactobacillus |
| | | Bo | | johnsonii |

MTKNLLLGIAAVCGSTFQAVACTSIVYSSNNHHYFGRNLDLEISFGEHPVITPRNYEFQYRKLPSKKAKYAMVGMAIVENNYPLYFDAA
NEEGLGIAGLNFDGPCHYFPENAEKNNVTPFELIPYLLSQCTTVAEVKDALKDVSLVNINFSEKLPLSPLHWLMADKTGESIVVESTLS
GLHVYDNPVHVLTNNPEFPGQLRNLANYSNIAPAQPKNTLVPGVDLNLYSRGLGTHFLPGGMDSASRFVKIAFVRAHSPQGNNELSSVT
NYFHILHSVEQPKGTDEVGPNSYEYTIYSDGTNLETGTFYYTNYENNQINAIELNKENLNGDELTDYKLIEKQTINYQN*

| | | | | |
|---|---|---|---|---|
| SEQ ID NO: 50 | DNA coding sequence of | Lj_La1_bsh12_co_SP- | from | Lactobacillus |
| | | Bo | | johnsonii |

ATGACCAAGAACTTGTTGTTGGGTATTGCTGCTGTTTGTGGTTCTACrTTTCAAGCTGTTGCrTGTACCTCTATCGTCTACrCTTCTAA
CAACCATCATTACTTCGGTAGGAACTTGGACTTGGAAATTTCTTTTGGTGAACACCCAGTTATCACCCCAAGAAATTATGAATTCCAGT
ACAGAAAGCTGCCCTCCAAAAAAGCTAAATATGCTATGGTTGGTATGGCCATCGTTGAAAACAATTACCCCTTGTATTTTGATGCCGCT
AACGAAGAAGGTTTAGGTATTGCAGGTTTGAATTTCGATGGTCCATGTCACTACTTTCCAGAAAATGCTGAAAAGAACAACGTCACCCC
ATTCGAATTGATTCCATACTTGTTGTCTCAATGTACCACTGTTGCTGAAGTTAAGGATGCTTTGAAGGATGTTTCCTTGGTCAACATCA
ACTTCTCTGAAAAGTTGCCATTGTCTCCATTGCATTGGTTGATGGCTGATAAGACTGGTGAATCTATCGTTGTTGAATCTACTTTGTCT
GGCTTGCACGTTTACGATAATCCAGTTCATGTTCTGACTAACAACCCAGAATTTCCAGGTCAATTGAGAAACTTGGCTAACTACTCTAA
TATTGCTCCAGCTCAACCTAAGAACACTTTGGTTCCAGGTGTTGATTTGAACTTGTACTCTAGAGGTTTGGGTACTCATTTTTTGCCAG
GTGGTATGGATTCTGCTTCTAGATTTGTTAAGATTGCCTTCGTTAGAGCCCATTCTCCACAAGGTAACAATGAATTGTCATCCGTTACC
AACTACTTCCACATCTTGCATTCTGTTGAACAACCTAAGGGTACTGATGAAGTTGGTCCAAATTCTTACGAGTACACCATCTATTCTGA
CGGCACTAATTTGGAAACTGGCACTTTTTACTACACCAACTACGAAAACAACCAGATCAACGCCATCGAGTTGAACAAAGAAAATTTGA
ACGGTGACGAACTGACCGACTACAAGTTGATTGAAAAGCAAACCATCAACTACCAGAACTAA

| | | | | |
|---|---|---|---|---|
| SEQ ID NO: 51 | protein sequence of | Lj_NCC533_bsh1_co_SP- | from | Lactobacillus |
| | | Bo | | johnsonii 533 |

MTKNLLLGIAAVCGSTFQAVACTSILYSPKDNYFGRNLDYEIAYGQKVVITPRNYQLNYRHLPTQDTHYAMIGVSVVANDYPLYCDAIN
EKGLGIAGLNFTGPGKYFSVDESKKNVTSFELIPYLLSNCETIEDVKKLLSETNITDESFSKDLPVTTLHWLMGDKSGKSIVIESTETG
LHVYDNPVNTLTNNPVFPAQVETLANFASVSPAQPKNTLVPNADINLYSRGLGTHHLPGGTDSNSRFIKASFVLAHSPKGNDEVENVTN
FFHVLHSVEQAKGTDEVEDNVFEFTMYSDCMNLDKGILYFTTYDNNQINAVDMNNEDLGTSDLITYELFKDQAIKFEN*

| | | | | |
|---|---|---|---|---|
| SEQ ID NO: 52 | DNA coding sequence of | Lj_NCC533_bsh1_co_SP- | from | Lactobacillus |
| | | Bo | | johnsonii 533 |

ATGACCAAGAACTTGTTGTTGGGTATTGCTGCTGTTTGTGGTTCTACTTTTCAAGCTGTTGCTTGTACCTCTATCTTGTACTCTCCAAA
GGATAACTACTTCGGTAGAAACTTGGATTACGAAATTGCCTACGGTCAAAAGGTTGTTATTACCCCAAGAAACTACCAGTTGAACTACA
GACATTTGCCAACTCAAGATACCCATTACGCTATGATTGGTGTTTCTGTTGTTGCTAATGATTACCCCTTGTACTGTGATGCCATTAAC
GAAAAAGGTTTGGGTATCGCTGGTTTGAATTTTACTGGTCCAGGTAAGTACTTCTCCGTTGATGAATCTAAGAAGAACGTCACCTCCTT
CGAATTGATTCCATACTTGTTGTCTAACTGCGAAACCATCGAAGATGTCAAGAAGTTGCTGTCCGAAACTAACATTACCGACGAATCTT
TCTCAAAGGATTTGCCAGTTACTACCTTGCATTGGTTGATGGGTGATAAGTCTGGTAAGTCCATCGTTATTGAATCTACTGAAACTGGC
TTGCACGTTTACGATAATCCAGTTAACACTCTGACTAACAACCCAGTTTTTCCAGCTCAAGTTGAAACTTTGGCTAACTTCGCTTCTGT
TTCACCAGCTCAACCTAAGAATACTTTGGTTCCAAACGCCGATATCAACTTGTATTCTAGAGGTTTAGGTACTCATCATTTGCCAGGTG
GTACTGATTCTAACTCCAGATTCATTAAGGCCTCATTCGTTTTGGCTCATTCTCCAAAAGGTAACGATGAAGTTGAAAACGTGACCAAC
TTTTTCCATGTCTTGCACTCTGTTGAACAAGCTAAAGGTACTGACGAAGTCGAAGATAACGTTTTCGAATTCACTATGTACTCCGACTG
TATGAACTTGGACAAGGGTATCTTATACTTCACCACCTACGACAACAATCAAATCAACGCTGTTGACATGAACAACGAGGATTTGGGTA
CTTCCGATTTGATTACTTACGAGTTGTTCAAGGATCAGGCCATCAAGTTCGAAAACTGA

| | | | | |
|---|---|---|---|---|
| SEQ ID NO: 53 | protein sequence of | Lj_NCC533_bsh2_co_SP- | from | Lactobacillus |
| | | Bo | | johnsonii 533 |

MTKNLLLGIAAVCGSTFQAVACTGLRFTDDQGNLYFGRNLDVGQDYGEGVIITPRNYPLPYKFLDNTTTKKAVIGMGIVVDGYPSYFDC
YNEDGLGIAGLNFPHFAKFSDGPIDGKINLASYEIMLWVTQNFTHVSEVKEALKNVNLVNEAINTSFAVAPLHWIISDSDEAIIVEVSK
QYGMKVFDDKVGVLTNSPDFNWHLTNLGNYTGLNPHDATAQSWNGQKVAPWGVGTGSLGLPGDSIPADRFVKAAYLNVNYPTAKGEKAN
VAKFFNILKSVAMIKGSVVNDQGKDEYTVYTACYSSGSKTYYCNFEDDFELKTYKLDDHTMNSTSLVTY*

| | | | | |
|---|---|---|---|---|
| SEQ ID NO: 54 | DNA coding sequence of | Lj_NCC533_bsh2_co_SP- | from | Lactobacillus |
| | | Bo | | johnsonii 533 |

ATGACCAAGAACTTGTTGTTGGGTATTGCTGCTGTTTGTGGTTCTACTTTTCAAGCTGTTGCTTGTACrGGTTTGAGATTCACTGATGA
TCAAGGCAACTTGTACTTCGGTAGAAATTTGGATGTTGGTCAAGATTATGGTGAGGGTGTTATTATCACTCCAAGAAATTATCCCTTGC
CATACAAGTTTTTGGATAACACTACTACCAAAAAGGCCGTTATTGGTATGGGTATAGTTGTTGATGGTTACCCATCTTACTTCGACTGT
TACAATGAAGATGGTTTGGGTATCGCTGGTTTGAATTTTCCACATTTCGCTAAGTTTTCCGATGGTCCAATTGATGGTAAGATTAACTT
GGCCTCCTACGAAATTATGTTGTGGGTTACTCAAAACTTCACCCACGTTTCTGAAGTCAAAGAAGCTTTGAAGAACGTCAACTTGGTTA
ACGAAGCTATCAACACTTTCTTTTGCTGTTGCTCCATTGCATTGGATCATCTCTGATTCTGATGAAGCCATCATCGTTGAAGTCTCTAA
ACAATACGGTATGAAGGTTTTCGATGATAAGGTTGGTGTTTTGACTAACTCCCCAGATTTTAACTGGCATTTGACCAATTTGGGTAACT
ACACAGGTTTGAATCCACATGATGCFACTGCTCAATCTTGGAATGGTCAAAAAGTTGCTCCTTGGGGTGTTGGTACTGGTTCTTTGGGT
TTGCCAGGTGATTCTATTCCAGCFGATAGATTTGTTAAGGCTGCCTACTTGAATGTTAACTACCCAACTGCTAAAGGTGAAAAGGCTAA
TGTTGCTAAGTTCTTCAACATCTTGAAGTCCGTTGCTATGATTAAGGGTTCCGTTGTTAACGATCAAGGTAAGGATGAGTACACTGTCT
ATACTGCTTGTTACTCTTCTGGTTCTAAGACCTACTACTGCAACTTCGAAGATGACTTCGAACTGAAAACCTACAAGTTGGATGATCAC
ACCATGAACTCTACATCTTTGGTTACCTACTAA

| | | | | |
|---|---|---|---|---|
| SEQ ID NO: 55 | protein sequence of | Bv_BSH_co | from | Bacteroides |
| | | | | vulgatus |

MAKTIFINDRIMKLKKGSIALLTLAGMFYMSVQKADACTRAVYIGPDHMVVTGRTMDWKEDIMSNIYVFPRGIQRAGYNKENAVKWTSK
YGSVIATGYDIGTCDGMNEKGLVASLLFLPESVFDRPGDTRPVMGISIWTQYVLDNFATVHEAVEELKKESFRIDAPHMPNGSASTLHL
AITDETGNTAVLEYLDGNLSIHEGKEFQVMTNSPRYDYQLAINNYWKEVGGLQMLPGTNRSSDRFVRASFYIHAIPQTPDAKIAVPSVL
SVMRNVSVPFGITTPDKPHISSTRWRSVSDQKNKIYYFESVMTPNLFWLDLKKIDFSPKAGIKKLTLTNGKIYAGDAVKDLKDSDSFVF
LFQTPVM*

| | | | | |
|---|---|---|---|---|
| SEQ ID NO: 56 | DNA coding sequence of | Bv_BSH_co | from | Bacteroides |
| | | | | vulgatus |

ATGGCCAAGACCATTTTCATCAACGACAGAATCATGAAGTTGAAGAAGGGTTCCATTGCHTGHGACTTTGGCTGGTATGTTCTACATGT
CTGTTCAAAAAGCTGATGCTTGTACCAGAGCTGTTTACATTGGTCCAGATCATATGGTTGTTACTGGTAGAACTATGGATTGGAAAGAG
GACATCATGTCCAACATCTACGTTTTCCCAAGAGGTATTCAAAGAGCCGGTTACAACAAAGAAAATGCTGTTAAGTGGACCTCCAAGTA
CGGTTCTGTTATTGCTACTGGTTACGATATCGGTACTTGTGATGGTATGAACGAAAAAGGTTTGGTTGCCTCCTTGTTGTTTTTGCCAG
AATCTCTTTTTGATAGACCAGGTGATACAAGACCAGTTATGGGTATTTCTATTTGGACCCAATACGTCTTGGATAACTTCGCTACTGTT
CATGAAGCTGTCGAGGAATTGAAGAAAGAATCCTTCAGAATTGATGCCCCACATATGCCAAATGGTTCTGCTTCTACTTTACATTTGGC
TATTACTGACGAAACTGGTAACACTGCTCTTTTGGAATATTTGGACGGTAACTTGTCCATCCACGAAGGTAAAGAATTCCAAGTTATGA
CCAACTCTCCAAGATACGATTACCAATTGGCCATTAACAACTACTGGAAAGAAGTTGGCGGTCTACAAATGTTGCCAGGTACTAATAGA
TCCTCCGATAGATTTGTTAGAGCCTCCTTTTACATTCACGCCATTCCACAAACTCCAGATGCTAAAATTGCTGTTCCATCCGTTTTGTC
CGTTATGAGAAATGTTTCTGTTCCATTCGGTATTACTACCCCAGATAAGCCACATATTTCTTCTACTAGATGGCGTTCCGTTTCCGATC
AAAAAAACAAGATCTACTACTTCGAGTCTGTCATGACTCCAAATTTGTTCTGGTTGGACTTGAAGAAGATCGACTTTTCACCAAAGGCT
GGTATCAAAAAGTTGACCTTGACTAACGGTAAAATCTACGCTGGTGATGCAGTTAAGGATTTGAAAGATTCCGACTCCTTCGTGTTCTT
GTTTCAAACTCCTGTTATGTAA

| | | | | |
|---|---|---|---|---|
| SEQ ID NO: 57 | protein sequence of | Er_BSH_co_SP-Bo | from | Eubacterium |
| | | | | rectale |

MTKNLLLGIAAVCGSTFQAVACTAATYKTKDFYFGRTLDYEFSYGDEIAVTPRNYVFDFRHAGKLENHYAIIGMAHVAGDYPLYYDAIN
EKGLGMAGLNFVGNAAYAAADENSSCENGICGIAKTKVAQFEFIPWILSLCATVADAKEKLNRILLVDTPFSSQLPVAQLHWIIADKNE
CIVVESMADGMHVYDNPVGVLTNNPPFTYQMAALNNYRGLSTKQPENTFAPGVELSAYSRGMGGLGLPGDLSSQSRFVRVAFTKQNSKS
DDSENASVSQFFHILGSVDQQRGLCEVTEGKYEITLYTSCCNCDKGIYYYTTYDNSQITAVDMNRENLDEKLLIRYPVITEGKICWQN*

| | | | | |
|---|---|---|---|---|
| SEQ ID NO: 58 | DNA coding sequence of | Er_BSH_co_SP-Bo | from | Eubacterium |
| | | | | rectale |

ATGACCAAGAACTTGTTGTTGGGTATTGCTGCTGTTTGTGGTTCTACTTTTCAAGCTGTTGCTTGTACTGCTGCTACTTACAAAACTAA
GGATTTCTACTTCGGTAGGACCTTGGATTACGAATTTTCTTACGGTGACGAAATTGCTGTTACCCCAAGAAATTACGTTTTCGATTTTA
GACATGCCGGCAAGTTGGAAAACCATTACGCTATTATTGGTATGGCTCATGTTGCTGGTGATTATCCCTTGTATTACGATGCCATTAAC
GAGAAAGGTTTAGGTATGGCTGGTTTGAATTTTGTTGGTAACGCTGCTTATGCTGCTGCTGACGAAAATTCTTCTTGTGAAAATGGTAT
TTGCGGTATCGCTAAGACTAAGGTTGCTCAGTTTGAATTCATTCCCTGGATTTTGTCTTTGTGTGCTACTGTTGCTGATGCCAAAGAAA
AGTTGAACAGAATCTTGTTGGTTGACACCCCATTCTCTTCACAATTGCCAGTTGCTCAATTGCATTGGATTATTGCCGATAAGAACGAA
TGCATCGTTGTTGAATCTATGGCTGATGGTATGCACGTTTACGATAATCCAGTTGGTGTTTTGACTAACAACCCACCATTCACTTATCA
AATGGCTGCCTTGAACAATTACAGAGGTTTGTCTACAAAGCAGCCAGAAAACACTTTTGCTCCAGGTGTTGAATTGTCTGCTTATTCTA
GAGGTATGGGTGGTCTAGGTTTGCCAGGTGATTTGTCATCTCAATCCAGATTTGTTAGAGTTGCCTTCACCAAGCAAAACTCCAAATCT
GATGATTCCGAAAACGCCTCTGTTTCCCAATTCTTTCATATCTTGGGTTCCGTAGATCAACAAAGGGGTTTGTGTGAAGTTACCGAAGG
TAAATACGAGATTACCTTGTACACCTCTTGTTGCAACTGTGATAAGGGTATCTACTACTACACTACCTACGACAACTCTCAAATTACCG
CTGTTGATATGAACAGGGAAAACTTGGACGAAAAGCTGTTGATTAGATACCCAGTCATTACCGAGGGTAAGATTTGTTGGCAAAACTAA

| | | | | |
|---|---|---|---|---|
| SEQ ID NO: 59 | protein sequence of | Fp_bsh_co_SP-Bo | from | Faecalibacterium |
| | | | | prausnitzii |

MTKNLLLGIAAVCGSTFQAVACTAATYKTKDFYMGRTLDYEFSYGEQITITPRNYEFDFRFAGKIKSHYALIGMAFVAEGYPLYYDAVN
EKGLGMAGLNFVGNAAYEEALPEDETEVSQVAQFEFIPWTLTQCATVADAREKLAAMRLTGTAFSEQLPTAQLHWIIADKDSCIVVESM
KDGLHVYDNPVGVLTNNPPFPSQMFALNNYAGVSRKQPESTFAGVLQLDAYSRGMGGMGIPGDLSSQSRFVKVAFTKLNSISGEEEDES
VSQFFHILGSVDQQRGCCEVTEGKYEITIYTSCCNTAKGIYYYTTYDNHQITAVDMHAENLDSDQLICYPLLSKGEVRWQNK*

| | | | | |
|---|---|---|---|---|
| SEQ ID NO: 60 | DNA coding sequence of | Fp_bsh_co_SP-Bo | from | Faecalibacterium |
| | | | | prausnitzii |

ATGACCAAGAACTTGTTGTTGGGTATTGCTGCTGTTTGTGGTTCTACTTTTCAAGCTGTTGCTTGTACTGCTGCTACTTACAAAACTAA
GGATTTCTACATGGGTCGTACCTTGGATTACGAATTTTCTTACGGTGAACAGATCACTATCACCCCAAGAAACTACGAATTCGATTTTA
GATTCGCCGGCAAGATCAAGTCTCATTATGCTTTGATTGGTATGGCTTTCGTTGCTGAAGGTTACCCACTATATTATGATGCCGTTAAC
GAAAAAGGTTTAGGTATGGCTGGTTTGAACTTTCTTGGTAACGCTGCTTATGAAGAGGCTTTGCCAGAAGATGAGACTGAAGTTTCTCA
AGTTGCCCAGTTTGAATTCATTCCATGGACTTTGACTCAATGTGCTACTGTTGCTGATGCTAGAGAAAAATTGGCTGCTATGAGATTGA
CTGGTACTGCTTTTTCTGAACAATTGCCAACTGCTCAATTGCATTGGATTATTGCCGATAAGGATTCCTGCATCGTTGTCGAATCTATG
AAGGATGGTTTACACGTTTACGATAACCCAGTTGGTGTTTTGACTAACAATCCACCATTTCCATCTCAAATGTTCGCCTTGAACAATTA
CGCTGGTGTTTCAAGAAAACAGCCAGAATCTACTTTTGCTGGTGTCTTGCAATTGGATGCTTATTCTAGAGGTATGGGTGGTATGGGAA
TTCCAGGTGATTTGTCATCTCAATCCAGATTCGTTAAGGTTGCTTTCACCAAGTTGAACTCCATTTCTGGTGAAGAGGAAGATGAATCC
GTTTCTCAATTCTTCCATATCTTGGGTTCTGTCGATCAACAAAGAGGTTGTTGCGAAGTTACTGAAGGCAAGTACGAAATTACTATCTA
CACCTCTTGTTGTAACACCGCTAAAGGTATCTACTACTACACTACTTACGACAACCATCAAATTACCGCCGTTGATATGCATGCTGAAA
ACTTGGATTCTGACCAATTGATTTGCTACCCCTTGTTGTCTAAAGGTGAAGTTAGATGGCAAAACAAGTAA

| | | | | |
|---|---|---|---|---|
| SEQ ID NO: 61 | protein sequence of | Ba_BSH_co_SP-Bo | from | Bifidobacterium |
| | | | | adolescentis |

MTKNLLLGIAAVCGSTFQAVACTGVRFSDEEGNMYFGRNLDWSFSYGESILATPRGYHYDNVFGAERKATPNAVIGVGVVMADRPMYFD
CANEHGLAIAGLNFPGYAEFVHEPVEGTDNVATFEFPLWVARNFDSVDEVEEALKNVTLVSQIVPGQQESLLHWIIGDSERSIVVEQMA
DGMHVHHDDVDVLTNQPTFGFHMENLRNYMCVGNEMAEPATWGKASLSAWGAGVSMHGIPGDVSSPSRFVRVAYANTHYPQQEGEAANV
SRLFHTLGSVQMVDGMAKMSNGQFERTLFTSGYSSKTNTYYMNTYDDPAIRSYAMADFDMDSSELITAA*

| | | | | |
|---|---|---|---|---|
| SEQ ID NO: 62 | DNA coding sequence of | Ba_BSH_co_SP-Bo | from | Bifidobacterium |
| | | | | adolescentis |

ATGACCAAGAACTTGTTGTTGGGTATTGCTGCTGTTTGTGGTTCTACTTTTCAAGCTGTTGCTTGTACTGGTGTTAGATTCTCTGATGA
AGAGGGTAATATGTACTTCGGTAGAAACTTGGATTGGTCCTTTTCTTACGGTGAATCTATTTTGGCTACTCCAAGAGGTTACCACTACG
ATAATGTTTTTGGTGCTGAAAGAAAGGCTACCCCAAATGCTGTTATTGGTGTTGGTGTTGTTATGGCTGATAGACCAATGTACTTTGAT
TGCGCTAATGAACATGGTTTGGCTATTGCTGGTTTGAATTTTCCAGGTTACGCTGAATTTGTTCACGAACCAGTTGAAGGTACTGATAA
CGTTGCTACTTTTGAATTCCCATTGTGGGTTGCTAGAAACTTCGATAGTGTTGATGAAGTTGAAGAGGCCTTGAAGAACGTTACTTTGG
TTTCTCAAATAGTCCCAGGTCAGCAAGAATCTTTGTTGCATTGGATTATCGGTGACTCCGAAAGATCTATCGTTGTTGAACAAATGGCT
GATGGTATGCATGTTCATCACGATGATGTTGATGTTTTGACTAACCAGCCAACTTTCGGTTTCCACATGGAAAACTTGAGAAACTACAT
GTGCGTTGGTAACGAAATGGCAGAACCAGCTACTTGGGGTAAAGCTTCTTTGTCTGCTTGGGGTGCTGGTGTTTCTATGCATGGTATTC
CAGGTGATGTTTCTTCACCATCTAGATTCGTTAGAGTTGCTTACGCTAATACTCACTACCCTCAACAAGAGGGTGAAGCTGCTAATGTT
TCTAGGTTGTTTCATACCTTGGGTTCCGTTCAAATGGTAGACGGTATGGCTAAAATGTCTAACGGTCAATTCGAAAGAACCTTGTTCAC
TTCTGGTTACTCTTCTAAGACTAACACCTACTACATGAACACCTATGATGATCCAGCCATTAGATCTTATGCTATGGCCGATTTTGATA
TGGACTCCTCTGAATTGATTACCGCTGCTTAA

| | | | | |
|---|---|---|---|---|
| SEQ ID NO: 63 | protein sequence of | Bd_BSH_co_SP-Bo | from | Bifidobacterium |
| | | | | dentium |

MTKNLLLGIAAVCGSTFQAVACTGVRFSDAEGNMYFGRNLDWSFSYGETILVTPRAYKYDYVFGAEAKAEPNAVIGVGVVMADKPMYFD
CANENGLAIAGLNFPGYASFAHEPIDGTINVATFEFPLWVARNFNTVDEVEEALKNVTLVSQIVPGQQESLLHWFIGDGTRSIVIEQMA
DGMHIHHDNVDVLTNQPTFDFHMENLRNYMCAGNEMAEPTTWGKAELTAWGAGVSMHGIPGDVSSPSRFVRVAYTNTHYPQQADEQSNV
SRLFHTLGSVQMVDGCAKMANGKFERTLFTSGYSAKTKTYYMNTYDDPAIRSYAMADFDMDSSELITAE*

| | | | | |
|---|---|---|---|---|
| SEQ ID NO: 64 | DNA coding sequence of | Bd_bsh_co_SP-Bo | from | Bifidobacterium |
| | | | | dentium |

ATGACCAAGAACTTGTTGTTGGGTATTGCTGCTGTTTGTGGTTCTACTTTTCAAGCTGTTGCTTGTACTGGTGTTAGATTCTCTGATGC
TGAAGGTAATATGTACTTCGGTAGAAACTTGGACTGGTCTTTTTCTTACGGTGAAACCATTTTGGTTACCCCAAGAGCTTACAAGTACG
ATTATGTTTTTGGTGCTGAAGCTAAGGCTGAACCTAATGCTGTTATTGGTGTTGGTGTTGTTATGGCTGATAAGCCAATGTACTTTGAT
TGCGCTAACGAAAACGGTTTGGCTATTGCTGGTTTGAATTTTCCAGGTTACGCTTCTTTTGCCCATGAACCTATTGATGGTACTATTAA
CGTTGCCACTTTCGAATTTCCATTGTGGGTTGCTAGAAACTTCAACACCGTTGATGAAGTTGAAGAAGCCTTGAAGAACGTTACCTTGG
TTTCTCAAATAGTCCCAGGTCAACAAGAATCTTTTGTTGCATTGGTTCATTGGTGACGGTACTAGATCCATCGTTATTGAACAAATGGC
TGATGGTATGCATATCCACCATGATAACGTTGATGTTTTGACTAACCAGCCAACCTTCGATTTTCACATGGAAAACTTGAGAAACTACA
TGTGCGCTGGTAACGAAATGGCAGAACCTACTACATGGGGTAAAGCTGAATTGACTGCTTGGGGTGCTGGTGTTTCTATGCATGGTATT
CCAGGTGATGTTTCTTCACCATCTAGATTCGTTAGAGTTGCTTACACTAACACTCACTATCCACAACAAGCTGACGAACAATCTAATGT
CTCTAGGTTGTTTCATACCTTGGGTTCCGTTCAAATGGTTGACGGTTGTGCTAAAATGGCTAACGGTAAATTTGAAAGGACCTTGTTCA
CTTCTGGTTACTCTGCTAAAACTAAGACGTACTACATGAACACCTATGATGATCCAGCCATTAGATCTTATGCTATGGCCGATTTTGAT
ATGGACTCCTCTGAATTGATTACCGCCGAATGA

| | | | | |
|---|---|---|---|---|
| SEQ ID NO: 65 | protein sequence of | Bu_BSH_co | from | Bacteroides |
| | | | | uniformis |

MNKPIKTIAVALAAIASMSVQQTEACTRATYIGPDNTVITGRTMDWKEDLMSNIYVFPRGMQRAGYNKGNTVNWTSKYGSVIAAGYDIG
TCDGMNEKGLVASLLFLPESVYHRPGDNRPIMGISIWTQYVLDNFATVREAVDELKKESFRIDAPDLPNGAASTLHLAITDETGNTAIL
EYIDGNLEIHEGKQYQVMTNSPKYELQLAINDYWKEVGGLNMLPGTNRSSDRFVRASFYINAIPQTADAKIAVPSVLSVMRNVSVPFGI
TTPDKPHISSTRWRSVSDQKNKVYYFESTLTPNLFWLDLKKIDFSPNAGIKKLSLTNGEIYAGDAIKDLKDSKGFVFLFQTPVM*

| | | | | |
|---|---|---|---|---|
| SEQ ID NO: 66 | DNA coding sequence of | Bu_BSH_co | from | Bacteroides |
| | | | | uniformis |

ATGAACAAGCCCATTAAGACCATTGCTGTTGCTTTGGCTGCTATTGCTTCTATGTCTGTTCAACAAACTGAAGCTTGTACTAGAGCTAC
TTACATTGGTCCAGATAACACTGTTATTACCGGTAGAACTATGGATTGGAAAGAGGACTTGATGTCCAACATCTACGTTTTTCCAAGAG
GTATGCAAAGAGCTGGTTACAACAAAGGTAATACTGTTAACTGGACCTCCAAGTACGGTTCTGTTATTGCTGCTGGTTATGATATCGGT
ACTTGTGATGGTATGAACGAGAAAGGTTTGGTTGCCTCTTTGTTGTTTTTGCCAGAATCCGTTTATCACAGACCAGGTGATAATAGACC
AATCATGGGTATTTCTATTTGGACCCAATACGTCTTGGATAACTTCGCTACTGTTAGAGAAGCTGTTGACGAATTGAAGAAAGAATCCT
TCAGAATTGATGCCCCAGATTTGCCAAATGGTGCTGCTTCTACTTTACATTTGGCTATTACTGACGAAACTGGTAACACTGCTATTTTG
GAGTACATCGATGGTAACTTGGAAATCCATGAAGGTAAGCAATACCAAGTCATGACCAATTCTCCAAAGTACGAATTACAATTGGCCAT
CAACGACTACTGGAAAGAAGTTGGAGGTTTGAATATGTTGCCAGGCACTAATAGATCCTCCGATAGATTTGTTAGAGCCTCCTTTTACA
TTAACGCCATTCCACAAACTGCTGATGCTAAAATTGCTGTTCCATCCGTTTTGTCCGTTATGAGAAATGTTTCTGTTCCATTCGGTATT
ACTACCCCAGATAAGCCACATATTTCTTCTACTAGATGGCGTTCCGTTTCCGATCAAAAAAACAAGGTTTACTACTTCGAGTCCACTTT
GACTCCAAATTTGTTCTGGTTGGACTTGAAGAAGATCGACTTTTCTCCAAACGCTGGTATCAAAAAGTTGTCTTTGACTAACGGTGAAA
TCTACGCTGGTGATGCTATTAAGGATTTGAAAGACTCTAAGGGCTTCGTGTTCTTGTTTCAAACTCCAGTTATGTAA

| | | | | |
|---|---|---|---|---|
| SEQ ID NO: 67 | protein sequence of | Ca_BSH_co_SP-Bo | from | Collinsella |
| | | | | aerofaciens |

MTKNLLLGIAAVCGSTFQAVACTSIRFTDNSGHMYLGRNLDWSFDYGQQVRVMPRGFHIPYSFIDDASAAHAVIGMCIDYKNYPMFFDC
GNDAGLAVAGLNFPGYAEYAEDPVDGKTNIAAYEFPLWVAATFSTVDEVEAALRDTVIVAKSAGEGLGVSLLHWIIGDNQRSIVVEMMA
DGLHVYDDPVDTLANQPTFPWHMENLRTYITATSDFPQTATWRGAELKPYGAGAGMRGIPGDCYSPSRFVKAAYLNANYPQKESETENV
VRMFRSLEGVVMIEGASRMGDGKFEKTIYTGCFSARTGNYYYAMYGDPSIRYVSLMDAEGASPDRLVVPEPRRS*

| | | | | |
|---|---|---|---|---|
| SEQ ID NO: 68 | DNA coding sequence of | Ca_BSH_co_SP-Bo | from | Collinsella |
| | | | | aerofaciens |

ATGACCAAGAACTTGTTGTTGGGTATTGCTGCTGTTTGTGGTTCTACTTTTCAAGCTGTTGCTTGTACCTCTATCAGATTCACTGATAA
CTCTGGTCATATGTACTTGGGTAGAAACTTGGATTGGTCTTTCGATTACGGTCAACAAGTTAGAGTTATGCCAAGAGGTTTCCACATTC
CATACAGCTTTATTGATGATGCTTCTGCTGCTCATGCTGTTATTGGTATGTGTATCGATTACAAGAACTACCCCATGTTCTTCGATTGT
GGTAATGATGCTGGTTTGGCTGTTGCAGGTTTGAATTTTCCAGGTTATGCTGAATACGCTGAAGATCCAGTTGATGGTAAGACTAACAT
TGCTGCTTACGAATTTCCATTGTGGGTTGCTGCTACTTTCTCTACTGTTGATGAAGTTGAAGCTGCTTTGAGAGATACTGTTATCGTTG
CTAAATCTGCTGGTGAAGGTTTGGGTGTTTCTlTGTTGCATTGGATCATCGGTGACAATCAAAGATCCATCGTTGTTGAAATGATGGCT
GATGGTTTACACGTTTACGATGATCCTGTTGATACTTTGGCTAATCAACCTACTTTTCCTTGGCACATGGAAAACTTGAGAACCTACAT
TACTGCTACCTCTGATTTTCCACAAACTGCTACTTGGAGAGGTGCTGAATTGAAACCATACGGTGCTGGTGCTGGTATGAGAGGTATTC
CAGGTGATTGCTATTCTCCATCTAGATTTGTTAAGGCTGCTTACTTGAATGCTAACTACCCACAGAAAGAATCCGAAACCGAAAACGTT
GTTAGAATGTTCAGATCCTTGGAAGGTGTTGTTATGATTGAAGGTGCTTCTAGAATGGGTGACGGTAAATTTGAAAAGACTATCTACAC
CGGTTGCTTCTCTGCTAGAACTGGTAATTACTATTACGCCATGTATGGTGACCCATCCATTAGATACGTTTCTTTGATGGATGCTGAAG
GTGCATCTCCAGATAGATTGGTTGTTCCAGAACCTAGAAGATCCTGA

| | | | | |
|---|---|---|---|---|
| SEQ ID NO: 69 | protein sequence of | Blo_BSH1_co_SP-Bo | from | Blautia obeum |

MTKNLLLGIAAVCGSTFQAVACTAATYQTKDFYFGRTLDYEFSYGDQIAITPRNYPFSFRHAGEMATHYAIIGMAHVAGSYPLYYDAIN
EKGVGMAGLNFVGNAAYADVSPDRDNVAQFEFIPWILSQCATLSEVRTLLERMALVATPFNEQFPLAQLHWIISDKTGSITVESMADGL
HIYENPVGVLTNNPPFPQQMFQLNNFMHLSPKQPVNTFSDDLALTAYSRGMGALGLPGDLSSASRFVRVAFTKMNAFSGDSEKESVSQF
FHILGSVDQQRGCCEVADGKYEITLYTSCCNVTKGIYYYTTYENHQISAVDMHRENLDGGALICYPVIQGEQINYQN*

| | | | | |
|---|---|---|---|---|
| SEQ ID NO: 70 | DNA coding sequence of | Blo_BSH1_co_SP-Bo | from | Blautia obeum |

ATGACCAAGAACTTGTTGTTGGGTATTGCTGCTGTTTGTGGTTCTACTTTTCAAGCTGTTGCTTGTACTGCTGCTACTTACCAAACTAA
GGATTTCTACTTCGGTAGAACCTTGGACTACGAATTTTCTTACGGTGATCAAATTGCTATCACCCCAAGAAATTACCCATTCTCTTTTA
GACATGCTGGTGAAATGGCTACCCATTACGCTATTATTGGTATGGCTCATGTTGCTGGTTCTTATCCCTTGTATTACGATGCCATTAAC
GAAAAAGGTGTAGGTATGGCTGGTTTGAATTTTGTTGGTAATGCTGCATACGCTGATGTTTCTCCAGATAGAGATAATGTTGCTCAGTT
CGAATTCATCCCATGGATTTTGTCTCAATGTGCTACCTTGTCTGAAGTCAGAACHTGHGGAAAGAATGGCTTTGGTTGCTACTCCATTC
AATGAACAATTTCCATTGGCTCAATTGCACTGGATCATTTCTGATAAGACTGGTTCCATTACCGTTGAATCTATGGCrGATGGTTTACA
CATCTACGAAAATCCAGTTGGTGTCTTGACTAACAATCCACCATTTCCACAACAAATGTTCCAGCTGAACAACTTCATGCATTTGTCTC
CAAAGCAACCAGTTAACACCTTCTCTGATGATTTGGCTTTGACTGCTTATTCTAGAGGTATGGGTGCTTTGGGTTTGCCAGGTGATTTG
TCATCTGCTTCTAGATTTGTTAGAGTTGCCTTCACTAAGATGAACGCTTTTTCTGGTGACTCCGAAAAAGAATCCGTTTCTCAATTCTT
CCACATCTTGGGTTCTGTTGACCAACAAAGAGGTTGTTGCGAAGTTGCTGATGGTAAATACGAAATTACCTTGTACACCTCTTGTTGCA
ACGTTACTAAGGGTATCTACTACTACACTACCTACGAAAACCATCAAATCTCCGCTGTTGATATGCACAGAGAAAATTTGGATGGTGGT
GCTTTGATTTGCTACCCAGTTATTCAAGGTGAACAGATCAACTACCAGAACTAA

| | | | | |
|---|---|---|---|---|
| SEQ ID NO: 71 | protein sequence of | Blo_BSH2_co_SP-Bo | from | Blautia obeum |

MTKNLLLGIAAVCGSTFQAVACTAATYKSKDFYFGRTLDYEFSYGDQIVITPRNYSFHFRYIGDKKKHYAMIGMAHVAENYPLYYDAMN
EKGVAIAGLNFVGNAVYSEVQSDVENIAQFEFIPWILSQCASLAEVRELLDRINIVNTPFSEQLPLAQLHWIISDENESITVESMSHGL
HIYDNPVGVLTNNPPFPQQMFQLNNYMHLSPKQPENTFSENLALDAYSRGMGGIGLPGDLSSSSRFVRVAFTKIHAISGESEKESVSQF
FHILGSVDQQRGCCEVADGKYEITLYTSCCNVTKGIYYYNTYENHQISAVDMYAEDLDGNNLICYPVIQGEQINYQNK*

| | | | | |
|---|---|---|---|---|
| SEQ ID NO: 72 | DNA coding sequence of | Blo_BSH2_co_SP-Bo | from | Blautia obeum |

ATGACCAAGAACTTGTTGTTGGGTATTGCTGCTGTTTGTGGTTCTACTTTTCAAGCTGTTGCTTGTACTGCTGCTACTTACAAATCTAA
GGATTTCTACTTCGGTAGGACCTTGGATTACGAATTTTCTTACGGTGATCAAATCGTCATCACCCCAAGAAACTATTCCTTCCATTTCA
GATATATCGGCGACAAGAAAAAGCACTACGCTATGATTGGTATGGCTCATGTTGCTGAAAATTACCCCTTGTATTACGATGCCATGAAC
GAAAAAGGTGTTGCTATTGCTGGTTTGAACTTTGTTGGTAACGCTGTTTACTCCGAAGTTCAATCTGATGTTGAAAACATTGCCCAGTT
CGAATTCATTCCATGGATTTTGTCTCAATGCGCTTCTTTGGCTGAAGTTAGAGAATTATTGGACAGGATCAACATCGTGAACACTCCAT
TTTCTGAACAATTGCCATTGGCTCAATTGCACTGGATTATTTCTGACGAAAACGAATCCATCACCGTCGAATCTATGTCTCATGGTTTA
CACATCTACGATAACCCAGTTGGTGTTTTGACTAACAATCCACCATTTCCACAACAGATGTTCCAGTTGAACAACTACATGCATTTGTC
TCCAAAGCAACCAGAAAACACCTTCTCTGAAAATTTGGCTTTGGATGCTTACTCTAGAGGTATGGGTGGTATTGGTTTGCCAGGTGATT
TGTCATCTTCTTCAAGATTCGTTAGAGTTGCCTTCACCAAGATTCATGCTATTTCrGGTGAATCCGAAAAAGAGTCCGTTTCTCAATTC
TTCCATATCTTGGGTTCTGTCGATCAACAAAGAGGTTGTTGCGAAGTTGCTGATGGTAAATACGAAATTACCTTGTACACCTCTTGTTG
CAACGTTACTAAGGGTATCTACTACTACAACACCTACGAAAACCATCAAATCTCCGCTGTTGATATGTACGCTGAAGATTTGGATGGTA
ACAACTTGATTTGCTACCCAGTTATTCAAGGTGAGCAGATCAATTACCAGAACAAGTGA

| | | | | |
|---|---|---|---|---|
| SEQ ID NO: 73 | protein sequence of | PdBSHco | from | Parabacteroides |
| | | | | distasonis |

MKSKNLTLASLFAGLFMFMNPMGSEACTRAVYLGPDDMVVTGRTMDWKEDPQSNLYLFPQGVQRRGAISDNTIQWTSKYGSVVTAGYDI
GTCDGMNEKGLVANLLFLTESSYFRPDDNRPVMGLSIWTQYVLDNFATVDEAVAELSKEVFRIDDPDLPNGAKSTLHLSISDASGNSAI
FEYLNGNLVIHEGRECQVMTNSPTYDKQLTLNDYWQQIGGLVMLPGTNRASDRFVRASFYIHAIPQTSNFREAVAGVFSVMRNVSVPLG
ITTPDQPNISSTRWRTVADQKNKVYYFESTLSPDIFWVDFKSLDFKAGTPIKKLTLTNGEIYAGNTAKDFKDSKPLPFLFGI*

| | | | | |
|---|---|---|---|---|
| SEQ ID NO: 74 | DNA coding sequence of | PdBSHco | from | Parabacteroides |
| | | | | distasonis |

ATGAAGTCCAAGAACTTGACCTTGGCTTCTTTGTTTGCTGGTTTGTTCATGTTTATGAACCCAATGGGTAGTGAAGCTTGTACTAGAGC
TGTTTATTTGGGTCCAGATGATATGGTTGTTACTGGTAGAACTATGGATTGGAAAGAAGATCCACAGTCCAACTTGTACTTGTTTCCAC
AAGGTGTTCAAAGAAGAGGTGCCATTTCTGATAACACTATTCAATGGACTTCCAAGTACGGTTCTGTTGTTACAGCTGGTTATGATATC
GGTACTTGTGATGGTATGAACGAGAAAGGTTTGGTTGCTAACTTGTTGTTCTTGACCGAATCCTCTTACTTCAGACCAGATGACAATAG
ACCAGTTATGGGTTTGTCTATTTGGACCCAATACGTTTTGGATAACTTCGCTACTGTTGATGAAGCTGTTGCCGAATTGTCTAAAGAAG
TTTTCAGAATCGACGATCCAGATTTGCCAAATGGTGCTAAATCTACCTTGCACTTGTCAATTTCTGATGCCTCTGGTAACTCTGCCATT
TTCGAATACTTGAACGGTAACTTGGTTATCCACGAAGGTAGAGAATGTCAAGTTATGACTAACTCTCCAACCTACGATAAGCAATTGAC
CTTGAATGATTACTGGCAACAAATCGGTGGTTTGGTTATGTTGCCAGGTACTAATAGAGCTTCCGATAGATTTGTTAGAGCCTCCTTTT
ACATTCACGCTATTCCACAAACTTCCAACTTCAGAGAAGCAGTTGCTGGTGTTTTTTCCGTTATGAGAAATGTTTCTGTGCCATTGGGT
ATTACTACTCCAGATCAACCTAACATTTCTTCTACTAGATGGCGTACTGTTGCTGACCAAAAAAACAAGGTTTACTACTTCGAGTCTAC
CTTGTCTCCAGATATTTTCTGGGTTGACTTCAAGAGCTTGGATTTTAAAGCTGGTACTCCCATCAAAAAGCTGACTTTGACTAATGGTG
AAATCTACGCTGGTAACACTGCTAAGGATTTCAAAGATTCTAAGCCCTTGCCATTCTTGTTCGGTATTTGA

| | | | | |
|---|---|---|---|---|
| SEQ ID NO: 75 | protein sequence of | Pd_bsh_co_SP-Bo | from | Parabacteroides |
| | | | | distasonis |

MTKNLLLGIAAVCGSTFQAVACTRAVYLGPDDMVVTGRTMDWKEDPQSNLYLFPRGVQRRGAISDNTIQWTSKYGSVVTAGYDIGTCDG
MNEKGLVANLLFLTESSYFRSDDNRPVMGLSIWTQYVLDNFATVDDAVAELSKEVFRIDDPDLPNGAKSTLHLSISDASGNSAIFEYLN
GNLVIHEGRECQVMTNSPTYDKQLTLNDYWQQIGGLVMLPGTNRASDRFVRASFYIHAIPQTSNFREAVAGVFSVMRNVSVPLGITTPD
QPNISSTRWRTVADQKNKVYYFESTLSPDIFWVDFKSLDFKAGTPIKKLTLTNGEIYAGNTTKDFKDSKPLPFLFGI*

| | | | | |
|---|---|---|---|---|
| SEQ ID NO: 76 | DNA coding sequence of | Pd_BSH_co_SP-Bo | from | Parabacteroides |
| | | | | distasonis |

ATGACCAAGAACTTGTTGTTGGGTATTGCTGCTGTTTGTGGTTCTACTTTTCAAGCTGTTGCTTGTACTAGAGCTGTTTATTTGGGTCC
AGATGATATGGTTGTTACTGGTAGAACTATGGATTGGAAAGAAGATCCACAGTCCAACTTGTACTTGTTTCCAAGAGGTGTTCAAAGAA
GAGGTGCCATTTCTGATAACACTATTCAATGGACTTCCAAGTACGGTTCTGTTGTTACAGCTGGTTATGATATCGGTACTTGTGATGGT
ATGAACGAGAAAGGTTTGGTTGCCAATTTGTTGTTCTTGACCGAATCCTCTTACTTCAGGTCTGATGATAATAGACCAGTCATGGGTTT
GTCTATTTGGACCCAATACGTTTTGGATAACTTCGCTACTGTTGATGATGCTGTTGCCGAATTGTCTAAAGAAGTTTTCAGAATCGACG
ATCCAGATTTGCCAAATGGTGCTAAATCTACCTTGCACTTGTCAATTTCTGATGCCTCTGGTAACTCTGCCATTTTCGAATACTTGAAC
GGTAACTTGGTTATCCACGAAGGTAGAGAATGTCAAGTTATGACTAACTCTCCAACCTACGATAAGCAATTGACCTTGAATGATTACTG
GCAACAAATCGGTGGTTTGGTTATGTTGCCAGGTACTAATAGAGCTTCCGATAGATTTGTTAGAGCCTCCTTTTACATTCACGCTATTC
CACAAACTTCCAACTTCAGAGAAGCAGTTGCTGGTGTTTTTTCCGTTATGAGAAATGTTTCTGTCCCATTGGGTATTACTACTCCAGAT
CAACCTAACATTTCTTCTACTAGATGGCGTACTGTTGCTGACCAAAAAAACAAGGTTTACTACTTCGAGTCTACCTTGTCTCCAGATAT
TTTCTGGGTTGACTTCAAGTCCTTGGATTTTAAAGCTGGTACTCCCATCAAAAAGCTGACTTTGACTAATGGTGAAATCTACGCTGGTA
ACACTACCAAGGATTTCAAAGATTCTAAGCCCTTGCCATTCTTGTTCGGTATTTGA

| | | | | |
|---|---|---|---|---|
| SEQ ID NO: 77 | protein sequence of | Lm_BSH_co_SP-Bo | from | Lactobacillus |
| | | | | mucosae |

MTKNLLLGIAAVCGSTFQAVACTAATYTAGDHYFGRNLDLELSYGEKVTITPRNFPLTFRKMPTLEHHYALIGMATTVDNYPLYFDATN
EKGLSMAGLNYPGNADYKPYAEGKDNVSPFEFVSWILGQCATLAEAKVLLDKINLVKIDFSEQLPLSPLHWLMAETASEKTLTIECDRD
GLHVYDNPVGVLTNNPQFDKQLFNLNNYQFLSPQQPENKFSQDLELDNYSRGLGTRGLPGGMDSMSRFVKVAFTKLNAPKGETENENVG
NFFHILHSVEQQKNLDGVENNQFEFTIYSSCVNADRGIYYYTTYDNNQINAVDMHKVDLDGRELIDYDLANEQNINWQN*

| | | | | |
|---|---|---|---|---|
| SEQ ID NO: 78 | DNA coding sequence of | Lm_BSH_co_SP-Bo | from | Lactobacillus |
| | | | | mucosae |

ATGACCAAGAACTTGTTGTTGGGTATTGCTGCTGTTTGTGGTTCTACTTTTCAAGCTGTTGCTTGTACTGCTGCTACTTATACTGCTGG
TGATCATTACTTTGGTAGGAACTTGGATTTGGAGTTGTCTTACGGTGAAAAGGTTACTATTACCCCAAGAAACTTCCCATTGACCTTTA
GAAAAATGCCAACCTTGGAACATCATTACGCCTTGATTGGTATGGCTACTACTGTTGATAACTACCCCTTGTACTTTGATGCTACCAAC
GAAAAAGGTTTGTCTATGGCTGGTTTGAATTACCCAGGTAATGCTGATTACAAACCATACGCTGAAGGTAAGGATAACGTTTCTCCATT
CGAATTCGTTTCTTGGATTTTGGGTCAATGTGCTACFTTGGCFGAAGCTAAAGTTTTGTTGGACAAGATCAACTTGGTCAAGATCGACT
TCTCTGAACAATTGCCATTGTCTCCATTGCATTGGTTGATGGCTGAAACTGCTTCTGAAAAGACTTTGACCATTGAATGTGATAGAGAT
GGCTTGCACGTTTACGATAATCCAGTTGGTGTTTTGACTAACAACCCACAATTTGACAAGCAGCTGTTCAACCTGAACAACTACCAATT
TTTGTCTCCACAACAGCCAGAGAACAAGTTTTCTCAAGATTTGGAACTGGACAACTACTCTAGAGGTTTGGGTACAAGAGGTTTGCCAG
GTGGTATGGATTCTATGTCTAGATTTGTTAAGGTTGCCTTCACTAAGTTGAATGCTCCAAAAGGTGAAACCGAAAACGAAAACGTTGGT
AACTTCTTCCATATCTTGCACTCTGTCGAACAGCAAAAGAATTTGGATGGTGTTGAAAACAACCAGTTCGAGTTCACTATCTACTCCTC
TTGTGTTAATGCAGACAGAGGTATCTACTACTACACTACCTATGATAACAACCAGATTAACGCCGTTGATATGCACAAGGTTGATTTGG
ACGGTAGAGAATTGATCGATTACGATTTGGCTAACGAGCAGAACATTAACTGGCAAAACTGA

| | | | | |
|---|---|---|---|---|
| SEQ ID NO: 79 | protein sequence of | Bo_ATCC_BSH_co | from | Bacteroides ovatus |
| | | | | ATCC 8483 |

MMKMTKNLLLGIAAVCGSTFQAVACFGISLTSRDGSYVQARTIEWARGVLQSEYVIIPRGQQLTSFTPTGVNGLTFTAKYGVVGLAVVQ
KEFIAEGINEAGLSAGLFFFPHYGGYKTYDAAQNQRTLADLQVTEWLLSQFSTIDEVKAALSSVRVVGLEKTAVVHWRIGEPSGRQVVL
EIVGGVPHFYENEVGVLTNAPGFEWQLTNLNNYVNLHPGDASVQKLSGITLQPTGGNSGFLGIPGDATPPSRFVRAAFYRGTAPQRATG
FDTVQQCFHLLNNFDIPIGIEHSQGDIPDIPSATQWTSAIDLTNRKVYYKTAYNNSIRCIDLKAIDFSKVKYQSQPLDKIQEQPVEMVK
IPR*

| | | | | |
|---|---|---|---|---|
| SEQ ID NO: 80 | DNA coding sequence of | Bo_ATCC_BSH_co | from | Bacteroides ovatus |
| | | | | ATCC 8483 |

ATGATGAAGATGACCAAGAACCTGTTGTTGGGTATTGCTGCTGTTTGTGGTTCTACTTTTCAAGCTGTTGCTTGTACCGGTATTTCTTT
GACTTCTAGAGATGGTTCTTACGTTCAAGCCAGAACTATTGAATGGGCTAGAGGTGTTTTACAGTCCGAATACGTTATTATCCCAAGAG
GTCAACAATTGACTTCTTTCACTCCAACTGGTGTTAACGGTTTGACTTTTACTGCTAAGTATGGTGTTGTTGGTTTGGCCGTTGTTCAG
AAAGAATTCATTGCTGAAGGTATTAACGAAGCTGGTTTGTCTGCTGGTTTATTCTTTTTTCCACATTACGGTGGTTACAAGACTTACGA
TGCTGCTCAAAATCAAAGAACTTTGGCCGACTTACAAGTTACCGAATGGTTGTTGTCTCAATTCTCCACTATCGATGAAGTTAAGGCTG
CTTTGTCATCCGTTAGAGTTGTAGGTTTGGAAAAGACTGCTGTTGTTCATTGGAGAATAGGTGAACCATCTGGTAGACAAGTTGTTTTG
GAAATAGTTGGTGGTGTTCCACACTTCTACGAAAATGAAGTTGGTGTTTTGACTAACGCTCCAGGTTTTGAATGGCAATTGACTAACTT
GAACAACTACGTTAACTTGCATCCAGGTGATGCCTCTGTTCAAAAATTGTCTGGTATTACCTTGCAACCTACTGGTGGTAATTCTGGTT
TTTTGGGTATTCCTGGTGATGCTACTCCACCATCTAGATTTGTTAGAGCTGCTTTTTACAGAGGTACTGCTCCACAAAGAGCTACTGGT
TTTGATACTGTTCAACAATGCTTCCACCTGTTGAACAATTTCGATATTCCAATCGGTATCGAACACTCCCAAGGTGATATTCCAGATAT
TCCTTCTGCTACTCAATGGACCTCTGCTATTGATTTGACCAACAGAAAGGTTTACTACAAGACCGCTTACAACAACTCCATTAGATGCA
TCGATTTGAAGGCCATCGATTTCTCTAAGGTCAAGTATCAATCTCAGCCATTGGACAAGATTCAAGAACAACCAGTTGAAATGGTCAAG
ATCCCAAGATGA

| | | | | |
|---|---|---|---|---|
| SEQ ID NO: 81 | protein sequence of | Bu_ATCC_BSH_co | from | Bacteroides |
| | | | | uniformis |
| | | | | ATCC 8492 |

MAKRIPILLAAMTALTFAPQTAEACTGITLTAKDNAHVVARTIEWGGSELNSQYVIVPRGYSQQSLTPGGSLSGMEFTSRYGYVGLAVE
QKEFVAEGLNEAGLSAGLFYFPGYGKYEEYDPTRKASSIADLQLVSWLLGSFSTVDEVKEAIENVTVIAIDPRASTVHWRIADKTGRQL
VLEFIDGQPRFYENKLGVLTNSPGFDWQMTNLNNYVNLYPGSAESKSLGNVTIAPFGSGSGFLGIPGDVTPPSRFIRAAFYQTSTPPQN
TAQETVLQCFQILNNFDIPVGIEFPLGQVPANIPSATQWTSATDMTNGIIYYRTMYNSTIRSIDLRQIDFGRIEYHAAPLDKVKRQPIA
PVKIE*

| | | | | |
|---|---|---|---|---|
| SEQ ID NO: 82 | DNA coding sequence of | Bu_ATCC_BSH_co | from | Bacteroides |
| | | | | uniformis |
| | | | | ATCC 8492 |

ATGGCCAAGAGAATTCCAATTTTGTTGGCTGCTATGACTGCTTTGACTTTTGCTCCACAAACTGCTGAAGCTTGTACTGGTATTACTTT
GACTGCTAAGGATAACGCTCATGTTGTTGCTAGAACTATTGAATGGGGTGGTTCTGAATTGAACTCCCAATATGTTATAGTCCCAAGAG
GTTACTCCCAACAATCTTTGACTCCAGGTGGTTCTTTGTCTGGTATGGAATTCACTTCTAGATACGGTTATGTTGGTTTGGCCGTCGAA
CAAAAAGAATTTGTTGCTGAAGGTTTGAACGAAGCTGGTTTGTCTGCTGGTTTATTCTATTTTCCAGGTTACGGCAAGTACGAAGAATA
CGATCCAACTAGAAAGGCTTCTTCTATTGCTGACTTGCAATTGGTTTCTTGGTTGTTGGGTTCTTTCTCTACTGTTGACGAAGTCAAAG
AAGCCATTGAAAACGTTACCGTTATTGCCATTGATCCAAGAGCTTCTACTGTTCATTGGAGAATTGCTGATAAGACCGGTAGACAATTG
GTCTTGGAATTCATTGATGGTCAGCCAAGATTCTACGAAAACAAGTTGGGTGTTTTGACTAACTCTCCAGGTTTTGATTGGCAAATGAC
CAACTTGAACAACTACGTTAACTTGTATCCAGGTTCCGCTGAATCTAAGTCTTTGGGTAATGTTACTATTGCCCCATTTGGTTCTGGTT
CAGGTTTTTTGGGTATTCCAGGTGATGTTACTCCACCATCTAGATTCATTAGAGCTGCTTTCTACCAAACTTCTACACCACCACAAAAC
ACTGCTCAAGAAACCGTTTTACAATGCTTCCAGATCCTGAACAACTTCGATATTCCAGTTGGTATCGAATTCCCATTGGGTCAAGTTCC
AGCTAATATTCCATCTGCTACTCAATGGACTTCTGCTACTGATATGACTAACGGTATCATCTACTACAGGACCATGTACAACTCTACCA
TCAGATCCATTGACTTGAGACAAATTGACTTCGGTAGAATCGAATATCATGCTGCTCCATTGGATAAGGTTAAGAGACAACCTATTGCT
CCAGTCAAGATCGAATGA

| | | | | |
|---|---|---|---|---|
| SEQ ID NO: 83 | protein sequence of | Eve_BSH_co_SP-Bo | from | Eubacterium |
| | | | | ventriosum |
| | | | | ATCC 27560 |

MTKNLLLGIAAVCGSTFQAVACTAATYKTKDFYMGRTLDYEFSYGEQITITPRNYEFDFRFAGKIKSHYALIGMAFVAGGYPLYYDAVN
EKGLGMAGLNFVGNAAYEEALPEDETEVSQVAQFEFIPWILTQCATVAEAREKLAAMRLTGTAFSEQLPTAQLHWIIADKDACIVVESM
KDGLHVYDNPVGVLTNNPPFLGQMFALNNYAGVSRKQPESTFAGVLQLDAYSRGMGGMGIPGDLSSQSRFVKVAFTKLNSISGEEEGES
VSQFFHILGSVDQQRGCCEVTEGKYEITIYTSCCNTAKGIYYYTTYDNHQITAVDMHAENLDSDQLICYPLLSKGEVRWQNK*

| | | | | |
|---|---|---|---|---|
| SEQ ID NO: 84 | DNA coding sequence of | Eve_BSH_co_SP-Bo | from | Eubacterium |
| | | | | ventriosum |
| | | | | ATCC 27560 |

ATGACCAAGAACTTGTTGTTGGGTATTGCTGCTGTTTGTGGTTCTACTTTTCAAGCTGTTGCTTGTACTGCTGCTACTTACAAAACTAA
GGATTTCTACATGGGTCGTACCTTGGATTACGAATTTTCTTACGGTGAACAGATCACTATCACCCCAAGAAACTACGAATTCGATTTTA
GATTCGCCGGCAAGATCAAGTCTCATTATGCTTTGATTGGTATGGCTTTTGTTGCTGGTGGTTACCCACTATATTATGATGCCGTTAAC
GAAAAAGGTTTAGGTATGGCTGGTTTGAACTTTGTTGGTAACGCTGCTTATGAAGAGGCTTTGCCAGAAGATGAGACTGAAGTTTCTCA
AGTTGCCCAGTTTGAATTCATTCCATGGATTTTGACTCAATGCGCTACTGTTGCTGAAGCTAGAGAAAAATTGGCTGCTATGAGATTGA
CTGGTACTGCTTTTTCTGAACAATTGCCAACTGCTCAATTGCATTGGATTATTGCTGATAAGGATGCCTGCATCGTTGTCGAATCTATG
AAGGATGGTTTACACGTTTACGATAACCCAGTTGGTGTTTTGACTAACAATCCACCATTTTTGGGTCAAATGTTCGCCTTGAACAATTA
CGCTGGTGTTTCAAGAAAACAGCCAGAATCTACTTTTGCTGGTGTCTTGCAATTGGATGCTTATTCTAGAGGTATGGGTGGTATGGGAA
TTCCAGGTGATTTGTCATCTCAATCCAGATTCGTTAAGGTTGCTTTCACCAAGTTGAACTCCATTTCTGGTGAAGAGGAAGGTGAATCT
GTTTCCCAATTCTTCCATATCTTGGGTTCTGTAGATCAACAAAGAGGTTGTTGCGAAGTTACCGAAGGTAAATACGAGATTACTATCTA
CACCTCTTGTTGTAACACTGCCAAAGGTATCTACTACTACACTACTTACGACAACCATCAAATTACCGCCGTTGATATGCATGCTGAAA
ACTTGGATTCTGACCAATTGATTTGCTACCCCTTGTTGTCTAAAGGTGAAGTTAGATGGCAAAACAAGTAA

| | | | | |
|---|---|---|---|---|
| SEQ ID NO: 85 | protein sequence of | Blo_ATTC_BSH_co_SP-Bo | from | Blautia obeum |
| | | | | ATCC 29174 |

MTKNLLLGIAAVCGSTFQAVACTAATYKTKDFYFGRTLDYEFSYGDQIVITPRNYAFNFRHVGDMKNHYAIIGMAHVAEDYPLYYDAMN
EKGVAMAGLNFVGNAVYAAIKPDVENIAQFEFIPWILSQCSSLVEVRELLERINIVNTPFSEQLPLAQLHWIISDENESITVESMSDGL
HIYDNPVGVLTNNPPFPQQMFQLNNYMYLSPKQPRNTFCENLALDAYSRGMGGLGLPGDLSSSSRFVRVAFTKVNAISGESEEESVSQF
FHILGSVDQQRGCCEVADGKYEITLYTSCCNVTKGIYYYNTYENHQISAVDMHVENLDSDKMICYPVIQGERINYQNK*

| | | | | |
|---|---|---|---|---|
| SEQ ID NO: 86 | DNA coding sequence of | Blo_ATTC_BSH_co_SP-Bo | from | Blautia obeum |
| | | | | ATCC 29174 |

ATGACCAAGAACTTGTTGTTGGGTATTGCTGCTGTTTGTGGTTCTACTTTTCAAGCTGTTGCTTGTACTGCTGCTACTTACAAAACTAA
GGATTTCTACTTCGGTAGGACCTTGGATTACGAATTTTCTTACGGTGATCAAATCGTCATCACCCCAAGAAATTACGCTTTCAACTTTA
GACACGTTGGCGATATGAAGAACCATTACGCTATTATTGGTATGGCTCATGTTGCTGAAGATTACCCCTTGTATTATGATGCCATGAAC
GAAAAAGGTGTTGCTATGGCTGGTTTGAATTTTGTTGGTAATGCTGTTTACGCTGCCATCAAACCAGATGTTGAAAACATTGCCCAGTT
CGAATTCATTCCATGGATCTTGTCTCAATGCTCCTCATTGGTTGAAGTTAGAGAACTGTTGGAAAGGATCAACATCGTTAACACTCCAT
TCTCTGAACAATTGCCATTGGCTCAATTGCATTGGATTATCTCTGACGAAAACGAATCCATCACCGTCGAATCTATGTCTGATGGTTTA
CACATCTACGATAACCCAGTTGGTGTTTTGACTAACAATCCACCATTTCCACAACAGATGTTCCAGTTGAACAACTACATGTACTTATC
TCCAAAGCAGCCCAGAAACACTTTCTGTGAAAATTTGGCTTTGGACGCTTACTCTAGAGGTATGGGTGGTTTGGGTTTGCCAGGTGATT
TGTCATCTTCTTCAAGATTCGTTAGAGTTGCCTTCACTAAGGTTAACGCTATTTCTGGTGAATCCGAAGAAGAATCCGTTTCTCAATTC
TTCCATATCTTGGGTTCTGTTGACCAACAAAGAGGTTGTTGCGAAGTTGCTGATGGTAAATACGAAATTACCTTGTACACCTCTTGTTG
CAACGTTACTAAGGGTATCTACTACTACAACACCTACGAAAACCATCAAATCTCCGCTGTTGATATGCACGTTGAAAACTTGGATTCCG
ATAAGATGATTTGCTACCCAGTTATTCAGGGTGAGAGAATCAATTACCAGAACAAGTGA

| | | | | |
|---|---|---|---|---|
| SEQ ID NO: 87 | protein sequence of | Ca_ATTC_BSH_co_SP-Bo | from | Collinsella |
| | | | | aerofaciens |
| | | | | ATCC 25986 |

MTKNLLLGIAAVCGSTFQAVAYLGRNLDWSFDYGQQVRVMPRGFHIPYSFIDDATAAHAVIGMCIDYRNYPMFFDCGNDAGLAVAGLNF
PGYAEYAEGPVDGKTNIAAYEFPLWVAATFSTVDEVEAALRDTVIVAKSAGEGLGVSLLHWIIGDSQRSIVVEVMADGLHVYDDPVDTL
ANQPTFPWHMENLRTYITATSDFPQTATWRGAELKPYGAGAGMRGIPGDCYSPSRFVKAAYLNANYPQKESETENVVRMFRSLEGVVMI
EGASRMGDGKFEKTIYTGCFSARTGNYYYAMYGDPSIRYVSLMDAEGASPDRLVVPEPRRS*

| | | | | |
|---|---|---|---|---|
| SEQ ID NO: 88 | DNA coding sequence of | Ca_ATTC_BSH_co_SP-Bo | from | Collinsella |
| | | | | aerofaciens |
| | | | | ATCC 2S986 |

ATGACCAAGAACTTGTTGTTGGGTATTGCTGCTGTTTGTGGTTCTACTTTTCAAGCTGTTGCTTACTTGGGTAGAAACTTGGATTGGTC
TTTTGACTACGGTCAACAAGTTAGAGTTATGCCAAGAGGTTTCCACATTCCATACTCATTCATTGATGATGCTACTGCTGCTCATGCTG
TTATTGGTATGTGTATCGATTACAGAAACTACCCCATGTTCTTCGATTGTGGTAATGATGCTGGTTTGGCTGTTGCAGGTTTGAATTTT
CCAGGTTATGCTGAATACGCTGAAGGTCCAGTTGATGGTAAAACTAACATTGCTGCTTACGAATTCCCATTGTGGGTTGCTGCTACTTT
TTCTACTGTTGATGAAGTTGAAGCTGCCTTGAGAGATACTGTTATCGTTGCTAAATCTGCTGGTGAAGGTTTGGGTGTTTCTTTGTTGC
ATTGGATTATCGGTGACTCCCAAAGATCTATCGTTGTTGAAGTTATGGCCGATGGTTTACACGTTTATGATGATCCTGTTGATACTTTG
GCTAACCAGCCAACTTTTCCATGGCATATGGAAAACTTGAGGACCTACATTACTGCTACCTCTGATTTTCCACAAACTGCTACTTGGAG
AGGTGCTGAATTGAAACCATACGGTGCTGGTGCTGGTATGAGAGGTATTCCAGGTGATTGCTATTCTCCATCTAGATTTGTTAAGGCTG
CCTACTTGAATGCTAACTACCCACAAAAAGAATCCGAAACCGAAAACGTTGTCAGAATGTTCAGATCATTGGAAGGTGTTGTTATGATT
GAAGGTGCTTCTAGAATGGGTGACGGTAAATTTGAAAAGACTATCTACACCGGTTGCTTCTCTGCTAGAACTGGTAATTACTATTACGC
CATGTATGGTGACCCATCCATTAGATACGTTTCTTTGATGGATGCTGAAGGTGCATCTCCAGATAGATTGGTTGTTCCAGAACCTAGAA
GATCCTGA

| | | | | |
|---|---|---|---|---|
| SEQ ID NO: 89 | protein sequence of | Ba_L2-32_BSH_co_SP-Bo | from | Bifidobacterium |
| | | | | adolescentis |
| | | | | L2-32 |

MTKNLLLGIAAVCGSTFQAVAFIRRLKPWLVQPSMRMMNAPGQSNKGRKSIMCTGVRFSDEEGNMYFGRNLDWSFSYGESILATPRGYH
YDNVFGASGKATPNAVIGVGVVMADRPMYFDCANEHGLAIAGLNFPGYAEFVHEPVEGTDNVATFEFPLWVARNFDSVDEVEEALKNVT
LVSQIVPGQQESLLHWIIGDSERSIWEQMADGMHVHHDDVDVLTNQPTFGFHMENLRNYMCVGNEMAEPATWGKASLSAWGAGVSMHGI
PGDVSSPSRFVRVAYANTHYPQQEGEAANVSRLFHTLGSVQMVDGMAKMGNGQFERTLFTSGYSSKTNTYYMNTYDDPAIRSYAMADFD
MDSSELITAA*

| | | | | |
|---|---|---|---|---|
| SEQ ID NO: 90 | DNA coding sequence of | Ba_L2-32_BSH_co_SP-Bo | from | Bifidobacterium |
| | | | | adolescentis |
| | | | | L2-32 |

ATGACCAAGAACTTGTTGTTGGGTATTGCTGCTGTTTGTGGTTCTACTTTTCAAGCTGTTGCCTTCATTAGAAGATTGAAGCCATGGTT
GGTTCAGCCATCTATGAGAATGATGAATGCTCCAGGTCAATCTAACAAGGGTAGAAAGTCTATTATGTGCACCGGTGTTAGATTCTCTG
ATGAAGAGGGTAATATGTACTTCGGTAGAAACTTGGATTGGTCCTTTTCTTACGGTGAATCTATTTTGGCTACTCCAAGAGGTTACCAC
TACGATAATGTTTTTGGTGCTTCTGGTAAAGCTACCCCAAATGCTGTTATTGGTGTTGGTGTTGTTATGGCTGATAGACCAATGTACTT
TGATTGCGCTAATGAACATGGTTTGGCTATTGCTGGTTTGAATTTTCCAGGTTACGCTGAATTTGTTCACGAACCAGTTGAAGGTACTG
ATAACGTTGCTACTTTTGAATTCCCATTGTGGGTTGCTAGAAACTTCGATAGTGTTGATGAAGTTGAAGAGGCCTTGAAGAACGTTACT
TTGGTTTCTCAAATTGTCCCAGGTCAGCAAGAATCTTTGTTGCATTGGATTATCGGTGACTCCGAAAGATCTATCGTTGTTGAACAAAT
GGCTGATGGTATGCATGTTCATCACGATGATGTTGATGTTTTGACTAACCAGCCAACTTTCGGTTTCCACATGGAAAACTTGAGAAACT
ACATGTGCGTTGGTAACGAAATGGCAGAACCAGCTACTTGGGGTAAAGCTTCTTTGTCTGCTTGGGGTGCTGGTGTTTCTATGCATGGT
ATTCCAGGTGATGTTTCTTCACCATCTAGATTCGTTAGAGTTGCTTACGCTAATACTCACTACCCTCAACAAGAGGGTGAAGCTGCTAA
TGTTTCTAGGTTGTTTCATACCTTGGGTTCCGTTCAAATGGTAGACGGTATGGCTAAAATGGGTAATGGTCAATTCGAAAGAACCTTGT
TCACTTCCGGTTACTCTTCTAAGACTAACACCTACTACATGAACACCTATGATGATCCAGCCATTAGATCTTATGCTATGGCCGATTTT
GATATGGACTCCTCTGAATTGATTACCGCTGCTTAA

| | | | | |
|---|---|---|---|---|
| SEQ ID NO: 91 | protein sequence of | Bt_BSH_co | from | Bacteroides |
| | | | | thetaiotaomicron |

MKKKLTGVALVLAAVSLMGIQPAGACTRAVYLGPDRMVVTGRTMDWKEDIMSNIYVFPRGMQRAGHNKEKTVNWTSKYGSVIATGYDIG
TCDGMNEKGLVASLLFLPESVYSLPGDTRPAMGISIWTQYVLDNFATVREAVDEMKKETFRIDAPRMPNGGPESTLHMAITDETGNTAV
IEYLDGKLSIHEGKEYQVMTNSPRYELQLAVNDYWKEVGGLQMLPGTNRSSDRFVRASFYIHAIPQTADAKIAVPSVLSVMRNVSVPFG
INTPEKPHISSTRWRSVSDQKNKVYYFESTLTPNLFWLDLKKIDFSPKAGVKKLSLTKGEIYAGDAVKDLKDSQSFTFLFETPVM*

| | | | | |
|---|---|---|---|---|
| SEQ ID NO: 92 | DNA coding sequence of | Bt_BSH_co | from | Bacteroides |
| | | | | thetaiotaomicron |

ATGAAGAAGAAGTTGACTGGCGTTGCTTTGGTTTTGGCTGCTGTTTCTTTGATGGGTATTCAACCTGCTGGTGCTTGTACTAGAGCTGT
TTATTTGGGTCCAGATAGAATGGTTGTTACTGGTAGAACTATGGATTGGAAAGAGGACATCATGTCCAACATCTACGTTTTTCCAAGAG
GTATGCAAAGAGCCGGTCATAACAAAGAAAAGACTGTTAACTGGACCTCCAAGTACGGTTCTGTTATTGCTACTGGTTACGATATCGGT
ACTTGTGATGGTATGAACGAAAAAGGTTTGGTTGCCTCTTTGTTGTTCTTGCCAGAATCTGTTTATTCTTTGCCAGGTGATACAAGACC
AGCTATGGGTATTTCTATTTGGACTCAATACGTCTTGGATAACTTCGCTACTGTTAGAGAAGCTGTTGACGAAATGAAGAAAGAAACCT
TCAGAATTGATGCCCCAAGAATGCCAAATGGTGGTCCAGAATCTACATTGCATATGGCTATTACTGACGAAACTGGTAACACCGCTGTT
ATCGAATATTTGGACGGTAAATTGTCCATCCACGAGGGTAAAGAATACCAAGTTATGACTAACTCCCCAAGGTATGAATTACAATTGGC
CGTTAACGACTACTGGAAAGAAGTTGGAGGTTTACAAATGTTGCCAGGCACTAATAGATCCTCCGATAGATTTGTTAGAGCCTCCTTTT
ACATTCACGCTATTCCACAAACTGCTGATGCTAAAATTGCTGTTCCATCCGTTTTGTCCGTTATGAGAAATGTTTCTGTTCCATTCGGT
ATCAACACTCCAGAAAAGCCACATATTTCTTCTACTAGATGGCGTTCCGTTTCCGATCAAAAAAACAAGGTTTACTACTTCGAGTCCAC
TTTGACTCCAAATTTGTTCTGGTTGGACTTGAAGAAGATCGACTTTTCACCAAAAGCTGGTGTCAAGAAATTGTCTTTGACTAAGGGTG
AAATCTACGCTGGTGATGCTGTTAAGGATTTGAAGGATTCTCAATCCTTCACCTTCTTGTTCGAAACTCCAGTTATGTAA

| | | | | |
|---|---|---|---|---|
| SEQ ID NO: 93 | protein sequence of | Bc_BSH_co | from | Bacteroides |
| | | | | caecimuris |

MMKMTKNLLLGIAAVCGSTFQAVACTGISLTSRDGSYVQARTIEWARGVLQSEYVIIPRGQQLTSFTPTGVNGLTFTAKYGVVGLTVVQ
KEFIAEGINEAGLSAGLFFFPHYGGYETYDAAQNRRTLADLQVTEWMLSQFSTIDEVKAALSSVRVVGLEKTAVVHWRIGEPSGRQVVL
EIVGGVPHFYENGVGVLTNAPGFEWQLTNLNNYVNLYPGDASVRKLSGITLQPTGSNSGFLGIPGDATPPSRFVRAAFYRGTAPQRATG
FDTVQQCFHLLNNFDIPIGIEYSQVGDIPDIPSATQWTSAIDLTNRKVYYKTAYNNSIRCIDLKAIDFSKVKYQSQPLDKIQEQPIEMV
KIPR*

| | | | | |
|---|---|---|---|---|
| SEQ ID NO: 94 | DNA coding sequence of | Bc_BSH_co | from | Bacteroides |
| | | | | caecimuris |

ATGATGAAGATGACCAAGAACCTGTTGTTGGGTATTGCTGCTGTTTGTGGTTCTACTTTTCAAGCTGTTGCTTGTACCGGTATTTCTTT
GACTTCTAGAGATGGTTCTTACGTTCAAGCCAGAACTATTGAATGGGCTAGAGGTCTTTTACAGTCCGAATACGTTATTATCCCAAGAG
GTCAACAATTGACTTCTTTCACTCCAACTGGTGTTAACGGTTTGACTTTTACTGCTAAGTATGGTGTTGTTGGTTTGACCGTTGTCCAG
AAAGAATTCATTGCTGAAGGTATTAACGAAGCTGGTTTGTCTGCTGGTTTATTCTTTTTTCCACATTACGGTGGTTACGAAACTTATGA
TGCTGCTCAAAACAGAAGAACTTTGGCCGACTTACAAGTTACTGAATGGATGTTGTCTCAGTTCTCCACTATCGATGAAGTTAAGGCTG
CTTTGTCATCCGTTAGAGTTGTAGGTTTGGAAAAGACTGCTGTTGTTCATTGGAGAATTGGTGAACCATCTGGTAGACAAGTTGTTTTG
GAAATAGTTGGTGGTGTTCCACACTTTTACGAAAATGGTGTTGGTGTCTTGACTAATGCTCCAGGTTTTGAATGGCAATTGACCAACTT
GAACAACTACGTTAACTTGTATCCAGGTGATGCCTCTGTTAGAAAGTTGTCTGGTATTACATTGCAACCTACCGGTTCTAATTCTGGTT
TTTTGGGTATTCTTGGTGATGCTACTCCACCATCTAGATTTGTTAGAGCTGCTTTTTACAGAGGTACTGCTCCACAAAGAGCTACTGGT
TTTGATACTGTTCAACAATGCTTCCACCTGTTGAACAATTTCGATATTCCAATCGGTATCGAGTACTCCCAAGTTGGTGATATTCCAGA
TATTCCTTCTGCTACTCAATGGACCTCTGCTATTGATTTGACTAACAGAAAGGTCTACTACAAGACCGCTTACAACAACTCCATTAGAT
GCATCGATTTGAAGGCCATCGATTTCTCTAAGGTCAAGTATCAATCTCAGCCATTGGACAAGATTCAAGAACAGCCAATTGAAATGGTC
AAGATCCCAAGATGA

| | | | | |
|---|---|---|---|---|
| SEQ ID NO: 95 | protein sequence of | Aa_BSH_co | from | Alistipes sp. An66 |

MKLKYGLLGVAALCGTMQALACTGISLTAADGSYVQSRTIEWGSSALESMYVVIPRGQQLRSLTPTGGQGLAYTARYGVVGLAVVEQAF
IAEGINEAGLSAGLFFFPQYGGYETYDPAQEARTLVDLQVAGWILTQFSTIDEVKAAIGKVRIVGLEKNAVVHWRIGEPSGRQVVLEIV
GGVPHFYENEVGVLTNAPGFEWQVTNLNNYVNLRPGEVEPYQVGEKTLRAFGATAGFLGLPGDDTPPSRFVRAAFFRATAPQRATAFET
VQECFHLLNNFDVPIGLEHPAGQCPDIPSATQWTSAIDLTHRTVYYKTAYNNTIRCIDLSQIDFGKVRYQSHPLDRVQQQPVERITVK*

| | | | | |
|---|---|---|---|---|
| SEQ ID NO: 96 | DNA coding sequence of | Aa_BSH_co | from | Alistipes sp. An66 |

ATGAAGCTGAAGTACGGTTTGTTGGGTGTTGCTGCTTTGTGTGGTACTATGCAAGCTTTGGCTTGTACTGGTATTTCTTTGACTGCTGC
TGATGGTTCTTACGTTCAATCTAGAACTATTGAGTGGGGTTCTTCTGCTTTGGAATCTATGTATGTTGTTATCCCAAGAGGTCAGCAGT
TGAGATCTTTGACTCCAACTGGTGGTCAAGGTTTAGCTTATACTGCTAGATATGGTGTTGTTGGTTTGGCCGTTGTTGAACAAGCTTTT
ATTGCTGAAGGTATTAACGAAGCTGGTTTGTCTGCTGGTTTATTCTTTTTTCCACAATACGGTGGTTACGAAACTTACGATCCAGCTCA
AGAAGCTAGAACTTTGGTTGACTTACAAGTTGCTGGTTGGATTTTGACTCAATTCTCCACTATCGATGAAGTTAAGGCTGCTATTGGTA
AGGTTAGAATTGTCGGTTTGGAAAAGAACGCTGTTGTTCATTGGAGAATAGGTGAACCATCTGGTAGACAAGTTGTTTTGGAAATAGTT
GGTGGTGTTCCACACTTCTACGAAAATGAAGTTGGTGTTTTGACAAACGCTCCAGGTTTTGAATGGCAAGTTACCAATTTGAACAACTA
CGTTAATTTGAGGCCAGGTGAAGTTGAACCATATCAAGTTGGTGAAAAGACCTTGAGAGCTTTTGGTGCTACAGCTGGTTTTTTGGGTT
TGCCAGGTGATGATACTCCACCATCTAGATTTGTTAGAGCTGCTTTTTTTAGAGCTACCGCTCCACAAAGAGCrACTGCTTTTGAAACT
GTTCAAGAATGCTTCCACTTGCTGAACAATTTCGATGTTCCAATTGGTTTAGAACATCCAGCTGGTCAATGTCCAGATATTCCATCTGC
TACTCAATGGACTTCTGCTATTGATTTGACTCACAGAACCGTTTACTACAAGACTGCTTACAACAACACTATCAGATGCATCGACTTGT
CCCAAATTGATTTCGGTAAAGTCAGATACCAATCTCACCCATTGGATAGAGTCCAACAACAACCAGTTGAAAGAATCACCGTTAAGTAA

| | | | | |
|---|---|---|---|---|
| SEQ ID NO: 97 | protein sequence of | Bvu_BSH_co | from | Bacteroides |
| | | | | vulgatus |
| | | | | (strain |
| | | | | ATCC 8482/ |
| | | | | DSM 1447/ |
| | | | | JCM 5826/ |
| | | | | NBRC 14291/ |
| | | | | NCTC 11154) |

MKKILIALALLLTGIASGSACTGISFLAEDGGYVQARTIEWGNSYLPSEYVIVPRGQDLVSYTPTGVNGLRFRAKYGLVGLAIIQKEFV
AEGLNEVGLSAGLFYFPHYGKYEEYDEAQNAITLSDLQVVNWMLSQFATIDEVREAIEGVKVVSLDKPGKSSTVHWRIGDAKGNQMVLE
FVGGVPYFYENKVGVLTNSPDFPWQVINLNNYVNLYPGAVTPQQWGGVTIFPFGAGAGFHGIPGDVTPPSRFVRVAFYKATAPVCPTAY
DAILQSFHILNNFDIPIGIEYALGKAPDIPSATQWTSAIDLTNRKVYYKTAYNNNIRCISMKKIDFDKVKYQSYPLDKELKQPVEEIIV
K*

| | | | | |
|---|---|---|---|---|
| SEQ ID NO: 98 | DNA coding sequence of | Bvu_BSH_co | from | Bacteroides |
| | | | | vulgatus |
| | | | | (strain |
| | | | | ATCC 8482/ |
| | | | | DSM 1447/ |
| | | | | JCM 5826/ |
| | | | | NBRC 14291/ |
| | | | | NCTC 11154) |

ATGAAGAAGATCTTGATCGCCTTGGCTTTGTTGTTGACTGGTATTGCTTCTGGTTCTGCTTGTACTGGTATTTCATTTTTGGCTGAAGA
TGGTGGTTACGTTCAAGCTAGAACTATTGAATGGGGTAACTCTTACTTGCCATCCGAATATGTTATAGTCCCAAGAGGTCAAGACTTGG
TTTCTTATACTCCAACTGGTGTTAACGGTTTGAGATTCAGAGCTAAATATGGTTTGGTTGGTTTGGCCATCATCCAGAAAGAATTTGTT
GCTGAAGGTTTGAACGAAGTTGGTTTGTCTGCTGGTTTGTTTTACTTTCCACACTACGGTAAATACGAAGAATACGATGAAGCTCAAAA
CGCCATTACTTTGTCCGACTTACAAGTTGTTAACTGGATGTTGTCTCAATTCGCCACCATTGATGAAGTTAGAGAAGCTATTGAAGGTG
TCAAGGTTGTTTCTTTGGATAAGCCAGGTAAATCGTCTACTGTTCATTGGAGAATTGGTGATGCTAAGGGTAATCAAATGGTCTTGGAA
TTCGTTGGTGGTGTTCCATACTTTTACGAAAACAAGGTTGGTGTCTTGACCAACTCTCCAGATTTTCCATGGCAAGTTATCAACTTGAA
CAACTACGTTAACTTGTACCCAGGTGCTGTTACTCCACAACAATGGGGTGGTGTTACTATTTTTCCATTTGGTGCTGGTGCAGGTTTTC
ATGGTATTCCAGGTGATGTAACTCCTCCATCTAGATTTGTTAGAGTTGCTTTCTACAAAGCTACCGCTCCAGTTTGTCCAACTGCTTAT
GATGCTATCTTGCAATCCTTCCACATCCTGAACAATTTCGATATTCCAATCGGTATCGAATACGCTTTGGGTAAAGCTCCAGATATTCC
TTCTGCTACTCAATGGACTTCTGCTATTGATTTGACCAACAGAAAGGTCTACTACAAGACTGCTTACAACAACAACATTAGGTGCATCT
CCATGAAGAAAATCGACTTCGATAAGGTCAAGTACCAGTCTTATCCATTGGACAAAGAATTGAAGCAACCAGTCGAAGAAATCATCGTC
AAGTGA

| | | | | |
|---|---|---|---|---|
| SEQ ID NO: 99 | protein sequence of | Ma_BSH_co | from | Muribaculum |
| | | | | sp. An287 |

MKTSLILKSALVSAVAALSFHLSASACTGISFRAADGSVVLARTIEWGDSELPSMYVIVPRGMEFISYTPTGQNGMKFKVKYGYAGVSV
VKDEFVAEGLNEAGLSAGLFYFPGYGKYPEYKEAYNGRTIADLQFVSWMLSSFATVDELLKELDRVRLVAIEGTSTVHWRVGDATGRQI
VIEIVDGRTNVYENTLGVLTNAPGFEWQVVNLNNYVNLYSGGAQPKTVDGVLIHPFGAGSGMLGIPGDVTPPSRFVRAFFYKTTAPQQK
TGRDCVMQCFHILNNFDLPVGVEHEPGKSPDIPSATQWTAASDLTGKKLYFRTMYNSSIRCIDLSAIDFSTVEYVSRPLDEAKEEPVEM
LQV*

| | | | | |
|---|---|---|---|---|
| SEQ ID NO: 100 | DNA coding sequence of | Ma_BSH_co | from | Muribaculum |
| | | | | sp. An287 |

ATGAAGACCTCCTTGATCTTGAAGTCCGCTTTGGTTTCTGCTGTTGCTGCTTTGTCTTTTGATTTGTCTGCTTCAGCTTGTACCGGTAT
TTCTTTTAGAGCTGCTGATGGTTCTGTTGTTTTGGCTAGAACTATTGAATGGGGTGATTCTGAATTGCCATCCATGTATGTTATAGTCC
CAAGAGGTATGGAATTCATCTCTTATACTCCAACTGGTCAGAACGGTATGAAGTTCAAAGTTAAGTATGGTTACGCCGGTGTTTCCGTT
GTTAAGGATGAATTTGTTGCTGAAGGTTTGAACGAAGCTGGTTTATCTGCTGGTTTGTTTTACTTTCCAGGTTACGGTAAATACCCCGA
GTACAAAGAAGCTTATAACGGTAGAACAATTGCCGACTTGCAATTCGTTTCTTGGATGTTGTCATCTTTCGCTACTGTTGACGAGTTGT
TGAAAGAATTGGATAGAGTTAGATTGGTCGCCATCGAAGGTACTTCTACTGTTCATTGGAGAGTTGGTGATGCTACTGGTAGACAAATC
GTTATCGAAATCGTTGATGGTAGGACTAACGTTTACGAAAACACTTTGGGTGTTTTGACTAATGCTCCAGGTTTTGAATGGCAAGTCGT
TAACTTGAACAATTACGTCAACTTGTATTCTGGTGGTGCTCAACCTAAAACTGTTGATGGTGTTTTAATCCATCCATTCGGTGCTGGTT
CTGGTATGTTGGGTATTCCAGGTGATGTTACTCCACCATCTAGATTTGTTAGAGCCTTCTTCTACAAAACTACCGCTCCACAACAAAAG
ACTGGTAGAGATTGTGTTATGCAATGCTTCCACATCTTGAACAACTTCGATTTGCCAGTTGGTGTTGAACATGAACCAGGTAAATCTCC
AGATATTCCATCTGCTACTCAATGGACTGCTGCTTCTGATTTGACTGGTAAGAAGTTGTACTTCAGGACCATGTACAACTCCTCCATTA
GATGCATTGACTTGTCCGCTATTGATTTCTCTACCGTCGAATATGTTTCCAGACCATTGGATGAAGCTAAAGAAGAACCAGTCGAAATG
TTGCAAGTCTAA

| | | | | |
|---|---|---|---|---|
| SEQ ID NO: 101 | protein sequence of | BCAG_BSH_co | from | Bacteroides |
| | | | | sp. CAG:633 |

MKSRILLAIAMGIGSMFAYPLPAEACTGITLKSKDSVTVVARTIEWGGSDLNSQYVIVPRGYEQRSYTPAGVTGMTFKARYGYVGLAVE
QKDFVAEGLNEAGLSAGLFYFPNYGEYEKFDEALQDKSIADLQLVSWILGQCATVDEVKQAVSEVHIINIDPRASTVHWRFTEPSGRQI
VLEMIDGKPKFYENPLGVLTNSPSFDWQLTNLNNYVNLYPGTAPAQSLGGAKLASFGAGSGFLGIPGDVTPPSRFVRAAFYQATAPVQD
TALKTVLQGFQILNNFDIPVGIEFQAGKIPADIPSATQWTSATDMADRIIYYRTMYNSAIRSIDLKTIDFSKVKYRAVPLDAEKQQPIV
SVKIANK*

| | | | | |
|---|---|---|---|---|
| SEQ ID NO: 102 | DNA coding sequence of | BCAG_BSH_co | from | Bacteroides |
| | | | | sp. CAG:633 |

ATGAAGTCCAGAATCTTGTTGGCTATTGCCATGGGTATTGGTTCTATGTTTGCTTATCCATTGCCAGCTGAAGCTTGTACTGGTATTAC
TTTGAAGTCCAAGGATTCCGTTACTGTTGTTGCTAGAACTATTGAATGGGGTGGTTCTGATTTGAACTCCCAATATGTTATAGTCCCAA
GAGGTTACGAACAGAGATCTTATACTCCAGCTGGTGTTACAGGTATGACTTTTAAAGCTAGATACGGTTACGTTGGTTTGGCCGTTGAA
CAAAAAGATTTTGTTGCTGAAGGTTTGAACGAAGCTGGTTTGTCTGCTGGTTTATTCTACTTTCCAAACTACGGTGAATACGAGAAGTT
TGATGAAGCCTTGCAAGATAAGTCTATTGCCGACTTGCAATTGGTTTCTTGGATTTTGGGTCAATGTGCTACTGTTGATGAAGTTAAGC
AAGCCGTTTCCGAAGTTCATATCATTAACATTGATCCAAGAGCCTCTACTGTCCATTGGAGATTCACTGAACCATCTGGTAGACAAATC
GTCTTGGAAATGATTGATGGTAAGCCAAAGTTCTACGAAAACCCATTGGGTGTTTTGACTAACTCTCCATCTTTTGATTGGCAGTTGAC
CAACTTGAACAACTACGTTAACTTGTATCCAGGTACTGCTCCAGCTCAATCTTTAGGTGGTGCTAAATTGGCTTCTTTTGGTGCTGGTT
CTGGTTTTTTGGGTATTCCAGGTGATGTTACTCCACCATCTAGATTTGTTAGAGCTGCTTTTTATCAAGCTACCGCTCCAGTTCAAGAT
ACTGCTTTGAAAACTGTGTTGCAAGGTTTCCAGATCCTGAACAATTTCGATATTCCAGTCGGTATTGAATTCCAAGCTGGTAAAATTCC
AGCTGATATTCCATCTGCTACTCAATGGACTTCTGCTACTGATATGGCTGATAGAATCATCTACTACAGGACCATGTACAACTCCGCCA
TTAGATCCATTGATTTGAAAACCATCGACTTCTCCAAGGTTAAGTATAGAGCTGTTCCATTGGATGCTGAAAAGCAACAACCTATCGTT
TCCGTTAAGATTGCCAACAAGTGA

| | | | | |
|---|---|---|---|---|
| SEQ ID NO: 103 | protein sequence of | Bvi_BSH_co | from | Barnesiella |
| | | | | viscericola |
| | | | | DSM 18177 |

MKRLFLLGIAAMLLSTNTFSWACTGITLTAKDGSKIVARTIEWGGSDLNSQYVIVPRGYQQQSFIPGGEQTGLKFTARYGYVGLAVEQK
EFIAEGLNEAGLSAGLFYFPGYGQYEAYNPTHQATSIADLQLVAWILGSFSTIDEAVKGLKTVHVVSIDPRASTVHWRMTDVSGRQVVL
EIVGGQLNFYENKLGVLTNSPGFEWQITNLNNYINLYPGTTDTKSFGDITLSSFGAGSGFIGLPGDITPPSRFVRAAFYQTTAPEQNDA
IGAVTQSFHILNNFDIPIGAEFLAGQTPTDIPSATQWTSSADVTHRVLYYRTMYNSAIRSFDLKSIDFGRVKYQVQPLDVNKQQPIQTI
SIK*

| | | | | |
|---|---|---|---|---|
| SEQ ID NO: 104 | DNA coding sequence of | Bvi_BSH_co | from | Barnesiella |
| | | | | viscericola |
| | | | | DSM 18177 |

ATGAAGAGGTTGTTCTTGTTGGGTATTGCTGCTATGTTGTTGTCTACCAATACTTTCTCTTGGGCTTGTACTGGTATTACTTTGACTGC
TAAAGACGGTTCTAAGATCGTTGCTAGAACTATTGAATGGGGTGGTTCTGATTTGAACTCCCAATATGTTATAGTCCCAAGAGGTTACC
AACAGCAATCTTTTATTCCAGGTGGTGAACAAACTGGTTTGAAGTTTACTGCTAGATACGGTTATGTTGGTTTGGCCGTTGAACAGAAA
GAATTCATTGCTGAAGGTTTGAACGAAGCTGGTTTGTCTGCTGGTTTATTCTATTTTCCAGGTTACGGTCAATACGAGGCTTACAATCC
AACTCATCAAGCTACTTCTATTGCCGACTTGCAATTGGTTGCTTGGATTTTGGGTTCTTTCTCCACTATTGATGAAGCCGTTAAGGGTT
TGAAAACCGTTCATGTTGTTTCCATTGATCCAAGAGCTTCTACTGTTCATTGGAGAATGACTGATGTTTCCGGTAGACAAGTTGTTTTG
GAAATAGTTGGTGGTCAGTTGAACTTCTACGAAAACAAATTGGGTGTCTTGACTAACTCTCCAGGTTTCGAATGGCAAATTACCAACTT
GAACAACTACATCAACTTGTACCCAGGTACTACTGATACCAAGTCTTTCGGTGATATTACCTTGTCATCTTTTGGTGCTGGTTCTGGTT
TTATTGGTTTGCCAGGTGACATTACTCCACCATCTAGATTTGTTAGAGCTGCTTTCTATCAAACTACCGCTCCAGAACAAAATGATGCT
ATTGGTGCTGTTACCCAATCCTTCCATATCTTGAACAATTTCGATATTCCAATCGGTGCTGAATTCTTGGCTGGTCAAACTCCAACTGA
TATTCCATCTGCTACTCAATGGACTTCTTCTGCTGATGTTACTCATAGAGTCTTGTACTACAGAACCATGTACAACTCCGCTATCAGAT
CTTTCGATTTGAAGTCTATCGATTTCGGTAGAGTCAAGTACCAAGTTCAACCATTGGATGTTAACAAGCAACAGCCAATTCAAACCATC
TCCATCAAGTGA

| | | | | |
|---|---|---|---|---|
| SEQ ID NO: 105 | protein sequence of | Lho_BSH_co_SP-Bo | from | Lactobacillus |
| | | | | hominis |
| | | | | DSM 23910 = |
| | | | | CRBIP 24.179 |

MTKNLLLGIAAVCGSTFQAVACTSILYSPKDHYFGRNLDYEIAYGQKVVITPRNYEFEFTDLPAEKSHYAMIGVAAVADNTPLYCDAIN
EKGLGVAGLSFAGQGKYFPNAADKKNIASFEFISYLLATYETVDQVKESLTNANISNVSFAKNTPASELHWLVGDKTGKSIVVESDEKG
LHVYDNPVNALTNAPLFPEQLTNLANYASVVPGEPDNNFLPGVNLKLYSRSLGTHHLPGGMDSESRFVKVCFALNHAPKDSNEVENVTN
FFHILESVEQAKGMDQVGPNSFEYTMYTSCMNLEKGILYFNCYDDSRISAVDMNKEDLDSSDLVVYDLFKKQDINFVN*

| | | | | |
|---|---|---|---|---|
| SEQ ID NO: 106 | DNA coding sequence of | Lho_BSH_co_SP-Bo | from | Lactobacillus |
| | | | | hominis |
| | | | | DSM 23910 = |
| | | | | CRBIP 24.179 |

ATGACCAAGAACCTGTTGTTGGGTATTGCTGCTGTTTGTGGTTCTACTTTTCAAGCTGTTGCTTGTACCTCTATCTTGTACTCTCCAAA
GGATCATTACTTCGGTAGAAACTTGGATTACGAAATTGCCTACGGTCAAAAGGTTGTTATTACCCCAAGAAACTACGAGTTCGAATTCA
CTGATTTGCCAGCTGAAAAATCCCATTACGCTATGATTGGTGTTGCTGCAGTTGCTGATAATACCCCATTATATTGTGATGCCATCAAC
GAGAAAGGTTTGGGTGTTGCAGGTTTGTCTTTTGCTGGTCAAGGTAAATACTTTCCAAACGCTGCTGACAAGAAGAATATTGCCTCCTT
TGAATTCATCTCCTACTTGTTGGCTACTTACGAAACTGTTGACCAGGTCAAAGAATCCTTGACTAACGCTAACATCTCCAACGTTTCTT
TCGCTAAGAATACTCCAGCTTCTGAATTGCATTGGTTGGTTGGTGATAAGACTGGTAAGTCTATCGTTGTTGAATCTGACGAAAAAGGC
TTGCACGTTTACGATAATCCAGTTAACGCTTTGACTAATGCTCCTTTGTTTCCAGAACAATTGACCAACTTGGCTAACTACGCTTCTGT
TGTTCCAGGTGAACCAGATAACAATTTTTTGCCAGGTGTCAACCTGAAGTTGTACTCTAGATCTTTGGGTACTCATCATTTGCCTGGTG
GTATGGATTCTGAATCAAGATTCGTTAAGGTTTGCTTCGCTTTGAACCATGCTCCAAAAGATTCTAACGAAGTTGAAAACGTTACCAAC
TTCTTCCACATCTTGGAATCTGTTGAACAAGCCAAAGGTATGGATCAAGTAGGTCCAAATTCTTTCGAGTACACTATGTACACCTCTTG
CATGAATTTGGAGAAAGGCATCTTGTATTTCAACTGCTACGATGACTCTAGAATCTCCGCTGTTGATATGAACAAAGAGGATTTGGATT
CCTCCGATTTGGTTGTTTACGACTTGTTCAAGAAGCAGGACATTAACTTCGTCAACTAA

| | | | | |
|---|---|---|---|---|
| SEQ ID NO: 107 | protein sequence of | Lt_BSH_co_SP-Bo | from | Lactobacillus |
| | | | | tucceti |
| | | | | DSM 20183 |

MTKNLLLGIAAVCGSTFQAVANQKGVVAMCTSISYEALDGAKFLSRTMDFAFELNGRPTFLPRDYEWISSFDKKTYKADYAILGTGAKY
GNNYMVADGTNEYGLAAAELYFAHKAEYDHEPTDGAINLVSEEFILWALGNNKSLAELKDNIKNVHIIESDKGVMGKNQPLHWIFSDLT
GATYVIEPEGDGLILQEDKTGVMTNTPDYQWHETNLANYLGAQPNNFGSKKFGDLEVQPLGQNGTFRLPGGYTAVDRFVRTAFNREVIE
KAADATEAVNTIMHILDSVDVPKGANTKPDGPSYTQYQSIFDMTNRIMYYIPYGNRTVFKVRMTDEMIGAQKTPKEFPVALTQEFNE
LN*

| | | | | |
|---|---|---|---|---|
| SEQ ID NO: 108 | DNA coding sequence of | Lt_BSH_co_SP-Bo | from | Lactobacillus |
| | | | | tucceti |
| | | | | DSM 20183 |

ATGACCAAGAACCTGTTGTTGGGTATTGCTGCTGTTTGTGGTTCTACTTTTCAAGCTGTTGCTAATCAAAAGGGTGTTGTTGCTATGTG
CACCTCCATTTCTTATGAAGCTTTGGATGGTGCTAAGTTCTTGTCTAGAACTATGGATTTCGCCTTCGAATTGAATGGTAGACCAACTT
TTTTGCCAAGAGACTACGAATGGATTTCCAGCTTTGATAAGAAAACCTACAAGGCCGATTACGCCATTTTAGGTACTGGTGCTAAATAC
GGTAACAACTACATGGTTGCTGATGGCACTAATGAATATGGTTTGGCTGCTGCTGAATTATACTTTGCTCATAAGGCTGAATACGATCA
CGAACCTACTGATGGTGCAATCAATTTGGTTTCCGAAGAATTCATTTTGTGGGCCTTGGGTAACAACAAATCTTTGGCTGAATTGAAGG
ACAACATCAAGAACGTTCACATCATCGAATCTGATAAGGGTGTCATGGGTAAGAATCAACCATTGCATTGGATCTTCTCTGATTTGACA
GGTGCTACCTATGTTATTGAACCTGAAGGTGATGGTTTGATCTTACAAGAGGATAAGACTGGTGTTATGACTAACACTCCAGATTATCA
ATGGCACGAAACTAACTTGGCTAATTACTTGGGTGCTCAACCTAACAATTTCGGCTCTAAAAAGTTCGGTGATTTGGAAGTTCAACCCT
TGGGTCAAAATGGTACTTTTAGATTGCCAGGTGGTTACACTGCTGTTGATAGATTTGTTAGAACCGCCTTCAACAGAGAAGTTATTGAA
AAAGCTGCTGATGCTACCGAAGCTGTTAACACCATTATGCATATCTTGGATTCCGTTGATGTTCCAAAAGGTGCTAATACCAAACCAGA
TGGTCCATCTTATACCCAATACCAATCCATTTTCGACATGACCAACAGGATCATGTACTACATTCCATACGGTAATAGGACCGTTTTCA
AGGTTAGAATGACCGACGAAATGATTGGTGCTCAAAAGACTCCAAAAGAATTCCCAGTTGCTTTGACTCAAGAATTCAACGAGTTGAAC
TAA

| | | | | |
|---|---|---|---|---|
| SEQ ID NO: 109 | protein sequence of | Ts_BSH_co_SP-Bo | from | Turicibacter |
| | | | | sanguinis PC909 |

MTKNLLLGIAAVCGSTFQAVASSKLMFYFNRKMGLKNMCFGLSLVTKDNKHLFGRNLDVPATYGQAVHIVPRNYDWFNIVSDETYTSKY
ACIGMGIVVDRLPLLFDAVNEKGLAGAGLNFTHFAKFNEKAVDGKTNISGSTFLYWALSNFSNLDELKEALKQLIITNIPVKEGLPVAG
LHWMFTDLSGKSIVIEYMEDGMHVYDNPVGALTNDPTFPWHLTNLCQYVTLDTKTPAPKQFGEYVAKPFGHGLNMCGIPGDASPAARFV
RTVYFRDTVVEADDEVSGVSAFFQVLTGVTVIKGSEIDQNGDMNYTVYKSCMCQESGTYYYTDYKNRRISAVCLSKADLDGKEIISFEY
QGKQDILFQN*

| | | | | |
|---|---|---|---|---|
| SEQ ID NO: 110 | DNA coding sequence of | Ts_BSH_co_SP-Bo | from | Turicibacter |
| | | | | sanguinis PC909 |

ATGACCAAGAACCTGTTGTTGGGTATTGCTGCTGTTTGTGGTTCTACTTTTCAAGCTGTTGCTTCCTCTAAGCTGATGTTCTACTTCAA
TAGAAAGATGGGCTTGAAGAACATGTGTACCGGTTTGTCTTTGGTTACGAAAGATAACAAGCACTTGTTCGGTAGAAACTTGGATGTTC
CAGCTACTTATGGTCAAGCAGTTCATATCGTTCCAAGAAACTACGATTGGTTCAACATCGTTTCTGACGAAACTTACACTTCTAAGTAC
GCTTGTATTGGTATGGGTATCGTTGTTGATAGATTGCCTTTGTTGTTCGATGCCGTTAACGAAAAAGGTTTGGCTGGTGCTGGTTTGAA
TTTTACTCATTTCGCTAAGTTCAACGAAAAGGCCGTTGATGGTAAGACTAACATTTCTGGTTCTACGTTCTTGTACTGGGCTTTGTCTA
ATTTCTCTAACCTGGACGAATTGAAAGAAGCCTTGAAGCAATTGATCATCACCAACATTCCAGTCAAAGAAGGTTTGCCAGTTGCAGGT
TTACATTGGATGTTTACTGATTTGTCCGGTAAGTCCATCGTTATCGAGTATATGGAAGATGGTATGCACGTTTACGATAATCCAGTTGG
TGCTTTGACTAACGATCCAACTTTTCCATGGCATTTGACCAACTTGTGTCAATACGTTACTTTGGATACCAAAACTCCAGCTCCAAAGC
AATTTGGTGAATACGTTGCTAAACCATTCGGTCATGGTTTAAACATGTGTGGTATTCCAGGTGATGCTTCTCCAGCTGCTAGATTTGTT
AGAACTGTTTACTTCAGAGATACCGTTGTTGAAGCCGATGATGAAGTTTCTGGTGTTTCTGCATTTTTCCAAGTTTTGACTGGTGTTAC
CGTTATCAAGGGTTCCGAAATTGATCAAAACGGTGACATGAACTACACCGTTTACAAGTCTTGTATGTGCCAAGAATCTGGCACTTACT
ACTACACTGATTACAAGAACAGAAGAATCTCCGCTGTTTGTTTGTCTAAGGCTGATTTGGATGGTAAAGAGATCATCTCCTTTGAATAC
CAAGGTAAGCAAGACATCFTGTTCCAGAACTGA

| | | | | |
|---|---|---|---|---|
| SEQ ID NO: 111 | protein sequence of | Ls_bsh_co_SP-Bo | from | Lactobacillus sakei |
| | | | | subsp. sakei |
| | | | | (strain 23K) |

MTKNLLLGIAAVCGSTFQAVACTSLTYSTVDGHHFLARTMDFSFELNGNPLFLPRQHQWQPVLEKQAIQNQYALMGAGAKLGDQYLVAD
GFNEHGLGCAELYFAHAAVYEPQPVPNKLNLVAEEFIVWVLGNHQTLEEVAADLDNVRIIESDQGVMGANQPLHWILSDRSGKTMVIEP
RGNGLQLIDDPVGVMTNTPDLDWHIKNLSNYLNLQPQPFMERPFGNYQAGLFSQGTGTQALPGSYTPPDRFVRAAYSRQYMPEANNVAE
GVNHILHILDNVTIPKGVNIGSGGSSDYTQYQGISGLNNLAYYMVNYDNRHVYQTNLTTDLIENQKEPLIYHLPQGQQNTILN*

| | | | | |
|---|---|---|---|---|
| SEQ ID NO: 112 | DNA coding sequence of | Ls_BSH_co_SP-Bo | from | Lactobacillus sakei |
| | | | | subsp. sakei |
| | | | | (strain 23K) |

ATGACCAAGAACCTGTTGTTGGGTATTGCTGCTGTTTGTGGTTCTACTTTTCAAGCTGTTGCTTGTACTTCTTTGACCTACTCTACTGT
TGATGGTCATCATTTCTTGGCTAGAACTATGGACTTCTCCTTTGAATTGAATGGTAACCCTTTGTTCTTGCCAAGACAACATCAATGGC
AACCAGTTTTGGAAAAGCAAGCCATCCAAAATCAATACGCTTTGATGGGTGCTGGTGCTAAATTGGGTGATCAATATTTGGTTGCTGAC
GGTTTTAACGAACATGGTTTGGGTTGTGCTGAATTATACTTTGCTCATGCTGCAGTTTATGAACCACAACCAGTTCCAAACAAGTTGAA
TTTGGTAGCCGAAGAATTCATCGTTTGGGTTTTGGGTAATCACCAAACCTTGGAAGAAGTTGCTGCAGATTTGGATAACGTCAGAATCA
TCGAATCTGATCAAGGTGTTATGGGTGCTAATCAACCATTGCATTGGATCTTGTCTGATAGATCTGGTAAGACCATGGTTATTGAACCT
AGAGGTAACGGCTTGCAATTGATTGATGATCCAGTTGGTGTTATGACTAACACTCCTGATTTGGATTGGCACATCAAGAACTTGTCTAA
CTACTTGAACTTGCAACCACAGCCATTCATGGAAAGACCATTTGGTAATTATCAAGCCGGCCTATTTTCTCAAGGTACTGGTACTCAAG
CTTTGCCAGGTTCTTATACTCCACCAGATAGATTTGTTAGAGCTGCTTATTCTAGACAGTACATGCCrGAAGCTAACAATGTTGCTGAA
GGTGTTAATCACATCCTGCATATTTTGGACAACGTGACTATTCCAAAGGGTGTTAATATTGGTTCTGGTGGTTCTTCTGATTACACCCA
ATATCAAGGTATCTCCGGTTTGAACAATTTGGCCTACTACATGGTTAACTACGATAACAGACACGTTTACCAGACTAACTTGACCACCG
ATTTGATCGAAAATCAGAAAGAGCCATTGATCTACCATTTGCCACAAGGTCAACAAAACACCATCTTGAACTGA

### EXAMPLES

### Materials and Methods

Chemicals used in the examples herein, e.g. for buffers and substrates, are commercial products of at least reagent grade.

### Example 1

### Construction of Saccharomyces boulardii Strains for Production of BSH Enzyme

Kirkman (Kirkman Lab) and CNCM I-745 (Florastor®, Biocodex Inc) S. boulardii strains were modified to delete one or more of the native genes URA3, LEU2 and HIS3 in order to obtain auxotrophic mutants for the pathway insertion. URA3, LEU2 and HIS3 were deleted by removing the entire open reading frame (ORF) encoding the genes. The gRNA vectors were designed according to Vanegas et al. 2017, and were constructed using uracil excision reaction-based cloning (Mikkelsen et al. 2012). The donor fragments were designed with sequences upstream and downstream of the ORF to be deleted and constructed by PCR. These S. boulardii Biocodex and S. boulardii Kirkman auxotrophic strains were further modified to express a selection of codon-optimized bile salt hydrolases (BSH) and codon-optimized S. cerevisiae genes that have been shown to improve protein production/secretion as disclosed in Hou et al. 2012 and Huang et al. 2018. Bacterial BSH sequences were analyzed and identified using SignalP-5.0 (Almagro Armenteros et al., 2019) to detect putative bacterial secretion peptides that can be recognized in eukaryotes. If no bacterial pre-peptide was recognized (which is the case in all genes sourced from Gram-positive bacteria), the first methionine was replaced by the B. ovatus BSH pre-peptide (MTKNLLLGIAAVCGSTFQAVA).

An integration system, “Recombinator”, was used, which is similar to the S. cerevisiae gene integration and expression system developed by Mikkelsen et al., 2012, where either three or five expression cassettes with overlapping homology arms are integrated at site XI-5 with the well-known marker gene K. lactis LEU2 as selection marker for growth on media lacking leucine. K. lactis LEU2 is available e.g. from EUROSCARF (http://www.euroscarf.de). The integration system is build based on S. cerevisiae, so integration sites were used, which are highly conserved between S. cerevisiae and S. boulardii (Khatri et al., 2017). Genes were synthesized by Twist Bioscience, San Francisco, CA. A combination of the expression cassettes containing codon-optimized versions of the native genes encoding the S. cerevisiae subtilisin-like protease Kexin (a proprotein convertase) KEX2; YNL238W; SGD:S000005182 (Sc_KEX2_co-SEQ ID NO: 94), an S. cerevisiae non-chaperonin molecular chaperone ATPase KAR2; YJL034W; SGD:S000003571 (Sc_BiP_co-SEQ ID NO: 96), an S. cerevisiae disulfide bond isomerase PDI1; YCL043C; SGD:S000000548 (Sc_PDI_co-SEQ ID NO: 98), an S. cerevisiae basic leucine zipper (bZIP) transcription factor HAC1; YFL031W; SGD:S000001863 (Sc_HAC1_co-SEQ ID NO: 100), an S. cerevisiae plasma membrane t-SNARE SSO2; YMR183C; SGD:S000004795 (Sc_SSO2_co-SEQ ID NO: 102), an S. cerevisiae thiol oxidase ERO1; YML130C; SGD:S000004599 (Sc_ERO1_co-SEQ ID NO: 104), an S. cerevisiae component of the conserved oligomeric Golgi complex COGS; YNL051W; SGD:S000004996 (Sc_COGS_co-SEQ ID NO: 106), as disclosed in the Saccharomyces genome database (SGD) at www.yeastgenome.org, and a gene encoding the bile salt hydrolase of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, or 112 were integrated into the site XI-5 of the Biocodex S. boulardii strain auxotrophic for leucine described above.

The BSH genes were expressed using the well-known S. cerevisiae TDH3 promoter, Sc_KEX2_co/Sc_ERO1_co/Sc_SSO2_co/Sc_COG5_co were expressed using the S. cerevisiae TEF1 promoter, Sc_HAC1_co/Sc_SSO2_co were expressed using the S. cerevisiae TEF2 promoter, Sc_BiP_co/Sc_PDI_co were expressed using the S. cerevisiae PGK1 promoter, or Sc_BiP_co were expressed using the S. cerevisiae FBA1 promoter and Sc_PDI_co were expressed using the S. cerevisiae PGK1 promoter.

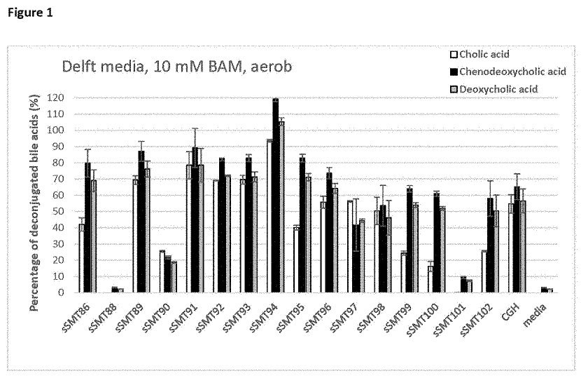

All BSH candidates were tested for bile acid hydrolase activity in the strain background described. The genes were selected for testing based upon similar enzymatic capability to the Bacteroides ovatus BSH enzyme (Bo_BSH_co-SEQ ID NO: 1) based on research where Bacteroides ovatus broth inhibits growth of C. difficile only in the presence of bile salts (Yoon et al., 2017). Selection for transformants was done using the well-known Kluyveromyces lactis LEU2 marker available e.g. from EUROSCARF (http://www.euroscarf.de) and growth on media lacking leucine. When BSH wasexpressed and active, the recombinant S. boulardii strain produced active secreted bile salt hydrolase, which was analyzed by the deconjugation of conjugated bile acids. A selection of active BSH strains is listed in Tablel: Selection of Sb^{BSH} strains, and the respective enzyme activity results of a cultivation with growth conditions described in Example 2 is shown in FIG. 1 (Delft, aerob).

TABLE 1
Selection of Sb^{BSH} strains

| | | | Yeast | Yeast | Yeast |
|---|---|---|---|---|---|
| Strain | BSH | BSH origin | Gene 1 | Gene 2 | Gene 3 |
| sSMT85 | Bo_bsh_co | Bacteroides ovatus | Sc_KEX2_co | Sc_BiP_co | Sc_PDI_co |
| sSMT86 | Bo_bsh_co | Bacteroides ovatus | Sc_KEX2_co | Sc_BiP_co | — |
| sSMT87 | Bo_bsh_co | Bacteroides ovatus | Sc_KEX2_co | Sc_BiP_co | — |
| sSMT88 | No BSH | — | — | — | — |
| sSMT89 | Er_BSH_co_SP-Bo | Eubacterium rectale | Sc_KEX2_co | Sc_BiP_co | — |
| sSMT90 | Fp_BSH_co_SP-Bo | Faecalibacterium | Sc_KEX2_co | Sc_BiP_co | — |
| | | prausnitzii | | | |
| sSMT91 | Ba_BSH_co_SP-Bo | Bifidobacterium | Sc_KEX2_co | Sc_BiP_co | — |
| | | adolescentis | | | |
| sSMT92 | Bd_BSH_co_SP-Bo | Bifidobacterium dentium | Sc_KEX2_co | Sc_BiP_co | — |
| sSMT93 | Blo_BSH1_co_SP-Bo | Blautia obeum | Sc_KEX2_co | Sc_BiP_co | — |
| sSMT94 | Lm_BSH_co_SP-Bo | Lactobacillus mucosae | Sc_KEX2_co | Sc_BiP_co | — |
| sSMT95 | Bo_ATCC_BSH_co | Bacteroides ovatus | Sc_KEX2_co | Sc_BiP_co | — |
| | | ATCC 8483 | | | |
| sSMT96 | Bu_ATCC_BSH_co | Bacteroides uniformis | Sc_KEX2_co | Sc_BiP_co | — |
| | | ATCC 8492 | | | |
| sSMT97 | Eve_BSH_co_SP-Bo | Eubacterium ventriosum | Sc_KEX2_co | Sc_BiP_co | — |
| | | ATCC 27560 | | | |
| sSMT98 | Blo_ATTC_BSH_co_ | Blautia obeum | Sc_KEX2_co | Sc_BiP_co | — |
| | SP-Bo | ATCC 29174 | | | |
| sSMT99 | Bc_BSH_co | Bacteroides caecimuris | Sc_KEX2_co | Sc_BiP_co | — |
| sSMT100 | Aa_BSH_co | Alistipes sp. An66 | Sc_KEX2_co | Sc_BiP_co | — |
| sSMT101 | Ma_BSH_co | Muribaculum sp. An287 | Sc_KEX2_co | Sc_BiP_co | — |
| sSMT102 | BCAG_BSH_co | Bacteroides sp. CAG: 633 | Sc_KEX2_co | Sc_BiP_co | — |

### Example 2

### Growth Conditions for Recombinant Saccharomyces boulardii

For initial screening, the recombinant S. boulardii transformants of Example 1 were tested in a deconjugation experiment in 96-well plates. All incubations were carried out at 30° C. with shaking at 230 rpm in a Kuhner Climo-Shaker ISF1-X. On day 0, a preculture of the BSH candidates was set up in 96 deep-well plates in 500 pi liquid synthetic complete medium lacking leucing (SC-leu) for 24 hours so all clones reach a steady state growth plateau in order to start out at a similar OD for the subsequent testing. On day 1, 50 μl of this preculture were transferred to 450 μl mineral medium (Delft media) described in Jensen et al., 2014, and incubated for 24 hours at 30° C. with shaking at 230 rpm. On day 2, 200 μl of this cell cultivation was transferred to a new plate, and 100 μl 2× mineral media, 60 μl water and 40 μl of a 100 mM stock solution of each bile acid mix (BAM) (taurocholic acid, taurodeoxycholic acid, taurochenodeoxycholic acid, glycocholic acid, glycodeoxycholic acid, glycochenodeoxycholic acid) were added, yielding an end concentration of 10 mM BAM. The deconjugation reaction was incubated for 24 hours at 30° C. with shaking at 230 rpm, and supernatant was harvested by centrifuging the cultivation for 10 min at 4000 g, and then diluting with water 1:200 for LC-MS measurement (see method in Example 4).

S. boulardii is absent from the natural gut microbiota, however when administrated it has the capacity to colonize the colon (Blehaut et al.; 1989; Margret I Moré & Vandenplas; 2018; Margret Irmgard Moré & Swidsinski; 2015). For this reason, we tested BSH producers of S. boulardii for growth with fed-state simulated colonic fluid (FeSSCoF) in order to evaluate the production/secretion of BSH and enzyme specificity in gut-like conditions. The preparation of FeSSCoF medium was performed following the protocol described by Marques, Loebenberg and Almukainzi (2011) with minor modifications. 450 mL of a Tris/maleate buffer solution (3.7 g of tris(hydroxymethyl) aminomethane (Tris), and 3.5 g of maleic acid were added to Milli-Q water, the pH adjusted to 6.0 with about 33 mL of 0.5 M sodium hydroxide, and the final volume was adjusted to 1 L. Importantly, neither ox nor porcine bile extract was added. 0.370 g of lecithin was dissolved in 3 mL of dichloro-methane, and 0.051 g of palmitic acid was dissolved in 3 mL of dichloromethane. The dichloromethane was evaporated under vacuum at 40° C. until a clear solution having no perceptible odor of dichloromethane was obtained. The volume was adjusted to 1 L with tris/maleate buffer. 2 g of sodium chloride, 14 g of glucose, and 3 g of bovine serum albumin were added, and dissolved by gentle agitation with a magnetic stirrer. The final solution was lightly turbid, and the pH of this medium is about 6.0.

For growing S. boulardii in FeSSCoF under tight anaerob conditions, a pre-culture in rich media was prepared. InSitu Sb^{BSH}cultures sSMT85, sSMT86 and sSMT87 plus wild type sSMT88were pre-grown in 5 mL of yeast peptone dextrose (YPD), overnight at 37° C. with shaking at 180 rpm. For media change, S. boulardii cultures were washed three times with sterile Milli-Q water and resuspended in 5 mL FeSSCoF medium in a 50 mL falcon tube. In order to grow under anaerobic conditions, the falcon tubes were flushed with argon gas and the falcon tube lids were closed tightly. InSitu™ Sb^{BSH} cultures were then grown at 37° C. with shaking at 180 rpm for 3 days prior analysis, with 100 μl samples taken every 24 h. As expected, the BSH enzyme was also catalytically active under unaerob conditions in gut like media (FIG. 3). The combination of secretion boost genes only seems to have a minor effect on the deconjugation pattern, and the overall pattern and efficiency of deconjugation did not change discernably over time.

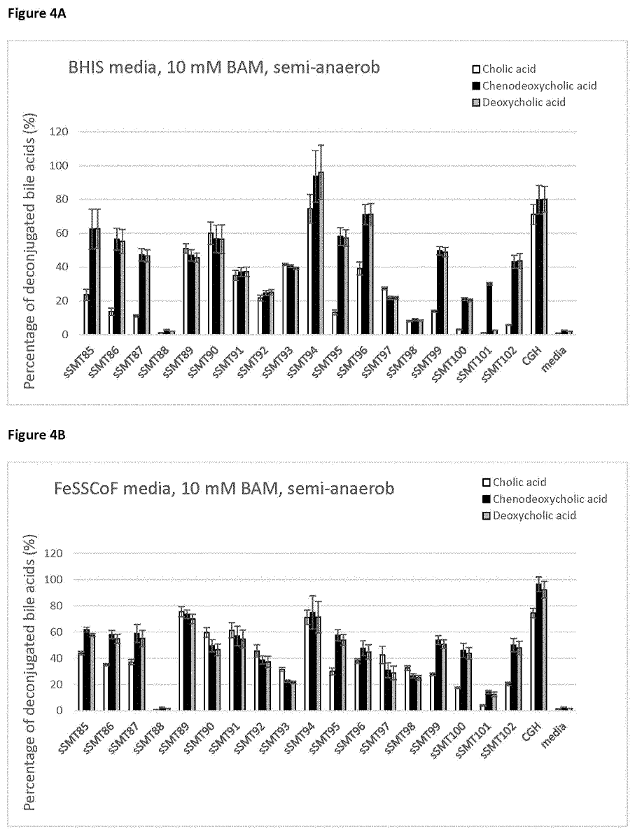

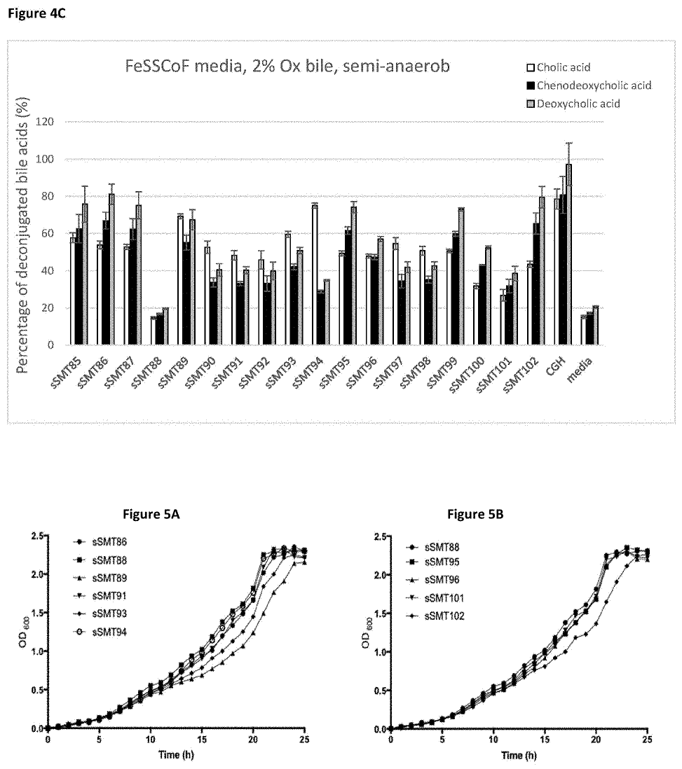

In order to verify the deconjugation activity of InSitu Sb^{BSH}clones in a high throughput manner, the recombinant S. boulardii strains of Table 1 were tested in a deconjugation experiment in 96-well plates. All incubations were carried out at 37° C. with shaking at 230 rpm in a Kuhner Climo-Shaker ISF1-X. On day 0, a preculture of the BSH candidates was set up in 96 deep-well plates in 500 μL liquid synthetic complete medium lacking leucing (SC-leu) or YPD for 24 hours so all clones reach a steady state growth plateau in order to start out at a similar OD for the subsequent testing. On day 1, 50 μl of this preculture was transferred to 450 μl FeSSCoF media, and incubated for 24 hours at 37° C. with shaking at 230 rpm under semi-anaerobic conditions. On day 2, 200 μl of this cell cultivation was transferred to a new plate, and 160 μl FeSSCoF and 40 μl of a 100 mM stock solution of each bile acid mix (BAM) (taurocholic acid, taurodeoxycholic acid, taurochenodeoxycholic acid, glycocholic acid, glycodeoxycholic acid, glycochenodeoxycholic acid) were added, yielding an end concentration of 10 mM conjugated bile acid. The deconjugation reaction was incubated for up to 24 hours at 37° C. with shaking at 230 rpm under semi-anaerobic conditions, and supernatant was harvested by centrifuging the cultivation for 10 min at 4000 g, and then diluting with water 1:200 for LC-MS measurement (see method below). The deconjugation activity of the selected strains was working very well under anaerob conditions in FeSSCoF media (FIG. 4B (FeSSCoF, semi-anaerob)).

### Example 3

### Analysis of Protein Production, Secretion and Localization of Bile Salt Hydrolase

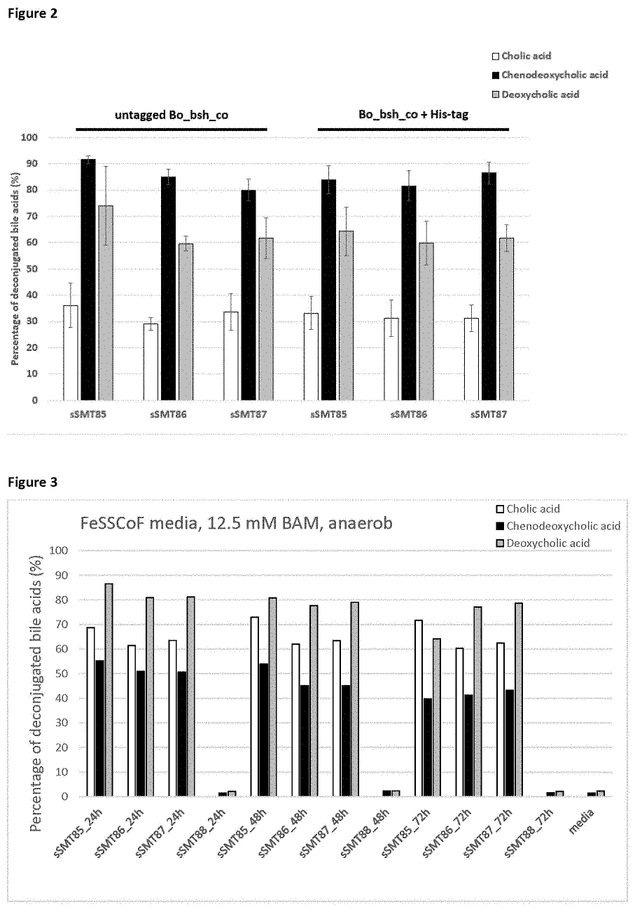

In order to assess protein secretion of BSH in yeast, we tested the BSH enzyme from Bacteroides ovatus with or without a functional His-tag in an auxotrophic Biocodex S. boulardii strain made in Example 1. As described in Example 1, the bacterial BSH sequences were analyzed using SignalP-5.0 (Almagro Armenteros et al., 2019) for the detection of putative bacterial secretion peptides. The Bacteroides ovatus pre-peptide was kept since it is recognized in Eukaryotes (Bo_bsh_co-SEQ ID NO: 2). We tested both His-tagged and non-His-tagged B. ovatus BSH in Biocodex, and detected very good deconjugation activity of B. ovatus BSH with its intrinsic bacterial pre-peptide (FIG. 2). An equally excellent deconjugation was observed in strains carrying the combinations Bo_BSH_co/Sc_KEX2_co/Sc_BiP_co/Sc_PDI_co, Bo_BSH_co/Sc_KEX2_co/Sc_BiP_co and Bo_BSH_co/Sc_KEX2_co/Sc_PDI_co. We also found that the His-tag did not affect the enzymatic BSH activity negatively (FIG. 2). The strains expressing His-tagged Bo_bsh_co can be used to analyze protein localization in different cell fractions by Western blotting or by fluorescence microscopy of fixed cells.

Heterologous protein production, localization and secretion is analyzed in a similar way as described in (Wentz & Shusta, 2008) by inoculating the BSH expressing S. boulardii strains in liquid Mineral media, grown overnight at 37° C. with shaking at 230 rpm in a Kuhner Climo-Shaker ISF1-X prior to dilution to a uniform OD₆₀₀ of 0.1 and regrowth for 3 days to an OD₆₀₀ between 8 and 10. Samples are taken by removing 100 μL of the culture for measurement of cell density (OD₆₀₀), analysis of intracellular content, and determination of secretion yield. Protein levels are measured according to Wentz & Shusta, 2008 with sodium dodecyl sulfate polyacrylamide gel electrophoresis (SDS-PAGE) and quantitative Western blotting. Protein concentration is measured using Bradford Assay (Bio-Rad), using bovine serum albumin (BSA) as a standard. The cell-free BSH supernatants are resolved on a 12% polyacrylamide-SDS gel and transferred to nitrocellulose membranes. The membranes are probed with the 6x-His Tag Monoclonal Antibody (3D5) antibody (1:1000; ThermoFisher Scientific) followed by a horseradish peroxidase (HRP)-conjugated anti-mouse secondary antibody (1:2000; Sigma, St. Louis, Mo.).

### Example 4

### Analysis of BSH Activity and Evaluation of BSH Substrate Specificity of Saccharomyces boulardii Recombinantly Expressing Different BSH Enzymes (Sb^{BSH})

BSH enzyme activity is analyzed via three different assays with different sensitivity.

### a) BSH Activity Plate Assay.

The qualitative enzyme activity of the S. boulardii strains with BSH is determined on solid media, derived from the CBH plate assay described in (Christiaens, Leer, Pouwels, & Verstraete, 1992). Strains are incubated at 30° C. for 72 h on synthetic complete (SC) solid media available e.g. from Sigma Aldrich containing 2% (w/v) glucose, 0.5% (w/v) TC (taurocholic acid, 5 g/L) or GC (glycocholic acid, 5 g/L), and 0.37 g/L CaCl₂ under aerobic or anaerobic conditions. Due to medium acidification and deconjugation of the bile acids, hydrolase-active colonies are expected to produce copious amounts of precipitated cholic acid, which is visible by eye as colonies surrounded with big halos of a white precipitate in bacteria.

### b) BSH Assay for Quantification of BSH Activity on Plate Reader—Ninhydrin Assay.

BSH activity is measured by determining the amount of amino acids released from conjugated bile salts as previously described (Liong & Shah, 2005), with several modifications described in (Jiang et al., 2010). Briefly, 4 ml stationary phase cells are centrifuged at 4,000 g and 4° C. for 20 min. The cell pellet is then washed twice and resuspended in 3.5 ml 0.1 M phosphate buffer (pH 6.0). Next, 0.5 ml 2% sodium thioglycolate is added to the cell suspension and the mixture is sonicated for 4 min while constantly cooling on ice. The samples are then centrifuged at 13,000 g and 4° C. for 20 min. Next, 0.1 ml supernatant is mixed with 0.8 ml 0.1 M sodium phosphate buffer (pH 6.0) and 0.1 ml 50 mmol/1 conjugated bile salt. The mixture is then incubated at 37° C. for 30 min, after which 0.75 ml aliquots are mixed immediately with 0.75 ml 15% (w/v) trichloroacetic acid. Next, the samples were centrifuged at 13,000 g and 4° C. for 10 min, and then 1 ml of the supernatant is mixed with 2 ml ninhydrin reagent [0.5 ml 1% ninhydrin in 0.5 M citrate buffer (pH 5.5), 1.2 ml 30% glycerol, 0.2 ml 0.5 M citrate buffer pH 5.5). The mixture is then boiled for 30 min and subsequently cooled for 3 min in ice water. Finally, the absorption is recorded at 570 nm with a GloMax Discover microplate reader (Promega) and the amount of product formed is estimated from a calibration curve produced using glycine or taurine separately. One unit of BSH activity is defined as the amount of enzyme that liberates 1 μmol amino acid from the substrate per minute. The specific activity is defined as the number of units of activity per milligram of protein. The protein concentrations of the supernatant are determined by the Bradford Agent (Bio-Rad) using bovine serum albumin as the standard. All experiments in this study are repeated three times. To determine the substrate specificity of purified enzymes, the six major human bile salts are selected (Tanaka, Hashiba, Kok, & Mierau, 2000):

taurocholic acid (TCA), taurochenodeoxycholic acid (TCDCA), taurodeoxycholic acid (TDCA), glycocholic acid (GCA), glycochenodeoxycholic acid (GCDCA), and glycodeoxycholic acid (GDCA) obtained from Sigma. The rate of hydrolysis of conjugated bile salts is measured at 37° C. and at pH 6.5, which is similar to that of the small intestine in a healthy human (Corzo & Gilliland, 1999). The released amount of amino acid from the substrates by enzymatic reaction is measured by ninhydrin assay and compared with the standard curve prepared by using either glycine or taurine. These experiments are conducted in triplicate.

### c) BSH Activity LC-MS Assay—LC-MS Analysis of Conjugated and Free Bile Salts.

Protein concentrations are measured with the Bradford Agent (Bio-Rad), using bovine serum albumin (BSA) as a standard. The BSH activities are determined against the six major human conjugated bile salts according to (Hay & Carey, 1990; Staggers, Hernell, Stafford, & Carey, 1990): taurocholic acid (TCA), taurochenodeoxycholic acid (TCDCA), taurodeoxycholic acid (TDCA), glycocholic acid (GCA), glycochenodeoxycholic acid (GCDCA), and glycodeoxycholic acid (GDCA) obtained from Sigma. For BSH activity measurement, deconjugation of these conjugated bile acids added to a S. boulardii culture is determined by measuring the amounts of the conjugated and deconjugated compounds.

Samples from Example 2 were prepared for LC-MS analysis by adjusting the pH to 7.5 with 10 mM NaOH to stop BSH activity. The samples were centrifuged at 4000 g in a microcentrifuge for 10 min, and for LC-MS quantification the supernatant was diluted 1:200 in water.

Targeted LC-MS analysis was performed to quantify conjugated and deconjugated bile acids. Liquid chromatography was performed on an Agilent 1290 Infinity II UHPLC with a binary pump and multisampler (Agilent Technologies, Palo Alto, Calif., USA). Separation was achieved on a Luna Omega PS C18 column (50×2.1 mm, 1.6 μm, 100 Å, Phenomenex, Torrance, Calif., USA) using 0.1% (v/v) formic acid in H₂O with 5 mM ammonium acetate and 0.1% (v/v) formic acid in acetonitrile as mobile phases A and B, respectively. Gradient conditions were: 0.0-0.5 min 25% B; 0.5-8 min 25-50% B; 8-10 min 50-98% B; 10-11 min 98% B; 11-11.1 min 98-25% B, 11.1-12 min 25% B. The injection volume was 2μL and the mobile phase flow rate was 400 μL/min. The column temperature was maintained at 30° C. The liquid chromatography system was coupled to an Ultivo-Triple Quadrupole mass spectrometer (Agilent Technologies, Palo Alto, Calif., USA) equipped with an electrospray ion source (ESI) operated in positive and negative mode. The Capillary voltage was maintained at 3500 V and the Nozzle voltage at 500 V. Source gas temperature was set at 340° C. and source gas flow was set at 12 L/min. Source sheath gas temperature was set at 380° C. and source sheath gas flow set at 12 L/min. Nebulizing gas was set to 30 psi. Nitrogen used as a dry gas, nebulizing gas and collision gas. Conjugated and deconjugated bile acids were detected in MRM mode in which the MRM transitions and mass spectrometer parameters (fragmentation voltage, collision energy, dwell time) were optimized for each metabolite. Calculation of BSH activity is based on the release of deconjugated bile acid (CA, CDCA and DCA) from the hydrolysis of the amide bond of taurine- or glycine-conjugated bile acids. The free bile salts are released linearly during the 24 h incubation period. Standard stock solutions of the six conjugated bile acids were made by dissolving 100 mM in 10 ml of water. The stock solutions were diluted in water to obtain a concentration of 10 mM. For quantification of conjugated bile acids, the standards were used as a reference, and the concentration (mg/ml) of both the reference and samples was calculated by multiplication of the area of the plotted curve with the response factor and the dilution factor. The means from duplicate samples in two replicate experiments can be determined and used to plot bile salt deconjugation curves. The slope of the linear regression is related to the parallel determination of heterologous protein, and the BSH activity is expressed as nanomoles of deconjugated bile acid produced per microgram of heterologous protein per hour.

### Example 5

### Inhibition of C. difficile Growth By Recombinant Sb^{BSH} Culture Supernatants (In Vitro Assay)

a) Evaluation of Growth of Sb^{VSH} Strains in Different Media and with Different Bile Acid Substrates

C. difficile is usually grown in the specific rich media BHIS (Brain Heart Infusion Supplemented) that is not typically used for culturing yeast, so in order to be sure that the Sb^{BSH} strains exhibit satisfying deconjugation activity in this unusual media we conducted a deconjugation test of the strains in Table 1. The BHIS used in this study had the following recipe: 37 g Brain heart infusion (Oxoid), 5 g Yeast extract (Oxoid), 1 g L-cysteine (Sigma-Aldrich), and optional 1000 μl of 1000× Resazurin (1 meml in dH₂O, Sigma-Aldrich). The Sb^{BSH}strains listed in Table 1 were grown as described in Example 2 in either FeSSCoF or BHIS media in high throughput formate.

Strains were inoculated from YPD plates into 3 ml YPD (=preculture), grown in KOhner shaker for O/N at 37° C., 250 rpm. 50 μl cells were transferred to 450 μl 1×BHIS (=cultivation), grown in Köhner shaker for 24 h at 37° C., 250 rpm. 200 μl cells were diluted with 160 μl BHIS and 40 μl 10×BAM, incubated in Kühner shaker for 24 h at 37° C., 250 rpm. Samples were harvested at 24 h, and SUP (supernatant) was prepared by water extraction for analytics, dilution 1:200. Sb^{BSH} strains displayed comparable deconjugation activity as in Delft media (FIG. 4A, compare to FIG. 1).

In the literature, the substrate of deconjugation for BSH enzymes is often undefined ox bile or porcine bile extracted from animal guts (used amongst others in Yoon et al., 2017). In order to confirm the usage of ox bile as a deconjugation substrate, we carried out a test in FeSSCoF media under semi-anaerobic conditions. Strains listed in Table 1 were inoculated from YPD plates into 3 ml YPD (=preculture), grown in KOhner shaker for O/N at 37° C., 180 rpm. 50 μl cells were transfered to 450 μl FeSSCoF w/out bile extract (=cultivation), grown in Kühner shaker for 24 h at 37° C., 250 rpm, semi-anaerobic. 200 μl cells were diluted with 160 μl FeSSCoF +40 μl 10× ox bile (2% end concentration, Sigma Aldrich), and incubated in KOhner shaker for 24 h at 37° C., 250 rpm, semi-anaerobic. Samples were harvested with water extraction for analytics, dilution 1:200. Sb^{BSH} strains displayed comparable deconjugation activity with 2% ox bile as in FeSSCoF media with BAM as substrate (FIG. 4C, compare to FIG. 4B).

### b) Sb^{BSH} Strain Fitness

To assess strain fitness, growth curves of strains listed in Table 2 were generated.

TABLE 2
Selection of Sb^{BSH} strains tested in in vitro assay

| Strain | BSH | origin | Gram | Reasoning |
|---|---|---|---|---|
| sSMT86 | Bo_BSH_co | Bacteroides | negative | Published |
| | | ovatus | | inhibition of |
| | | | | C. difficile |
| | | | | (Yoon et al., 2017) |
| sSMT89 | Er_BSH_ | Eubacterium | positive | Even profile, high |
| | co_SP-Bo | rectale | | deconjugation |
| sSMT91 | Ba_BSH_ | Bifidobacterium | positive | Good |
| | co_SP-Bo | adolescentis | | deconjugation |
| sSMT93 | Blo_BSH1_ | Blautia obeum | positive | Good |
| | co_SP-Bo | | | deconjugation |
| sSMT94 | Lm_BSH_ | Lactobacillus | positive | Fastest kinetics |
| | co_SP-Bo | mucosae | | |
| sSMT95 | Bo_ATCC_ | Bacteroides | negative | Low T/GCA |
| | BSH_co | ovatus | | deconjugation |
| | | ATCC 8483 | | |
| sSMT96 | Bu_ATCC_ | Bacteroides | negative | Second fastest |
| | BSH_co | uniformis | | kinetics |
| | | ATCC 8492 | | |
| sSMT101 | Ma_BSH_ | Muribaculum sp. | negative | Low |
| | co | An287 | | deconjugation |
| sSMT102 | BCAG_ | Bacteroides sp. | negative | Low T/GCA |
| | BSH_co | CAG: 633 | | deconjugation |
| sSMT88 | No BSH | — | — | Wild type |
| | | | | control |

To assess strain fitness aerobically, overnight S. boulardii cultures of wild type and mutant strains listed in Table 2 were set up in 250 ml baffled culture flasks in 25 ml BHIS media and incubated aerobically at 37° C. with shaking at 250 rpm. The next day 250 μl (1/100 dilution) of these yeast cultures were diluted in 250 ml baffled culture flasks in 25 ml BHIS media and incubated aerobically at 37° C. with shaking at 250 rpm in a water bath. At every hour, 1 ml of culture was removed and the OD₆₀₀ was measured until stationary phase was reached. BHIS was used as a blank. The aerobic growth curves generated show that although there are minor differences between strains, growth is comparable with all strains reaching an equivalent OD at stationary phase at ˜25 h from subculture (FIG. 5). Of the 10 strains, sSMT89, sSMT93 and sSMT102 displayed the greatest divergence from wild type, all of which grew at a slower rate but settled upon a comparable final OD.

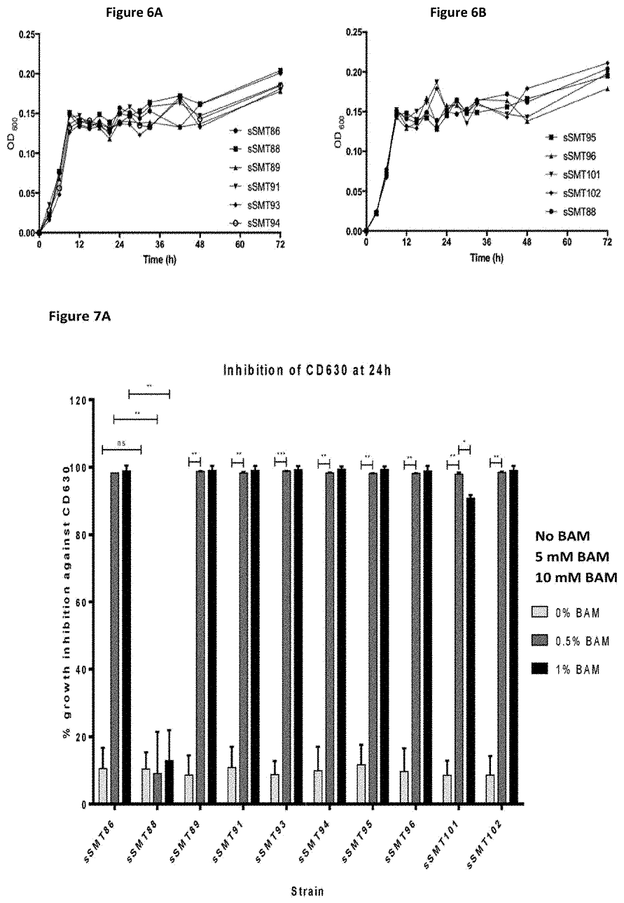

To assess strain fitness anaerobically, overnight S. boulardii cultures listed in Table 2 were set up in 250 ml baffled culture flasks in 25 ml BHIS media and incubated aerobically at 37° C. with shaking at 250 rpm. The next day 30 μl (1/100 dilution) of these yeast cultures were diluted in 14 ml culture tubes in 3 ml pre-reduced BHIS media and incubated anaerobically (Whitley A35 anaerobic workstation with anaerobic gas; 5% CO₂, 5% H₂, 90% N₂) at 37° C. with shaking at 250 rpm. Periodically, 100 μl of culture was removed and the OD₆₀₀ was measured using a plate reader. BHIS was used as a blank. In comparison with their aerobic growth, the yeast strains grew notably slower and to a lesser extent in anaerobic conditions (FIG. 6). After 72 h of anaerobic growth the yeast strains had only reached an OD of ˜0.2. As was the case in aerobic conditions, anaerobically all 10 strains grew at a similar rate and to an equivalent degree with each other, with all OD measurements at 72 h falling between 0.17 and 0.22. Interestingly, the majority of logarithmic growth occurred between 0 and 12 h after subculture. In summary, although the yeast strains grew notably slower in anaerobic compared to aerobic conditions, there were no significant fitness issues between the wild type and mutant strains.

c) Preparation of C. difficile Spores

All work involving C. difficile was performed by SporeGen® Ltd, UK. Spores of C. difficile were prepared by growth on SMC agar plates using an anaerobic incubator (Don Whitley, UK) as described previously (Hong et al., 2017; Permpoonpattana et al., 2011). After growth for seven days at 37° C. spores were harvested and the spore pellet further purified using centrifugation through a 20% to 50% Histodenz gradient (Sigma) as described elsewhere (Phetcharaburanin et al., 2014). Spore CFU was determined by heat treatment (60° C., 20 min.) and plating on BHISS agar plates (Brain heart infusion agar) containing 0.1% (w/v) L-cysteine, 5 mg/ml yeast extract and the spore germinant sodium taurocholate (0.1% w/v).

Evaluation of Inhibition of C. difficile Vegetative Ggrowth by Sb^{BSH} Culture Supernatants

To determine whether the Sb^{BSH} mutants would have increased inhibitory activity against C. difficile, supernatants grown in the presence or absence of BAM were tested against two different C. difficile strains in a plate well assay. The inhibition of C. difficile growth by S. boulardii culture supernatants was analyzed according to Yoon et al., 2017 with the following adjustments. One or more of the following C. difficile strains can be used: 630 [tcdA⁺tcdB⁺, ribotype 012] (Mist et al., 1982, ST11 [ribotype 078] (Knight et al., 2019), R20291 [ribotype 027] (Valiente et al., 2014), 90556-M6S [ribotype 001] (Type strain 9689, American Type Culture Collection (ATCC)), VPI 10463 [ribotype 087] (strain 43255, ATCC), M68 [ribotype 017] (Drudy, Harnedy, Fanning, O′Mahony, & Kyne, 2007; Lawley et al., 2009), isolate B117-6443 of strain NAP1/131/027 [ribotype 027] (Chen et al.; 2008; Fatima & Aziz, 2019).

The following C. difficile (CD) strains were used: CD630 and R20291. Overnight S. boulardii cultures in 3 ml BHIS media were set up in 14 ml culture tubes and incubated aerobically at 37° C. with shaking at 250 rpm. The next day, 500 μl of these yeast cultures were diluted in 14 ml culture tubes in 5 ml BHIS media containing 0 mM bile acid mix (BAM), i.e., no supplements. 120 μl of these yeast cultures were also diluted in deep well plates in 1.2 ml BHIS media containing 0.5× (5 mM) or lx (10 mM) BAM. These subcultures were incubated anaerobically (in a Don-Whitley anaerobic workstation) for 3 days at 37° C. and shaking at 250 rpm. Cultures were centrifuged for 10 min at 4000 g and the supernatant was transferred by aspiration and stored at −20° C. until further processing. CD vegetative cells were anaerobically cultured in BHIS medium at 37° C. to early stationary phase and adjusted to the same OD at OD₆₀₀. CD cultures were then inoculated into 200 μl fresh BHIS broth at a 1:100 ratio in a deep-well 96-well plate; subsequently 200 μl of yeast supernatant obtained after 3 days of cultivation in BHIS media containing OmM, 5 mM or 10 mM BAM (from −20 ° C.) were added to the CD cultures. Vegetative cell growth of CD was assessed after anaerobic incubation at 37° C. for 24 h, samples were taken at time points 0, 6 h and 24 h. OD₆₀₀ was measured using a microplate reader. The blank was 200 μl of BHIS broth, and control wells included BAM (5 mM and 10 mM) to control for its effect on CD growth. Each reaction was tested in duplicate (200 μl measurements) or in triplicate (100 μl measurements). Growth inhibition (%) was calculated using the following formula:

100−((OD₆₀₀ of reaction/OD₆₀₀ of media control)×100)

Although CD OD₆₀₀ measurements were taken at 6 h and 24 h for the inhibition assay, CD usually reaches stationary phase at 10-12 hours so the latter measurement is more representative of the inhibitory activity.

To determine whether the Sb^{BSH} mutants would have increased inhibitory activity against C. difficile, supernatants grown in the presence or absence of BAM were tested against two different C. difficile strains in a plate well assay. The inhibition of C. difficile growth by S. boulardii culture supernatants was analyzed according to Yoon et al., 2017 with the following adjustments. One or more of the following C. difficile strains can be used: 630 [tcdA⁺tcdB⁺, ribotype 012] (Mist et al., 1982, ST11 [ribotype 078] (Knight et al., 2019), R20291 [ribotype 027] (Valiente et al., 2014), 90556-M6S [ribotype 001] (Type strain 9689, American Type Culture Collection (ATCC)), VPI 10463 [ribotype 087] (strain 43255, ATCC), M68 [ribotype 017] (Drudy, Harnedy, Fanning, O'Mahony, and Kyne, 2007; Lawley et al., 2009), isolate Bl17-6443 of strain NAP1/Bl/027 [ribotype 027] (Chen et al.; 2008; Fatima & Aziz, 2019).

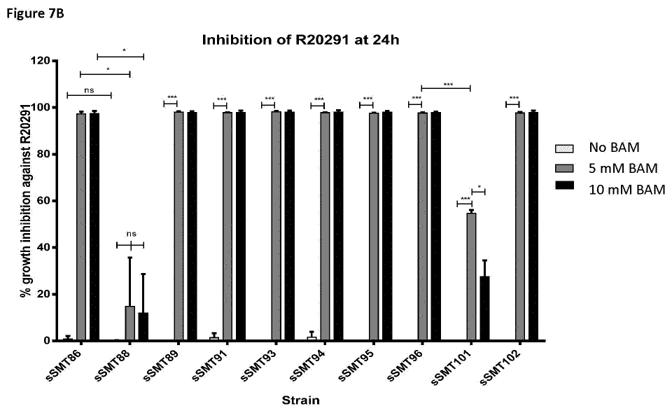

In the experiments, the amount of growth inhibition exerted by the yeast supernatants against CD630 (FIG. 7A) and R20291 (FIG. 7B) is shown. None of the 10 yeast strains showed greater than 20% inhibitory activity against CD630 in the absence of supplementary BAM (FIG. 7A). However, if supplemented with 5 mM BAM all strains but wild type (sSMT88) demonstrated significantly higher inhibitory activity, with all supernatants suppressing greater than 90% of CD growth. If 10 mM BAM was supplemented then the amount of CD630 growth inhibition was of the same extent as that of 5 mM supplementation, and no significant differences could be observed by increasing the concentration. Supplementation of the wild type strain (sSMT88) bestowed no additional inhibitory activity upon the supernatant, with no significant differences being observed between 0, 5 and 10 mM BAM supplementation.

The pattern with R20291, was much the same as CD630 (FIG. 7B). With the exception of wild type strain sSMT88 and mutant strain sSMT101, supernatants from all strains supplemented with 5 mM and 10 mM BAM exhibited greater than 95% inhibition against hypervirulent strain R20291. The low levels of growth inhibition against R20291 demonstrated by wild type sSMT88 supernatants showed no significant differences regardless of BAM supplementation. Although the sSMT101 supernatant displayed increased inhibition when growth was supplemented with BAM, inhibitory activity was significantly lower compared to the other mutant strains. To summarise, the addition of BAM to growth media significantly increased the activity against C. difficile of all Sb^{BSH} mutant strains while having no discernible effect on the wild type.

### d) Verification of Inhibitory Efect of Bile Acid Metabolites

In order to determine whether the inhibitory effect of the yeast supernatant was indeed due to the bile acid metabolites produced during culture, the bile acid sequestrant, cholestyramine, was used as described previously (Giel et al.,2010). Cholestyramine resin (Sigma-Aldrich) was added to yeast supernatants supplemented with 0% and 1% bile acid to a final concentration of 50 mg/ml. The mixture was rocked at room temperature (RT) for 1 h, centrifuged, filtered through a 0.22 μm pore size filter (Advantec), and stored at −20° C. until use.

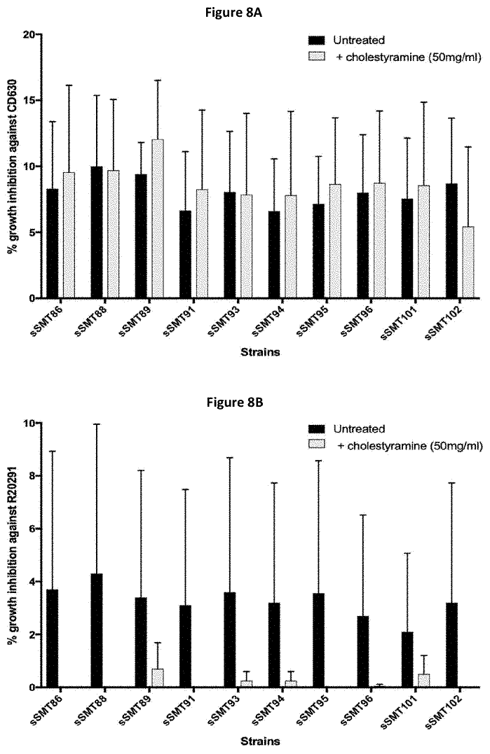

CD growth was measured by determining OD₆₀₀ after 24 h of anaerobic incubation at 37° C. in the presence of the cholestyramine-treated yeast supernatant. Samples were taken at time points 0, 6 h and 24 h. The blank was 200 μl of BHIS broth, and the effect of Ox bile on CD growth was also controlled for. Each reaction was tested in duplicate (200 μl measurements). All yeast strains listed in Table 2 were supplemented with 0 or 1% Ox bile during growth, and the supernatants underwent cholestyramine treatment (50 mg/ml) for an hour. Cholestyramine treated and untreated supernatants (0 or 1% Ox bile) were then tested for their inhibitory activity against CD630 and R20291. Samples were taken at 6 h and 24 h to measure the OD₆₀₀ of CD in the presence of the supernatants. In the absence of Ox bile supplementation (0%), the difference between the inhibitory activity of cholestyramine treated and untreated supernatants was insignificant (FIG. 8). The amount of inhibitory activity exhibited by the supernatants against CD630 (FIG. 8A) was less than 15% and less than 10% against R20291 (FIG. 8B). Although there seemed to be an observable difference between the activity of cholestyramine-treated and untreated supernatants against R20291 (FIG. 8B), due to the large variance and the low numbers the differences were not significant. In addition, there was no signficant differences observed between the wild type (sSMT88) and any of the mutant strains. This suggests that in the absence of Ox bile supplementation, there was no production of inhibitory metabolites by the Sb^{BSH} strains, and so cholestyramine sequestering had no effect on CD inhibition. Strains supplemented with Ox bile during growth (1%), and then treated with cholestyramine showed a significant difference to untreated supernatants (FIG. 9). With the exception of the wild type (sSMT88) and sSMT101, all untreated supernatants showed high inhibitory activity (greater than 75%) against CD630 (FIG. 9A). If treated with cholestyramine however, this activity was abolished, and growth inhibition of CD dropped to below 20%. Cholestyramine treatment had no effect on sSMT88 and sSMT101 supernatant. The pattern was similar with R20291 (FIG. 9B), where untreated supernatants from all strains except for sSMT88 and sSMT101 showed high inhibitory activity against the pathogen. This activity was abrogated and significantly dropped in all strains (except sSMT88 and sSMT101) after cholestyramine treatment. This confirmed that supplemented Ox bile elicited the production of CD inhibitory metabolites in the Sb^{BSH} mutant supernatants, which are sequesterable by cholestyramine. We therefore concluded that the increased inhibitory effect of engineered Sb^{BSH} on CD growth was mediated by bile acid metabolites produced during culture. Therefore, growth of C. difficile in the presence of bile acid without cholestyramine (bile acid+/cholestyramine−) was inhibited, and growth in the presence of bile acid with cholestyramine (bile acid+/cholestyramine+) was not inhibited.

### Example 6

### Inhibition of C. difficile Spore Germination by Sb^{BSH} Culture Supernatants

CD spores were produced and their concentration determined as described previously (Hong et al., 2017a). The detailed protocol for preparation of C. difficile spores is described above. After the vegetative bacteria are killed by incubation at 60° C. for 20 min, spores were incubated in yeast supernatant obtained as follows: Overnight S. boulardii cultures of strains listed in Table 2 were set up in 14 ml culture tubes in 3 ml BHIS media and incubated aerobically at 37° C. with shaking at 250 rpm. The next day, these yeast cultures were pelleted for 5 min at 4000 g and diluted in 14 ml culture tubes in 5 ml fed-state simulated colonic fluid (FeSSCoF) media containing 0 mM BAM. In the case of 10 mM BAM cultures, 0.75 ml of overnight yeast cultures were pelleted and diluted in deep well plates in 1.25 ml FeSSCoF media containing 10 mM BAM. These cultures were incubated anaerobically (in an anaerobic workstation) for 3 days at 37° C. and shaking at 250 rpm. Cultures were centrifuged for 10 min at 4000 g and supernatant was transferred by aspiration and stored at −20° C. until further processing. 1×10⁷ CD spores in 100 μl of BH IS with 1% (w/v) taurocholic acid (TCA) were incubated with an equal volume of yeast supernatant for 15 min. After incubation, spores were serially diluted and plated on BHIS agar with or without supplementation of 0.1% (v/v) TCA. After overnight anaerobic growth at 37° C., colonies were enumerated. As a control, spores incubated in 100 μl of BH IS with 1% TCA (v/v) for 15 min were plated on BHIS containing 0.1% TCA (v/v) or on BHIS agar alone, respectively. Germination rate (%) was calculated using the following formula:

BHIS count/BHIS with 0.1% TCA×100.

The BHIS count would be expected to include only vegetative cells while the BHIS containing 0.1% (w/v) TCA would include vegetative cells and spores.

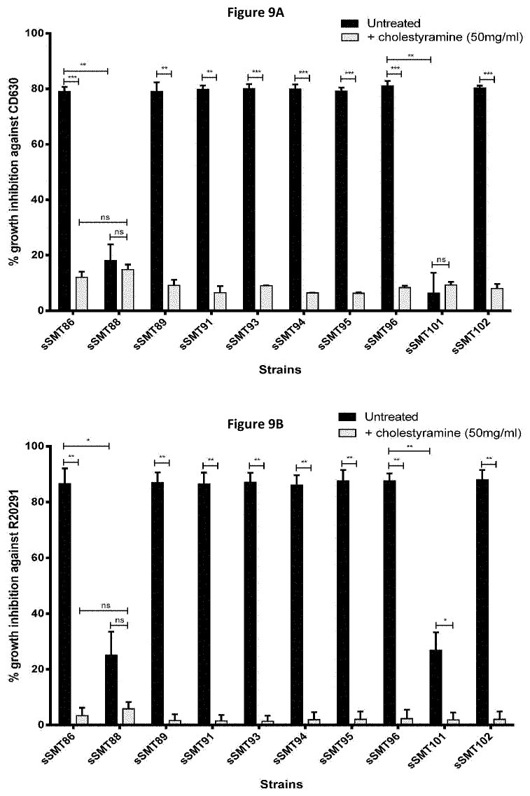

To determine whether Sb^{BSH} strains could inhibit CD spore germination, supernatants from strains grown in either 0 or 10 mM BAM supplemented FeSSCoF medium were incubated with CD spores in the presence of 1% taurocholic acid (FIG. 10). The reactions were serially diluted and then plated on BHIS plates for vegetative cell counts, or on BHISS for total counts, and the germination rate was calculated. Supernatants from all strains supplemented with 10 mM BAM with the exception of wild type (sSMT88) and sSMT101 showed appreciable inhibition of CD germination for CD630 (FIG. 10A) and R20291 (FIG. 10B) compared to non-supplemented media. This suggests that BAM supplementation elicited the production of metabolites in the supernatant of Sb^{BSH} strains that had an inhibitory effect upon the germination of C. difficile spores.

### Example 7

### Growth Profile of Sb^{BSH} Strains Selected for In Vivo Study

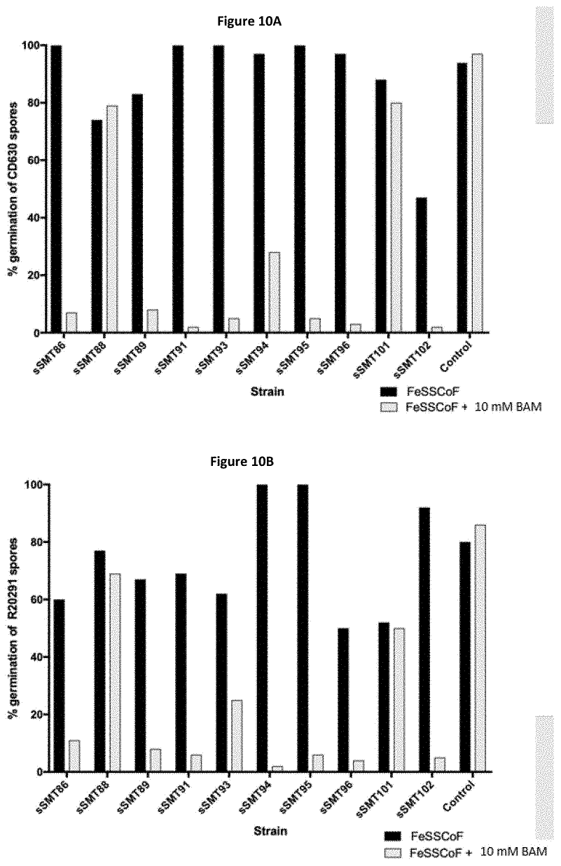

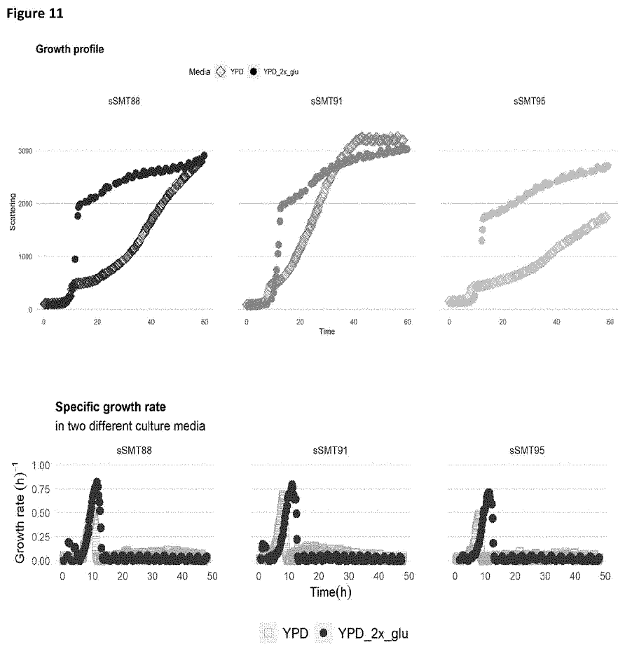

The growth profile of selected Sb^{BSH} strains was characterized in different complex media in order to improve strain health and performance, and in order to validate them as suitably for an ensuing animal study. Yeast strains sSMT88, sSMT91, and sSMT95 were re-activated from glycerol stocks on YPD agar plates. Then, the plates were incubated at 30° C. for two days. Subsequently, single colonies were inocculated in 5mL of YPD and incubated overnight at 30° C. and 180 rpm. These cultures were used as a seed to inoculate 50 mL of complex media contained in 250 ml shake flasks. We evaluated the growth of the strains in YPD and YPD/2× glucose (YPD_2×_glu). Growth cultures were standardized to an initial cell density of OD₆₀₀ 0.1, and the shake flasks were incubated at 30° C. and 180 rpm for three days. One experimental unit from each strain was used for online monitoring the biomass formation, measured as light scattering, by non-invasive CQG sensors (aquila biolabs GmbH). R Studio version 3.6.1 (RStudio Team, 2016) and the corresponding packages, including ggplot2 v.3.2.0 (Wickham, 2016) wesanderson v0.3.6 (Ram and Wickham, 2018), among others, were used for the data analysis and visualization. As presented in FIG. 11, the biomass formation (FIG. 11, top panel) and the growth rate (FIG. 11, bottom panel) were significantly increased by the addition of extra glucose to the media.

### Example 8

### Formulation of Recombinant S. boulardii Strains as Microsphere for GI Delivery

The S. boulardii strains as described in Example 1 are washed in sodium phosphate buffer pH 6.8 and suspended at a ratio of 75% water and 25% cells and lactose to a final cell density of 3-9×10¹⁰ CFU/gram and 4 g/L lactose. 10-20 g/L of sodium alginate in sterile water are added at a ratio of 100 mL alginate solution to 10-40 g of the yeast suspension. The mixture is extruded using a syringe through a 26 G needle using a dynamometer DY20B at a constant speed of 1 mm/min. Nitrogen gas flow (60 mBar) is used to generate small drops which form microspheres in 0.1 M CaCl₂ solution. The microspheres are left to harden for at least 30 min in 0.1 M CaCl₂ solution while mixing continuously. The microspheres are collected, rinsed with sterile water and dried in an oven for 3-4 days at 25° C.

### Example 9

### In Vivo Testing of Sb^{BSH} Strains in the Hamster CDI Model

All animal experiments adhered to the standards of EU Directive 2010/63/EU. All work with infected animals was performed in biosafety level 2 facilities. Female Golden Syrian hamsters (Envigo, UK), 9 to 10 weeks old, minimum 120 g in weight, were used in all animal experiments. After their arrival, the hamsters were permitted to adapt to their environment for at least 7 days before the experiments. They were housed in groups of 3/cage in IVCs (independently ventilated cages). Food (chow), water, bedding and cages were autoclaved or irradiated prior to use. Starting at day −3, animals were housed individually in IVC chambers (1 animal/cage). After C. difficile inoculation, animals were monitored for symptoms of disease progression and culled upon reaching the clinical endpoint. The symptoms of CDI were scored as severe/clinical endpoint (wet tail greater than 2 cm, high lethargy), mild (wet tail greater than 2 cm), or healthy.

Clostridioides difficile Strains and Sporulation

Per animal experiment, one of the following C. difficile strains can be used:
a) 630 [ribotype 012, tcdA+ tcdB+] (Wüst et al., 1982)
b) ST11 [ribotype 078] (Knight et al., 2019)
c) R20291 [ribotype 027, tcdA+ tcd B+] (Valiente et al., 2014)
d) 90556-M6S [ribotype 001] (Type strain 9689, American Type Culture Collection (ATCC))
e) VPI 10463 [ribotype 087] (strain 43255, ATCC); and
f) Isolate Bl17-6443 of strain NAP1/Bl/027 [ribotype 027] (Chen et al.; 2008; Fatima & Aziz, 2019).

For the following hamster experiment, CD630 was used. 630 is a highly infectious strain of C. difficile (Goulding et al., 2009). Spores of CD630 were prepared previously and purified through a 20-50% Histodenz gradient (Phetcharaburanin et al., 2014) and stored at 4° C. The spores were pre-validated to ensure infectivity.

Preparation of S. boulardii for Oral Gavage Administration

2-3 days prior to in vivo study, fresh S. boulardii cells were prepared by streaking the S. boulardii strains (sSMT88, sSMT91, sSMT95) onto YPD agar plates. The agar plates were then incubated at 37° C. for 2-3 days.

The day before administration by oral gavage to hamsters, overnight S. boulardii cell cultures were prepared by inoculating a full loop of cells into 48 ml of Yeast Extract-Peptone-Dextrose (YPD) liquid media in 250 ml baffled Erlenmeyer flasks. Culture flasks with Sb were incubated aerobically at 37° C. with shaking, overnight, at 200 rpm. CFU/ml for each strain was determined and 6 ml was found to be sufficient for oral gavage of one animal. The next day, the overnight culture of each Sb strain was centrifugated at 4,000 rpm for 5 minutes. After that, the supernatant was removed, and cells washed 3-times with sterile MilliQ water. Next, the cells were resuspended in PBS (3.5 ml PBS per strain). The OD600 of the Sb strains was measured and adjusted for dosage. Doses of ^{˜}1-3×109 CFU in 500 μl of PBS of recombinant S. boulardii strain per hamster were administered daily by oral gavage, at the same time each day. Fresh cell cultures were prepared each day for oral gavage administration for the duration of the experiment.

Hamster Model of CDI with Sb^{BSH} Treatment

The hamster model of CDI was adapted from Hong et al., 2017, (Freeman et al., 2005), and (Sambol, Tang, Merrigan, Johnson, & Gerding, 2001) with the following alterations. In this study, a single dose of 30 mg/kg body weight clindamycin (clindamycin 2-phosphate, Sigma, C6427) was administered by oral gavage (Day −1), and individual doses of 1-3×10⁹ CFU of respective recombinant S. boulardii strain in 500 μL 1× PBS (137 mM NaCl, 2.7 mM KCl, 8 mM Na₂HPO₄, and 2 mM KH₂PO₄) were administered by oral gavage (Day −3 to end of study). 24 hamsters were divided into 4 groups (1, 2, 3, 4) with 6 animals per group, listed in Table 3. Starting on day −3 and for the remainder of the study, animals received one daily dose of 1× PBS (Group 1, infection control), one daily dose of S. boulardii wild type (Group 2, Sb^{WT}), or one daily dose of the two best performing S. boulardii strains producing BSH of Example 6 (Group 3 and 4, Sb^{BSH1} and Sb^{BSH2}). On day −1, all groups were administered clindamycin. On day 0, all hamsters were infected with 100 C. difficle spores of the CD630 strain by oral gavage 16 h after clindamycin challenge. The study was terminated at day 3, when the last animal had perished. Animals were monitored for symptoms of disease progression and culled upon reaching the clinical endpoint.

In Vivo Study Specifications.

Model: Acute hamster model with CD630 challenge 20-hours post clindamycin administration (Hong et al., 2017a and b)

Species: Hamsters, Golden Syrian.

Supplier: Envigo

Sex: Females

Age: 9-10 weeks at study start (≥120 g)

Total number: 24

Group size: 6/group (n =6)

TABLE 3
Experimental groups in hamster CDI model with Sb^{BSH}

| | | Relevant | |
|---|---|---|---|
| Group | Number | Administration | Dosing route |
| 1 | 6 | PBS | D3 until end of study, Sham (PBS) |
| | | | dosed by intra-gastric (i.g.) gavage; |
| | | | 0.5 ml 1×/day |
| 2 | 6 | Sb^{WT} wild type | D3 until end of study, sSMT88 |
| | | (sSMT88) | dosed by intra-gastric (i.g.) gavage; |
| | | suspended in | 0.5 ml 1×/day |
| | | PBS | |
| 3 | 6 | Sb^{BSH1} (sSMT91) | D3 until end of study, sSMT91 |
| | | suspended in | dosed by intra-gastric (i.g.) gavage; |
| | | PBS | 0.5 ml 1×/day |
| 4 | 6 | Sb^{BSH2} (sSMT95) | D3 until end of study, sSMT95 |
| | | suspended in | dosed by intra-gastric (i.g.) gavage; |
| | | PBS | 0.5 ml 1×/day |

Study plan. The test study was conducted as follows:
Day −3 to study end
   Animals housed individually in IVC chambers (1 animal/cage)
   Intra-gastric dosing of Sb (1 dose/day, 1-3×10⁹ CFU in PBS, 0.5 ml/dose)
   Intra-gastric dosing of Sb (as stated in Table 3) maintained daily (1 dose/day, ˜13×10⁹ CFU in PBS, 0.5 ml/dose)
Day −1 Animals administered a single dose of clindamycin-2-phosphate (30 mg/kg body weight) by oral gavage (intra-gastric gavage)
Day 0: Animals administered a single oral (intra-gastric gavage) dose of CD630 spores (100 spores)
Days 0-2 Monitoring of symptoms (last hamster succumbed to CDI at 50 h)

Sample analysis. Correlates of CDI to be determined:
C. difficile spore counts (CFU) in cecum on ChromID agar (BioMerieux)
Collect content of cecum and colon. Store at −20° C. for LC-MS analysis of bile acid pool, performed by Alderley Analytical Ltd, UK
Levels of toxins A in cecum (determined by ELISA)
% animals colonized with CD630
Time between challenge and colonization
Graded and temporal assessment of symptoms prior to endpoint
Monitor size of appendix, harvest if visibly enlarged
Correlates of retention and survival of Sb^{wT} and Sb^{BSH}to be determined:
CFU enumeration of S. boulardii in feces collected from cage at time of death

### Procedural Notes

Animals (4-weeks of age) were purchased from Envigo, UK. Hamsters were housed in groups of 3 for 4 weeks in a secure animal house with climate controlled (temperature, humidity, light) and electronic access until they reached a sufficient weight (≥120 g).

On day −3 animals were transferred to IVC cages (1 animal per cage). This is critical since C. difficile can easily cross-contaminate between animals. All cages, water, bedding and food were sterilized (autoclaving, UV treatment as appropriate). All work with C. difficile and infected animals was conducted at Biosafety Level 2.

On day −3 animals were administered the appropriate S. boulardii suspension or PBS sham treatment as specified in Table 3, once per day (0.5 ml/dose). This was repeated daily until the end of the experiment on day 3 (50 hours). On day −1 clindamycin 2-phosphate was given to hamsters by single i.g. gavage at 30 mg/kg.

Adverse effects and/or CDI symptoms were recorded daily from day −3. Upon death, hamsters were immediately frozen at −20 ° C. Once a hamster had succumbed to infection, feces was collected from the cage and tested for S. boulardii CFU. On the day of analysis, hamsters were thawed, cecum removed and used for immediate toxin and CFU analysis. Samples were taken from the colon and cecum and stored at −20° C. for later LCMS analysis.

On day 0 all hamsters were challenged with 1×10² spores of C. difficile strain 630 (200μl oral gavage) 20 h after clindamycin treatment. The study endpoint was reached at 50 h when all hamsters had succumbed to infection.

### Extraction and Measurement of Toxins

Hamsters were frozen upon death and for analysis were thawed and cecum samples removed and analyzed immediately. The wet weight of material was determined, and samples were suspended in 3-5 volumes of toxin extraction buffer (2 ml Pierce Protease Inhibitor tablets (Thermo Scientific, 88265), 500 μl EDTA (ethylenediaminetetraacetic acid, 0.5M), 48.5 ml PBS, pH 7.4). Samples were then macerated, and toxins extracted for 2 h at 4° C. with gentle agitation. Tubes were centrifuged (microfuge, 4° C., 15 min, 10.000 rpm) and the supernatant filtered through a 0.2 μm Corning syringe filter. Filtered supernatant was stored at 4° C. prior to use.

Measurement of C. difficile Toxin A by capture ELISA. ELISA analysis of toxin A was performed in order to assess disease progression as described previously in Hong et al., 2017a. ELISA plates (GREINER, high binding type) were coated with Rabbit—anti-ToxA (1/6.000) in 0.01M PBS buffer pH 7.4, 50 μl per well. Plates were incubated overnight at RT, and washed 3× with PBS+0.05% Tween20 (PBS+Tween). Blocking was performed with 2% BSA in PBS (150 μl/well) for 1 hr at 37° C., with 3 subsequent short washes with PBS+Tween. Samples were added (50 ul/well), starting with 1/2 -1/4 sample dilution of caecal extraction in diluent. After an incubation for 2 h at RT, plates were washed 3× with PBS+Tween, with 1 min in between. Then the detection anti-toxin antibody was added, mouse anti-CDTA14 (1/1000) in diluent, 50 μl/well. After an incubation for 1 h at 30° C., plates were washed 3×, with 2 minutes interval. For detection, anti-mouse HRP antibody (Biolegend) (1/4000 in diluent) was added and incubated for 1 h at RT. After washing (3×) with 3 minute intervals, the substrate (Biolegend) was added, and the reaction stopped with 2N H₂SO₄ and the plate was read at 450 nm, and the amount of toxins per g of sample determined.

Determination of C. difficile Spore Counts (CFU) to Analyze Colonization Efficiency

Enumeration of the total C. difficile spore counts in cecum samples was performed as described by Lawley, et al. (2009). After toxin extraction, samples were centrifuged as described in the extraction method and pellets suspended in 3-5 volumes of 80% (v/v) ethanol, vortexed vigorously for 5 min and incubated for 1 hour at RT before serial dilution and plating on selective media (CHROMID® C. difficile, bioMerieux, Ref. 43871). Plates were incubated for 24 h at 37° C. under anaerobic conditions. Colonies were counted and C. difficile CFU per g of sample calculated. Thereby, the percentage of animals colonized by C. difficile was determined.

Determination of S. boulardii Counts (CFU) to Verify Colonization

CFU analysis of S. boulardii from hamster fecal samples. Enumeration of the total viable S. boulardii in fresh feces samples was performed as described in Chen et al., 2015 and Toothaker and Elmer 1984, with the following adjustments. The feces were collected from each cage once the residing hamster had succumbed to CDI. Each sample was weighed and homogenised in 10 volumes of 0.9M saline solution with occasional vortexing for 1 h. A ten-fold serial dilution series corresponding to ^{˜}10¹⁰ CFU/mL to 0 CFU/mL was tested per sample. The number of S. boulardii CFU in fecal content was determined by plating the serial dilutions on two sets of solid Sabouraud plates. Set A contains cycloheximide (0.5 g/L), and both sets A and B contain gentamicin (80 mg/L). Set B supports the unselective growth of various yeast species, including S. boulardii. Set A is selective for interfering Candida species. Colonies were counted after incubation in an aerobic incubator for 3 days. For each sample, the difference in colony counts between the two plate sets was used to calculate the CFU spiked into each sample.

### Bioanalysis

Analytical quantification of the BSH substrate (conjugated bile salts) and conversion product (deconjugated bile salts) levels is carried out by LC-MS as described above, and performed at.

Detection of C. difficile Toxin A By Capture ELISA

ELISA analysis of toxin A was performed in order to assess disease progression as described previously in Hong et al., 2017a. ELISA plates (GREINER, high binding type) were coated with Rabbit anti-ToxA (1/6.000) in 0.01M PBS buffer pH 7.4, 50 μl per well. Plates were incubated overnight at RT and washed three times with PBS+0.05% Tween20 (PBS+Tween). Blocking was performed with 2% BSA in PBS (150 μl/well) for 1 h at 37° C., with three subsequent short washes with PBA+Tween. Samples were added (50 μl/well), starting with one half sample dilution of caecal extraction in diluent. After an incubation for 2 h at RT, plates were washed three times with PBS+Tween, with 1 min in between. Then the detection anti-toxin antibody was added, mouse anti-CDTA14 (1/1000) in diluent, 50 μl/well. After an incubation for 1 h at 30° C., plates were washed three times, with two minutes interval. For detection, anti-mouse HRP antibody (Biolegend) (1/4000 in diluent) was added and incubated for 1 h at RT. After washing (three times) with 3 minutes intervals, the substrate (Biolegend) was added and the raction stopped with 2N H₂SO₄ and the plate was read at 450 nm.

### Histopathology

Caecum samples are prepared for simple histology as described by (Buckley et al., 2013; Goulding et al., 2009) to evaluate mucosal damage and inflammation induced by the C. difficile toxins. Caecal pathology is scored in a blinded fashion, grading neutrophil margination (0, no neutrophil accumulation; 1, local acute neutrophil accumulation; 2, extensive submucosal neutrophil accumulation; 3, transmural neutrophilic infiltrate), haemorrhagic congestion (0, normal tissue; 1, engorged mucosal capillaries; 2, submucosal congestion with unclotted blood; 3, transmural congestion with unclotted blood), hyperplasia (0, no epithelial hyperplasia; 1, twofold increase in thickness; 2, threefold increase in thickness; 3, fourfold or greater increase in thickness), and percent of epithelial barrier involvement (0, no damage; 1, less than 10% of mucosal barrier involved; 2, less than 50% of mucosal barrier involved; 3, more than 50% mucosal barrier involved). Results are expressed as mean pathology score per strain for each criterion.

### Statistical Analysis

Graphs and statistics were generated using the Prism software (GraphPad Software, version 6.1). Survival data were analyzed by Kaplan—Meier survival analysis. Significance between treatment groups was calculated using the Post-hoc Power analysis (https://clincalc.com/stats/Power.aspx.).

Results of the Hamster Model of CDI with Sb^{BSH}

The following results were obtained from the in vivo hamster CDI model.

Observed hamster symptoms. During CDI hamsters often develop diarrhoea due to haemorrhagic caecitis which manifests as a ‘wet tail’, the diameter of which can indicate the severity and progression of disease (Buckley et al., 2011). The CDI symptom grading of animals is subjective and the following symptom details were used as a guideline. CDI symptoms were scored and grouped into three distinct categories. Mild diarrhoea and a wet tail (diameter less than 2 cm; i.e. the diameter of the visible external wet area on the hamster's underbelly) were considered to be mild symptoms (+). Heavy diarrhoea, a wet tail (diameter less than 2 cm) and lethargy were considered to be moderate symptoms (++). A wet tail (diameter greater than 2 cm), extreme lethargy and visible weakness were considered to be severe symptoms (+++). Hamsters were observed closely after symptoms had developed and moribundity and death often rapidly followed the onset of severe symptoms.

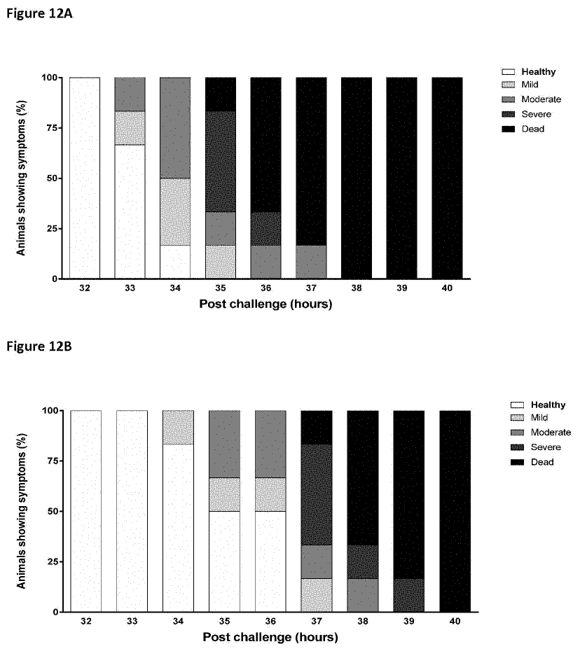

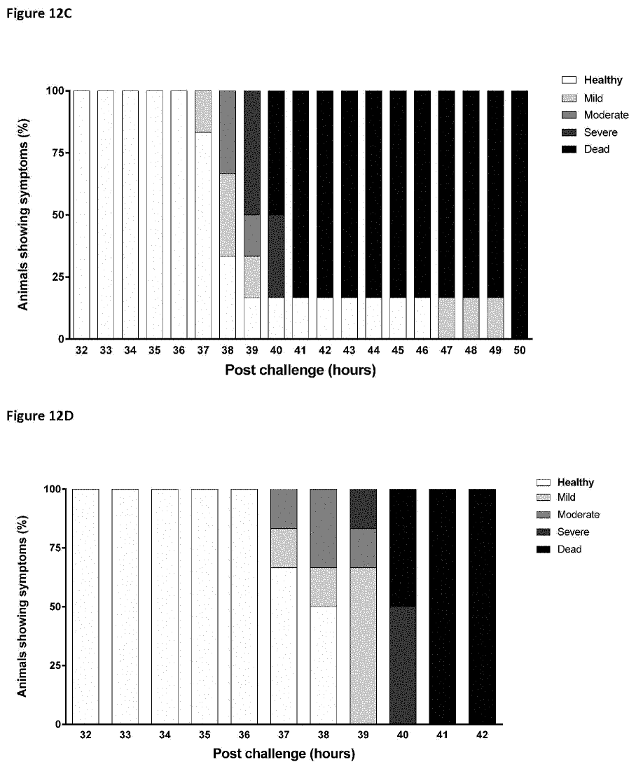

Analysis of symptoms (FIG. 12A-D, Table 4-5) revealed a severe and fulminant CDI infection, culminating in the death of all hamsters in the PBS placebo group by 38 hours post-challenge. Group 2 (sSMT88) began to show symptoms at 34 hours post-challenge and by 38 hours, 4 hamsters had succumbed to CDI (67%) and the remaining 2 hamsters were showing symptoms. By 40 hours post-challenge all hamsters in group 2 had succumbed to CDI, demonstrating a slightly delayed infection time course compared to the placebo group. Both group 3 (sSMT91) and group 4 (sSMT95) showed a delayed time course compared to groups 1 and 2. The sSMT91 group began to show symptoms at 37 hours post-challenge, and by 41 hours 5 hamsters had succumbed to CDI (83%) with the remaining hamster surviving until 50 hours post challenge. The sSMT95 group started to develop symptoms at 37 hours post-challenge and all hamsters were deceased by 41 hours. The time course in infection for groups 3 and 4 were similar to one another. There was a noticeable difference in disease progression and symptom presentation between the placebo group (group 1) and sSMT88 (group 2, SbWT, wild type). There was a further delay in symptom presentation and progression between these 2 groups and the two treatment groups, sSMT91 (group 3, SbBSH1) and sSMT95 (group 4, SbBSH2).

TABLE 4
Observed hamster symptoms until 50 hours post-challenge (average values).

| | Time from challenge to colonization | | | Time from challenge to death | | |
|---|---|---|---|---|---|---|
| | | Post-hoc | Post-hoc | | Post-hoc | Post-hoc |
| | Average ± | Power¹ vs | Power vs | Average ± | Power vs | Power vs |
| Group | SD (h) | placebo (%) | Sb^{WT} (%) | SD (h) | placebo (%) | Sb^{WT} (%) |
| Group 1 | 34 ± 1 | — | — | 36 ± 1 | — | — |
| (infection control) | | | | | | |
| Group 2 | 36 ± 1 | 93 | — | 38 ± 1 | 93 | — |
| (Sb^{WT}, sSMT88) | | | | | | |
| Group 3 | 40 ± 4 | 95 | 66 | 42 ± 4 | 94 | 66 |
| (Sb^{BSH1}, sSMT91) | | | | | | |
| Group 4 | 38 ± 1 | 100 | 93 | 41 ± 1 | 100 | 100 |
| (Sb^{BSH2}, sSMT95) | | | | | | |

¹Post-hoc power was calculated using the website https://clincalc.com/stats/Power.aspx.

TABLE 5
Observed hamster symptoms until 41 hours post-challenge¹ (individual values).

| | | Hours post CD630 challenge | | | | | | | | | |
|---|---|---|---|---|---|---|---|---|---|---|---|
| Group | Hamster | 32 h | 33 h | 34 h | 35 h | 36 h | 37 h | 38 h | 39 h | 40 h | 41 h |
| 1-PBS | 1 | − | − | + | ++ | +++ | DEAD | DEAD | DEAD | DEAD | DEAD |
| | 2 | − | + | ++ | DEAD | DEAD | DEAD | DEAD | DEAD | DEAD | DEAD |
| | 3 | − | − | − | + | ++ | ++ | DEAD | DEAD | DEAD | DEAD |
| | 4 | − | ++ | ++ | +++ | DEAD | DEAD | DEAD | DEAD | DEAD | DEAD |
| | 5 | − | − | + | +++ | DEAD | DEAD | DEAD | DEAD | DEAD | DEAD |
| | 6 | − | − | ++ | +++ | DEAD | DEAD | DEAD | DEAD | DEAD | DEAD |
| 2-sSMT88 | 7 | − | − | + | ++ | ++ | +++ | DEAD | DEAD | DEAD | DEAD |
| | 8 | − | − | − | − | − | +++ | +++ | DEAD | DEAD | DEAD |
| | 9 | − | − | − | − | − | DEAD | DEAD | DEAD | DEAD | DEAD |
| | 10 | − | − | − | − | − | + | ++ | +++ | DEAD | DEAD |
| | 11 | − | − | − | ++ | ++ | +++ | DEAD | DEAD | DEAD | DEAD |
| | 12 | − | − | − | + | + | ++ | DEAD | DEAD | DEAD | DEAD |
| 3-sSMT91 | 13 | − | − | − | − | − | − | − | − | − | − |
| | 14 | − | − | − | − | − | − | − | + | +++ | DEAD |
| | 15 | − | − | − | − | − | − | ++ | +++ | DEAD | DEAD |
| | 16 | − | − | − | − | − | − | ++ | +++ | DEAD | DEAD |
| | 17 | − | − | − | − | − | + | + | ++ | DEAD | DEAD |
| | 18 | − | − | − | − | − | − | + | +++ | +++ | DEAD |
| 4-sSMT91 | 19 | − | − | − | − | − | − | + | + | DEAD | DEAD |
| | 20 | − | − | − | − | − | + | ++ | ++ | DEAD | DEAD |
| | 21 | − | − | − | − | − | − | − | + | +++ | DEAD |
| | 22 | − | − | − | − | − | − | − | + | +++ | DEAD |
| | 23 | − | − | − | − | − | − | − | + | +++ | DEAD |
| | 24 | − | − | − | − | − | ++ | ++ | +++ | DEAD | DEAD |

¹Symptoms designated according to severity; + mild, ++ moderate, +++ severe
² Hamster 13 survived until 50 h post-challenge

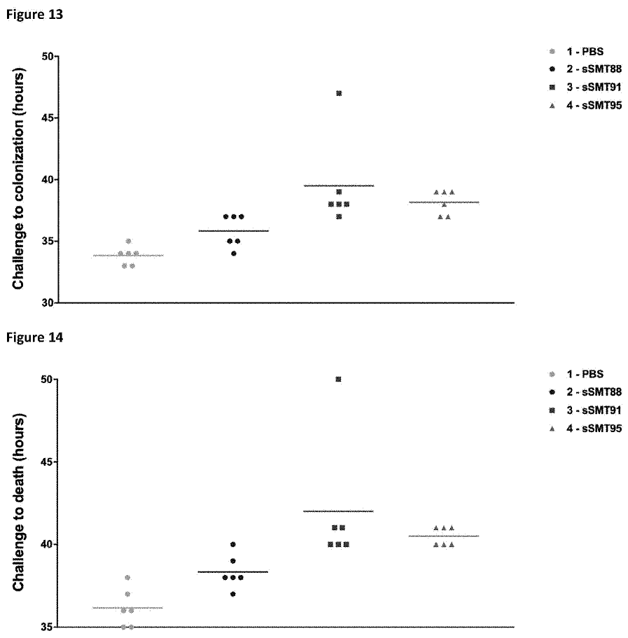

Time from challenge to colonization. Colonization is herein defined by the presence of symptoms. Comparison of the time of challenge to colonization revealed there to be substantial differences between the treatment groups and the placebo group (FIG. 13, Table 4-5). For the placebo group, average time from challenge to colonization was 34 hours, while it was 36 hours for sSMT88 (wild type), 40 hours for sSMT91 and 38 hours for sSMT95. sSMT91 and sSMT95 displayed a very similar time course, with the time difference for challenge to colonization differing principally due to hamster 13 which only started to display symptoms at 47 hours.

Time from challenge to death. A similar pattern emerged when analyzing time from colonization to death (FIG. 14), with an average time for the PBS placebo group of 36 hours, 38 hours for sSMT88, 42 hours for sSMT91 and 41 hours for sSMT95.

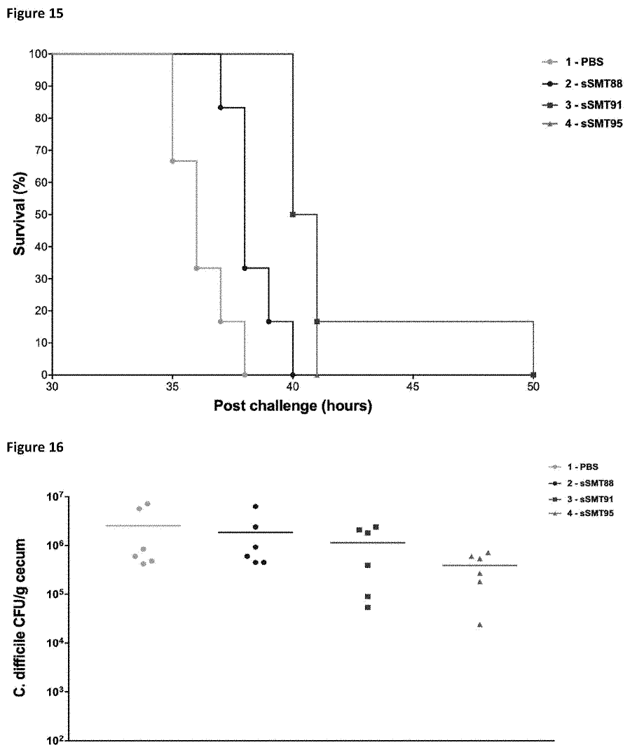

The Kaplan-Meier survival curve (FIG. 15) illustrates the differences in survival over the study for the different groups, with hamsters from both treatment groups (sSMT91 and sSMT95) outlasting the animals in groups 1 (PBS placebo) and group 2 (sSMT88, wild type).

C. difficile spore counts. C. difficile in spore form in cecum samples confirms infection/colonization and reflects progression of disease over time. Analysis of C. difficile CFU in cecum samples (FIG. 16) did not show any significant differences between the placebo and test groups. None of the S. boulardii strains prevented C. difficile colonization, which was defined above as 102 CFU/g.

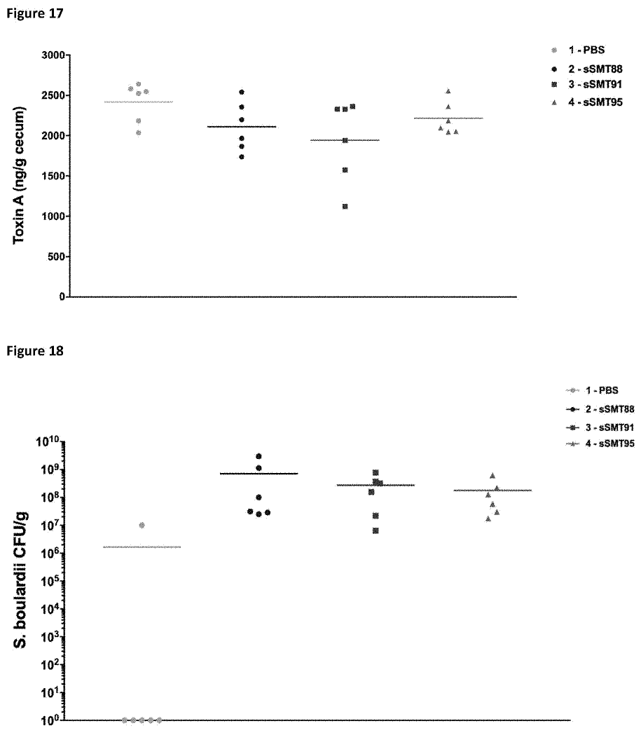

ELISA analysis of toxin A. Levels of toxins A and B in cecum reflect progression of disease. The toxin A level within the ceca of the hamsters was analysed using ELISA (FIG. 17). Toxin A levels were indistinguishable between the treatment groups and the placebo. In hamster models of CDI, once infection has begun, hamsters will suffer from a severe and fulminant infection and upon death will have a large quantity of C. difficile spores and toxins present in the cecum (Heidebrecht et al., 2019). Therefore, if a hamster has succumbed to CDI it is unlikely that toxin and spore counts will differ substantially between animals regardless of the time of death within the study. The presence of toxins and CFU within the ceca of hamsters confirms morbidity by CDI.

S. boulardii enumeration. S. boulardii in vegetative form in facal amples confirms colonization with probiotic yeast cells. Upon death, feces were collected from the cages of the hamsters and stored at 4° C. for 24 hours, prior to analysis. All hamsters were moved into new cages at 24 hours post C. difficile challenge so all feces were collected after this point in time. S. boulardii counts in these fecal samples revealed the presence of high numbers of viable CFU (^{˜}10⁸ to 10⁹ CFU/g) in all treatment groups, and an absence of CFU in the PBS placebo group (FIG. 18). The CFU count for hamster 5 (PBS placebo group) seems to be anomalous and might be due to a large number of interfering Candida species present within the sample.

Bioanalytical analysis of bile salts. Analytical quantification of the BSH substrate (conjugated bile salts) and conversion product (deconjugated bile salts) levels present in the colon and caecum is carried out by LC-MS as described above, and performed at Alderley Analytical, UK. The detection of the altered bile salt composition in groups 3 and 4 compared to group 1 and 2 confirms in situ production and active presence of API.

Histopathology. Histopathological analysis shows more severe mucosal damage and inflammation induced by the C. difficile toxins in the controls versus the BSH producing yeast strain.

### Example 10

### In Vivo Testing of a Combination Therapy of Sb^{BSH} Strains and Antibiotics Treatment in the Hamster CDI Model

Hamster Model of CDI with Combined Sb^{BSH}and Antibiotics Treatment

It has been shown that there was a significant decrease in recurrent C. difficile disease (RCDD) in patients treated with high-dose vancomycin (2 g/day) and S. boulardii (16.7%), compared with those who received high-dose vancomycin and placebo (50%; P=0.05), with no serious adverse reactions observed in these patients (Surawicz et al., 2000). Also, S. boulardii prevented the development of high counts of C. difficile and high titers of toxin B of vancomycin treated hamsters (Elmer et al., 1987).

The current treatment recommendations for clinical patients are treatment with vancomycin orally 4 times a day or fidaxomicin twice daily, both for a total of 10 days. For recurrent CDI, potential treatment options include prolonged tapered and pulsed vancomycin regimen for up to 8 weeks (McDonald et al., 2018). Therefore, in some embodiments a combined therapy of vancomycin and Sb^{BSH} will provide even more efficacy in treating CDD and/or RCDD.

The hamster model of CDI is adapted from Hong et al., 2017, Freeman et al., 2005, and Sambol, Tang, Merrigan, Johnson, & Gerding, 2001 with the following alterations. In this study, a single dose of 30 mg/kg body weight clindamycin (clindamycin 2-phosphate, Sigma) is administered by oral gavage (Day −1), individual doses of 2 mg/L vancomycin (vancomycin hydrochloride, Sigma) are administered by oral gavage (Day 1 to end of study), and individual doses of 1-3×10⁹ CFU of respective recombinant Sb^{BSH} strain in 500 μL 1× PBS are administered by oral gavage (Day −3 to end of study). 30 hamsters are divided into 5 groups (1, 2, 3, 4, 5) with 6 animals per group, listed in Table 6. Starting on day −3 and for the remainder of the study, animals receive one daily dose of 1× PBS (Group 1, infection control), one daily dose of S. boulardii wild type (Group 2, Sb^{WT}), one daily dose of the best performing S. boulardii strain producing BSH of Example 6 (Group 3, Sb^{BSH}), one daily dose of vancomycin (Group 4, vancomycin), or one daily dose of Sb^{BSH} and vancomycin (Group 5, Sb^{BSH}+vancomycin). On day −1, all groups are administered clindamycin. On day 0, all hamsters are infected with 100 C. difficle spores of the CD630 strain by oral gavage 16 h after clindamycin challenge. The study is terminated at day 12, depending on disease progression and recovery in the different groups. Animals are monitored for symptoms of disease progression and culled upon reaching the clinical endpoint (wet tail diameter 2 cm, high lethargy).

TABLE 6
Treatment groups in hamster CDI model with Sb^{BSH} + drug treatment

| Group | Number | Relevant Administration | Dosing route |
|---|---|---|---|
| 1 | 6 | PBS | intra-gastric gavage |
| 2 | 6 | Sb^{WT} suspended in PBS | intra-gastric gavage |
| 3 | 6 | Sb^{BSH} suspended in PBS | intra-gastric gavage |
| 4 | 6 | vancomycin | intra-gastric gavage |
| 5 | 6 | Sb^{BSH} suspended in | intra-gastric gavage |
| | | PBS + vancomycin | |

Correlates of CDI to Be Determined:
C. difficile spore counts (CFU) in cecum
Collect content of cecum and colon. Store at −20 ° C. for LC-MS analysis of bile acid pool, to be performed by Alderley Analytical Ltd, UK
Levels of toxins A in cecum (determined by ELISA)
% animals colonized with CD630
Time between challenge and colonization
Graded and temporal assessment of symptoms prior to endpoint (end point is the point at which animals enter Stage 3 of symptoms and where one is legally obliged to kill the animals)
Monitor size of appendix, harvest if visibly enlarged

Correlates of Retention and Survival of Sb^{wT} and Sb^{BSH} to be determined:

CFU enumeration of S. boulardii in feces collected from cage at time of death

### Sample Collection

After euthanization, hamsters are frozen immediately. Hamster cecum content samples are collected for CFU analysis, colon and cecum content is collected for LC-MS analysis, and colon and cecum tissues are stored for optional resection for histopathological analysis.

C. difficile Enumeration to Analyse Colonization Efficiency

Enumeration of the total C. difficile spore counts in cecum samples is performed as described by Lawley, et al. (2009). Thereby, the colonization efficiency of C. difficile is determined.

S. boulardii Eumeration

Enumeration of the total viable S. boulardii in feces is performed as described in (L. A. Chen et al., 2015) and (Toothaker & Elmer, 1984) with the following adjustments. The feces are collected from cage at time of death. Each sample is dissolved by adding 5 mL 0.9 M saline solution with occasional vortexing for 1-2 h. A ten-fold serial dilution series corresponding to 10¹⁰ CFU/mL to 0 CFU/mL is tested per experiment. The number of S. boulardii CFU in feces is determined by plating the serial dilutions on two sets of solid Sabouraud plates. Set A contains cycloheximide (0.5 g/L), and both sets A and B contain gentamicin (80 mg/L). Set B supports the unselective growth of various yeast species, including S. boulardii. Set A is selective for interfering Candida species. Colonies are counted after incubation in an aerobic incubator at 37° C. for 2-3 days. For each experiment the difference in colony counts between the two plate sets is used to calculate the CFU spiked into each sample.

### Bioanalysis

Analytical quantification of the BSH substrate (conjugated bile salts) and conversion product (deconjugated bile salts) levels present in the colon and caecum is carried out by LC-MS as described above, and performed at Alderley Analytical, UK.

### Histopathology

Caecum samples are prepared for simple histology as described by (Buckley et al., 2013; Goulding et al., 2009) to evaluate mucosal damage and inflammation induced by the C. difficile toxins. Caecal pathology is scored in a blinded fashion, grading neutrophil margination (0, no neutrophil accumulation; 1, local acute neutrophil accumulation; 2, extensive submucosal neutrophil accumulation; 3, transmural neutrophilic infiltrate), haemorrhagic congestion (0, normal tissue; 1, engorged mucosal capillaries; 2, submucosal congestion with unclotted blood; 3, transmural congestion with unclotted blood), hyperplasia (0, no epithelial hyperplasia; 1, twofold increase in thickness; 2, threefold increase in thickness; 3, fourfold or greater increase in thickness), and percent of epithelial barrier involvement (0, no damage; 1, less than 10% of mucosal barrier involved; 2, less than 50% of mucosal barrier involved; 3, more than 50% mucosal barrier involved). Results are expressed as mean pathology score per strain for each criterion.

### Statistical Analysis

Graphs and statistics are generated using Microsoft Excel or Prism software (GraphPad

Software, version 6.1). Survival data are analyzed by Kaplan-Meier survival analysis. Significance between treatment groups is calculated using the Post-hoc Power analysis (https://clincalc.com/stats/Power.aspx.).

### Results

The following results are obtainable from the in vivo hamster CDI model experiments.

TABLE 7
Experimental outcomes of the hamster model for CDI and combined therapy
with Sb^{BSH} and antibiotic.

| | | Survival at | Weight | | |
|---|---|---|---|---|---|
| | Colonization | day 3 after | loss at | Diarrhea | Histopathology |
| | with | infection | day 3 after | over time | (severity of |
| Group | C. difficile | (% animals) | infection | (% animals) | enteritis) |
| Group 1 | 100% | 0% | High | Majority | Severe |
| (infection control) | | | | | |
| Group 2 | <Group 1 | >Group 1 | <Group 1 | <Group 1 | Less severe |
| (Sb^{WT}) | | | | | than Group 1 |
| Group 3 | <<Group 1 | >>Group 1 | <<Group 1 | <<Group 1 | <<Group 1 |
| (Sb^{BSH}) | <Group 2 | >Group 2 | <Group 2 | <Group 2 | <Group 2 |
| Group 4 | <<Group 1 | >>Group 1 | <<Group 1 | <<Group 1 | <<Group 1 |
| (vancomycin) | <Group 2 | >Group 2 | <Group 2 | <Group 2 | <Group 2 |
| Group 5 | <<<Group 1 | >>>Group 1 | <<Group 1 | <<<Group 1 | <<<Group 1 |
| (Sb^{BSH} + vancomycin) | <<Group 2 | >>Group 2, 3, 4 | <Group 2 | <<Group 2, 3, 4 | <<Group 2, 3, 4 |

Results from Analyses Performed on Samples Collected from the Hamster Model of CDI.

C. difficile spore counts. C. difficile in spore form in cecum samples confirms infection/colonization and reflects progression of disease over time.

S. boulardii enumeration. S. boulardii in vegetative form in faecal samples confirms colonization with probiotic yeast cells.

Bioanalytical analysis. Detection of the altered bile salt composition confirms in situ production and active presence of API.

ELISA analysis of toxin A and/or B. Levels of toxins A and/or B in cecum reflect progression of disease, and are lower in the animals treated with Sb^{BSH}, or even lower in Sb^{BSH}+drug treatment.

Histopathology. Histopathological analysis shows more severe mucosal damage and inflammation induced by the C. difficile toxins in the controls versus the Sb^{BSH} yeast strain.

### REFERENCEES

Almagro Armenteros, J. J., Tsirigos, K. D., Sønderby, C. K., Petersen, T. N., Winther, O., Brunak, S., Nielsen, H. (2019). SignalP 5.0 impoves signal peptide predictions using deep neural networks. Nature Biotechnology, 37(4), 420-423. https://doi.org/10.1038/s41587-019-0036-z
Bagherpour, G., Ghasemi, H., Zand, B., Zarei, N., Roohvand, F., Ardakani, E. M., Azizi, M., & Khalaj, V., (2018). Oral Administration of Recombinant Saccharomyces boulardii Expressing Ovalbumin-CPE Fusion Protein Induces Antibody Response in Mice. Front. Microbiol., 13 Apr. 2018. https://doi.org/10.3389/fmicb.2018.00723
Barlowe, C. K., Miller, E. A. (2013). Secretory Protein Biogenesis and Traffic in the Early Secretory Pathway. Genetics Feb. 1, 2013 vol. 193 no. 2 383-410; https://doi.org/10.1534/genetics.112.142810 Begley, M., Hill, C., Gahan, C. G. M. (2006). Bile Salt Hydrolase Activity in Probiotics.
Blehaut, H., Massot, J., Elmer, G. W., & Levy, R. H. (1989). Disposition kinetics of Saccharomyces boulardii in man and rat. Biopharmaceutics & Drug Disposition, 10(4), 353-364. https://doi.org/10.1002/bdd.2510100403
Brandvold, K. R., Weaver, J. M., Whidbey, C., & Wright, A. T. (2019). A continuous fluorescence assay for simple quantification of bile salt hydrolase activity in the gut microbiome. Scientific Reports, 9(1), 1359. https://doi.org/10.1038/541598-018-37656-7
Buckley A M, Spencer J, Candlish D, Irvine J J, Douce G R. Infection of hamsters with the UK Clostridium difficile ribotype 027 outbreak strain R20291. J Med Microbiol. 2011;60(Pt 8):1174-1180
Buckley, A. M., Spencer, J., Maclellan, L. M., Candlis, D., Irvine, J. J., Douce, G. R. (2013). Susceptibility of Hamsters to Clostridium difficile Isolates of Differing Toxinotype. Public Library of Science 8(5): e64121. DOI: 10.1128/AAC.26.4.552
Buffie at al.; Profound Alterations of Intestinal Microbiota following a Single Dose of Clindamycin Results in Sustained Susceptibility to Clostridium difficile-Induced Colitis; Infection and Immunity; 2010; vol 80(1); pp. 62-73.
Castagliuolo, I., Riegler, M. F., Valenick, L., LaMont, J. T., Pothoulakis, C. (1999). Saccharomyces boulardii Protease Inhibits the Effects of Clostridium difficile Toxins A and B in Human Colonic Mucosa. Infect Immun; vol:67(1); pp 302-7.
Chen et al.; A Mouse Model of Clostridium difficile-Associated Disease; Gastroenterology; 2008; Volume 135, Issue 6, Pages 1984-1992
Chen, L. A., Van Meerbeke, S., Albesiano, E., Goodwin, A., Wu, S., Yu, H., . . . Sears, C. (2015). Fecal detection of enterotoxigenic Bacteroides fragilis. European Journal of Clinical Microbiology & Infectious Diseases: Official Publication of the European Society of Clinical Microbiology, 34(9), 1871-1877. https://doi.org/10.1007/510096-015-2425-7
Christiaens, H., Leer, R. J., Pouwels, P. H., & Verstraete, W. (1992). Cloning and expression of a conjugated bile acid hydrolase gene from Lactobacillus plantarum by using a direct plate assay. Applied and Environmental Microbiology, 58(12), 3792-3798. Retrieved from http://www.ncbi.nlm.nih.gov/pubmed/1476424
Corzo, G., & Gilliland, S. E. (1999). Bile Salt Hydrolase Activity of Three Strains of Lactobacillus acidophilus. Journal of Dairy Science, 82(3), 472-480. https://doi.org/10.3168/jds.S0022-0302(99)75256-2
Fatima, R., & Aziz, M. (2019). The Hypervirulent Strain of Clostridium Difficile: NAP1/B1/027-A Brief Overview. Cureus, 11(1), e3977. https://doi.org/10.7759/cureus.3977
Franz, C. M. A. P., Specht, I., Haberer, P., & Holzapfel, W. H. (2001). Bile Salt Hydrolase Activity of Enterococci Isolated from Food: Screening and Quantitative Determination, Journal of Food Protection (Vol. 64). Retrieved from https://jfoodprotection.org/doi/pdfplus/10.4315/0362-028X-64.5.725
Giel, J. L., Sorg, J. A., Sonenshein, A. L., & Zhu, J. (2010). Metabolism of Bile Salts in Mice Influences Spore Germination in C. difficile. PLoS ONE, 5(1), 8740. https://doi.org/10.1371/journal.pone.0008740
Goulding, D., Thompson, H., Emerson, J., Fairweather, N. F., Dougan, G., Douce, G. R. (2009). Distinctive Profiles of Infection and Pathology in Hamsters Infected with Clostridium difficile Strains 630 and B1. Infect Immun. 77(12): 5478-5485. doi: 10.1128/IAI.00551-09.
Hansen E H. Enzymatic glycosylation of small molecule drugs: A strategy to improve absorption and distribution [Ph.D thesis]. University of Copenhagen, Faculty of Pharmaceutical Science 2009(b); 28.
Hay, D. W., & Carey, M. C. (1990). Chemical species of lipids in bile. Hepatology (Baltimore, Md.), 12(3 Pt 2), 6S-14S; discussion 14S-16S. Retrieved from http://www.ncbi.nlm.nih.gov/pubmed/2210659
Heidebrecht, H. J., Weiss, W. J., Pulse, M., Lange, A., Gisch, K., Kliem, H., von Eichel-Streiber, C.: Treatment and Prevention of Recurrent Clostridium difficile Infection with Functionalized Bovine Antibody-Enriched Whey in a Hamster Primary Infection Model. Toxins 2019, 11(2), 98.

### Hong 2017a

Hong H A, Hitri K, Hosseini S, Kotowicz N, Bryan D, Mawas F, Wilkinson A J, van Broekhoven A, Kearsey J, Cutting S M: Mucosal Antibodies to the C Terminus of Toxin A Prevent Colonization of Clostridium difficile. Infect Immun 2017, 85(4).

### Hong 2017b

Hong, H. A., Ferreira, W. T., Hosseini, S., Anwar, S., Hitri, K., Wilkinson, A. J., Cutting, S. M: The Spore Coat Protein CotE Facilitates Host Colonization by Clostridium difficile. The Journal of infectious diseases 2017, 216(11), 1452-1459.
Hou, J., Tyo, K. E. J., Liu, Z., Petranovic, D., & Nielsen, J. (2012). Metabolic engineering of recombinant protein secretion by Saccharomyces cerevisiae. FEMS Yeast Research, 12(5), 491-510. https://doi.org/10.1111/j.1567-1364.2012.00810.x
Huang, M., Wang, G., Qin, J., Petranovic, D., Nielsen, J., (2018). Engineering the protein secretory pathway of Saccharomyces cerisiae improved protein production. PNAS November 20, 2018 115 (47) E11025-E11032.
Hudson, L. E., Fasken, M. B., McDermott, C. D., McBride, S. M., Kuiper, E. G., Guiliano, D. B., Corbett, A. H., Lamb, T. J. (2014). Functional Heterologous Protein Expression by Genetially Engineered Probiotic Yeast Saccharomyces bouladii. PLOS ONE. Nov 2014; vol.9; issue 11: e112660.
Ichihara K, Fukubayashi Y. Preparation of fatty acid methyl esters for gas-liquid chromatography. J Lipid Res 2010;51:635-40.
Jensen, N. B., Strucko, T., Kildegaard, K. R., David, F., Maury, J., Mortensen, U. H., . . . Borodina, I. (2014). EasyClone: method for iterative chromosomal integration of multiple genes in Saccharomyces cerevisiae. FEMS Yeast Research, 14(2), 238-248. https://doi.org/10.1111/1567-1364.12118
Jia, B., Park, D., Hahn, Y., &Jeon, C. O. (2020). Metagenomic analysis of the human microbiome reveals the association between the abundance of gut bile salt hydrolases and host health. Gut Microbes, Volume 11, 2020 - Issue 5, Pages 1300-1313. https://doi.org/10.1080/19490976.2020.1748261
Jiang, J., Hang, X., Zhang, M., Liu, X., Li, D., & Yang, H. (2010). Diversity of bile salt hydrolase activities in different lactobacilli toward human bile salts. Annals of Microbiology, 60(1), 81-88. https://doi.org/10.1007/s13213-009-0004-9
Khatri, I., Tomar, R., Ganesan, K., Prasad, G. S., & Subramanian, S. (2017). Complete genome sequence and comparative genomics of the probiotic yeast Saccharomyces boulardii. Scientific Reports, 7(1), 371. https://doi.org/10.1038/541598-017-00414-2
Kjeldsen, T. (2000). Yeast secretory expression of insulin precursors. Applied Microbiology and Biotechnology, 54(3), 277-286. https://doi.org/10.1007/s002530000402
Knight, D. R., Kullin, B., Androga, G. O., Barbut, F., Eckert, K., Johnson, S., Spigaglia, P., Tateda, K., Tsai P. J., Riley, T. V., 2019. Evolutionary and Genomic Insights into Clostridioides difficile Sequence Type 11: a Diverse Zoonotic and Antimicrobial-Resistant Lineage of Global One Health Importance. mBio March/April 2019 Volume 10 Issue 2 e00446-19. doi.org/10.1128/mBio.00446-19.
Lawley et al.; Antibiotic Treatment of Clostridium difficile Carrier Mice Triggers a Supershedder State, Spore-Mediated Transmission, and Severe Disease in Immunocompromised Hosts; Infection and Immunity; 2009; Vol. 77, No. 9; pp. 3661-3669
Liong, M. T., & Shah, N. P. (2005). Bile salt deconjugation ability, bile salt hydrolase activity and cholesterol co-precipitation ability of lactobacilli strains. International Dairy Journal, 15(4), 391-398. https://doi.org/10.1016/J.IDAIRYJ.2004.08.007
Lupp et al.; Host-Mediated Inflammation Disrupts the Intestinal Microbiota and Promotes the Overgrowth of Enterobacteriaceae; Cell Host & Microbe; 2007; vol 2; pp. 119-129.
Marques, M. R. C., Loebenberg, R., & Almukainzi, M. (n.d.). Simulated Biological Fluids with Possible Application in Dissolution Testing. https://doi.org/10.14227/DT180311P15
McDonald, L. C., Gerding, D. N., Johnson, S., Bakken, J. S., Carroll, K. C., Coffin, S. E., Dubberke, E. R., Garey, K. W., Gould, C. V., Kelly, C., Loo, V., Sammons, J. S., Sandora, T. J., Wilcox, M. H. (2018). Clinical Practice Guidelines for Clostridium difficile Infection in Adults and Children: 2017 Update by the Infectious Diseases Society of America (IDSA) and Society for Healthcare Epidemiology of America (SHEA). Clinical Infectious Diseases, Volume 66, Issue 7, 1 Apr. 2018, Pages el—e48.
Mikkelsen, M. D., Buron, L. D., Salomonsen, B., Olsen, C. E., Hansen, B. G., Mortensen, U. H., & Halkier, B. A. (2012). Microbial production of indolylglucosinolate through engineering of a multi-gene pathway in a versatile yeast expression platform. Metabolic Engineering, 14(2), 104-111. https://doi.org/10.1016/j.ymben.2012.01.006
Moré, Margret I, & Vandenplas, Y. (2018). Saccharomyces boulardii CNCM 1-745 Improves Intestinal Enzyme Function: A Trophic Effects Review. Clinical Medicine Insights: Gastroenterology, 11, 117955221775267. https://doi.org/10.1177/1179552217752679
Moré, Margret Irmgard, & Swidsinski, A. (2015). Saccharomyces boulardii CNCM 1-745 supports regeneration of the intestinal microbiota after diarrheic dysbiosis &amp;ndash; a review. Clinical and Experimental Gastroenterology, 8, 237. https://doi.org/10.2147/CEG.S85574
Nielsen, H. (2019). SignalP 5.0 improves signal peptide predictions using deep neural networks. Nature Biotechnology, 37(4), 420-423. https://doi.org/10.1038/s41587-019-0036-z
Permpoonpattana, P., Hong, H. A., Phetcharaburanin, J., (2011). Immunization with Bacillus spores expressing toxin A peptide repeats protects against infection with Clostridium difficile strains producing toxins A and B. Infection and immunity 2011; 79:2295-302.
Phetcharaburanin, J., Hong, H. A., Colenutt, C., (2014). The spore-associated protein BclA1 affects the susceptibility of animals to colonization and infection by Clostridium difficile. Mol Microbiol 2014; 92:1025-38.
Pothoulakis, C., Theoharides, T. C., Dickey, B. F., (1991). Characterization of rabbit ileal receptors for Clostridium difficile toxin A. Evidence for a receptor-coupled G protein. J Clin Invest. 1991; 88(1):119-125.
Ram, K., & Wickham, H. (2018). wesanderson: A Wes Anderson Palette Generator. Retrieved from https://cran.r-project.org/package=wesanderson
Roth, J. S., Losty, T., Wierbicki, E., (1971). Assay of proteolytic enzyme activity using a ¹⁴C-labeled hemoglobin. Analytical Biochemistry. Volume 42, Issue 1, July 1971, Pages 214-221.
RStudio Team. (2016). RStudio: Integrated Development Environment for R. Retrieved from http://www.rstudio.com/
Sambol, S. P., Merrigan, M. M., Johnson, S., Gerding, D. N., (2001). Infection of Hamsters with Epidemiologically Important Strains of Clostridium difficile. The Journal of Infectious Diseases, Volume 183, Issue 12, 15 Jun. 2001, Pages 1760-1766.
Sorg, J. A., & Sonenshein, A. L. (2008). Bile salts and glycine as cogerminants for Clostridium difficile spores. Journal of Bacteriology, 190(7), 2505-2512.
Sorg, J. A., & Sonenshein, A. L. (2009). Chenodeoxycholate is an inhibitor of Clostridium difficile spore germination. Journal of Bacteriology, 191(3), 1115-1117.
Staggers, J. E., Hernell, O., Stafford, R. J., & Carey, M. C. (1990). Physical-chemical behavior of dietary and biliary lipids during intestinal digestion and absorption. 1. Phase behavior and aggregation states of model lipid systems patterned after aqueous duodenal contents of healthy adult human beings. Biochemistry, 29(8), 2028−20 40. https://doi.org/10.1021/bi00460a011
Sun et al.; Mouse Relapse Model of Clostridium difficile Infection; INFECTION AND IMMUNITY; 2011; Vol. 79; No. 7; pp. 2856-2864.
Surawicz, C. M., McFarland, L. V., Greenberg, R. N., Rubin, M., Fekety, R., Mulligan, M. E., Garcia, R. J., Brandmarker, S., Bowen, K., Borjal, D., Elmer, G. W. (2000). The search for a better treatment for recurrent Clostridium difficile disease: use of high-dose vancomycin combined with Saccharomyces boulardii. Clin Infect Dis. 2000 Oct;31(4):1012-7. doi: 10.1086/318130.
Tanaka, H., Hashiba, H., Kok, J., & Mierau, I. (2000). Bile salt hydrolase of Bifidobacterium longum-biochemical and genetic characterization. Applied and Environmental Microbiology, 66(6), 2502-2512. https://doi.org/10.1128/aem.66.6.2502-2512.2000
Theriot C. M., Koenigsknecht M. J., Carlson P. E. et al. Antibiotic-induced shifts in the mouse gut microbiome and metabolome increase susceptibility to Clostridium difficile infection. Nat Commun 2014; 5:3114.
Toothaker, R. D., & Elmer, G. W. (1984). Prevention of clindamycin-induced mortality in hamsters by Saccharmoyces boulardii. American society for microbiology. DOI: 10.1128/AAC.26.4.552
Vanegas, K. G., Lehka, B. J. & Mortensen, U. H. SWITCH: a dynamic CRISPR tool for genome engineering and metabolic pathway control for cell factory construction in Saccharomyces cerevisiae. Microb Cell Fact 16, 25 (2017). https://doi.org/10.1186/s12934-017-0632-x
Wentz, A. E., & Shusta, E. V. (2008). Enhanced Secretion of Heterologous Proteins from Yeast by Overexpression of Ribosomal Subunit RPP0. Biotechnology Progress, 24(3), 748-756. https://doi.org/10.1021/bp070345m
Wickham, H. (2016). ggplot2: Elegant Graphics for Data Analysis. Retrieved from https://ggplot2.tidyverse.org
Yoon, S., Yu, J., McDowell, A., Kim, S. H., You, H. J., & Ko, G. (2017). Bile salt hydrolase-mediated inhibitory effect of Bacteroides ovatus on growth of C. difficile. Journal of Microbiology, 55(11), 892-899. https://doi.org/10.1007/512275-017-7340-4

All references are incorporated herein by reference.

### Items of the Disclosure

The present disclosure further discloses the following embodiments and items:

Item 1. A delivery vehicle comprising a genetically modified S. boulardii host cell comprising one or more heterologous polynucleotides encoding and producing a heterologous enzyme API capable of converting a compound which is inactive in the treatment of one or more diseases in a mammal into a compound which is active in the treatment of the one or more diseases in said mammal, wherein the vehicle is suitable for administering to the mammal and wherein the host cell is capable of producing and delivering the produced enzyme API in situ of the location in the mammal body in need of preventing, treating and/or relieving the disease.

Item 2. The delivery vehicle of item 1, wherein the API is an organic molecule having a molecular weight of more than 200 g/mol.

Item 3. The delivery vehicle of any preceding item, wherein the API is a polypeptide.

Item 4. The delivery vehicle of any preceding item, wherein the API is an enzyme.

Item 5. The delivery vehicle of any preceding item, wherein the API is capable of in situ converting a compound which is inactive in the prevention, treatment and/or relief of one or more diseases into a compound which is active in the prevention, treatment and/or relief of one or more diseases.

Item 6. The delivery vehicle of any preceding item comprising one or more polynucleotides encoding the API.

Item 7. The delivery vehicle of any preceding item comprising a microbial host cell comprising the one or more polynucleotides encoding the API.

Item 8. The delivery vehicle of item 7, wherein vehicle is the microbial host cell.

Item 9. The delivery vehicle of item 8, wherein the microbial host cell is genetically modified.

Item 10. The delivery vehicle of items 7 to 9, wherein the one or more polynucleotides encoding the API is heterologous to the genetically modified cell.

Item 11. The delivery vehicle of any preceding item, wherein the API is a Bile Salt Hydrolase (BSH), capable of converting a conjugated bile salt into deconjugated bile salt active in the prevention, treatment and/or relief of Clostridioides infection and/or Clostridioides infection induced colitis.

Item 12. The delivery vehicle of item 11, wherein the pathogenic strain is of the species C. difficile.

Item 13. The delivery vehicle of item 11 or 12, wherein the conjugated bile salt is selected from glycocholic acid (GCA); taurocholic acid (TCA); glycodeoxycholic acid (GDCA), taurodeoxycholic acid (TDCA); taurochenodeoxycholic acid (TCDCA); and glycochenodeoxycholic acid (GCDCA) and the deconjugated bile salt is selected from cholic acid (CA); deoxycholic acid (DCA), and chenodeoxycholic acid (CDCA) respectively.

Item 14. The delivery vehicle of any preceding item, wherein the API is a BSH comprising a polypeptide selected from:
a) a polypeptide which is at least 50%, such as at least 60%, such as at least 70%, such as at least 80%, such as at least 90%, such as at least 95%, such as at least 96%, such as at least 97%, such as at least 98%, such as at least 99%, such as 100% identical to the mature BSH enzyme of anyone of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, or 111;
b) a polypeptide encoded by a polynucleotide which is at least 50%, such as at least 60%, such as at least 70%, such as at least 80%, such as at least 90%, such as at least 95%, such as at least 96%, such as at least 97%, such as at least 98%, such as at least 99%, such as 100% identical to the polynucleotide comprised in anyone of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, or 112 or genomic DNA thereof encoding the mature polypeptide of anyone of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 or 111 respectively;
c) a functional variant of the mature polypeptide of (a) or (b) having BSH activity.

Item 15. The delivery vehicle of any preceding item, wherein the API is a BSH comprising a polypeptide selected from:

a) a polypeptide which is at least 90% identical to the mature BSH enzyme of SEQ ID NO: 1; or

b) a polypeptide encoded by a polynucleotide which is at least 90% identical to the polynucleotide comprised in SEQ ID NO: 2 encoding the mature polypeptide of SEQ ID NO: 1.

Item 16. The delivery vehicle of any preceding item comprising a microbial host cell, wherein the cell is a fungus or a bacterium.

Item 17. The delivery vehicle of item 16, wherein the fungus is selected from the phylas consisting of Ascomycota, Basidiomycota, Neocallimastigomycota, Glomeromycota, Blastocladiomycota, Chytridiomycota, Zygomycota, Oomycota and Microsporidia.

Item 18. The delivery vehicle of item 17, wherein the yeast is selected from the genera consisting of Saccharomyces, Kluveromyces, Candida, Pichia, Debaromyces, Hansenula, Yarrowia, Zygosaccharomyces, and Schizosaccharomyces.

Item 19. The delivery vehicle of item 18, wherein the yeast host cell is selected from the species consisting of Kluyveromyces lactis, Saccharomyces boulardii, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Zygosaccharomyces spp., Schizosaccharomyces pombe, and Yarrowia lipolytica.

Item 20. The delivery vehicle of item 19, wherein the yeast host cell is Saccharomyces boulardii.

Item 21. The delivery vehicle of item 16, wherein the bacterium is selected from the genera consisting of Lactobacillus, Leuconostoc, streptomyces, Pediococcus, Lactococcus, Bifidobacterium, Weissella, Streptococcus, Komagataeibacter, Acetobacter, and Gluconacetobacter.

Item 22. The delivery vehicle of item 21, wherein the bacterium host cell is selected from the species consisting of Lactobacillus acidophilus, Lactobacillus bulgaricus, Bacteroides ovatus, Bacteroides fragilis, Lactobacillus casei, Lactobacillus gasseri, Lactobacillus gallinarum, Lactobacillus reuteri, Lactobacillus plantarum, Lactobacillus brevis, Lactobacillus paraplantarum, Lactobacillus coryniformis, Lactobacillus pentosus, and Lactobacillus fermentum, Lactobacillus delbrueckii subsp. bulgaricus, Lactobacillus helveticus, Lactobacillus kefiranofaciens, Lactobacillus paracasei, Lactococcus lactis, Bifidobacterium bifidum, Leuconostoc mesenteroides, Leuconostoc citreum, Leuconostoc argentinum, Pediococcus pentosaceus, Weissella spp., Streptococcus thermophilus, Streptomycess spp., Gluconacetobacter xylinus, Acetobacter pasteurianus, Acetobacter aceti and Gluconobacter oxydans.

Item 23. The delivery vehicle of item 20, wherein the vehicle is a genetically modified yeast cell of the species S. boulardii comprising and expressing a heterologous gene encoding a BSH enzyme and thereby producing said BSH enzyme.

Item 24. The delivery vehicle of any preceding item comprising a microbial host cell, wherein the cell further comprises at least one transporter molecule facilitating secretion of the API.

Item 25. The delivery vehicle of any preceding item comprising a microbial host cell, wherein one or more native genes of the cell is overexpressed, attenuated, disrupted and/or deleted.

Item 26. The delivery vehicle of any preceding item, comprising a genetically modified Saccharomyces boulardii modified by overexpressing one or more native genes KEX2, BIP, PDI, HAC1, SSO2, ERO1, COG5, and/or a functional deletion or downregulation of hda2, vps5 and tda3.

Item 27. The delivery vehicle of any preceding item comprising a microbial host cell, wherein the cell comprises at least 2 copies of a polynucleotide encoding the API.

Item 28. The delivery vehicle of any preceding item, wherein the vehicle is coated by a protective coating.

Item 29. The delivery vehicle of any preceding item, wherein the vehicle is encapsulated by a membrane, in a capsule, microcapsule, sphere and/or microsphere.

Item 30. The delivery vehicle of items 28 to 29, wherein the coating, membrane, capsule, microcapsule, sphere and/or microsphere is enteric.

Item 31. The delivery vehicle of items 28 to 30, wherein the enteric coating or membrane is triggered to release the vehicle and/or the API by pH, by osmotic pressure, by enzymatic digestion and/or by time-release.

Item 32. The delivery vehicle of items 28 to 31, wherein the coating, capsule, microcapsule, sphere and/or microsphere comprise one or more materials selected from gums, proteins, waxes, polyols, alginates, starches, dextrans and chitosans.

Item 33. The delivery vehicle of items 28 to 32, wherein the coating or membrane is insoluble in mammal gastro-intestinal juices.

Item 34. The delivery vehicle of items 28 to 33, wherein the coating or membrane is permeable to the API.

Item 35. The delivery vehicle of items 28 to 34, wherein the coating or membrane is impermeable to the system.

Item 36. The delivery vehicle of items 28 to 35, wherein the vehicle comprise a microbial cell and the coating or membrane is impermeable to the cell.

Item 37. The delivery vehicle of items 28 to 36, wherein the coating or membrane is made of alginate-poly lysine-alginate (APA).

Item 38. The delivery vehicle of item 37, wherein the coating or membrane is made of materials selected from Alginate/Poly-L-lysine/Alginate (APA), Alginate/Poly-L-lysine/Pectin/Poly-L-lysine/Alginate (APPPA), Alginate/Poly-L-lysine/Pectin/Poly-L-lysine/Pectin (APPPP), and Alginate/Poly-L-lysine/Chitosan/Poly-L-lysine/Alginate (APCPA).

Item 39. The delivery vehicle of any preceding item, wherein the vehicle is a genetically modified S. boulardii host cell comprising one or more heterologous polynucleotides encoding and producing a heterologous enzyme API capable of converting a compound which is inactive in the treatment of one or more diseases in a mammal into a compound which is active in the treatment of the one or more diseases in said mammal, wherein the vehicle is suitable for administering to the mammal and wherein the host cell is capable of producing and delivering the produced enzyme API in situ of the location in the mammal body in need of preventing, treating and/or relieving the disease.

Item 40. A polynucleotide construct comprising a polynucleotide sequence encoding an API of any preceding item, operably linked to one or more control sequences.

Item 41. The polynucleotide construct of item 40, wherein the control sequence is heterologous to the polynucleotide.

Item 42. The polynucleotide construct of item 41, wherein the construct is an expression vector.

Item 43. The vehicle of any preceding item comprising a microbial host cell comprising the polynucleotide construct of items 40 to 42.

Item 44. A cell culture, comprising the microbial host cell of any preceding item and a growth medium.

Item 45. A method for producing the cell culture of item 43 comprising
a) culturing the microbial host cell of any preceding item at conditions allowing growth of the microbial host cell; and
b) optionally recovering and/or isolating the cell culture.

Item 46. The method of items 45, further comprising one or more elements selected from:
a) culturing the cell culture in a nutrient medium;
b) culturing the cell culture under aerobic or anaerobic conditions;
c) culturing the cell culture under agitation;
d) culturing the cell culture at a temperature of between 25° C. to 50° C.;
e) culturing the cell culture at a pH of between 3-9; and
f) culturing the cell culture for between 10 hours to 30 days.
and having a cell density of at least 1-3×10⁹CFU/ml.

Item 47. A fermentation composition comprising the cell culture of item 44.

Item 48. The fermentation composition of item 47, comprising the API and one or more compounds selected from trace metals, vitamins, salts, yeast nitrogen base, carbon source, YNB, and/or amino acids of the fermentation; wherein the concentration of the API is at least 1 mg/l composition.

Item 49. The fermentation composition of items 47 to 48, having a cell density of at least 10⁷ CFU/ml.

Item 50. A composition comprising the vehicle, and/or cell culture of any preceding item and one or more carriers, agents, additives and/or excipients.

Item 51. A pharmaceutical composition comprising the vehicle, and/or cell culture of any preceding item and one or more pharmaceutical grade excipient, additives and/or adjuvants.

Item 52. The pharmaceutical composition of item 51, wherein the pharmaceutical composition is in form of a powder, a tablet or a capsule.

Item 53. The pharmaceutical composition of item 51, wherein the pharmaceutical composition is in form of a pharmaceutical solution, suspension, lotion or ointment.

Item 54. The pharmaceutical composition of items 51 to 53 for use as a medicament for prevention, treatment and/or relief of a disease in a mammal.

Item 55. The pharmaceutical composition of item 54 for use in the prevention, treatment and/or relief of CDI and/or CDI induced colitis.

Item 56. The pharmaceutical composition of item 55, wherein the API is BSH and the treatment comprises contacting a pathogenic strain of the genus Clostridioides in the presence of a conjugated bile acid with the pharmaceutical composition at conditions allowing the vehicle and/or cell culture to produce and deliver BSH in amounts converting the conjugated bile acid into therapeutically effective amounts of deconjugated bile acids inhibiting germination and/or proliferation of the pathogenic strain.

Item 57. The pharmaceutical composition of item 56, wherein the prevention, treatment and/or relief is performed in situ of the location of the pathogenic strain in and/or on the body of the mammal.

Item 58. The pharmaceutical composition of items 54 to 57, wherein the composition is administered daily in an amount of at least 10 mg or at least 10⁶ CFU per kg body mass of the mammal to be treated.

Item 59. The pharmaceutical composition of items 54 to 58, wherein the composition is administered parenterally.

Item 60. The pharmaceutical composition of item 59, wherein the parenteral administration is ocular administration.

Item 61. The pharmaceutical composition of item 59, wherein the parenteral administration is pulmonary administration.

Item 62. The pharmaceutical composition of items 54 to 58, wherein the composition is administered enterally.

Item 63. The pharmaceutical composition of items 54 to 58, wherein the composition is administered topically.

Item 64. A method for preparing the pharmaceutical composition of item 51 to 63 comprising mixing the vehicle, and/or the cell culture of any preceding item with one or more pharmaceutical grade excipient, additives and/or adjuvants.

Item 65. A method for preventing, treating and/or relieving a disease comprising administering the pharmaceutical composition of items 51 to 53 to a mammal in an amount for the vehicle and/or cell culture to produce and deliver in situ a therapeutically effective amount of the API.

Item 66. The method of item 65, wherein the disease is CDI and/or CDI induced colitis.

Item 67. The method of item 66, wherein the prevention, treatment and/or relief is inhibiting germination or proliferation of a pathogenic strain of the genus Clostridioides, the API is BSH and the prevention, treatment and/or relief comprises contacting the pathogenic strain in the presence of a conjugated bile acid with the pharmaceutical composition at conditions allowing the vehicle and/or cell culture to produce and deliver BSH in amounts converting the conjugated bile acid into therapeutically effective amounts of deconjugated bile acids inhibiting germination and/or proliferation of the pathogenic strain.

Item 68. The method of item 67, wherein the method is performed in situ of the pathogenic strain in and/or on the body of a mammal.

Item 69. The method of items items 65 to 68, wherein the composition is administered daily in an amount of at least 10 mg or and least 10⁶ CFU per kg body mass of the mammal to be treated.

Item 70. The method of items 65 to 69, wherein the composition is administered parenterally.

Item 71. The method of item 70, wherein the parenteral administration is ocular administration. Item 72. The method of item 70, wherein the parenteral administration is pulmonary administration.

Item 73. The method of items 65 to 69, wherein the composition is administered enterally.

Item 74. The method of items 65 to 69, wherein the composition is administered topically.

Item 75. A delivery vehicle comprising a genetically modified microbial host cell comprising one or more heterologous polynucleotides encoding and producing a one or more enzyme active pharmaceutical ingredient (API), wherein the vehicle is suitable for administering to the mammal and wherein the modified microbial host cell is capable of producing and delivering the one or more enzyme API in situ of the location in the body of a mammal in need of preventing, treating and/or relieving a disease.

Item 76. The delivery vehicle of item 75, wherein at least one of the one or more heterologous polynucleotides of the genetically modified microbial host cell encodes a heterologous enzyme API or overexpresses a native enzyme API compared to an unmodified microbial host cell.

Item 77. The delivery vehicle of item 75 or 76, wherein the one or more enzyme API is capable of enzymatic activity in conditions found within the mammalian gastrointestinal tract.

Item 78. The delivery vehicle of any of items 75 to 77, wherein the API is capable of in situ converting a compound which is inactive in the prevention, treatment and/or relief of one or more diseases into a compound which is active in the prevention, treatment and/or relief of one or more diseases.

Item 79. The delivery vehicle of any of items 75 to 78, wherein the one or more diseases is a Clostridioides infection, Clostridioides infection induced colitis, obesity, type 2 diabetes, cardiovascular disease, colon cancer, polycystic ovary syndrome, a neurological disease, diseases of the liver including nonalcoholic steatohepatitis, cirrhosis and/or liver cancer.

Item 80. The delivery vehicle of item 79, wherein the Clostridioides species is C. difficile.

Item 81. The delivery vehicle of any of items 75 to 80, wherein the one or more API is a Bile Salt Hydrolase (BSH), capable of converting a conjugated bile salt into deconjugated bile salt active in the prevention, treatment and/or relief of Clostridioides infection and/or Clostridioides infection induced colitis.

Item 82. The delivery vehicle of item 81, wherein the conjugated bile salt is selected from glycocholic acid (GCA); taurocholic acid (TCA); glycodeoxycholic acid (GDCA), taurodeoxycholic acid (TDCA); taurochenodeoxycholic acid (TCDCA); and glycochenodeoxycholic acid (GCDCA) and the deconjugated bile salt is selected from cholic acid (CA); deoxycholic acid (DCA), and chenodeoxycholic acid (CDCA) respectively.

Item 83. The delivery vehicle of any of items 75 to 82, wherein the API is a BSH comprising a polypeptide selected from:
a) a polypeptide which is at least 50%, such as at least 60%, such as at least 70%, such as at least 80%, such as at least 90%, such as at least 95%, such as at least 96%, such as at least 97%, such as at least 98%, such as at least 99%, such as 100% identical to the mature BSH enzyme of anyone of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, or 111;
b) a polypeptide encoded by a polynucleotide which is at least 50%, such as at least 60%, such as at least 70%, such as at least 80%, such as at least 90%, such as at least 95%, such as at least 96%, such as at least 97%, such as at least 98%, such as at least 99%, such as 100% identical to the polynucleotide comprised in anyone of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, or 112, or genomic DNA thereof encoding the mature polypeptide of anyone of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 or 111 respectively;
c) a functional variant of the mature polypeptide of (a) or (b) having BSH activity.

Item 84. The delivery vehicle of any of items 75 to 83, wherein the heterologous enzyme API is a BSH comprising a polypeptide selected from:
a) a polypeptide which is at least 90% identical to the mature BSH enzyme of SEQ ID NO: 1; or
b) a polypeptide encoded by a polynucleotide which is at least 90% identical to the polynucleotide comprised in SEQ ID NO: 2 encoding the mature polypeptide of SEQ ID NO: 1.

Item 85. The delivery vehicle of any of items 75 to 84 comprising a microbial host cell, wherein the cell is a fungus or a bacterium.

Item 86. The delivery vehicle of item 85, wherein the fungus is selected from the phylas consisting of Ascomycota, Basidiomycota, Neocallimastigomycota, Glomeromycota, Blastodadiomycota, Chytridiomycota, Zygomycota, Oomycota and Microsporidia.

Item 87. The delivery vehicle of item 85 or 86, wherein the fungus is a yeast is selected from the genera consisting of Saccharomyces, Kluveromyces, Candida, Pichia, Debaromyces, Hansenula, Yarrowia, Zygosaccharomyces, and Schizosaccharomyces.

Item 88. The delivery vehicle of item 87, wherein the yeast host cell is selected from the species consisting of Kluyveromyces lactis, Saccharomyces boulardii, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Zygosaccharomyces spp., Schizosaccharomyces pombe, and Yarrowia lipolytica.

Item 89. The delivery vehicle of item 88, wherein the yeast host cell is Saccharomyces boulardii.

Item 90. The delivery vehicle of item 85, wherein the bacterium is selected from the genera consisting of Lactobacillus, Leuconostoc, Streptomyces, Pediococcus, Lactococcus, Bifidobacterium, Weissella, Streptococcus, Komagataeibacter, Acetobacter, and Gluconacetobacter.

Item 91. The delivery vehicle of item 90, wherein the bacterium host cell is selected from the species consisting of Lactobacillus acidophilus, Lactobacillus bulgaricus, Bacteroides ovatus, Bacteroides fragilis, Lactobacillus casei, Lactobacillus gasseri, Lactobacillus gallinarum, Lactobacillus reuteri, Lactobacillus plantarum, Lactobacillus brevis, Lactobacillus paraplantarum, Lactobacillus coryniformis, Lactobacillus pentosus, and Lactobacillus fermentum, Lactobacillus delbrueckii subsp. bulgaricus, Lactobacillus helveticus, Lactobacillus kefiranofaciens, Lactobacillus paracasei, Lactococcus lactis, Bifidobacterium bifidum, Leuconostoc mesenteroides, Leuconostoc citreum, Leuconostoc argentinum, Pediococcus pentosaceus, Weissella spp., Streptococcus thermophilus, Streptomycess spp., Gluconacetobacter xylinus, Acetobacter pasteurianus, Acetobacter aceti and Gluconobacter oxydans.

Item 92. The delivery vehicle of any of items 75 to 91 comprising a microbial host cell, wherein the cell further comprises at least one transporter molecule facilitating secretion of an API of any of the preceding items.

Item 93. The delivery vehicle of any of items 75 to 92 comprising a microbial host cell, wherein one or more native genes of the microbial host cell is overexpressed, attenuated, disrupted and/or deleted.

Item 94. The delivery vehicle of item 89, wherein the microbial host comprises a genetically modified Saccharomyces boulardii modified by overexpressing one or more native genes KEX2, BIP, PDI, HAC1, SSO2, ERO1, COG5, and/or a functional deletion or downregulation of had2, vps5 and tda3 and the the API is a BSH.

Item 95. The delivery vehicle of any of items 75 to 94 comprising a microbial host cell, wherein the cell comprises at least 2 copies of a polynucleotide encoding an API of any of the preceding items.

Item 96. The delivery vehicle of any of items 75 to 95, wherein the vehicle is coated by a protective coating.

Item 97. The delivery vehicle of any of items 75 to 96, wherein the vehicle is encapsulated by a membrane, in a capsule, microcapsule, sphere and/or microsphere.

Item 98. The delivery vehicle of items 96 to 97, wherein the coating, membrane, capsule, microcapsule, sphere and/or microsphere is enteric.

Item 99. The delivery vehicle of items 96 to 98, wherein the enteric coating or membrane is triggered to release the vehicle and/or the API by pH, by osmotic pressure, by enzymatic digestion and/or by time-release.

Item 100. The delivery vehicle of items 96 to 99, wherein the coating, capsule, microcapsule, sphere and/or microsphere comprise one or more materials selected from gums, proteins, waxes, polyols, alginates, starches, dextrans and chitosans.

Item 101. The delivery vehicle of items 96 to 100, wherein the coating or membrane is insoluble in mammal gastro-intestinal juices.

Item 102. The delivery vehicle of items 96 to 101, wherein the coating or membrane is permeable to the API.

Item 103. The delivery vehicle of items 96 to 102, wherein the coating or membrane is impermeable to the microbial host cell.

Item 104. The delivery vehicle of items 96 to 103, wherein the coating or membrane is made of materials selected from Alginate/Poly-L-lysine/Alginate (APA), Alginate/Poly-L-lysine/Pectin/Poly-L-lysine/Alginate (APPPA), Alginate/Poly-L-lysine/Pectin/Poly-L-lysine/Pectin (APPPP), and Alginate/Poly-L-lysine/Chitosa n/Poly-L-lysine/Alginate (APCPA).

Item 105. A polynucleotide construct comprising a polynucleotide sequence encoding an API of any preceding item, operably linked to one or more control sequences.

Item 106. The polynucleotide construct of item 105, wherein the control sequence is heterologous to the polynucleotide.

Item 107. The polynucleotide construct of item 106, wherein the construct is an expression vector.

Item 108. The vehicle of any preceding item comprising a microbial host cell comprising the polynucleotide construct of items 106 to 107.

Item 109. A cell culture, comprising the microbial host cell of any preceding item and a growth medium or fermentation medium.

Item 110. The cell culture of item 109, comprising the one or more API and one or more compounds selected from trace metals, vitamins, salts, yeast nitrogen base, carbon source, YNB, and/or amino acids of the fermentation; wherein the concentration of the one or more API is at least 1 mg/l composition.

Item 111. The cell culture of items 109 to 110, having a cell density of at least 10′ CFU/ml.

Item 112. A method for producing the cell culture of any of items 109 to 111, comprising
a) culturing the microbial host cell of any preceding item at conditions allowing growth of the microbial host cell; and
b) optionally recovering and/or isolating the cell culture.

Item 113. The method of items 112, further comprising one or more elements selected from:
c) culturing the cell culture in a nutrient medium;
d) culturing the cell culture under aerobic or anaerobic conditions;
e) culturing the cell culture under agitation;
f) culturing the cell culture at a temperature of between 25° C. to 50° C.;
g) culturing the cell culture at a pH of between 3-9; and
h) culturing the cell culture for between 10 hours to 30 days. and having a cell density of at least 1-3×10⁹CFU/ml.

Item 114. A composition comprising the delivery vehicle, and/or cell culture of any preceding item and one or more carriers, agents, additives and/or excipients.

Item 115. A pharmaceutical composition comprising the delivery vehicle, and/or cell culture of any preceding item and one or more pharmaceutical grade excipient, additives and/or adjuvants.

Item 116. The pharmaceutical composition of item 115, wherein the pharmaceutical composition is in form of a powder, a tablet or a capsule.

Item 117. The pharmaceutical composition of item 115, wherein the pharmaceutical composition is in form of a pharmaceutical solution, suspension, lotion or ointment.

Item 118. The pharmaceutical composition of items 115 to 117 for use as a medicament for prevention, treatment and/or relief of a disease in a mammal.

Item 119. The pharmaceutical composition of item 118 for use in the prevention, treatment and/or relief of CDI and/or CDI induced colitis, obesity, type 2 diabetes, cardiovascular disease, colon cancer, polycystic ovary syndrome, a neurological disease, diseases of the liver including nonalcoholic steatohepatitis, cirrhosis and/or liver cancer.

Item 120. The pharmaceutical composition of item 119, wherein the API is a BSH, and the treatment comprises contacting a pathogenic strain of the genus Clostridioides in the presence of a conjugated bile acid with the pharmaceutical composition at conditions allowing the vehicle and/or cell culture to produce and deliver BSH in situ of the location of the pathogenic strain in amounts converting the conjugated bile acid into therapeutically effective amounts of deconjugated bile acids inhibiting germination and/or proliferation of the pathogenic strain.

Item 121. The pharmaceutical composition of any of items 115 to 120, wherein the composition is administered daily in an amount of at least 10 mg or at least 10⁶ CFU per kg body mass of the mammal to be treated.

Item 122. The pharmaceutical composition of any of items 115 to 121, wherein the composition is administered enterally, topically, orally or rectally.

Item 123. A method for preparing the pharmaceutical composition of item 115 to 122 comprising mixing the vehicle, and/or the cell culture of any preceding item with one or more pharmaceutical grade excipient, additives and/or adjuvants.

Item 124. A method for preventing, treating and/or relieving a disease comprising administering the pharmaceutical composition of items 115 to 123 to a mammal in an amount for the vehicle and/or cell culture to produce and deliver in situ a therapeutically effective amount of the one or more API.

Item 125. The method of item 124, wherein the disease is a Clostridioides infection or Clostridioides infection induced colitis.

Item 126. The method of item 125, wherein the prevention, treatment and/or relief of the disease is performed by inhibiting germination or proliferation of a pathogenic strain of the genus Clostridioides, the API is BSH and the prevention, treatment and/or relief comprises contacting the pathogenic strain in the presence of a conjugated bile acid with the pharmaceutical composition at conditions allowing the vehicle and/or cell culture to produce and deliver BSH in amounts converting the conjugated bile acid into therapeutically effective amounts of deconjugated bile acids inhibiting germination and/or proliferation of the pathogenic strain.

Item 127. The method of item 125 or 126, wherein the method is performed in situ of the Clostridioides infection in and/or on the body of a mammal.

Item 128. The method of any of items 124 to 127, wherein the composition is administered daily in an amount of at least 10 mg or and least 10⁶ CFU per kg body mass of the mammal to be treated.

Item 129. The method of any of items 124 to 128, wherein the composition is administered enterally, orally, topically or rectally.

1. A delivery vehicle comprising a genetically modified microbial host cell comprising one or more heterologous polynucleotides encoding and producing a one or more enzyme active pharmaceutical ingredient (API), wherein the vehicle is suitable for administering to the mammal and wherein the modified microbial host cell is capable of producing and delivering the one or more enzyme API in situ of the location in the body of a mammal in need of preventing, treating and/or relieving a disease.

2. The delivery vehicle of claim 1, wherein at least one of the one or more heterologous polynucleotides of the genetically modified microbial host cell encodes a heterologous enzyme API.

3. The delivery vehicle of any preceding claim, wherein the one or more enzyme API is capable of enzymatic activity in conditions found within the mammalian gastrointestinal tract.

4. The delivery vehicle of any preceding claim, wherein the API is capable of in situ converting a compound which is inactive in the prevention, treatment and/or relief of one or more diseases into a compound which is active in the prevention, treatment and/or relief of one or more diseases.

5. The delivery vehicle of any preceding claim, wherein the one or more diseases is a Clostridioides infection, Clostridioides infection induced colitis, obesity, type 2 diabetes, cardiovascular disease, colon cancer, polycystic ovary syndrome, a neurological disease, diseases of the liver including nonalcoholic steatohepatitis, cirrhosis and/or liver cancer.

6. The delivery vehicle of claim 5, wherein the Clostridioides species is C. difficile.

7. The delivery vehicle of any preceding claim, wherein the one or more API is a Bile Salt Hydrolase (BSH), capable of converting a conjugated bile salt into deconjugated bile salt active in the prevention, treatment and/or relief of Clostridioides infection and/or Clostridioides infection induced colitis.

8. The delivery vehicle of claim 7, wherein the conjugated bile salt is selected from glycocholic acid (GCA); taurocholic acid (TCA); glycodeoxycholic acid (GDCA), taurodeoxycholic acid (TDCA);
taurochenodeoxycholic acid (TCDCA); and glycochenodeoxycholic acid (GCDCA) and the deconjugated bile salt is selected from cholic acid (CA); deoxycholic acid (DCA), and chenodeoxycholic acid (CDCA) respectively.

9. The delivery vehicle of any preceding claim, wherein the API is a BSH comprising a polypeptide selected from:
a) a polypeptide which is at least 50%, such as at least 60%, such as at least 70%, such as at least 80%, such as at least 90%, such as at least 95%, such as at least 96%, such as at least 97%, such as at least 98%, such as at least 99%, such as 100% identical to the mature BSH enzyme of anyone of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, or 111;
b) a polypeptide encoded by a polynucleotide which is at least 50%, such as at least 60%, such as at least 70%, such as at least 80%, such as at least 90%, such as at least 95%, such as at least 96%, such as at least 97%, such as at least 98%, such as at least 99%, such as 100% identical to the polynucleotide comprised in anyone of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, or 112, or genomic DNA thereof encoding the mature polypeptide of anyone of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109 or 111 respectively;
c) a functional variant of the mature polypeptide of (a) or (b) having BSH activity.

10. The delivery vehicle of any preceding claim, wherein the heterologous enzyme API is a BSH comprising a polypeptide selected from:
a) a polypeptide which is at least 90% identical to the mature BSH enzyme of SEQ ID NO: 1; or
b) a polypeptide encoded by a polynucleotide which is at least 90% identical to the polynucleotide comprised in SEQ ID NO: 2 encoding the mature polypeptide of SEQ ID NO: 1.

11. The delivery vehicle of any preceding claim comprising a microbial host cell, wherein the cell is a fungus or a bacterium.

12. The delivery vehicle of claim 11, wherein the fungus is selected from the phylas consisting of Ascomycota, Basidiomycota, Neocallimastigomycota, Glomeromycota, Blastocladiomycota, Chytridiomycota, Zygomycota, Oomycota and Microsporidia.

13. The delivery vehicle of claim 11 or 12, wherein the fungus is a yeast is selected from the genera consisting of Saccharomyces, Kluveromyces, Candida, Pichia, Debaromyces, Hansenula, Yarrowia, Zygosaccharomyces, and Schizosaccharomyces.

14. The delivery vehicle of claim 13, wherein the yeast host cell is selected from the species consisting of Kluyveromyces lactis, Saccharomyces boulardii, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Zygosaccharomyces spp., Schizosaccharomyces pombe, and Yarrowia lipolytica.

15. The delivery vehicle of claim 14, wherein the yeast host cell is Saccharomyces boulardii.

16. The delivery vehicle of claim 11, wherein the bacterium is selected from the genera consisting of Lactobacillus, Leuconostoc, Streptomyces, Pediococcus, Lactococcus, Bifidobacterium, Weissella, Streptococcus, Komagataeibacter, Acetobacter, and Gluconacetobacter.

17. The delivery vehicle of claim 16, wherein the bacterium host cell is selected from the species consisting of Lactobacillus acidophilus, Lactobacillus bulgaricus, Bacteroides ovatus, Bacteroides fragilis, Lactobacillus casei, Lactobacillus gasseri, Lactobacillus gallinarum, Lactobacillus reuteri, Lactobacillus plantarum, Lactobacillus brevis, Lactobacillus paraplantarum, Lactobacillus coryniformis, Lactobacillus pentosus, and Lactobacillus fermentum, Lactobacillus delbrueckii subsp. bulgaricus, Lactobacillus helveticus, Lactobacillus kefiranofaciens, Lactobacillus paracasei, Lactococcus lactis, Bifidobacterium bifidum, Leuconostoc mesenteroides, Leuconostoc citreum, Leuconostoc argentinum, Pediococcus pentosaceus, Weissella spp., Streptococcus thermophiles, Streptomyces spp., Gluconacetobacter xylinus, Acetobacter pasteurianus, Acetobacter aceti and Gluconobacter oxydans.

18. The delivery vehicle of any preceding claim comprising a microbial host cell, wherein the cell further comprises at least one transporter molecule facilitating secretion of an API of any of the preceding claims.

19. The delivery vehicle of any preceding claim comprising a microbial host cell, wherein one or more native genes of the microbial host cell is overexpressed, attenuated, disrupted and/or deleted.

20. The delivery vehicle of claim 15, wherein the microbial host comprises a genetically modified Saccharomyces boulardii modified by overexpressing one or more native genes KEX2, BIP, PDI, HAC1, SSO2, ERO1, COG5, and/or a functional deletion or downregulation of had2, vps5 and tda3 and the the API is a BSH.

21. The delivery vehicle of any preceding claim comprising a microbial host cell, wherein the cell comprises at least 2 copies of a polynucleotide encoding an API of any of the preceding claims.

22. The delivery vehicle of any preceding claim, wherein the vehicle is coated by a protective coating.

23. The delivery vehicle of any preceding claim, wherein the vehicle is encapsulated by a membrane, in a capsule, microcapsule, sphere and/or microsphere.

24. The delivery vehicle of claims 22 to 23, wherein the coating, membrane, capsule, microcapsule, sphere and/or microsphere is enteric.

25. The delivery vehicle of claims 22 to 24, wherein the enteric coating or membrane is triggered to release the vehicle and/or the API by pH, by osmotic pressure, by enzymatic digestion and/or by time-release.

26. The delivery vehicle of claims 22 to 25, wherein the coating, capsule, microcapsule, sphere and/or microsphere comprise one or more materials selected from gums, proteins, waxes, polyols, alginates, starches, dextrans and chitosans.

27. The delivery vehicle of claims 22 to 26, wherein the coating or membrane is insoluble in mammal gastro-intestinal juices.

28. The delivery vehicle of claims 22 to 27, wherein the coating or membrane is permeable to the API.

29. The delivery vehicle of claims 22 to 28, wherein the coating or membrane is impermeable to the microbial host cell.

30. The delivery vehicle of claims 22 to 29, wherein the coating or membrane comprises materials selected from Alginate/Poly-L-lysine/Alginate (APA), Alginate/Poly-L-lysine/Pectin/Poly-L-lysine/Alginate (APPPA), Alginate/Poly-L-lysine/Pectin/Poly-L-lysine/Pectin (APPPP), and Alginate/Poly-L-lysine/Chitosan/Poly-L-lysine/Alginate (APCPA).

31. A polynucleotide construct comprising a polynucleotide sequence encoding an API of any preceding claim, operably linked to one or more control sequences.

32. The polynucleotide construct of claim 31, wherein the control sequence is heterologous to the polynucleotide.

33. The polynucleotide construct of claim 32, wherein the construct is an expression vector.

34. The vehicle of any preceding claim comprising a microbial host cell comprising the polynucleotide construct of claims 31 to 33.

35. A cell culture, comprising the microbial host cell of any preceding claim and a growth medium or fermentation medium.

36. The cell culture of claim 35, comprising the one or more API and one or more compounds selected from trace metals, vitamins, salts, yeast nitrogen base, carbon source, YNB, and/or amino acids of the fermentation; wherein the concentration of the one or more API is at least 1 mg/l composition.

37. The cell culture of claims 35 to 36, having a cell density of at least 10⁷ CFU/ml.

38. A method for producing the cell culture of any of claims 35 to 37, comprising
i) culturing the microbial host cell of any preceding claim at conditions allowing growth of the microbial host cell; and
j) optionally recovering and/or isolating the cell culture.

39. The method of claims 38, further comprising one or more elements selected from:
k) culturing the cell culture in a nutrient medium;
l) culturing the cell culture under aerobic or anaerobic conditions;
m) culturing the cell culture under agitation;
n) culturing the cell culture at a temperature of between 25° C. to 50° C.;
o) culturing the cell culture at a pH of between 3-9; and
p) culturing the cell culture for between 10 hours to 30 days. and having a cell density of at least 1-3×10⁹ CFU/ml.

40. A composition comprising the delivery vehicle, and/or cell culture of any preceding claim and one or more carriers, agents, additives and/or excipients.

41. A pharmaceutical composition comprising the delivery vehicle, and/or cell culture of any preceding claim and one or more pharmaceutical grade excipient, additives and/or adjuvants.

42. The pharmaceutical composition of claim 41, wherein the pharmaceutical composition is in form of a powder, a tablet or a capsule.

43. The pharmaceutical composition of claim 41, wherein the pharmaceutical composition is in form of a pharmaceutical solution, suspension, lotion or ointment.

44. The pharmaceutical composition of claims 41 to 43 for use as a medicament for prevention, treatment and/or relief of a disease in a mammal.

45. The pharmaceutical composition of claim 44 for use in the prevention, treatment and/or relief of CDI and/or CDI induced colitis, obesity, type 2 diabetes, cardiovascular disease, colon cancer, polycystic ovary syndrome, a neurological disease, diseases of the liver including nonalcoholic steatohepatitis, cirrhosis and/or liver cancer.

46. The pharmaceutical composition of claim 45, wherein the API is a BSH, and the treatment comprises contacting a pathogenic strain of the genus Clostridioides in the presence of a conjugated bile acid with the pharmaceutical composition at conditions allowing the vehicle and/or cell culture to produce and deliver BSH in situ of the location of the pathogenic strain in amounts converting the conjugated bile acid into therapeutically effective amounts of deconjugated bile acids inhibiting germination and/or proliferation of the pathogenic strain.

47. The pharmaceutical composition of any of claims 41 to 46, wherein the composition is administered daily in an amount of at least 10 mg or at least 10⁶ CFU per kg body mass of the mammal to be treated.

48. The pharmaceutical composition of any of claims 41 to 47, wherein the composition is administered enterally, topically, orally or rectally.

49. A method for preparing the pharmaceutical composition of claims 41 to 48 comprising mixing the vehicle, and/or the cell culture of any preceding claim with one or more pharmaceutical grade excipient, additives and/or adjuvants.

50. A method for preventing, treating and/or relieving a disease comprising administering the pharmaceutical composition of claims 41 to 48 to a mammal in an amount for the vehicle and/or cell culture to produce and deliver in situ a therapeutically effective amount of the one or more API.

51. The method of claim 50, wherein the disease is a Clostridioides infection or Clostridioides infection induced colitis.

52. The method of claim 51, wherein the prevention, treatment and/or relief of the disease is performed by inhibiting germination or proliferation of a pathogenic strain of the genus Clostridioides, the API is BSH and the prevention, treatment and/or relief comprises contacting the pathogenic strain in the presence of a conjugated bile acid with the pharmaceutical composition at conditions allowing the vehicle and/or cell culture to produce and deliver BSH in amounts converting the conjugated bile acid into therapeutically effective amounts of deconjugated bile acids inhibiting germination and/or proliferation of the pathogenic strain.

53. The method of claim 51 or 52, wherein the method is performed in situ of the Clostridioides infection in and/or on the body of a mammal.

54. The method of any of claims 50 to 53, wherein the composition is administered daily in an amount of at least 10 mg or and least 10⁶ CFU per kg body mass of the mammal to be treated.

55. The method of any of claims 50 to 54, wherein the composition is administered enterally, orally, topically or rectally.


SEQUENCE LISTING (112 sequences)

SEQ ID NO: 1 - protein, 356 aa, Bacteroides ovatus
MTKNLLLGIAAVCGSTFQAVACTGISLTSRDGSYVQARTIEWARGVLQSEYVIIPRGQQLTSFTPTGVNGLTFTAKYGVVGLAVVQKEFIAEGINEAGLSAGLFFFPHYGGYKTYDAAQNQRTLADLQVTEWLLSQFSTIDEVKAALSSVRVVGLEKTAVVHWRIGEPSGRQVVLEIVGGVPHFYENEVGVLTNAPGFEWQLTNLNNYVNLHPGDASVQKLSGITLQPTGGNSGFLGIPGDATPPSRFVRAAFYRGTAPQRATGFDTVQQCFHLLNNFDIPIGIEHSQGDIPDIPSATQWTSAIDLTNRKVYYKTAYNNSIRCIDLKAIDFSKVKYQSQPLDKIQEQPVEMVKIPR

SEQ ID NO: 2 - DNA, 1071 bp, Bacteroides ovatus
atgaccaagaacttgttgttgggtattgctgctgtttgtggttctacttttcaagctgttgcttgtaccggtatttctttgacttctagagatggttcttacgttcaagccagaactattgaatgggctagaggtgttttacagtccgaatacgttattatcccaagaggtcaacaattgacttctttcactccaactggtgttaacggtttgacttttactgctaagtatggtgttgttggtttggccgttgttcagaaagaattcattgctgaaggtattaacgaagctggtttgtctgctggtttattcttttttccacattacggtggttacaagacttacgatgctgctcaaaatcaaagaactttggccgacttacaagttaccgaatggttgttgtctcaattctccactatcgatgaagttaaggctgctttgtcatccgttagagttgtaggtttggaaaagactgctgttgttcattggagaataggtgaaccatctggtagacaagttgttttggaaatagttggtggtgttccacacttctacgaaaatgaagttggtgttttgactaacgctccaggttttgaatggcaattgactaacttgaacaactacgttaacttgcatccaggtgatgcctctgttcaaaaattgtctggtattaccttgcaacctactggtggtaattctggttttttgggtattcctggtgatgctactccaccatctagatttgttagagctgctttttacagaggtactgctccacaaagagctactggttttgatactgttcaacaatgcttccacctgttgaacaatttcgatattccaatcggtatcgaacactcccaaggtgatattccagatattccttctgctactcaatggacctctgctattgatttgaccaacagaaaggtttactacaagaccgcttacaacaactccattagatgcatcgatttgaaggccatcgatttctctaaggtcaagtatcaatctcagccattggacaagattcaagaacaaccagttgaaatggtcaagatcccaagataa

SEQ ID NO: 3 - protein, 365 aa, Bacteroides ovatus
MTKNLLLGIAAVCGSTFQAVACTGISLTSRDGSYVQARTIEWARGVLQSEYVIIPRGQQLTSFTPTGVNGLTFTAKYGVVGLAVVQKEFIAEGINEAGLSAGLFFFPHYGGYKTYDAAQNQRTLADLQVTEWLLSQFSTIDEVKAALSSVRVVGLEKTAVVHWRIGEPSGRQVVLEIVGGVPHFYENEVGVLTNAPGFEWQLTNLNNYVNLHPGDASVQKLSGITLQPTGGNSGFLGIPGDATPPSRFVRAAFYRGTAPQRATGFDTVQQCFHLLNNFDIPIGIEHSQGDIPDIPSATQWTSAIDLTNRKVYYKTAYNNSIRCIDLKAIDFSKVKYQSQPLDKIQEQPVEMVKIPRCATHHHHHH

SEQ ID NO: 4 - DNA, 1098 bp, Bacteroides ovatus
atgaccaagaacttgttgttgggtattgctgctgtttgtggttctacttttcaagctgttgcttgtaccggtatttctttgacttctagagatggttcttacgttcaagccagaactattgaatgggctagaggtgttttacagtccgaatacgttattatcccaagaggtcaacaattgacttctttcactccaactggtgttaacggtttgacttttactgctaagtatggtgttgttggtttggccgttgttcagaaagaattcattgctgaaggtattaacgaagctggtttgtctgctggtttattcttttttccacattacggtggttacaagacttacgatgctgctcaaaatcaaagaactttggccgacttacaagttaccgaatggttgttgtctcaattctccactatcgatgaagttaaggctgctttgtcatccgttagagttgtaggtttggaaaagactgctgttgttcattggagaataggtgaaccatctggtagacaagttgttttggaaatagttggtggtgttccacacttctacgaaaatgaagttggtgttttgactaacgctccaggttttgaatggcaattgactaacttgaacaactacgttaacttgcatccaggtgatgcctctgttcaaaaattgtctggtattaccttgcaacctactggtggtaattctggttttttgggtattcctggtgatgctactccaccatctagatttgttagagctgctttttacagaggtactgctccacaaagagctactggttttgatactgttcaacaatgcttccacctgttgaacaatttcgatattccaatcggtatcgaacactcccaaggtgatattccagatattccttctgctactcaatggacctctgctattgatttgaccaacagaaaggtttactacaagaccgcttacaacaactccattagatgcatcgatttgaaggccatcgatttctctaaggtcaagtatcaatctcagccattggacaagattcaagaacaaccagttgaaatggtcaagattccaagatgtgctactcatcatcaccaccatcattaa

SEQ ID NO: 5 - protein, 344 aa, Enterococcus faecium
MTKNLLLGIAAVCGSTFQAVACTSITYVTSDHYFGRNFDYEISYNEVVTITPRNYKLNFRKVNDLDTHYAMIGIAAGIADYPLYYDATNEKGLSMAGLNFSGYADYKEIQEGKDNVSPFEFIPWILGQCSTVGEAKKLLKNINLANINYSDELPLSPLHWLLADKEKSIVIESMKDGLHIYDNPVGVLTNNPSFDYQLFNLNNYRVLSSETPKNNFSNQISLNAYSRGMGGIGLPGDLSSVSRFVKATFTKLNSVSGDSESESISQFFHILGSVEQQKGLCDVGDGKYEYTIYSSCCNVDKGIYYYRTYEDSQITAIDMNKEDLDSHKLISYPIIEKQQIKYIN

SEQ ID NO: 6 - DNA, 1035 bp, Enterococcus faecium
atgaccaagaacttgttgttgggtattgctgctgtttgtggttctacttttcaagctgttgcttgtacctctattacctacgttacttccgatcattacttcggtagaaacttcgactacgagatctcttacaacgaagttgttactatcacccctaggaactacaagttgaacttcagaaaggttaacgatttggatacccattacgccatgattggtattgcagctggtatagctgattatcccttgtattatgatgccaccaacgaaaagggtttgtctatggctggtttgaatttttctggttacgccgactacaaagaaatccaagaaggtaaggataacgtcagcccatttgaattcattccatggattttgggtcaatgctctactgttggtgaagctaaaaagctgttgaagaacattaacctggccaacatcaactactctgatgaattgccattgtctccattgcattggttgttggctgacaaagaaaagtccatcgttatcgaatctatgaaggatggcttgcacatctacgataatccagttggtgttttgactaacaacccatcttttgactaccagctgttcaacctgaacaactacagagttttgtcatctgaaaccccaaagaacaacttctccaatcagatctctttgaacgcttactctagaggtatgggtggtattggtttgccaggtgatttgtcatcagtttccagatttgttaaggctaccttcaccaagttgaattccgtttctggtgattccgaatccgaatccatttctcaattcttccatatcttgggttccgtcgaacaacaaaaaggtttgtgtgatgttggtgacggtaaatacgagtacactatctactcctcttgttgcaatgttgacaagggtatctactactacagaacctacgaagattctcaaattaccgccatcgacatgaacaaagaagatttggattctcacaagctgatctcgtacccaattattgaaaagcagcagatcaagtacatcaactga

SEQ ID NO: 7 - protein, 345 aa, Lactobacillus acidophilus
MTKNLLLGIAAVCGSTFQAVACTSIIFSPKDHYFGRNLDLEITFGQQVVITPRNYTFKFRKMPSLKKHYAMIGISLDMDDYPLYFDATNEKGLGMAGLNYPGNATYYEEKENKDNIASFEFIPWILGQCSTISEVKDLLSRINIADLNFSEKMQASSLHWLIADKTGTSLVVETDKDGMHIYDNPVGCLTNNPQFPKQLFNLNNYADVSPKMPKNNFSDKVNMAGYSRGLGSHNLPGGMDSESRFVRVAFNKFNAPIAETEEENIDTYFHILHSVEQQKGLDEVGPNSFEYTIYSDGTNLDKGIFYYTTYSNKQINVVDMNKEDLDSSNLITYDMLDKTKFNHQN

SEQ ID NO: 8 - DNA, 1038 bp, Lactobacillus acidophilus
atgaccaagaacttgttgttgggtattgctgctgtttgtggttctacttttcaagctgttgcttgcacctccattattttctcaccaaaggatcattacttcggcagaaacttggatttggaaattaccttcggtcaacaagttgttatcaccccaagaaactacaccttcaagtttagaaagatgccctccttgaagaaacattacgccatgattggtatctcattggatatggatgactaccccttgtactttgatgctacaaacgaaaaaggtttaggtatggctggtttgaattacccaggtaatgctacttactacgaagagaaagaaaacaaggacaatatcgcctccttcgaattcattccatggattttgggtcaatgctccactatctctgaagttaaggatttgttgtccaggatcaacattgccgatttgaacttctctgaaaagatgcaagcttcctcattgcattggttgattgctgataagactggtacttccttggttgttgaaactgataaggatggtatgcacatctacgataatccagttggttgtttgactaacaacccacaatttcctaagcagctgttcaacttaaacaactacgctgatgtttctcccaagatgccaaagaacaatttctccgataaggttaatatggccggttactctagaggtttgggttctcataatttgccaggtggtatggattctgaatctaggtttgttagagttgcctttaacaagttcaacgctccaattgctgaaaccgaagaagaaaacattgatacctacttccacatcttgcactccgtcgaacaacaaaagggtttagatgaagttggtccaaactctttcgagtacactatctattctgacggtacaaatttggacaagggtatcttctactacaccacctattctaacaagcaaatcaacgttgtcgacatgaacaaagaggatttggactcctctaacttgatcacctatgatatgttggacaagacgaagttcaaccaccaaaactga

SEQ ID NO: 9 - protein, 351 aa, Bacteroides fragilis
MRRKIMMATLIVIAVNFIWSGQSVKACTRAVYIGPDNMVITGRTMDWKEDIQSNLYLFPRGIKRAGYNKGNTVEWISKYGSIVATGYDIGTCDGMNEKGLVASLLFLPESIYVRRNDTRPVMGISIWTQYVLDNFATVSEAVEELKKQTFRIDAPDMPNGSASTLHMAITDETGNSAVLEYIDGNLIIHEGKEYQVMTNSPRYDLQLAVNDYWKEVGGLNMLPGTNRSSDRFVRASFYIHAIPQTSDAKIAVPSVLSVMRNVSVPFGITTPDKPYISSTRWRSVSDQKNRVYYFESTLTPNLFWIDLHKVDFSPNASIKKLSLTHGEVYAGDVVKDFKDSRSFTFMFELPQ

SEQ ID NO: 10 - DNA, 1056 bp, Bacteroides fragilis
atgaggcgtaagattatgatggctaccttgatcgttattgccgttaactttatttggtccggtcaatctgttaaggcttgtactagagctgtttacattggtccagataacatggttattactggtagaaccatggattggaaagaggacattcaatccaacttgtacttgttcccaagaggtattaagagagctggttacaacaagggtaataccgttgaatggatttccaagtacggttctatagttgctaccggttatgatatcggtacttgtgatggtatgaacgagaaaggtttggttgcttctttgttgttcttgccagaatccatctacgttagaagaaatgataccagaccagtcatgggtatttctatttggactcaatacgtcttggataacttcgctactgtttctgaagctgtcgaagaattgaagaagcaaaccttcagaattgatgctccagatatgccaaatggttctgcttctacattgcatatggctattactgacgaaactggtaactctgctgttttggagtacattgatggtaacttgatcatccacgagggtaaagaataccaagttatgactaactctccaaggtacgacttgcaattggctgttaatgattactggaaagaagttggcggtttgaatatgttgccaggtactaatagatcctccgatagatttgttagagcctccttttacattcacgctattccacaaacatccgatgctaaaattgctgttccatccgttttgtccgttatgagaaatgtttctgttccattcggtattactaccccagataagccatatatctcttctactagatggcgttctgtctccgatcaaaagaatagagtttactacttcgagtccactttgaccccaaatttgttttggattgacttgcacaaggttgacttttctccaaacgcttccatcaaaaagttgtctttgactcacggtgaagtttacgctggtgatgttgttaaggatttcaaggactctagaagcttcaccttcatgtttgaattgccacagtga

SEQ ID NO: 11 - protein, 344 aa, Enterococcus durans
MTKNLLLGIAAVCGSTFQAVACTSITYVTSDHYFGRNFDYEISYNEVVTVTPRNYKLNFRKVNDLDTHYAMIGIAAGIADYPLYYDATNEKGLSMAGLNFSGYADYKEIQEGKDNVSPFEFIPWILGQCSTVGEAKKLLKNINLANINYSDELPLSPLHWLLADKEKSIVIESMKDGLHIYDNPVGVLTNNPSFDYQLFNLNNYRVLSSETPKNNFSNQISLNAYSRGMGGIGLPGDLSSVSRFVKATFTKLNSVSGDSESESISQFFHILGSVEQQKGLCDVGDGKYEYTIYSSCCNVDKGIYYYRTYEDSQITAIDMNKEDLDSHKLISYPIIEKQQIKYIN

SEQ ID NO: 12 - DNA, 1035 bp, Enterococcus durans
atgaccaagaacttgttgttgggtattgctgctgtttgtggttctacttttcaagctgttgcttgtacctctattacctacgttacttccgatcattacttcggtagaaacttcgactacgagatctcttacaacgaagttgttactgttacccctaggaactacaagttgaacttcagaaaggttaacgatttggatacccattacgccatgattggtattgcagctggtatagctgattatcccttgtattatgatgccaccaacgaaaagggtttgtctatggctggtttgaatttttctggttacgccgactacaaagaaatccaagaaggtaaggataacgtcagcccatttgaattcattccatggattttgggtcaatgctctactgttggtgaagctaaaaagctgttgaagaacattaacctggccaacatcaactactctgatgaattgccattgtctccattgcattggttgttggctgacaaagaaaagtccatcgttatcgaatctatgaaggatggcttgcacatctacgataatccagttggtgttttgactaacaacccatcttttgactaccagctgttcaacctgaacaactacagagttttgtcatctgaaaccccaaagaacaacttctccaatcagatctctttgaacgcttactctagaggtatgggtggtattggtttgccaggtgatttgtcatcagtttccagatttgttaaggctaccttcaccaagttgaattccgtttctggtgattccgaatccgaatccatttctcaattcttccatatcttgggttccgtcgaacaacaaaaaggtttgtgtgatgttggtgacggtaaatacgagtacactatctactcctcttgttgcaatgttgacaagggtatctactactacagaacctacgaagattctcaaattaccgccatcgacatgaacaaagaagatttggattctcacaagctgatctcgtacccaattattgaaaagcagcagatcaagtacatcaactga

SEQ ID NO: 13 - protein, 344 aa, Enterococcus faecalis
MTKNLLLGIAAVCGSTFQAVACTAITYVSKDHYFGRNFDYEISYNEVVTITPRNYKFSFREVGNLDHHFAIIGIAAGIADYPLYYDAINEKGLGMAGLNFSGYADYKKIEEGKENVSPFEFIPWVLGQCSTVDEAKKLLKNLNLVNINFSDELPLSPLHWLLADKEQSIVVESTKEGLRVFDNPVGVLTNNPTFDYQLFNLNNYRVLSTRTPKNNFSDQIELDIYSRGMGGIGLPGDLSSVSRFVKATFTKLNSVSRSSEYESISQFFHILSSVEQQKGLCDVGDEKYEYTIYSSCCNLEKGIYYYRTYDNSQITAVDMNKENLEKDSLIVYPMVETQQINYAN

SEQ ID NO: 14 - DNA, 1035 bp, Enterococcus faecalis
atgaccaagaacttgttgttgggtattgctgctgtttgtggttctacttttcaagctgttgcttgtactgctatcacctacgtttctaaggatcattacttcggtagaaacttcgactacgagatctcttacaacgaagttgttactatcacccctaggaactacaagttctcattcagagaagttggtaacttggatcatcatttcgccattattggtattgcagctggtatagctgattaccccttgtattatgatgccatcaacgaaaaaggtttaggtatggctggtttgaacttttctggttacgctgactacaagaagatcgaagaaggtaaagaaaacgtcagcccattcgaattcattccatgggttttgggtcaatgttctactgttgatgaagccaagaagctgttgaagaacttgaacttggttaacatcaacttctccgacgaattgccattgtctccattgcattggttgttggctgacaaagaacaatccatcgttgtcgaatctaccaaagaaggtttgagagttttcgataacccagttggtgttttgactaacaacccaacttttgactaccagctgttcaacctgaacaactacagagttttgtctactagaacccctaagaacaacttctcagaccaaatcgaattggacatctactctagaggtatgggtggtattggtttgccaggtgatttgtcatctgtttccagatttgttaaggctaccttcaccaagttgaactccgtttctagatcttccgaatacgaatccatctctcaattcttccacatcttgtcctctgtcgaacaacaaaagggtttgtgtgatgttggtgacgaaaagtacgagtacactatctactcctcttgttgcaatttggagaagggtatctactactacagaacctacgataactcccaaattaccgctgttgacatgaacaaagaaaacctggaaaaggactccttgatcgtttatccaatggttgaaacccaacaaatcaactacgctaactaa

SEQ ID NO: 15 - protein, 345 aa, Lactobacillus acidophilus
MTKNLLLGIAAVCGSTFQAVACTSICYNPNDHYFGRNLDYEIAYGQKVVIVPRNYEFKYREMPSQKMHYAFIGVSVVNDDYPLLCDAINEKGLGIAGLNFQGPNHYFPKIEGKKNIASFELMPYLLSNCENTDDVKEILDNANILNISFSANYPAADLHWILSDKAGKSIAVESTNSGLHIYDNPVNVLTNNPEFPDQLIKLSDYADVTPHNPKNTLVPNVDLNLYSRGLGTHHLPGGMDSSSRFVKVAFVLAHTPQGKNEVENVTNYFHILHSVEQPDGLDEVEDNRYEYTMYTDCMNLDKGILYFTTYDNNRINAVDMHKADLGSEDLICYDLFKKQDIEYMN

SEQ ID NO: 16 - DNA, 1038 bp, Lactobacillus acidophilus
atgaccaagaacttgttgttgggtattgctgctgtttgtggttctacttttcaagctgttgcttgtacctctatttgctacaatccaaacgatcactacttcggtagaaacttggattacgaaattgcctacggtcaaaaggttgttatcgttccaagaaactacgagttcaagtacagagaaatgccctctcaaaaaatgcattacgccttcattggtgtttccgttgttaatgatgattaccctttgttgtgtgacgccatcaacgaaaaaggtttaggtattgccggtttgaatttccaaggtccaaatcattacttcccaaagatcgaaggcaagaagaacattgcttctttcgaattgatgccctacttgttgtctaactgtgaaaacaccgatgacgtcaaagaaattttggataacgccaacatcctgaacatttctttctctgctaattatccagctgctgacttgcattggattttgtctgataaggctggtaagtccattgctgttgaatctactaattctggcttgcacatctacgataacccagttaatgttctgactaacaacccagaattcccagaccaattgattaagttgtctgattacgctgatgttaccccacataatccaaagaatactttggttccaaacgtcgacttgaacttgtattctagaggtttgggtacacatcatttgccaggtggtatggattcttcatctagatttgttaaggtcgctttcgttttggctcatactccacaaggtaagaacgaagttgaaaacgttaccaactacttccacatcttgcactctgttgaacaaccagatggtttggatgaagtcgaagataatagatacgagtacactatgtacaccgactgtatgaatttggacaagggtatcttgtacttcactacctatgacaacaacagaatcaacgctgttgatatgcataaggctgatttgggttccgaagatttgatttgctacgacttgttcaagaagcaagacatcgagtacatgaactaa

SEQ ID NO: 17 - protein, 338 aa, Lactobacillus brevis
MTKNLLLGIAAVCGSTFQAVACTSLTYTSATGHHFFARTMDFPTTTPWRPVFLPRHYHWETALHVTRTTQYAILGGGRIAADFSAYLLADGFNESGISCAELYLPGVVDYAPAPVAGQLNLTPQDFINWALGEHATLAAIAADLPHVTLVDQPWGADRYVYPFHWILSDRSGQTLVIEPTGGPLTAQLNPAGVLTNTPILATHLRNLNDFLNVDAPDFSPATVAAARSYLVSGQPLPSGPVPTHRFIHTAIRRWGNPPATTTTAAVTHLFQWLAEVQLPYQPKRRHDRNHNYTHYRGICDLDTKSYLFIPRTTGRLQQITLTPEMAACRHFPHAFPAN

SEQ ID NO: 18 - DNA, 1017 bp, Lactobacillus brevis
atgaccaagaacttgttgttgggtattgctgctgtttgtggttctacttttcaagctgttgcttgtacttctttgacttacacttctgctactggtcatcatttcttcgctagaactatggatttcccaactactactccttggaggccagtttttttgccaagacattatcattgggaaaccgccttgcatgttactagaactactcaatacgctatcttaggtggtggtagaattgctgcagatttttctgcttatttgttggccgatggtttcaacgaatctggtatttcttgtgctgagttgtatttgccaggtgttgttgattatgctccagctccagttgctggtcaattgaatttgactccacaagatttcattaactgggccttgggtgaacatgctactttggctgctattgcagctgatttgccacatgttactttagttgaccaaccatggggtgctgatagatatgtttatccattccattggatcctgtccgatagatctggtcaaactttggttattgaacctactggtggtccattgactgcacaattgaatccagctggtgttttgactaacactccaattttggctacccacttgagaaacttgaacgatttcttgaatgttgatgccccagatttctctccagctactgttgcagctgctagatcttatttggtttctggtcaaccattgccatctggtccagttccaactcatagattcattcataccgctattagaagatggggtaatccaccagctactactacaacagctgctgttactcatttgtttcaatggttggctgaagtccaattgccatatcaacctaaaagaaggcatgacagaaaccataactacacccattacagaggtatttgcgatttggataccaagtcctacttgttcattccaagaactactggtagattgcaacagattactttgacaccagaaatggctgcttgtagacattttccacatgcttttccagctaactaa

SEQ ID NO: 19 - protein, 345 aa, Lactobacillus fermentum
MTKNLLLGIAAVCGSTFQAVACTSINVIAQDGYHVLGRTMDWDDLLVSPIFTPRHYQLASVFDHRVHENPYAIIGGGSITERRTDVSDGVNEFGLMAQKLTFKNGARLVDERHPDKVQLAAFELIFYLLGHFKSVADVAAHLDQIELMNDVNADVPFGYSEQHFVLSDPTGRCVVIEPSEHPLKLIDNPLGIMTNMPKFDHQLERLQDYLDFTPDFLNGTLAPNTFHVTTGKLSGKKTPPGAYTPKGRYVRAAYMKELADQPASKDEALATTWHLLDSVTVPKSKAHRPTFSVYRAATVAEDRTYYFQSYHQAQVTSVKLTDDLLKRATPLVFDTADVWAPVKLN

SEQ ID NO: 20 - DNA, 1038 bp, Lactobacillus fermentum
atgaccaagaacttgttgttgggtattgctgctgtttgtggttctacttttcaagctgttgcttgcacctccattaacgttattgctcaagatggttaccacgttttgggtagaactatggattgggatgatttgttggtttccccaattttcactccaagacattaccaattggcctctgttttcgatcatagagttcacgaaaatccatacgccattattggtggtggttctattactgaaagaaggactgacgtttctgatggtgttaatgaatttggtttgatggcccaaaagctgacttttaagaatggtgctagattggttgacgaaagacatccagataaggttcaattggctgctttcgaattgatcttctatttgttgggtcacttcaagtccgttgctgatgttgctgctcatttggatcaaattgaattgatgaacgatgtcaacgccgatgttccatttggttattctgaacaacacttcgttttgtctgatccaactggtagatgcgttgttattgaaccatctgaacatcccttgaagttgatcgataatccattgggtattatgaccaacatgccaaagttcgatcaccaattggaaagattgcaagactacttggatttcaccccagattttttgaatggtactttggctccaaacaccttccatgttactactggtaaattgtccggtaaaaaaactccaccaggtgcttatactccaaagggtagatatgttagagctgcctatatgaaggaattggctgatcaaccagcttctaaagatgaagctttggctactacatggcacttgttggattctgttactgttccaaaatccaaggctcatagaccaactttctctgtttacagagctgctactgttgctgaagatagaacttactacttccaatcctaccatcaagctcaagttacctctgttaagttgactgatgacttgttgaaaagagctaccccattggtttttgatacagctgatgtttgggctccagtcaagttgaactaa

SEQ ID NO: 21 - protein, 345 aa, Lactobacillus gallinarum
MTKNLLLGIAAVCGSTFQAVACTSIIFSPRDHYFGRNLDLEVTFGQQVVITPRDYVFKFRDMPKIDHHYAMVGIALNADSYPLYFDAANEKGLGMVGLNYPDNAIYYDVKEGKDNIASFEFIPWILGQAATVAEAKKLLSTINITKKNFSDKMQASPLHWIIADKTGVSIVVETDADGMHVYDNPVGCLTNNPQFPKQLFNLNNYADVSAAMPKNNFSDQVKMDGYSRGLGSRNLPGGMDSESRFVRVAFNKFNAPTSDSEEENVDTYFHILHSVEQQKGLDQVGPDAFEYTIYSDGINLDKGIFYYTTYTNKQINVVDMNKEDLDSSDLITYDMLTKGKFNHQN

SEQ ID NO: 22 - DNA, 1038 bp, Lactobacillus gallinarum
atgaccaagaacttgttgttgggtattgctgctgtttgtggttctacttttcaagctgttgcttgcacctccattattttctcaccaagagatcattacttcggcagaaacttggatttggaagttactttcggtcaacaagttgttatcaccccaagagattacgtgttcaagtttagagatatgccaaagatcgatcatcactacgctatggttggtattgctttgaatgctgattcttaccccttgtactttgatgctgctaacgaaaaaggtttaggtatggtcggtttgaactacccagataatgctatctactacgacgtcaaagaaggcaaggataacattgcttccttcgaattcattccatggattttgggtcaagctgctactgttgctgaagctaaaaagttgttgtccaccattaacatcaccaagaagaacttctccgataagatgcaagcttctccattgcattggattattgctgataagaccggtgtttccatcgttgttgaaactgatgctgatggtatgcacgtttacgataatccagttggttgtttgactaacaacccacaatttccaaagcagctgttcaacttaaacaactacgctgatgtttctgctgccatgccaaaaaacaatttctccgatcaagttaagatggacggttactctagaggtttgggttctagaaatttgccaggtggtatggattctgaatctaggtttgttagagttgcctttaacaagttcaacgctccaacttctgattccgaagaagaaaacgttgatacctacttccatatcttgcactccgtcgaacaacaaaagggtttagatcaagttggtccagatgcttttgagtacactatctattccgatggtatcaacttggacaagggtattttctactacactacctacaccaacaagcaaatcaacgttgtcgacatgaacaaagaggatttggattcctctgatttgatcacctacgatatgttgaccaagggtaaattcaaccaccagaactga

SEQ ID NO: 23 - protein, 345 aa, Lactobacillus gasseri Am1
MTKNLLLGIAAVCGSTFQAVACTSILYSPKDHYFGRNLDYEIAYGQKVVITPRNYEFEFTDLPVEKSHYAMIGVAAVADNTPLYCDAINEKGLGVAGLSFAGQGKYFPNAVNKKNIASFEFISYLLATYETVDQVKESLTNANISNVSFAKNTPASELHWLVGDKTGKSIVVESDEKGLHVYNNPVNALTNAPLFPEQLTNLVNFASVVPGEPDNNFLPGVNLKLYSRSLGTHHLPGGMDSESRFVKVCFALNHAPKDSDEVENVTNFFHILESVEQAKGMDQVGPNSFEYTMYTSCMNLEKGILYFNCYDDSRISAVDMNKEDLDSSDLVVYDLFKKQDISFIN

SEQ ID NO: 24 - DNA, 1038 bp, Lactobacillus gasseri Am1
atgaccaagaacttgttgttgggtattgctgctgtttgtggttctacttttcaagctgttgcttgtacctctatcttgtactctccaaaggatcattacttcggtagaaacttggattacgaaattgcctacggtcaaaaggttgttattaccccaagaaactacgagttcgaattcactgatttgccagttgaaaagtcccattacgctatgattggtgttgctgcagttgctgataataccccattatattgtgatgccatcaacgagaaaggtttgggtgttgcaggtttgtcttttgctggtcaaggtaaatactttccaaacgccgttaacaagaagaatattgcctccttcgagttcatctcttacttgttggctacttacgaaaccgttgaccaagtcaaagaatccttgactaacgccaacatctctaacgtttctttcgctaagaataccccagcttctgaattgcattggttggttggtgataagactggtaagtctatcgttgttgaatctgacgaaaagggcttgcatgtttacaacaatccagttaacgctttgaccaatgctcctttgtttccagaacaattgaccaacttggttaacttcgcttctgttgttccaggtgaaccagataacaattttttgccaggtgtcaacctgaagttgtactctagatctttgggtactcatcatttgcctggtggtatggattctgaatcaagattcgttaaggtttgcttcgctttgaatcatgccccaaaagattctgatgaagttgaaaacgttaccaacttcttccacatcttggaatctgttgaacaagccaaaggtatggatcaagtaggtccaaattctttcgagtacactatgtacacctcttgcatgaatttggagaaaggcatcttgtatttcaactgctacgatgactctagaatctccgctgttgatatgaacaaagaggatttggactcctccgatttggttgtttacgatttgttcaagaagcaggacatctccttcatcaactga

SEQ ID NO: 25 - protein, 257 aa, Lactobacillus helveticus
MTKNLLLGIAAVCGSTFQAVACSSIIFSPKDHYFGRNLDLEITFGQQVIITPRDYVFKFRDMPEIDHHYAMVGIALNAGGYPLYFDAANEKGLGMGGLNYPDNAIYYDVKEGKDNIASFEFIPWILSQAATVEEAKKLLAKINITKKNFSDKMHVSPLHWIIADKTGASIVVETDADGMHVYDNPVGCLTNNPQFPKQLFNLNNYQDVSPAMPKNNFSSKINMDGYSRGLGSRNLPGGMDAESRFVRVAFNKRAWIK

SEQ ID NO: 26 - DNA, 774 bp, Lactobacillus helveticus
atgaccaagaacttgttgttgggtattgctgctgtttgtggttctacttttcaagctgttgcttgctcctccattattttctcaccaaaggatcattacttcggcagaaacttggatttggaaattacctttggtcagcaagttatcatcaccccaagagattacgttttcaagttcagagatatgccagaaatcgatcatcattacgccatggttggtattgctttgaatgctggtggttacccattatactttgatgctgctaacgaaaaaggtctaggtatgggtggtttgaattacccagataatgctatctactacgacgtcaaagaaggcaaggataacattgcttccttcgaattcattccctggattttgtctcaagctgctactgttgaagaagccaaaaagttgttggccaagattaacatcaccaagaagaacttctccgacaagatgcatgtttctccattgcattggattattgctgataagactggtgcctctatcgttgttgaaactgatgctgatggtatgcacgtttacgataatccagttggttgtttgactaacaacccacaatttcctaagcagctgttcaacctgaacaactaccaagatgtttcaccagctatgccaaagaacaacttcagctctaagatcaacatggacggttattctagaggtttgggttctagaaatttgccaggtggtatggatgctgaatccagatttgttagagttgcctttaacaagagagcctggatcaagtaa

SEQ ID NO: 27 - protein, 336 aa, Lactobacillus johnsonii
MTKNLLLGIAAVCGSTFQAVACTGLRFTDDQGNLYFGRNLDVGQDYGEGVIITPRNYPLPYKFLDNTTTKKAVIGMGIVVDGYPSYFDCYNEDGLGIAGLNFPHFAKFSDGPIDGKINLASYEIMLWVTQNFTHVSEVKEALKNVNLVNEAINTSFAVAPLHWIISDSDEAIIVEVSKQYGMKVFDDKVGVLTNSPDFNWHLTNLGNYTGLNPHDATAQSWNGQKVAPWGVGTGSLGLPGDSIPADRFVKAAYLNANYPTVKGEKANVAKFFNILKSVAMIKGSVVNDQGSDEYTVYTACYSSGSKTYYCNFEDDFELKTYKLDDHTMNSTSLVTY

SEQ ID NO: 28 - DNA, 1011 bp, Lactobacillus johnsonii
atgaccaagaacttgttgttgggtattgctgctgtttgtggttctacttttcaagctgttgcttgtactggtttgagattcactgatgatcaaggcaacttgtacttcggtagaaatttggatgttggtcaagattatggtgagggtgttattatcactccaagaaattatcccttgccatacaagtttttggataacactactaccaaaaaggccgttattggtatgggtatagttgttgatggttacccatcttacttcgactgttacaatgaagatggtttgggtatcgctggtttgaattttccacatttcgctaagttttccgatggtccaattgatggtaagattaacttggcctcctacgaaattatgttgtgggttactcaaaacttcacccacgtttctgaagtcaaagaagctttgaagaacgtcaacttggttaacgaagctatcaacacttcttttgctgttgctccattgcattggatcatctctgattctgatgaagccatcatcgttgaagtctctaaacaatacggtatgaaggttttcgatgataaggttggtgttttgactaactccccagattttaactggcatttgaccaatttgggtaactacacaggtttgaatccacatgatgctactgctcaatcttggaatggtcaaaaagttgctccttggggtgttggtactggttctttgggtttgccaggtgattctattccagctgatagatttgttaaggctgcttacttgaatgctaactacccaactgttaagggtgaaaaggctaatgttgctaagttcttcaacatcttgaagtccgttgctatgattaagggttccgttgttaacgatcaaggttctgatgagtacactgtctatactgcttgttactcttctggttctaagacctactactgcaacttcgaagatgacttcgaactgaaaacctacaagttggatgatcacaccatgaactctacatctttggttacctactaa

SEQ ID NO: 29 - protein, 358 aa, Lactobacillus plantarum
MTKNLLLGIAAVCGSTFQAVACTSLTYTNSHGGHFLARTMDFNVDFETRIMFMPRHYRVTGDLGDFTTTYGFIGAGRQLNHEIFTDGVNECGVSIAALYFPNHAIYQPHSNQDKIDLAPHDFVAWVLGKITSVADLRERVKDVQLISSTAELINEIPPLHFIISDQTGETAVLEPTSGELRLINNPVGVLTNSPNLKWQLQNLSKYGTLTNTERPLNKFINYQPGSQGPGTGALGLPGDYTSMSRFARTVFLKHYAQVPATTTDTVNLLQHILNAVTIPKGAKVAANGQATYTEYRSYMDLNHQTYALELYENPGVIQQVNLTDHLLEKQTVPLEYALSRTPHVQLLTPDIATLPAAH

SEQ ID NO: 30 - DNA, 1077 bp, Lactobacillus plantarum
atgaccaagaacttgttgttgggtattgctgctgtttgtggttctacttttcaagctgttgcttgtacttctttgacctacactaattctcatggtggtcatttcttggctagaaccatggattttaacgttgacttcgaaaccaggatcatgttcatgccaagacattatagagttaccggtgatttgggtgatttcactactacttacggttttattggtgccggtagacaattgaaccacgaaattttcactgatggtgttaacgaatgcggtgtttctattgctgcattatactttccaaaccacgctatctatcagccacattccaatcaagataagattgatttggccccacatgattttgttgcttgggttttgggtaagattacctctgttgctgatttgagagaaagagttaaggacgtccaattgatttcttctaccgccgaattgattaacgaaatcccaccattgcatttcatcatctctgatcaaactggtgaaactgctgttttggaacctacttctggtgaattgagattgatcaacaatccagttggtgtcttgactaactctccaaatttgaagtggcaactgcagaacttgtctaagtatggtactttgactaacaccgaaaggccattgaacaagttcattaactatcaaccaggttctcaaggtccaggtactggtgctttgggtttgccaggtgattatacttctatgtctagattcgctaggaccgtttttttgaaacattacgctcaagttccagctactactactgatactgttaacttgttgcagcatatcttgaacgctgttactattccaaaaggtgctaaggttgctgctaatggtcaagctacttatactgagtacagatcctacatggatttgaaccatcaaacttacgctttggagttgtacgaaaacccaggtgttattcaacaagtcaacttgaccgatcacttgttggaaaaacaaaccgttccattggaatacgctttgtctagaactccacacgttcaattattgaccccagatattgctactttgccagctgctcattaa

SEQ ID NO: 31 - protein, 348 aa, Lactobacillus plantarum
MTKNLLLGIAAVCGSTFQAVACTSLTIQTTAGDQFLARTMDFAFELGGRPVAIPRNHHFDSVTNADGFDSPYSFVGTGRDLNGYIFVDGVNEHGVSAAALYFSGQAHFTQQTKAGKVNLAPHEVLMWILGNVKSTAELGERIADLNVMEAAAPLLNIVVPLHWIISDKSGSTYVLELENDGVHYMKNPVGVMTNTPDFEWHLKNLSNYVNLQPGPHPSRQYGDMTVNPFGPGTGASGMPGDYTSVARFVRTVFMREHTDAVTTDAEAVNALSHMLNSVEIPKGVKMQDNGTPDYTQYRAYMSMDEPAFYMQPYADQTITRVELTPALMTAAQPTEFELKTTQQFRLAN

SEQ ID NO: 32 - DNA, 1047 bp, Lactobacillus plantarum
atgaccaagaacttgttgttgggtattgctgctgtttgtggttctacttttcaagctgttgcttgtacctctttgactattcaaactactgctggtgatcaattcttggctagaactatggatttcgcctttgaattaggtggtagaccagttgctattccaagaaaccatcatttcgattctgttaccaacgccgatggttttgattctccatattcttttgttggcaccggtagagatttgaacggttacatttttgttgacggtgttaacgaacatggtgtttctgctgctgcattatacttttctggtcaagctcatttcactcaacaaactaaggctggtaaggttaatttggctccacatgaagttttgatgtggattttgggtaacgttaagtctactgctgaattgggtgaaagaattgctgacttgaatgttatggaagctgctgctcctttgttgaatatcgttgttccattgcattggatcatctccgataagtctggttctacctatgtcttggaattggaaaacgatggtgttcactacatgaagaatccagttggtgttatgactaacaccccagattttgaatggcacttgaagaacttgagcaactacgttaacttgcaaccaggtccacatccatctagacaatatggtgatatgactgttaatccattcggtccaggtactggtgcttctggtatgccaggtgattatacttctgttgctagattcgttaggaccgtgtttatgagagaacatactgatgctgttactaccgatgctgaagctgttaatgctttgtctcatatgttgaactccgtcgaaattccaaagggtgttaagatgcaagataacggtactccagattacactcagtatagagcttacatgtctatggatgaaccagccttttacatgcaaccatatgctgatcaaaccatcaccagagttgaattgactccagctttgatgactgctgctcaacctactgaatttgagttgaaaacaacccagcaattcagattggctaactaa

SEQ ID NO: 33 - protein, 337 aa, Lactobacillus plantarum
MTKNLLLGIAAVCGSTFQAVACTSLTYLDTDNHRYFARTMDFPTTTPWRPIFLPRRYPWPTGLATTRMTQYAILGGGRLPDHFKACLMADGINEAGLVCAELYLPHAVEYATQPQVNQINLTPQAFINWALGEHQSVAAVIADLPSVNLVGASWGDDTGEVYPFHWYLSDAHTSVVIEPTGGPLTAQPNPAGVLTNTPVLSDHQRRLNLYLAVSGNQITTATRQAAQHVIQTKQPLPSGPIPTDRFIHMALRRLGTPQLAPQQVPTTLFRWLQEVSLPYHADRHHLISHNYTHYRCLITLATRTYRFIPRTTGHEQRLTLTPEMAATWRTPYLFPAD

SEQ ID NO: 34 - DNA, 1014 bp, Lactobacillus plantarum
atgaccaagaacttgttgttgggtattgctgctgtttgtggttctacttttcaagctgttgcttgtacttctttgacctacttggataccgataaccatagatacttcgctagaactatggatttcccaactactactccttggaggccaattttcttgccaagaagatatccttggccaactggtttggctactactagaatgactcaatacgctattttaggtggtggtagattgccagatcatttcaaagcttgtttgatggccgatggtattaacgaagctggtttggtttgtgccgaattatacttgccacatgctgttgaatatgctactcaaccacaagttaaccagattaacttgactccacaagcctttattaactgggctttgggtgaacatcaatctgttgctgcagttattgctgatttgccatccgttaatttggttggtgcttcttggggtgatgatactggtgaagtttatccatttcactggtacttgtctgatgctcatacctctgttgttattgaacctactggtggtccattgactgctcaacctaatccagctggtgttttgactaatactccagttttgtccgatcaccagagaagattgaacttgtatttggccgtttccggtaatcaaattactactgctactagacaagctgcccaacatgttattcaaactaagcaaccattgccatctggtccaattccaactgatagattcattcatatggccttgagaaggttgggtactccacaattggctccacaacaagtgccaactactttgtttagatggttgcaagaagtctccttgccatatcatgctgatagacatcatttgatctcccataactacacccattacagatgcttgattactttggctacaagaacctacagattcatcccaagaactactggtcatgaacaaagattgactttgacaccagaaatggctgctacttggagaactccatatttgtttccagctgactga

SEQ ID NO: 35 - protein, 345 aa, Lactobacillus reuteri
MTKNLLLGIAAVCGSTFQAVACTSVIYTAGDYYFGRNLDLEVNLGQEVVITPRNKTLEFREMPNLEHHYAIIGMSIVRDDYPLYFDGVNEKGVGMAGLNFDGPAHYFPVQEGKDNIASFELVPYILVTASSVAEAKKLLSNANIANINFSDKLQAAPLHWIIADKTGASVTVESTAKGLNVYDNPVGVLTNNPEFPRQLLNLSNYRSVAPANPANVFAPDVDLPVYSRGLGTHFLPGGMDSESRFVKATFTKMHAPVGNSEVENITDYFHILQSVEQQKGLDEVAPDTFEYTIYSDGSNLKKGIFYYKTYENNQINAVDMHKEDLEASELITYPVQNKQIINQQN

SEQ ID NO: 36 - DNA, 1038 bp, Lactobacillus reuteri
atgaccaagaacttgttgttgggtattgctgctgtttgtggttctacttttcaagctgttgcttgtacctctgttatctatactgctggtgattactacttcggtagaaacttggatttggaagtcaacttgggtcaagaagttgttattaccccaagaaacaagaccttggaattcagagaaatgccaaacttggaacatcattacgccattatcggtatgtccatcgttagagatgattaccccttgtactttgatggtgtcaacgaaaaaggtgttggtatggctggtttgaattttgatggtccagctcattacttcccagttcaagaaggtaaggataatatcgcctcttttgaattggtcccctatattttggttaccgcttcttctgttgctgaggctaaaaagttgttgtccaatgctaatattgccaacatcaacttctccgataagttgcaagctgctccattgcattggattattgctgataagactggtgcttctgttaccgttgaatctactgctaaaggtttgaacgtttacgataacccagttggtgttttgactaacaatccagaatttccaaggcagctgttgaacttgtctaactatagatctgttgctccagctaacccagctaatgtttttgctccagatgttgatttgccagtctattctagaggtttgggtactcattttttgccaggtggtatggattctgaatctaggtttgttaaggctaccttcactaagatgcatgctccagttggtaattccgaagttgaaaacattaccgactacttccacatcttgcaatccgtcgaacaacaaaagggtttagatgaagttgccccagatacttttgagtacactatctattctgacggctccaatttgaagaagggtatcttttactacaagacctacgagaacaatcaaatcaacgctgttgacatgcacaaagaagatcttgaagcctctgaattgatcacataccctgttcaaaacaagcagatcatcaaccagcaaaactaa

SEQ ID NO: 37 - protein, 358 aa, Lactobacillus rhamnosus
MTKNLLLGIAAVCGSTFQAVACSSMTIKSLQGDIFWGRTMDYNTSFFHESPAGGVPGKIVSLPANQTLPAQTATWKTKYAAVGVGVDQSVALFDGVNSEGLAGDLQVLVECSWASAESLAKRNLKPIKGEEFVTLALTTCKNVAEVRALASQYGLLDEPFQFGGQGVKIPLHYTFVDPSGAGLVIELTGHGAFKLYDSVGAMTNSPEYGWHTTNLRNYVSLNDRDYPEGADLGDQHLEPIELGTGYGMFGLPGDYTSPSRFVRAMFVSRNLDPFNSNEGIRVLYNAFKTVLIPQGLGRDPKHSILTDYTQYWSGYDLSKRAIFVQDANTLTMTTKTLDPNLTEVTYDDLVKTEQFNQL

SEQ ID NO: 38 - DNA, 1077 bp, Lactobacillus rhamnosus
atgaccaagaacttgttgttgggtattgctgctgtttgtggttctacttttcaagctgttgcttgttcttccatgaccatcaaatctttacaaggtgatatcttctggggtcgtactatggattacaacacttcatttttccatgaatctccagctggtggtgttccaggtaaaatcgtttctttgccagctaatcaaacattgccagctcaaactgctacttggaaaactaagtacgctgctgttggtgttggtgtagatcaatcagttgctttgtttgatggtgtcaactctgaaggtttggctggtgacttacaagttttggttgaatgttcttgggcttccgctgaatctttggctaaaagaaatttgaagccaatcaagggtgaagagttcgttactttggctttgactacttgtaagaacgttgctgaagttagagctttggcttctcaatatggtttgttggatgaaccatttcaattcggtggtcaaggtgttaagattccattgcattacactttcgttgatccatctggtgctggtttggttattgaattgacaggtcatggtgctttcaagttgtatgattctgttggtgctatgaccaactctccagaatatggttggcataccactaacttgagaaactacgtttccttgaacgatagagattatcctgaaggtgctgatttgggtgatcaacatttggaacctatcgaattaggtactggttacggtatgtttggtttgccaggtgattatacttctccatccagatttgttagagccatgtttgtctctagaaacttggatccattcaactccaacgaaggtattagagtcttgtacaacgctttcaagaccgttttgattccacaaggtttgggtagagatccaaagcactctattttgactgattacacccaatactggtccggttacgatttgtctaagagagctattttcgttcaagatgccaacactttgactatgactaccaagactttggacccaaacttgactgaagttacctatgatgatttggtcaagaccgaacagtttaaccagttgtga

SEQ ID NO: 39 - protein, 334 aa, Bifidobacterium animalis
MTKNLLLGIAAVCGSTFQAVACTAVRFDDGQNNMYFGRNLDWSEDYGEKIVFAPHDYHYAPAFNAEDKNHPVIGIGIIVEDTPLYFDCMNDAGLAVAGLNFAKYCKYATEAVNFTTNVAAYEFPLWVTRNFTSVDDVQEALKNVTIVGKPINDRFPVATLHWIIADNTRSIVVECTEDGMHVYDDDVDVLTNQPPFPQQIEHLDNYAYVSPRTGKSVKWGSSELETNQDSNSSQGLPGGYGSMARFVRAAYNNTHYPTQSGENANVNRLFKTLSTAAVIEGTAISANAEFEKTLFSDCYSTATQTVYLKKYDDMAVHSYAVKDFDASSNQLQSK

SEQ ID NO: 40 - DNA, 1005 bp, Bifidobacterium animalis
atgaccaagaacttgttgttgggtattgctgctgtttgtggttctacttttcaagctgttgcttgtactgctgttagatttgatgatggtcagaacaacatgtacttcggtagaaatttggactggtctgaagattacggtgagaagatagtttttgccccacatgattatcattacgctccagcttttaacgccgaagataagaatcatccagttattggtatcggtatcatcgttgaagatactcccttgtactttgattgcatgaatgatgctggtttggctgttgcaggtttgaattttgctaaatactgcaagtacgctaccgaagctgttaatttcactactaatgttgctgcctacgaattcccattgtgggttactagaaatttcacctccgttgatgatgttcaagaagccttgaaaaacgttaccatcgttggtaagccaatcaacgatagatttccagttgctaccttgcattggattattgctgataacaccagatccatcgttgttgaatgtactgaagatggtatgcacgtttacgatgatgatgtcgatgttttgactaaccagccaccatttccacaacaaatcgaacatttggataactacgcttacgtttctccaagaactggtaaatctgttaagtggggttcttccgaattggaaactaatcaagactccaactcttctcaaggtttgccaggtggttatggttctatggctagatttgttagagctgcttacaacaacactcactacccaactcaatctggtgaaaatgctaatgtcaacaggttgttcaagactttgtctaccgctgctgttattgaaggtactgctatttctgctaacgctgaattcgaaaagaccttgttttctgattgctactctactgctactcaaaccgtctacttgaaaaagtacgatgatatggccgttcattcctacgctgttaaggattttgatgcttcttctaaccagctgcagtctaagtaa

SEQ ID NO: 41 - protein, 337 aa, Bifidobacterium breve
MTKNLLLGIAAVCGSTFQAVACTGFRFSDDEGNTYFGRNLDWSFSYGETILVTPRGYHYDTVFGAGGKAKPNAVIGVGVVMADRPMYFDCANEHGLAIAGLNFPGYASFVHEPVEGTENVATFEYPLWVARNFDSVDEVEEALRNVTLVSQIVPGQQESLLHWFIGDGKRSIVVEQMADGMHVHHDDVDVLTNQPTFDFHMENLRNYMCVSNEMAEPTSWGKASLTAWGAGVGMHGIPGDVSSPSRSVRVAYTNAHYPQQNDEAANVSRLFHTLGSVQMVDGMAKMGNGQFERTLYTSGYSSKTNTYYMNTYDDPAIRSYAMADYDMDSSELISVAR

SEQ ID NO: 42 - DNA, 1065 bp, Bifidobacterium breve
atgaccaagaacttgttgttgggtattgctgctgtttgtggttctacttttcaagctgttgccgatattatgaacgtccaagctaatcaatctgacggtagaaagtctgttatgtgtactggtttcagattctccgatgatgaaggtaatacttacttcggtagaaacttggactggtctttttcttacggtgaaaccattttggttaccccaagaggttatcattacgatactgtttttggtgctggtggtaaagctaaacctaatgctgttataggtgttggtgttgttatggctgatagaccaatgtactttgattgcgctaatgaacatggtttggctattgctggtttgaattttccaggttacgcttcctttgttcacgaaccagttgaaggtactgaaaacgttgctacttttgaatacccattgtgggttgctagaaacttcgattctgttgatgaagttgaagaagccttgagaaacgttaccttggtttctcaaatagtcccaggtcaacaagaatctttgttgcattggtttatcggtgacggtaagagatctatcgttgttgaacaaatggctgatggtatgcatgttcatcacgatgatgttgatgttttgactaaccagccaaccttcgattttcacatggaaaacttgaggaactacatgtgcgtttctaacgaaatggcagaacctacaagttggggtaaagcttctttgactgcttggggtgccggtgttggtatgcacggtattccaggtgatgtttcttcaccatctagatctgttagagttgcttacactaatgctcactacccacaacaaaatgatgaagctgctaatgtctctaggttgttccatactttgggttccgttcaaatggtagacggtatggctaaaatgggtaatggtcaatttgaaaggaccttgtacacttccggttactcttctaaaactaacacctactacatgaacacctacgatgatccagctattagatcttatgctatggccgattacgatatggactcctctgaattgatttccgttgctagatga

SEQ ID NO: 43 - protein, 337 aa, Bifidobacterium longum subsp. Infantis
MTKNLLLGIAAVCGSTFQAVACTGVRFSDDEGNTYFGRNLDWSFSYGETILVTPRGYHYDTVFGAGGKAKPNAVIGVGVVMADRPMYFDCANEHGLAIAGLNFPGYASFVHEPVEGTENVATFEFPLWVARNFDSVDEVEEALRNVTLVSQIVPGQQESLLHWFIGDGKRSIVVEQMADGMHVHHDDVDVLTNQPTFDFHMENLRNYMCVSNEMAEPTSWGKASLTAWGAGVGMHGIPGDVSSPSRFVRVAYTNAHYSQQNDEAANVSRLFHTLGAVQMVDGMAKMGNGQFERTLFTSGYSSKTNTYYMNTYDDPAIRSYAMADYDMDSSELISVAR

SEQ ID NO: 44 - DNA, 1014 bp, Bifidobacterium longum subsp. Infantis
atgaccaagaacttgttgttgggtattgctgctgtttgtggttctacttttcaagctgttgcttgtactggtgttagattctctgatgatgaaggtaacacttacttcggtagaaacttggattggtctttctcttacggtgaaaccattttggttactccaagaggttaccattacgatactgtttttggtgctggtggtaaagctaaacctaatgctgttataggtgttggtgttgttatggctgatagaccaatgtactttgattgcgctaatgaacatggtttggctattgctggtttgaattttccaggttacgcttcctttgttcacgaaccagttgaaggtactgaaaacgttgctacttttgaattcccattgtgggttgctagaaacttcgatagtgttgatgaagttgaagaagccttgagaaacgttaccttggtttctcaaatagtcccaggtcaacaagaatctttgttgcattggtttatcggtgacggtaagagatctatcgttgttgaacaaatggctgatggtatgcatgttcatcacgatgatgttgatgttttgactaaccagccaaccttcgattttcacatggaaaacttgaggaactacatgtgcgtttctaacgaaatggcagaacctacttcttggggtaaagcttctttgactgcttggggtgccggtgttggtatgcacggtattccaggtgatgtttcttcaccatctagattcgttagagttgcttacactaacgcccattactctcaacaaaatgatgaagctgctaacgtgtctaggttgtttcatactttgggtgctgttcaaatggtagacggtatggctaaaatgggtaatggtcaattcgaaagaaccttgttcacttccggttactcttctaagactaacacctactacatgaacacctatgatgatccagccattagatcttatgctatggcagattacgatatggactcctccgaattgatttctgttgctagatga

SEQ ID NO: 45 - protein, 337 aa, Bifidobacterium longum
MTKNLLLGIAAVCGSTFQAVACTGVRFSDDEGNTYFGRNLDWSFSYGETILVTPRGYHYDTVFGAGGKAKPNAVIGVGVVMADRPMYFDCANEHGLAIAGLNFPGYASFVHEPVEGTENVATFEFPLWVARNFDSVDEVEETLRNVTLVSQIVPGQQESLLHWFIGDGKRSIVVEQMADGMHVHHDDVDVLTNQPTFDFHMENLRNYMCVSNEMAEPTSWGKASLTAWGAGVGMHGIPGDVSSPSRFVRVAYTNAHYPQQNDEAANVSRLFHTLGSVQMVDGMAKMGDGQFERTLFTSGYSSKTNTYYMNTYDDPAIRSYAMADYDMDSSELISVAR

SEQ ID NO: 46 - DNA, 1014 bp, Bifidobacterium longum
atgaccaagaacttgttgttgggtattgctgctgtttgtggttctacttttcaagctgttgcttgtactggtgttagattctctgatgatgaaggtaacacttacttcggtagaaacttggattggtctttctcttacggtgaaaccattttggttactccaagaggttaccattacgatactgtttttggtgctggtggtaaagctaaacctaatgctgttataggtgttggtgttgttatggctgatagaccaatgtactttgattgcgctaatgaacatggtttggctattgctggtttgaattttccaggttacgcttcctttgttcacgaaccagttgaaggtactgaaaacgttgctacttttgaattcccattgtgggttgctagaaacttcgatagtgttgatgaagtcgaagaaaccttgagaaacgttaccttggtttctcaaatagtcccaggtcaacaagaatctttgttgcattggtttatcggtgacggtaagagatctatcgttgttgaacaaatggctgatggtatgcatgttcatcacgatgatgttgatgttttgactaaccagccaaccttcgattttcacatggaaaacttgaggaactacatgtgcgtttctaacgaaatggcagaacctacttcttggggtaaagcttctttgactgcttggggtgccggtgttggtatgcacggtattccaggtgatgtttcttcaccatctagattcgttagagttgcttacactaatgctcactacccacaacaaaatgatgaagctgctaatgtctctaggttgttccatactttgggttccgttcaaatggtagacggtatggctaagatgggtgatggtcaatttgaaagaaccttgttcacttccggttactcttctaagactaacacctactacatgaacacctatgatgatccagccattagatcttatgctatggcagattacgatatggactcctccgaattgatttctgttgctagatga

SEQ ID NO: 47 - protein, 349 aa, Clostridium perfringens
MTKNLLLGIAAVCGSTFQAVACTGLALETKDGLHLFGRNMDIEYSFNQSIIFIPRNFKCVNKSNKKELTTKYAVLGMGTIFDDYPTFADGMNEKGLGCAGLNFPVYVSYSKEDIEGKTNIPVYNFLLWVLANFSSVEEVKEALKNANIVDIPISENIPNTTLHWMISDITGKSIVVEQTKEKLNVFDNNIGVLTNSPTFDWHVANLNQYVGLRYNQVPEFKLGDQSLTALGQGTGLVGLPGDFTPASRFIRVAFLRDAMIKNDKDSIDLIEFFHILNNVAMVRGSTRTVEEKSDLTQYTSCMCLEKGIYYYNTYENNQINAIDMNKENLDGNEIKTYKYNKTLSINHVN

SEQ ID NO: 48 - DNA, 1050 bp, Clostridium perfringens
atgaccaagaacttgttgttgggtattgctgctgtttgtggttctacttttcaagctgttgcttgtactggtttggctttggaaacaaaggatggcttgcatttgtttggtagaaacatggacatcgagtactcattcaaccagtccattattttcatccccagaaacttcaagtgcgtcaacaagtctaacaagaaagaattgactaccaagtacgccgttttaggtatgggtactattttcgatgattacccaactttcgctgacggtatgaacgaaaaaggtttgggttgtgctggtttgaatttcccagtttacgtgtcctactccaaagaagatattgaaggcaagactaacatccccgtctacaattttttgttgtgggttttagccaacttcagctctgttgaagaagtcaaagaagctttgaagaacgccaacatcgttgatattccaatctccgaaaacattccaaacactaccttgcattggatgatctctgatatcactggtaagtccatcgttgttgaacagaccaaagaaaagttgaacgtgttcgataacaacatcggtgttttgactaactctccaacctttgattggcatgttgccaacttgaatcaatacgttggtttgagatacaaccaggtgccagaattcaaattgggtgatcaatctttgaccgctttaggtcaaggtacaggtttggttggtttgccaggtgattttactccagcttctagattcattagagtcgcttttttgagagatgccatgatcaagaacgataaggattccatcgatctgatcgaattcttccacatcttgaacaacgttgctatggttagaggttctaccagaaccgttgaagaaaagtctgatttgactcagtacacctcttgtatgtgcttggaaaaaggtatctactactacaacacctacgagaacaatcaaatcaacgccatcgacatgaacaaagaaaacttggatggtaacgagatcaagacctacaagtacaacaagaccttgtccatcaaccacgttaactga

SEQ ID NO: 49 - protein, 346 aa, Lactobacillus johnsonii
MTKNLLLGIAAVCGSTFQAVACTSIVYSSNNHHYFGRNLDLEISFGEHPVITPRNYEFQYRKLPSKKAKYAMVGMAIVENNYPLYFDAANEEGLGIAGLNFDGPCHYFPENAEKNNVTPFELIPYLLSQCTTVAEVKDALKDVSLVNINFSEKLPLSPLHWLMADKTGESIVVESTLSGLHVYDNPVHVLTNNPEFPGQLRNLANYSNIAPAQPKNTLVPGVDLNLYSRGLGTHFLPGGMDSASRFVKIAFVRAHSPQGNNELSSVTNYFHILHSVEQPKGTDEVGPNSYEYTIYSDGTNLETGTFYYTNYENNQINAIELNKENLNGDELTDYKLIEKQTINYQN

SEQ ID NO: 50 - DNA, 1041 bp, Lactobacillus johnsonii
atgaccaagaacttgttgttgggtattgctgctgtttgtggttctacttttcaagctgttgcttgtacctctatcgtctactcttctaacaaccatcattacttcggtaggaacttggacttggaaatttcttttggtgaacacccagttatcaccccaagaaattatgaattccagtacagaaagctgccctccaaaaaagctaaatatgctatggttggtatggccatcgttgaaaacaattaccccttgtattttgatgccgctaacgaagaaggtttaggtattgcaggtttgaatttcgatggtccatgtcactactttccagaaaatgctgaaaagaacaacgtcaccccattcgaattgattccatacttgttgtctcaatgtaccactgttgctgaagttaaggatgctttgaaggatgtttccttggtcaacatcaacttctctgaaaagttgccattgtctccattgcattggttgatggctgataagactggtgaatctatcgttgttgaatctactttgtctggcttgcacgtttacgataatccagttcatgttctgactaacaacccagaatttccaggtcaattgagaaacttggctaactactctaatattgctccagctcaacctaagaacactttggttccaggtgttgatttgaacttgtactctagaggtttgggtactcattttttgccaggtggtatggattctgcttctagatttgttaagattgccttcgttagagcccattctccacaaggtaacaatgaattgtcatccgttaccaactacttccacatcttgcattctgttgaacaacctaagggtactgatgaagttggtccaaattcttacgagtacaccatctattctgacggcactaatttggaaactggcactttttactacaccaactacgaaaacaaccagatcaacgccatcgagttgaacaaagaaaatttgaacggtgacgaactgaccgactacaagttgattgaaaagcaaaccatcaactaccagaactaa

SEQ ID NO: 51 - protein, 345 aa, Lactobacillus johnsonii
MTKNLLLGIAAVCGSTFQAVACTSILYSPKDNYFGRNLDYEIAYGQKVVITPRNYQLNYRHLPTQDTHYAMIGVSVVANDYPLYCDAINEKGLGIAGLNFTGPGKYFSVDESKKNVTSFELIPYLLSNCETIEDVKKLLSETNITDESFSKDLPVTTLHWLMGDKSGKSIVIESTETGLHVYDNPVNTLTNNPVFPAQVETLANFASVSPAQPKNTLVPNADINLYSRGLGTHHLPGGTDSNSRFIKASFVLAHSPKGNDEVENVTNFFHVLHSVEQAKGTDEVEDNVFEFTMYSDCMNLDKGILYFTTYDNNQINAVDMNNEDLGTSDLITYELFKDQAIKFEN

SEQ ID NO: 52 - DNA, 1038 bp, Lactobacillus johnsonii
atgaccaagaacttgttgttgggtattgctgctgtttgtggttctacttttcaagctgttgcttgtacctctatcttgtactctccaaaggataactacttcggtagaaacttggattacgaaattgcctacggtcaaaaggttgttattaccccaagaaactaccagttgaactacagacatttgccaactcaagatacccattacgctatgattggtgtttctgttgttgctaatgattaccccttgtactgtgatgccattaacgaaaaaggtttgggtatcgctggtttgaattttactggtccaggtaagtacttctccgttgatgaatctaagaagaacgtcacctccttcgaattgattccatacttgttgtctaactgcgaaaccatcgaagatgtcaagaagttgctgtccgaaactaacattaccgacgaatctttctcaaaggatttgccagttactaccttgcattggttgatgggtgataagtctggtaagtccatcgttattgaatctactgaaactggcttgcacgtttacgataatccagttaacactctgactaacaacccagtttttccagctcaagttgaaactttggctaacttcgcttctgtttcaccagctcaacctaagaatactttggttccaaacgccgatatcaacttgtattctagaggtttaggtactcatcatttgccaggtggtactgattctaactccagattcattaaggcctcattcgttttggctcattctccaaaaggtaacgatgaagttgaaaacgtgaccaactttttccatgtcttgcactctgttgaacaagctaaaggtactgacgaagtcgaagataacgttttcgaattcactatgtactccgactgtatgaacttggacaagggtatcttatacttcaccacctacgacaacaatcaaatcaacgctgttgacatgaacaacgaggatttgggtacttccgatttgattacttacgagttgttcaaggatcaggccatcaagttcgaaaactga

SEQ ID NO: 53 - protein, 336 aa, Lactobacillus johnsonii
MTKNLLLGIAAVCGSTFQAVACTGLRFTDDQGNLYFGRNLDVGQDYGEGVIITPRNYPLPYKFLDNTTTKKAVIGMGIVVDGYPSYFDCYNEDGLGIAGLNFPHFAKFSDGPIDGKINLASYEIMLWVTQNFTHVSEVKEALKNVNLVNEAINTSFAVAPLHWIISDSDEAIIVEVSKQYGMKVFDDKVGVLTNSPDFNWHLTNLGNYTGLNPHDATAQSWNGQKVAPWGVGTGSLGLPGDSIPADRFVKAAYLNVNYPTAKGEKANVAKFFNILKSVAMIKGSVVNDQGKDEYTVYTACYSSGSKTYYCNFEDDFELKTYKLDDHTMNSTSLVTY

SEQ ID NO: 54 - DNA, 1011 bp, Lactobacillus johnsonii
atgaccaagaacttgttgttgggtattgctgctgtttgtggttctacttttcaagctgttgcttgtactggtttgagattcactgatgatcaaggcaacttgtacttcggtagaaatttggatgttggtcaagattatggtgagggtgttattatcactccaagaaattatcccttgccatacaagtttttggataacactactaccaaaaaggccgttattggtatgggtatagttgttgatggttacccatcttacttcgactgttacaatgaagatggtttgggtatcgctggtttgaattttccacatttcgctaagttttccgatggtccaattgatggtaagattaacttggcctcctacgaaattatgttgtgggttactcaaaacttcacccacgtttctgaagtcaaagaagctttgaagaacgtcaacttggttaacgaagctatcaacacttcttttgctgttgctccattgcattggatcatctctgattctgatgaagccatcatcgttgaagtctctaaacaatacggtatgaaggttttcgatgataaggttggtgttttgactaactccccagattttaactggcatttgaccaatttgggtaactacacaggtttgaatccacatgatgctactgctcaatcttggaatggtcaaaaagttgctccttggggtgttggtactggttctttgggtttgccaggtgattctattccagctgatagatttgttaaggctgcctacttgaatgttaactacccaactgctaaaggtgaaaaggctaatgttgctaagttcttcaacatcttgaagtccgttgctatgattaagggttccgttgttaacgatcaaggtaaggatgagtacactgtctatactgcttgttactcttctggttctaagacctactactgcaacttcgaagatgacttcgaactgaaaacctacaagttggatgatcacaccatgaactctacatctttggttacctactaa

SEQ ID NO: 55 - protein, 363 aa, Bacteroides vulgatus
MAKTIFINDRIMKLKKGSIALLTLAGMFYMSVQKADACTRAVYIGPDHMVVTGRTMDWKEDIMSNIYVFPRGIQRAGYNKENAVKWTSKYGSVIATGYDIGTCDGMNEKGLVASLLFLPESVFDRPGDTRPVMGISIWTQYVLDNFATVHEAVEELKKESFRIDAPHMPNGSASTLHLAITDETGNTAVLEYLDGNLSIHEGKEFQVMTNSPRYDYQLAINNYWKEVGGLQMLPGTNRSSDRFVRASFYIHAIPQTPDAKIAVPSVLSVMRNVSVPFGITTPDKPHISSTRWRSVSDQKNKIYYFESVMTPNLFWLDLKKIDFSPKAGIKKLTLTNGKIYAGDAVKDLKDSDSFVFLFQTPVM

SEQ ID NO: 56 - DNA, 1092 bp, Bacteroides vulgatus
atggccaagaccattttcatcaacgacagaatcatgaagttgaagaagggttccattgctttgttgactttggctggtatgttctacatgtctgttcaaaaagctgatgcttgtaccagagctgtttacattggtccagatcatatggttgttactggtagaactatggattggaaagaggacatcatgtccaacatctacgttttcccaagaggtattcaaagagccggttacaacaaagaaaatgctgttaagtggacctccaagtacggttctgttattgctactggttacgatatcggtacttgtgatggtatgaacgaaaaaggtttggttgcctccttgttgtttttgccagaatctgtttttgatagaccaggtgatacaagaccagttatgggtatttctatttggacccaatacgtcttggataacttcgctactgttcatgaagctgtcgaggaattgaagaaagaatccttcagaattgatgccccacatatgccaaatggttctgcttctactttacatttggctattactgacgaaactggtaacactgctgttttggaatatttggacggtaacttgtccatccacgaaggtaaagaattccaagttatgaccaactctccaagatacgattaccaattggccattaacaactactggaaagaagttggcggtctacaaatgttgccaggtactaatagatcctccgatagatttgttagagcctccttttacattcacgccattccacaaactccagatgctaaaattgctgttccatccgttttgtccgttatgagaaatgtttctgttccattcggtattactaccccagataagccacatatttcttctactagatggcgttccgtttccgatcaaaaaaacaagatctactacttcgagtctgtcatgactccaaatttgttctggttggacttgaagaagatcgacttttcaccaaaggctggtatcaaaaagttgaccttgactaacggtaaaatctacgctggtgatgcagttaaggatttgaaagattccgactccttcgtgttcttgtttcaaactcctgttatgtaa

SEQ ID NO: 57 - protein, 355 aa, Eubacterium rectale
MTKNLLLGIAAVCGSTFQAVACTAATYKTKDFYFGRTLDYEFSYGDEIAVTPRNYVFDFRHAGKLENHYAIIGMAHVAGDYPLYYDAINEKGLGMAGLNFVGNAAYAAADENSSCENGICGIAKTKVAQFEFIPWILSLCATVADAKEKLNRILLVDTPFSSQLPVAQLHWIIADKNECIVVESMADGMHVYDNPVGVLTNNPPFTYQMAALNNYRGLSTKQPENTFAPGVELSAYSRGMGGLGLPGDLSSQSRFVRVAFTKQNSKSDDSENASVSQFFHILGSVDQQRGLCEVTEGKYEITLYTSCCNCDKGIYYYTTYDNSQITAVDMNRENLDEKLLIRYPVITEGKICWQN

SEQ ID NO: 58 - DNA, 1068 bp, Eubacterium rectale
atgaccaagaacttgttgttgggtattgctgctgtttgtggttctacttttcaagctgttgcttgtactgctgctacttacaaaactaaggatttctacttcggtaggaccttggattacgaattttcttacggtgacgaaattgctgttaccccaagaaattacgttttcgattttagacatgccggcaagttggaaaaccattacgctattattggtatggctcatgttgctggtgattatcccttgtattacgatgccattaacgagaaaggtttaggtatggctggtttgaattttgttggtaacgctgcttatgctgctgctgacgaaaattcttcttgtgaaaatggtatttgcggtatcgctaagactaaggttgctcagtttgaattcattccctggattttgtctttgtgtgctactgttgctgatgccaaagaaaagttgaacagaatcttgttggttgacaccccattctcttcacaattgccagttgctcaattgcattggattattgccgataagaacgaatgcatcgttgttgaatctatggctgatggtatgcacgtttacgataatccagttggtgttttgactaacaacccaccattcacttatcaaatggctgccttgaacaattacagaggtttgtctacaaagcagccagaaaacacttttgctccaggtgttgaattgtctgcttattctagaggtatgggtggtctaggtttgccaggtgatttgtcatctcaatccagatttgttagagttgccttcaccaagcaaaactccaaatctgatgattccgaaaacgcctctgtttcccaattctttcatatcttgggttccgtagatcaacaaaggggtttgtgtgaagttaccgaaggtaaatacgagattaccttgtacacctcttgttgcaactgtgataagggtatctactactacactacctacgacaactctcaaattaccgctgttgatatgaacagggaaaacttggacgaaaagctgttgattagatacccagtcattaccgagggtaagatttgttggcaaaactaa

SEQ ID NO: 59 - protein, 349 aa, Faecalibacterium prausnitzii
MTKNLLLGIAAVCGSTFQAVACTAATYKTKDFYMGRTLDYEFSYGEQITITPRNYEFDFRFAGKIKSHYALIGMAFVAEGYPLYYDAVNEKGLGMAGLNFVGNAAYEEALPEDETEVSQVAQFEFIPWTLTQCATVADAREKLAAMRLTGTAFSEQLPTAQLHWIIADKDSCIVVESMKDGLHVYDNPVGVLTNNPPFPSQMFALNNYAGVSRKQPESTFAGVLQLDAYSRGMGGMGIPGDLSSQSRFVKVAFTKLNSISGEEEDESVSQFFHILGSVDQQRGCCEVTEGKYEITIYTSCCNTAKGIYYYTTYDNHQITAVDMHAENLDSDQLICYPLLSKGEVRWQNK

SEQ ID NO: 60 - DNA, 1050 bp, Faecalibacterium prausnitzii
atgaccaagaacttgttgttgggtattgctgctgtttgtggttctacttttcaagctgttgcttgtactgctgctacttacaaaactaaggatttctacatgggtcgtaccttggattacgaattttcttacggtgaacagatcactatcaccccaagaaactacgaattcgattttagattcgccggcaagatcaagtctcattatgctttgattggtatggctttcgttgctgaaggttacccactatattatgatgccgttaacgaaaaaggtttaggtatggctggtttgaactttgttggtaacgctgcttatgaagaggctttgccagaagatgagactgaagtttctcaagttgcccagtttgaattcattccatggactttgactcaatgtgctactgttgctgatgctagagaaaaattggctgctatgagattgactggtactgctttttctgaacaattgccaactgctcaattgcattggattattgccgataaggattcctgcatcgttgtcgaatctatgaaggatggtttacacgtttacgataacccagttggtgttttgactaacaatccaccatttccatctcaaatgttcgccttgaacaattacgctggtgtttcaagaaaacagccagaatctacttttgctggtgtcttgcaattggatgcttattctagaggtatgggtggtatgggaattccaggtgatttgtcatctcaatccagattcgttaaggttgctttcaccaagttgaactccatttctggtgaagaggaagatgaatccgtttctcaattcttccatatcttgggttctgtcgatcaacaaagaggttgttgcgaagttactgaaggcaagtacgaaattactatctacacctcttgttgtaacaccgctaaaggtatctactactacactacttacgacaaccatcaaattaccgccgttgatatgcatgctgaaaacttggattctgaccaattgatttgctaccccttgttgtctaaaggtgaagttagatggcaaaacaagtaa

SEQ ID NO: 61 - protein, 336 aa, Bifidobacterium adolescentis
MTKNLLLGIAAVCGSTFQAVACTGVRFSDEEGNMYFGRNLDWSFSYGESILATPRGYHYDNVFGAERKATPNAVIGVGVVMADRPMYFDCANEHGLAIAGLNFPGYAEFVHEPVEGTDNVATFEFPLWVARNFDSVDEVEEALKNVTLVSQIVPGQQESLLHWIIGDSERSIVVEQMADGMHVHHDDVDVLTNQPTFGFHMENLRNYMCVGNEMAEPATWGKASLSAWGAGVSMHGIPGDVSSPSRFVRVAYANTHYPQQEGEAANVSRLFHTLGSVQMVDGMAKMSNGQFERTLFTSGYSSKTNTYYMNTYDDPAIRSYAMADFDMDSSELITAA

SEQ ID NO: 62 - DNA, 1011 bp, Bifidobacterium adolescentis
atgaccaagaacttgttgttgggtattgctgctgtttgtggttctacttttcaagctgttgcttgtactggtgttagattctctgatgaagagggtaatatgtacttcggtagaaacttggattggtccttttcttacggtgaatctattttggctactccaagaggttaccactacgataatgtttttggtgctgaaagaaaggctaccccaaatgctgttattggtgttggtgttgttatggctgatagaccaatgtactttgattgcgctaatgaacatggtttggctattgctggtttgaattttccaggttacgctgaatttgttcacgaaccagttgaaggtactgataacgttgctacttttgaattcccattgtgggttgctagaaacttcgatagtgttgatgaagttgaagaggccttgaagaacgttactttggtttctcaaatagtcccaggtcagcaagaatctttgttgcattggattatcggtgactccgaaagatctatcgttgttgaacaaatggctgatggtatgcatgttcatcacgatgatgttgatgttttgactaaccagccaactttcggtttccacatggaaaacttgagaaactacatgtgcgttggtaacgaaatggcagaaccagctacttggggtaaagcttctttgtctgcttggggtgctggtgtttctatgcatggtattccaggtgatgtttcttcaccatctagattcgttagagttgcttacgctaatactcactaccctcaacaagagggtgaagctgctaatgtttctaggttgtttcataccttgggttccgttcaaatggtagacggtatggctaaaatgtctaacggtcaattcgaaagaaccttgttcacttctggttactcttctaagactaacacctactacatgaacacctatgatgatccagccattagatcttatgctatggccgattttgatatggactcctctgaattgattaccgctgcttaa

SEQ ID NO: 63 - protein, 336 aa, Bifidobacterium dentium
MTKNLLLGIAAVCGSTFQAVACTGVRFSDAEGNMYFGRNLDWSFSYGETILVTPRAYKYDYVFGAEAKAEPNAVIGVGVVMADKPMYFDCANENGLAIAGLNFPGYASFAHEPIDGTINVATFEFPLWVARNFNTVDEVEEALKNVTLVSQIVPGQQESLLHWFIGDGTRSIVIEQMADGMHIHHDNVDVLTNQPTFDFHMENLRNYMCAGNEMAEPTTWGKAELTAWGAGVSMHGIPGDVSSPSRFVRVAYTNTHYPQQADEQSNVSRLFHTLGSVQMVDGCAKMANGKFERTLFTSGYSAKTKTYYMNTYDDPAIRSYAMADFDMDSSELITAE

SEQ ID NO: 64 - DNA, 1011 bp, Bifidobacterium dentium
atgaccaagaacttgttgttgggtattgctgctgtttgtggttctacttttcaagctgttgcttgtactggtgttagattctctgatgctgaaggtaatatgtacttcggtagaaacttggactggtctttttcttacggtgaaaccattttggttaccccaagagcttacaagtacgattatgtttttggtgctgaagctaaggctgaacctaatgctgttattggtgttggtgttgttatggctgataagccaatgtactttgattgcgctaacgaaaacggtttggctattgctggtttgaattttccaggttacgcttcttttgcccatgaacctattgatggtactattaacgttgccactttcgaatttccattgtgggttgctagaaacttcaacaccgttgatgaagttgaagaagccttgaagaacgttaccttggtttctcaaatagtcccaggtcaacaagaatctttgttgcattggttcattggtgacggtactagatccatcgttattgaacaaatggctgatggtatgcatatccaccatgataacgttgatgttttgactaaccagccaaccttcgattttcacatggaaaacttgagaaactacatgtgcgctggtaacgaaatggcagaacctactacatggggtaaagctgaattgactgcttggggtgctggtgtttctatgcatggtattccaggtgatgtttcttcaccatctagattcgttagagttgcttacactaacactcactatccacaacaagctgacgaacaatctaatgtctctaggttgtttcataccttgggttccgttcaaatggttgacggttgtgctaaaatggctaacggtaaatttgaaaggaccttgttcacttctggttactctgctaaaactaagacgtactacatgaacacctatgatgatccagccattagatcttatgctatggccgattttgatatggactcctctgaattgattaccgccgaatga

SEQ ID NO: 65 - protein, 351 aa, Bacteroides uniformis
MNKPIKTIAVALAAIASMSVQQTEACTRATYIGPDNTVITGRTMDWKEDLMSNIYVFPRGMQRAGYNKGNTVNWTSKYGSVIAAGYDIGTCDGMNEKGLVASLLFLPESVYHRPGDNRPIMGISIWTQYVLDNFATVREAVDELKKESFRIDAPDLPNGAASTLHLAITDETGNTAILEYIDGNLEIHEGKQYQVMTNSPKYELQLAINDYWKEVGGLNMLPGTNRSSDRFVRASFYINAIPQTADAKIAVPSVLSVMRNVSVPFGITTPDKPHISSTRWRSVSDQKNKVYYFESTLTPNLFWLDLKKIDFSPNAGIKKLSLTNGEIYAGDAIKDLKDSKGFVFLFQTPVM

SEQ ID NO: 66 - DNA, 1056 bp, Bacteroides uniformis
atgaacaagcccattaagaccattgctgttgctttggctgctattgcttctatgtctgttcaacaaactgaagcttgtactagagctacttacattggtccagataacactgttattaccggtagaactatggattggaaagaggacttgatgtccaacatctacgtttttccaagaggtatgcaaagagctggttacaacaaaggtaatactgttaactggacctccaagtacggttctgttattgctgctggttatgatatcggtacttgtgatggtatgaacgagaaaggtttggttgcctctttgttgtttttgccagaatccgtttatcacagaccaggtgataatagaccaatcatgggtatttctatttggacccaatacgtcttggataacttcgctactgttagagaagctgttgacgaattgaagaaagaatccttcagaattgatgccccagatttgccaaatggtgctgcttctactttacatttggctattactgacgaaactggtaacactgctattttggagtacatcgatggtaacttggaaatccatgaaggtaagcaataccaagtcatgaccaattctccaaagtacgaattacaattggccatcaacgactactggaaagaagttggaggtttgaatatgttgccaggcactaatagatcctccgatagatttgttagagcctccttttacattaacgccattccacaaactgctgatgctaaaattgctgttccatccgttttgtccgttatgagaaatgtttctgttccattcggtattactaccccagataagccacatatttcttctactagatggcgttccgtttccgatcaaaaaaacaaggtttactacttcgagtccactttgactccaaatttgttctggttggacttgaagaagatcgacttttctccaaacgctggtatcaaaaagttgtctttgactaacggtgaaatctacgctggtgatgctattaaggatttgaaagactctaagggcttcgtgttcttgtttcaaactccagttatgtaa

SEQ ID NO: 67 - protein, 341 aa, Collinsella aerofaciens
MTKNLLLGIAAVCGSTFQAVACTSIRFTDNSGHMYLGRNLDWSFDYGQQVRVMPRGFHIPYSFIDDASAAHAVIGMCIDYKNYPMFFDCGNDAGLAVAGLNFPGYAEYAEDPVDGKTNIAAYEFPLWVAATFSTVDEVEAALRDTVIVAKSAGEGLGVSLLHWIIGDNQRSIVVEMMADGLHVYDDPVDTLANQPTFPWHMENLRTYITATSDFPQTATWRGAELKPYGAGAGMRGIPGDCYSPSRFVKAAYLNANYPQKESETENVVRMFRSLEGVVMIEGASRMGDGKFEKTIYTGCFSARTGNYYYAMYGDPSIRYVSLMDAEGASPDRLVVPEPRRS

SEQ ID NO: 68 - DNA, 1026 bp, Collinsella aerofaciens
atgaccaagaacttgttgttgggtattgctgctgtttgtggttctacttttcaagctgttgcttgtacctctatcagattcactgataactctggtcatatgtacttgggtagaaacttggattggtctttcgattacggtcaacaagttagagttatgccaagaggtttccacattccatacagctttattgatgatgcttctgctgctcatgctgttattggtatgtgtatcgattacaagaactaccccatgttcttcgattgtggtaatgatgctggtttggctgttgcaggtttgaattttccaggttatgctgaatacgctgaagatccagttgatggtaagactaacattgctgcttacgaatttccattgtgggttgctgctactttctctactgttgatgaagttgaagctgctttgagagatactgttatcgttgctaaatctgctggtgaaggtttgggtgtttctttgttgcattggatcatcggtgacaatcaaagatccatcgttgttgaaatgatggctgatggtttacacgtttacgatgatcctgttgatactttggctaatcaacctacttttccttggcacatggaaaacttgagaacctacattactgctacctctgattttccacaaactgctacttggagaggtgctgaattgaaaccatacggtgctggtgctggtatgagaggtattccaggtgattgctattctccatctagatttgttaaggctgcttacttgaatgctaactacccacagaaagaatccgaaaccgaaaacgttgttagaatgttcagatccttggaaggtgttgttatgattgaaggtgcttctagaatgggtgacggtaaatttgaaaagactatctacaccggttgcttctctgctagaactggtaattactattacgccatgtatggtgacccatccattagatacgtttctttgatggatgctgaaggtgcatctccagatagattggttgttccagaacctagaagatcctga

SEQ ID NO: 69 - protein, 344 aa, Blautia obeum
MTKNLLLGIAAVCGSTFQAVACTAATYQTKDFYFGRTLDYEFSYGDQIAITPRNYPFSFRHAGEMATHYAIIGMAHVAGSYPLYYDAINEKGVGMAGLNFVGNAAYADVSPDRDNVAQFEFIPWILSQCATLSEVRTLLERMALVATPFNEQFPLAQLHWIISDKTGSITVESMADGLHIYENPVGVLTNNPPFPQQMFQLNNFMHLSPKQPVNTFSDDLALTAYSRGMGALGLPGDLSSASRFVRVAFTKMNAFSGDSEKESVSQFFHILGSVDQQRGCCEVADGKYEITLYTSCCNVTKGIYYYTTYENHQISAVDMHRENLDGGALICYPVIQGEQINYQN

SEQ ID NO: 70 - DNA, 1035 bp, Blautia obeum
atgaccaagaacttgttgttgggtattgctgctgtttgtggttctacttttcaagctgttgcttgtactgctgctacttaccaaactaaggatttctacttcggtagaaccttggactacgaattttcttacggtgatcaaattgctatcaccccaagaaattacccattctcttttagacatgctggtgaaatggctacccattacgctattattggtatggctcatgttgctggttcttatcccttgtattacgatgccattaacgaaaaaggtgtaggtatggctggtttgaattttgttggtaatgctgcatacgctgatgtttctccagatagagataatgttgctcagttcgaattcatcccatggattttgtctcaatgtgctaccttgtctgaagtcagaactttgttggaaagaatggctttggttgctactccattcaatgaacaatttccattggctcaattgcactggatcatttctgataagactggttccattaccgttgaatctatggctgatggtttacacatctacgaaaatccagttggtgtcttgactaacaatccaccatttccacaacaaatgttccagctgaacaacttcatgcatttgtctccaaagcaaccagttaacaccttctctgatgatttggctttgactgcttattctagaggtatgggtgctttgggtttgccaggtgatttgtcatctgcttctagatttgttagagttgccttcactaagatgaacgctttttctggtgactccgaaaaagaatccgtttctcaattcttccacatcttgggttctgttgaccaacaaagaggttgttgcgaagttgctgatggtaaatacgaaattaccttgtacacctcttgttgcaacgttactaagggtatctactactacactacctacgaaaaccatcaaatctccgctgttgatatgcacagagaaaatttggatggtggtgctttgatttgctacccagttattcaaggtgaacagatcaactaccagaactaa

SEQ ID NO: 71 - protein, 345 aa, Blautia obeum
MTKNLLLGIAAVCGSTFQAVACTAATYKSKDFYFGRTLDYEFSYGDQIVITPRNYSFHFRYIGDKKKHYAMIGMAHVAENYPLYYDAMNEKGVAIAGLNFVGNAVYSEVQSDVENIAQFEFIPWILSQCASLAEVRELLDRINIVNTPFSEQLPLAQLHWIISDENESITVESMSHGLHIYDNPVGVLTNNPPFPQQMFQLNNYMHLSPKQPENTFSENLALDAYSRGMGGIGLPGDLSSSSRFVRVAFTKIHAISGESEKESVSQFFHILGSVDQQRGCCEVADGKYEITLYTSCCNVTKGIYYYNTYENHQISAVDMYAEDLDGNNLICYPVIQGEQINYQNK

SEQ ID NO: 72 - DNA, 1038 bp, Blautia obeum
atgaccaagaacttgttgttgggtattgctgctgtttgtggttctacttttcaagctgttgcttgtactgctgctacttacaaatctaaggatttctacttcggtaggaccttggattacgaattttcttacggtgatcaaatcgtcatcaccccaagaaactattccttccatttcagatatatcggcgacaagaaaaagcactacgctatgattggtatggctcatgttgctgaaaattaccccttgtattacgatgccatgaacgaaaaaggtgttgctattgctggtttgaactttgttggtaacgctgtttactccgaagttcaatctgatgttgaaaacattgcccagttcgaattcattccatggattttgtctcaatgcgcttctttggctgaagttagagaattattggacaggatcaacatcgtgaacactccattttctgaacaattgccattggctcaattgcactggattatttctgacgaaaacgaatccatcaccgtcgaatctatgtctcatggtttacacatctacgataacccagttggtgttttgactaacaatccaccatttccacaacagatgttccagttgaacaactacatgcatttgtctccaaagcaaccagaaaacaccttctctgaaaatttggctttggatgcttactctagaggtatgggtggtattggtttgccaggtgatttgtcatcttcttcaagattcgttagagttgccttcaccaagattcatgctatttctggtgaatccgaaaaagagtccgtttctcaattcttccatatcttgggttctgtcgatcaacaaagaggttgttgcgaagttgctgatggtaaatacgaaattaccttgtacacctcttgttgcaacgttactaagggtatctactactacaacacctacgaaaaccatcaaatctccgctgttgatatgtacgctgaagatttggatggtaacaacttgatttgctacccagttattcaaggtgagcagatcaattaccagaacaagtga

SEQ ID NO: 73 - protein, 349 aa, Parabacteroides distasonis
MKSKNLTLASLFAGLFMFMNPMGSEACTRAVYLGPDDMVVTGRTMDWKEDPQSNLYLFPQGVQRRGAISDNTIQWTSKYGSVVTAGYDIGTCDGMNEKGLVANLLFLTESSYFRPDDNRPVMGLSIWTQYVLDNFATVDEAVAELSKEVFRIDDPDLPNGAKSTLHLSISDASGNSAIFEYLNGNLVIHEGRECQVMTNSPTYDKQLTLNDYWQQIGGLVMLPGTNRASDRFVRASFYIHAIPQTSNFREAVAGVFSVMRNVSVPLGITTPDQPNISSTRWRTVADQKNKVYYFESTLSPDIFWVDFKSLDFKAGTPIKKLTLTNGEIYAGNTAKDFKDSKPLPFLFGI

SEQ ID NO: 74 - DNA, 1050 bp, Parabacteroides distasonis
atgaagtccaagaacttgaccttggcttctttgtttgctggtttgttcatgtttatgaacccaatgggtagtgaagcttgtactagagctgtttatttgggtccagatgatatggttgttactggtagaactatggattggaaagaagatccacagtccaacttgtacttgtttccacaaggtgttcaaagaagaggtgccatttctgataacactattcaatggacttccaagtacggttctgttgttacagctggttatgatatcggtacttgtgatggtatgaacgagaaaggtttggttgctaacttgttgttcttgaccgaatcctcttacttcagaccagatgacaatagaccagttatgggtttgtctatttggacccaatacgttttggataacttcgctactgttgatgaagctgttgccgaattgtctaaagaagttttcagaatcgacgatccagatttgccaaatggtgctaaatctaccttgcacttgtcaatttctgatgcctctggtaactctgccattttcgaatacttgaacggtaacttggttatccacgaaggtagagaatgtcaagttatgactaactctccaacctacgataagcaattgaccttgaatgattactggcaacaaatcggtggtttggttatgttgccaggtactaatagagcttccgatagatttgttagagcctccttttacattcacgctattccacaaacttccaacttcagagaagcagttgctggtgttttttccgttatgagaaatgtttctgtgccattgggtattactactccagatcaacctaacatttcttctactagatggcgtactgttgctgaccaaaaaaacaaggtttactacttcgagtctaccttgtctccagatattttctgggttgacttcaagagcttggattttaaagctggtactcccatcaaaaagctgactttgactaatggtgaaatctacgctggtaacactgctaaggatttcaaagattctaagcccttgccattcttgttcggtatttga

SEQ ID NO: 75 - protein, 344 aa, Parabacteroides distasonis
MTKNLLLGIAAVCGSTFQAVACTRAVYLGPDDMVVTGRTMDWKEDPQSNLYLFPRGVQRRGAISDNTIQWTSKYGSVVTAGYDIGTCDGMNEKGLVANLLFLTESSYFRSDDNRPVMGLSIWTQYVLDNFATVDDAVAELSKEVFRIDDPDLPNGAKSTLHLSISDASGNSAIFEYLNGNLVIHEGRECQVMTNSPTYDKQLTLNDYWQQIGGLVMLPGTNRASDRFVRASFYIHAIPQTSNFREAVAGVFSVMRNVSVPLGITTPDQPNISSTRWRTVADQKNKVYYFESTLSPDIFWVDFKSLDFKAGTPIKKLTLTNGEIYAGNTTKDFKDSKPLPFLFGI

SEQ ID NO: 76 - DNA, 1035 bp, Parabacteroides distasonis
atgaccaagaacttgttgttgggtattgctgctgtttgtggttctacttttcaagctgttgcttgtactagagctgtttatttgggtccagatgatatggttgttactggtagaactatggattggaaagaagatccacagtccaacttgtacttgtttccaagaggtgttcaaagaagaggtgccatttctgataacactattcaatggacttccaagtacggttctgttgttacagctggttatgatatcggtacttgtgatggtatgaacgagaaaggtttggttgccaatttgttgttcttgaccgaatcctcttacttcaggtctgatgataatagaccagtcatgggtttgtctatttggacccaatacgttttggataacttcgctactgttgatgatgctgttgccgaattgtctaaagaagttttcagaatcgacgatccagatttgccaaatggtgctaaatctaccttgcacttgtcaatttctgatgcctctggtaactctgccattttcgaatacttgaacggtaacttggttatccacgaaggtagagaatgtcaagttatgactaactctccaacctacgataagcaattgaccttgaatgattactggcaacaaatcggtggtttggttatgttgccaggtactaatagagcttccgatagatttgttagagcctccttttacattcacgctattccacaaacttccaacttcagagaagcagttgctggtgttttttccgttatgagaaatgtttctgtcccattgggtattactactccagatcaacctaacatttcttctactagatggcgtactgttgctgaccaaaaaaacaaggtttactacttcgagtctaccttgtctccagatattttctgggttgacttcaagtccttggattttaaagctggtactcccatcaaaaagctgactttgactaatggtgaaatctacgctggtaacactaccaaggatttcaaagattctaagcccttgccattcttgttcggtatttga

SEQ ID NO: 77 - protein, 346 aa, Lactobacillus mucosae
MTKNLLLGIAAVCGSTFQAVACTAATYTAGDHYFGRNLDLELSYGEKVTITPRNFPLTFRKMPTLEHHYALIGMATTVDNYPLYFDATNEKGLSMAGLNYPGNADYKPYAEGKDNVSPFEFVSWILGQCATLAEAKVLLDKINLVKIDFSEQLPLSPLHWLMAETASEKTLTIECDRDGLHVYDNPVGVLTNNPQFDKQLFNLNNYQFLSPQQPENKFSQDLELDNYSRGLGTRGLPGGMDSMSRFVKVAFTKLNAPKGETENENVGNFFHILHSVEQQKNLDGVENNQFEFTIYSSCVNADRGIYYYTTYDNNQINAVDMHKVDLDGRELIDYDLANEQNINWQN

SEQ ID NO: 78 - DNA, 1041 bp, Lactobacillus mucosae
atgaccaagaacttgttgttgggtattgctgctgtttgtggttctacttttcaagctgttgcttgtactgctgctacttatactgctggtgatcattactttggtaggaacttggatttggagttgtcttacggtgaaaaggttactattaccccaagaaacttcccattgacctttagaaaaatgccaaccttggaacatcattacgccttgattggtatggctactactgttgataactaccccttgtactttgatgctaccaacgaaaaaggtttgtctatggctggtttgaattacccaggtaatgctgattacaaaccatacgctgaaggtaaggataacgtttctccattcgaattcgtttcttggattttgggtcaatgtgctactttggctgaagctaaagttttgttggacaagatcaacttggtcaagatcgacttctctgaacaattgccattgtctccattgcattggttgatggctgaaactgcttctgaaaagactttgaccattgaatgtgatagagatggcttgcacgtttacgataatccagttggtgttttgactaacaacccacaatttgacaagcagctgttcaacctgaacaactaccaatttttgtctccacaacagccagagaacaagttttctcaagatttggaactggacaactactctagaggtttgggtacaagaggtttgccaggtggtatggattctatgtctagatttgttaaggttgccttcactaagttgaatgctccaaaaggtgaaaccgaaaacgaaaacgttggtaacttcttccatatcttgcactctgtcgaacagcaaaagaatttggatggtgttgaaaacaaccagttcgagttcactatctactcctcttgtgttaatgcagacagaggtatctactactacactacctatgataacaaccagattaacgccgttgatatgcacaaggttgatttggacggtagagaattgatcgattacgatttggctaacgagcagaacattaactggcaaaactga

SEQ ID NO: 79 - protein, 359 aa, Bacteroides ovatus
MMKMTKNLLLGIAAVCGSTFQAVACTGISLTSRDGSYVQARTIEWARGVLQSEYVIIPRGQQLTSFTPTGVNGLTFTAKYGVVGLAVVQKEFIAEGINEAGLSAGLFFFPHYGGYKTYDAAQNQRTLADLQVTEWLLSQFSTIDEVKAALSSVRVVGLEKTAVVHWRIGEPSGRQVVLEIVGGVPHFYENEVGVLTNAPGFEWQLTNLNNYVNLHPGDASVQKLSGITLQPTGGNSGFLGIPGDATPPSRFVRAAFYRGTAPQRATGFDTVQQCFHLLNNFDIPIGIEHSQGDIPDIPSATQWTSAIDLTNRKVYYKTAYNNSIRCIDLKAIDFSKVKYQSQPLDKIQEQPVEMVKIPR

SEQ ID NO: 80 - DNA, 1080 bp, Bacteroides ovatus
atgatgaagatgaccaagaacctgttgttgggtattgctgctgtttgtggttctacttttcaagctgttgcttgtaccggtatttctttgacttctagagatggttcttacgttcaagccagaactattgaatgggctagaggtgttttacagtccgaatacgttattatcccaagaggtcaacaattgacttctttcactccaactggtgttaacggtttgacttttactgctaagtatggtgttgttggtttggccgttgttcagaaagaattcattgctgaaggtattaacgaagctggtttgtctgctggtttattcttttttccacattacggtggttacaagacttacgatgctgctcaaaatcaaagaactttggccgacttacaagttaccgaatggttgttgtctcaattctccactatcgatgaagttaaggctgctttgtcatccgttagagttgtaggtttggaaaagactgctgttgttcattggagaataggtgaaccatctggtagacaagttgttttggaaatagttggtggtgttccacacttctacgaaaatgaagttggtgttttgactaacgctccaggttttgaatggcaattgactaacttgaacaactacgttaacttgcatccaggtgatgcctctgttcaaaaattgtctggtattaccttgcaacctactggtggtaattctggttttttgggtattcctggtgatgctactccaccatctagatttgttagagctgctttttacagaggtactgctccacaaagagctactggttttgatactgttcaacaatgcttccacctgttgaacaatttcgatattccaatcggtatcgaacactcccaaggtgatattccagatattccttctgctactcaatggacctctgctattgatttgaccaacagaaaggtttactacaagaccgcttacaacaactccattagatgcatcgatttgaaggccatcgatttctctaaggtcaagtatcaatctcagccattggacaagattcaagaacaaccagttgaaatggtcaagatcccaagatga

SEQ ID NO: 81 - protein, 361 aa, Bacteroides uniformis
MAKRIPILLAAMTALTFAPQTAEACTGITLTAKDNAHVVARTIEWGGSELNSQYVIVPRGYSQQSLTPGGSLSGMEFTSRYGYVGLAVEQKEFVAEGLNEAGLSAGLFYFPGYGKYEEYDPTRKASSIADLQLVSWLLGSFSTVDEVKEAIENVTVIAIDPRASTVHWRIADKTGRQLVLEFIDGQPRFYENKLGVLTNSPGFDWQMTNLNNYVNLYPGSAESKSLGNVTIAPFGSGSGFLGIPGDVTPPSRFIRAAFYQTSTPPQNTAQETVLQCFQILNNFDIPVGIEFPLGQVPANIPSATQWTSATDMTNGIIYYRTMYNSTIRSIDLRQIDFGRIEYHAAPLDKVKRQPIAPVKIE

SEQ ID NO: 82 - DNA, 1086 bp, Bacteroides uniformis
atggccaagagaattccaattttgttggctgctatgactgctttgacttttgctccacaaactgctgaagcttgtactggtattactttgactgctaaggataacgctcatgttgttgctagaactattgaatggggtggttctgaattgaactcccaatatgttatagtcccaagaggttactcccaacaatctttgactccaggtggttctttgtctggtatggaattcacttctagatacggttatgttggtttggccgtcgaacaaaaagaatttgttgctgaaggtttgaacgaagctggtttgtctgctggtttattctattttccaggttacggcaagtacgaagaatacgatccaactagaaaggcttcttctattgctgacttgcaattggtttcttggttgttgggttctttctctactgttgacgaagtcaaagaagccattgaaaacgttaccgttattgccattgatccaagagcttctactgttcattggagaattgctgataagaccggtagacaattggtcttggaattcattgatggtcagccaagattctacgaaaacaagttgggtgttttgactaactctccaggttttgattggcaaatgaccaacttgaacaactacgttaacttgtatccaggttccgctgaatctaagtctttgggtaatgttactattgccccatttggttctggttcaggttttttgggtattccaggtgatgttactccaccatctagattcattagagctgctttctaccaaacttctacaccaccacaaaacactgctcaagaaaccgttttacaatgcttccagatcctgaacaacttcgatattccagttggtatcgaattcccattgggtcaagttccagctaatattccatctgctactcaatggacttctgctactgatatgactaacggtatcatctactacaggaccatgtacaactctaccatcagatccattgacttgagacaaattgacttcggtagaatcgaatatcatgctgctccattggataaggttaagagacaacctattgctccagtcaagatcgaatga

SEQ ID NO: 83 - protein, 349 aa, Eubacterium ventriosum
MTKNLLLGIAAVCGSTFQAVACTAATYKTKDFYMGRTLDYEFSYGEQITITPRNYEFDFRFAGKIKSHYALIGMAFVAGGYPLYYDAVNEKGLGMAGLNFVGNAAYEEALPEDETEVSQVAQFEFIPWILTQCATVAEAREKLAAMRLTGTAFSEQLPTAQLHWIIADKDACIVVESMKDGLHVYDNPVGVLTNNPPFLGQMFALNNYAGVSRKQPESTFAGVLQLDAYSRGMGGMGIPGDLSSQSRFVKVAFTKLNSISGEEEGESVSQFFHILGSVDQQRGCCEVTEGKYEITIYTSCCNTAKGIYYYTTYDNHQITAVDMHAENLDSDQLICYPLLSKGEVRWQNK

SEQ ID NO: 84 - DNA, 1050 bp, Eubacterium ventriosum
atgaccaagaacttgttgttgggtattgctgctgtttgtggttctacttttcaagctgttgcttgtactgctgctacttacaaaactaaggatttctacatgggtcgtaccttggattacgaattttcttacggtgaacagatcactatcaccccaagaaactacgaattcgattttagattcgccggcaagatcaagtctcattatgctttgattggtatggcttttgttgctggtggttacccactatattatgatgccgttaacgaaaaaggtttaggtatggctggtttgaactttgttggtaacgctgcttatgaagaggctttgccagaagatgagactgaagtttctcaagttgcccagtttgaattcattccatggattttgactcaatgcgctactgttgctgaagctagagaaaaattggctgctatgagattgactggtactgctttttctgaacaattgccaactgctcaattgcattggattattgctgataaggatgcctgcatcgttgtcgaatctatgaaggatggtttacacgtttacgataacccagttggtgttttgactaacaatccaccatttttgggtcaaatgttcgccttgaacaattacgctggtgtttcaagaaaacagccagaatctacttttgctggtgtcttgcaattggatgcttattctagaggtatgggtggtatgggaattccaggtgatttgtcatctcaatccagattcgttaaggttgctttcaccaagttgaactccatttctggtgaagaggaaggtgaatctgtttcccaattcttccatatcttgggttctgtagatcaacaaagaggttgttgcgaagttaccgaaggtaaatacgagattactatctacacctcttgttgtaacactgccaaaggtatctactactacactacttacgacaaccatcaaattaccgccgttgatatgcatgctgaaaacttggattctgaccaattgatttgctaccccttgttgtctaaaggtgaagttagatggcaaaacaagtaa

SEQ ID NO: 85 - protein, 345 aa, Blautia obeum
MTKNLLLGIAAVCGSTFQAVACTAATYKTKDFYFGRTLDYEFSYGDQIVITPRNYAFNFRHVGDMKNHYAIIGMAHVAEDYPLYYDAMNEKGVAMAGLNFVGNAVYAAIKPDVENIAQFEFIPWILSQCSSLVEVRELLERINIVNTPFSEQLPLAQLHWIISDENESITVESMSDGLHIYDNPVGVLTNNPPFPQQMFQLNNYMYLSPKQPRNTFCENLALDAYSRGMGGLGLPGDLSSSSRFVRVAFTKVNAISGESEEESVSQFFHILGSVDQQRGCCEVADGKYEITLYTSCCNVTKGIYYYNTYENHQISAVDMHVENLDSDKMICYPVIQGERINYQNK

SEQ ID NO: 86 - DNA, 1038 bp, Blautia obeum
atgaccaagaacttgttgttgggtattgctgctgtttgtggttctacttttcaagctgttgcttgtactgctgctacttacaaaactaaggatttctacttcggtaggaccttggattacgaattttcttacggtgatcaaatcgtcatcaccccaagaaattacgctttcaactttagacacgttggcgatatgaagaaccattacgctattattggtatggctcatgttgctgaagattaccccttgtattatgatgccatgaacgaaaaaggtgttgctatggctggtttgaattttgttggtaatgctgtttacgctgccatcaaaccagatgttgaaaacattgcccagttcgaattcattccatggatcttgtctcaatgctcctcattggttgaagttagagaactgttggaaaggatcaacatcgttaacactccattctctgaacaattgccattggctcaattgcattggattatctctgacgaaaacgaatccatcaccgtcgaatctatgtctgatggtttacacatctacgataacccagttggtgttttgactaacaatccaccatttccacaacagatgttccagttgaacaactacatgtacttatctccaaagcagcccagaaacactttctgtgaaaatttggctttggacgcttactctagaggtatgggtggtttgggtttgccaggtgatttgtcatcttcttcaagattcgttagagttgccttcactaaggttaacgctatttctggtgaatccgaagaagaatccgtttctcaattcttccatatcttgggttctgttgaccaacaaagaggttgttgcgaagttgctgatggtaaatacgaaattaccttgtacacctcttgttgcaacgttactaagggtatctactactacaacacctacgaaaaccatcaaatctccgctgttgatatgcacgttgaaaacttggattccgataagatgatttgctacccagttattcagggtgagagaatcaattaccagaacaagtga

SEQ ID NO: 87 - protein, 328 aa, Collinsella aerofaciens
MTKNLLLGIAAVCGSTFQAVAYLGRNLDWSFDYGQQVRVMPRGFHIPYSFIDDATAAHAVIGMCIDYRNYPMFFDCGNDAGLAVAGLNFPGYAEYAEGPVDGKTNIAAYEFPLWVAATFSTVDEVEAALRDTVIVAKSAGEGLGVSLLHWIIGDSQRSIVVEVMADGLHVYDDPVDTLANQPTFPWHMENLRTYITATSDFPQTATWRGAELKPYGAGAGMRGIPGDCYSPSRFVKAAYLNANYPQKESETENVVRMFRSLEGVVMIEGASRMGDGKFEKTIYTGCFSARTGNYYYAMYGDPSIRYVSLMDAEGASPDRLVVPEPRRS

SEQ ID NO: 88 - DNA, 987 bp, Collinsella aerofaciens
atgaccaagaacttgttgttgggtattgctgctgtttgtggttctacttttcaagctgttgcttacttgggtagaaacttggattggtcttttgactacggtcaacaagttagagttatgccaagaggtttccacattccatactcattcattgatgatgctactgctgctcatgctgttattggtatgtgtatcgattacagaaactaccccatgttcttcgattgtggtaatgatgctggtttggctgttgcaggtttgaattttccaggttatgctgaatacgctgaaggtccagttgatggtaaaactaacattgctgcttacgaattcccattgtgggttgctgctactttttctactgttgatgaagttgaagctgccttgagagatactgttatcgttgctaaatctgctggtgaaggtttgggtgtttctttgttgcattggattatcggtgactcccaaagatctatcgttgttgaagttatggccgatggtttacacgtttatgatgatcctgttgatactttggctaaccagccaacttttccatggcatatggaaaacttgaggacctacattactgctacctctgattttccacaaactgctacttggagaggtgctgaattgaaaccatacggtgctggtgctggtatgagaggtattccaggtgattgctattctccatctagatttgttaaggctgcctacttgaatgctaactacccacaaaaagaatccgaaaccgaaaacgttgtcagaatgttcagatcattggaaggtgttgttatgattgaaggtgcttctagaatgggtgacggtaaatttgaaaagactatctacaccggttgcttctctgctagaactggtaattactattacgccatgtatggtgacccatccattagatacgtttctttgatggatgctgaaggtgcatctccagatagattggttgttccagaacctagaagatcctga

SEQ ID NO: 89 - protein, 367 aa, Bifidobacterium adolescentis
MTKNLLLGIAAVCGSTFQAVAFIRRLKPWLVQPSMRMMNAPGQSNKGRKSIMCTGVRFSDEEGNMYFGRNLDWSFSYGESILATPRGYHYDNVFGASGKATPNAVIGVGVVMADRPMYFDCANEHGLAIAGLNFPGYAEFVHEPVEGTDNVATFEFPLWVARNFDSVDEVEEALKNVTLVSQIVPGQQESLLHWIIGDSERSIVVEQMADGMHVHHDDVDVLTNQPTFGFHMENLRNYMCVGNEMAEPATWGKASLSAWGAGVSMHGIPGDVSSPSRFVRVAYANTHYPQQEGEAANVSRLFHTLGSVQMVDGMAKMGNGQFERTLFTSGYSSKTNTYYMNTYDDPAIRSYAMADFDMDSSELITAA

SEQ ID NO: 90 - DNA, 1104 bp, Bifidobacterium adolescentis
atgaccaagaacttgttgttgggtattgctgctgtttgtggttctacttttcaagctgttgccttcattagaagattgaagccatggttggttcagccatctatgagaatgatgaatgctccaggtcaatctaacaagggtagaaagtctattatgtgcaccggtgttagattctctgatgaagagggtaatatgtacttcggtagaaacttggattggtccttttcttacggtgaatctattttggctactccaagaggttaccactacgataatgtttttggtgcttctggtaaagctaccccaaatgctgttattggtgttggtgttgttatggctgatagaccaatgtactttgattgcgctaatgaacatggtttggctattgctggtttgaattttccaggttacgctgaatttgttcacgaaccagttgaaggtactgataacgttgctacttttgaattcccattgtgggttgctagaaacttcgatagtgttgatgaagttgaagaggccttgaagaacgttactttggtttctcaaattgtcccaggtcagcaagaatctttgttgcattggattatcggtgactccgaaagatctatcgttgttgaacaaatggctgatggtatgcatgttcatcacgatgatgttgatgttttgactaaccagccaactttcggtttccacatggaaaacttgagaaactacatgtgcgttggtaacgaaatggcagaaccagctacttggggtaaagcttctttgtctgcttggggtgctggtgtttctatgcatggtattccaggtgatgtttcttcaccatctagattcgttagagttgcttacgctaatactcactaccctcaacaagagggtgaagctgctaatgtttctaggttgtttcataccttgggttccgttcaaatggtagacggtatggctaaaatgggtaatggtcaattcgaaagaaccttgttcacttccggttactcttctaagactaacacctactacatgaacacctatgatgatccagccattagatcttatgctatggccgattttgatatggactcctctgaattgattaccgctgcttaa

SEQ ID NO: 91 - protein, 352 aa, Bacteroides thetaiotaomicron
MKKKLTGVALVLAAVSLMGIQPAGACTRAVYLGPDRMVVTGRTMDWKEDIMSNIYVFPRGMQRAGHNKEKTVNWTSKYGSVIATGYDIGTCDGMNEKGLVASLLFLPESVYSLPGDTRPAMGISIWTQYVLDNFATVREAVDEMKKETFRIDAPRMPNGGPESTLHMAITDETGNTAVIEYLDGKLSIHEGKEYQVMTNSPRYELQLAVNDYWKEVGGLQMLPGTNRSSDRFVRASFYIHAIPQTADAKIAVPSVLSVMRNVSVPFGINTPEKPHISSTRWRSVSDQKNKVYYFESTLTPNLFWLDLKKIDFSPKAGVKKLSLTKGEIYAGDAVKDLKDSQSFTFLFETPVM

SEQ ID NO: 92 - DNA, 1059 bp, Bacteroides thetaiotaomicron
atgaagaagaagttgactggcgttgctttggttttggctgctgtttctttgatgggtattcaacctgctggtgcttgtactagagctgtttatttgggtccagatagaatggttgttactggtagaactatggattggaaagaggacatcatgtccaacatctacgtttttccaagaggtatgcaaagagccggtcataacaaagaaaagactgttaactggacctccaagtacggttctgttattgctactggttacgatatcggtacttgtgatggtatgaacgaaaaaggtttggttgcctctttgttgttcttgccagaatctgtttattctttgccaggtgatacaagaccagctatgggtatttctatttggactcaatacgtcttggataacttcgctactgttagagaagctgttgacgaaatgaagaaagaaaccttcagaattgatgccccaagaatgccaaatggtggtccagaatctacattgcatatggctattactgacgaaactggtaacaccgctgttatcgaatatttggacggtaaattgtccatccacgagggtaaagaataccaagttatgactaactccccaaggtatgaattacaattggccgttaacgactactggaaagaagttggaggtttacaaatgttgccaggcactaatagatcctccgatagatttgttagagcctccttttacattcacgctattccacaaactgctgatgctaaaattgctgttccatccgttttgtccgttatgagaaatgtttctgttccattcggtatcaacactccagaaaagccacatatttcttctactagatggcgttccgtttccgatcaaaaaaacaaggtttactacttcgagtccactttgactccaaatttgttctggttggacttgaagaagatcgacttttcaccaaaagctggtgtcaagaaattgtctttgactaagggtgaaatctacgctggtgatgctgttaaggatttgaaggattctcaatccttcaccttcttgttcgaaactccagttatgtaa

SEQ ID NO: 93 - protein, 360 aa, Bacteroides caecimuris
MMKMTKNLLLGIAAVCGSTFQAVACTGISLTSRDGSYVQARTIEWARGVLQSEYVIIPRGQQLTSFTPTGVNGLTFTAKYGVVGLTVVQKEFIAEGINEAGLSAGLFFFPHYGGYETYDAAQNRRTLADLQVTEWMLSQFSTIDEVKAALSSVRVVGLEKTAVVHWRIGEPSGRQVVLEIVGGVPHFYENGVGVLTNAPGFEWQLTNLNNYVNLYPGDASVRKLSGITLQPTGSNSGFLGIPGDATPPSRFVRAAFYRGTAPQRATGFDTVQQCFHLLNNFDIPIGIEYSQVGDIPDIPSATQWTSAIDLTNRKVYYKTAYNNSIRCIDLKAIDFSKVKYQSQPLDKIQEQPIEMVKIPR

SEQ ID NO: 94 - DNA, 1083 bp, Bacteroides caecimuris
atgatgaagatgaccaagaacctgttgttgggtattgctgctgtttgtggttctacttttcaagctgttgcttgtaccggtatttctttgacttctagagatggttcttacgttcaagccagaactattgaatgggctagaggtgttttacagtccgaatacgttattatcccaagaggtcaacaattgacttctttcactccaactggtgttaacggtttgacttttactgctaagtatggtgttgttggtttgaccgttgtccagaaagaattcattgctgaaggtattaacgaagctggtttgtctgctggtttattcttttttccacattacggtggttacgaaacttatgatgctgctcaaaacagaagaactttggccgacttacaagttactgaatggatgttgtctcagttctccactatcgatgaagttaaggctgctttgtcatccgttagagttgtaggtttggaaaagactgctgttgttcattggagaattggtgaaccatctggtagacaagttgttttggaaatagttggtggtgttccacacttttacgaaaatggtgttggtgtcttgactaatgctccaggttttgaatggcaattgaccaacttgaacaactacgttaacttgtatccaggtgatgcctctgttagaaagttgtctggtattacattgcaacctaccggttctaattctggttttttgggtattcctggtgatgctactccaccatctagatttgttagagctgctttttacagaggtactgctccacaaagagctactggttttgatactgttcaacaatgcttccacctgttgaacaatttcgatattccaatcggtatcgagtactcccaagttggtgatattccagatattccttctgctactcaatggacctctgctattgatttgactaacagaaaggtctactacaagaccgcttacaacaactccattagatgcatcgatttgaaggccatcgatttctctaaggtcaagtatcaatctcagccattggacaagattcaagaacagccaattgaaatggtcaagatcccaagatga

SEQ ID NO: 95 - protein, 355 aa, Alistipes sp.
MKLKYGLLGVAALCGTMQALACTGISLTAADGSYVQSRTIEWGSSALESMYVVIPRGQQLRSLTPTGGQGLAYTARYGVVGLAVVEQAFIAEGINEAGLSAGLFFFPQYGGYETYDPAQEARTLVDLQVAGWILTQFSTIDEVKAAIGKVRIVGLEKNAVVHWRIGEPSGRQVVLEIVGGVPHFYENEVGVLTNAPGFEWQVTNLNNYVNLRPGEVEPYQVGEKTLRAFGATAGFLGLPGDDTPPSRFVRAAFFRATAPQRATAFETVQECFHLLNNFDVPIGLEHPAGQCPDIPSATQWTSAIDLTHRTVYYKTAYNNTIRCIDLSQIDFGKVRYQSHPLDRVQQQPVERITVK

SEQ ID NO: 96 - DNA, 1068 bp, Alistipes sp.
atgaagctgaagtacggtttgttgggtgttgctgctttgtgtggtactatgcaagctttggcttgtactggtatttctttgactgctgctgatggttcttacgttcaatctagaactattgagtggggttcttctgctttggaatctatgtatgttgttatcccaagaggtcagcagttgagatctttgactccaactggtggtcaaggtttagcttatactgctagatatggtgttgttggtttggccgttgttgaacaagcttttattgctgaaggtattaacgaagctggtttgtctgctggtttattcttttttccacaatacggtggttacgaaacttacgatccagctcaagaagctagaactttggttgacttacaagttgctggttggattttgactcaattctccactatcgatgaagttaaggctgctattggtaaggttagaattgtcggtttggaaaagaacgctgttgttcattggagaataggtgaaccatctggtagacaagttgttttggaaatagttggtggtgttccacacttctacgaaaatgaagttggtgttttgacaaacgctccaggttttgaatggcaagttaccaatttgaacaactacgttaatttgaggccaggtgaagttgaaccatatcaagttggtgaaaagaccttgagagcttttggtgctacagctggttttttgggtttgccaggtgatgatactccaccatctagatttgttagagctgctttttttagagctaccgctccacaaagagctactgcttttgaaactgttcaagaatgcttccacttgctgaacaatttcgatgttccaattggtttagaacatccagctggtcaatgtccagatattccatctgctactcaatggacttctgctattgatttgactcacagaaccgtttactacaagactgcttacaacaacactatcagatgcatcgacttgtcccaaattgatttcggtaaagtcagataccaatctcacccattggatagagtccaacaacaaccagttgaaagaatcaccgttaagtaa

SEQ ID NO: 97 - protein, 357 aa, Bacteroides vulgatus
MKKILIALALLLTGIASGSACTGISFLAEDGGYVQARTIEWGNSYLPSEYVIVPRGQDLVSYTPTGVNGLRFRAKYGLVGLAIIQKEFVAEGLNEVGLSAGLFYFPHYGKYEEYDEAQNAITLSDLQVVNWMLSQFATIDEVREAIEGVKVVSLDKPGKSSTVHWRIGDAKGNQMVLEFVGGVPYFYENKVGVLTNSPDFPWQVINLNNYVNLYPGAVTPQQWGGVTIFPFGAGAGFHGIPGDVTPPSRFVRVAFYKATAPVCPTAYDAILQSFHILNNFDIPIGIEYALGKAPDIPSATQWTSAIDLTNRKVYYKTAYNNNIRCISMKKIDFDKVKYQSYPLDKELKQPVEEIIVK

SEQ ID NO: 98 - DNA, 1074 bp, Bacteroides vulgatus
atgaagaagatcttgatcgccttggctttgttgttgactggtattgcttctggttctgcttgtactggtatttcatttttggctgaagatggtggttacgttcaagctagaactattgaatggggtaactcttacttgccatccgaatatgttatagtcccaagaggtcaagacttggtttcttatactccaactggtgttaacggtttgagattcagagctaaatatggtttggttggtttggccatcatccagaaagaatttgttgctgaaggtttgaacgaagttggtttgtctgctggtttgttttactttccacactacggtaaatacgaagaatacgatgaagctcaaaacgccattactttgtccgacttacaagttgttaactggatgttgtctcaattcgccaccattgatgaagttagagaagctattgaaggtgtcaaggttgtttctttggataagccaggtaaatcctctactgttcattggagaattggtgatgctaagggtaatcaaatggtcttggaattcgttggtggtgttccatacttttacgaaaacaaggttggtgtcttgaccaactctccagattttccatggcaagttatcaacttgaacaactacgttaacttgtacccaggtgctgttactccacaacaatggggtggtgttactatttttccatttggtgctggtgcaggttttcatggtattccaggtgatgtaactcctccatctagatttgttagagttgctttctacaaagctaccgctccagtttgtccaactgcttatgatgctatcttgcaatccttccacatcctgaacaatttcgatattccaatcggtatcgaatacgctttgggtaaagctccagatattccttctgctactcaatggacttctgctattgatttgaccaacagaaaggtctactacaagactgcttacaacaacaacattaggtgcatctccatgaagaaaatcgacttcgataaggtcaagtaccagtcttatccattggacaaagaattgaagcaaccagtcgaagaaatcatcgtcaagtga

SEQ ID NO: 99 - protein, 359 aa, Muribaculum sp.
MKTSLILKSALVSAVAALSFHLSASACTGISFRAADGSVVLARTIEWGDSELPSMYVIVPRGMEFISYTPTGQNGMKFKVKYGYAGVSVVKDEFVAEGLNEAGLSAGLFYFPGYGKYPEYKEAYNGRTIADLQFVSWMLSSFATVDELLKELDRVRLVAIEGTSTVHWRVGDATGRQIVIEIVDGRTNVYENTLGVLTNAPGFEWQVVNLNNYVNLYSGGAQPKTVDGVLIHPFGAGSGMLGIPGDVTPPSRFVRAFFYKTTAPQQKTGRDCVMQCFHILNNFDLPVGVEHEPGKSPDIPSATQWTAASDLTGKKLYFRTMYNSSIRCIDLSAIDFSTVEYVSRPLDEAKEEPVEMLQV

SEQ ID NO: 100 - DNA, 1080 bp, Muribaculum sp.
atgaagacctccttgatcttgaagtccgctttggtttctgctgttgctgctttgtcttttcatttgtctgcttcagcttgtaccggtatttcttttagagctgctgatggttctgttgttttggctagaactattgaatggggtgattctgaattgccatccatgtatgttatagtcccaagaggtatggaattcatctcttatactccaactggtcagaacggtatgaagttcaaagttaagtatggttacgccggtgtttccgttgttaaggatgaatttgttgctgaaggtttgaacgaagctggtttatctgctggtttgttttactttccaggttacggtaaataccccgagtacaaagaagcttataacggtagaacaattgccgacttgcaattcgtttcttggatgttgtcatctttcgctactgttgacgagttgttgaaagaattggatagagttagattggtcgccatcgaaggtacttctactgttcattggagagttggtgatgctactggtagacaaatcgttatcgaaatcgttgatggtaggactaacgtttacgaaaacactttgggtgttttgactaatgctccaggttttgaatggcaagtcgttaacttgaacaattacgtcaacttgtattctggtggtgctcaacctaaaactgttgatggtgttttaatccatccattcggtgctggttctggtatgttgggtattccaggtgatgttactccaccatctagatttgttagagccttcttctacaaaactaccgctccacaacaaaagactggtagagattgtgttatgcaatgcttccacatcttgaacaacttcgatttgccagttggtgttgaacatgaaccaggtaaatctccagatattccatctgctactcaatggactgctgcttctgatttgactggtaagaagttgtacttcaggaccatgtacaactcctccattagatgcattgacttgtccgctattgatttctctaccgtcgaatatgtttccagaccattggatgaagctaaagaagaaccagtcgaaatgttgcaagtctaa

SEQ ID NO: 101 - protein, 363 aa, Bacteroides sp.
MKSRILLAIAMGIGSMFAYPLPAEACTGITLKSKDSVTVVARTIEWGGSDLNSQYVIVPRGYEQRSYTPAGVTGMTFKARYGYVGLAVEQKDFVAEGLNEAGLSAGLFYFPNYGEYEKFDEALQDKSIADLQLVSWILGQCATVDEVKQAVSEVHIINIDPRASTVHWRFTEPSGRQIVLEMIDGKPKFYENPLGVLTNSPSFDWQLTNLNNYVNLYPGTAPAQSLGGAKLASFGAGSGFLGIPGDVTPPSRFVRAAFYQATAPVQDTALKTVLQGFQILNNFDIPVGIEFQAGKIPADIPSATQWTSATDMADRIIYYRTMYNSAIRSIDLKTIDFSKVKYRAVPLDAEKQQPIVSVKIANK

SEQ ID NO: 102 - DNA, 1092 bp, Bacteroides sp.
atgaagtccagaatcttgttggctattgccatgggtattggttctatgtttgcttatccattgccagctgaagcttgtactggtattactttgaagtccaaggattccgttactgttgttgctagaactattgaatggggtggttctgatttgaactcccaatatgttatagtcccaagaggttacgaacagagatcttatactccagctggtgttacaggtatgacttttaaagctagatacggttacgttggtttggccgttgaacaaaaagattttgttgctgaaggtttgaacgaagctggtttgtctgctggtttattctactttccaaactacggtgaatacgagaagtttgatgaagccttgcaagataagtctattgccgacttgcaattggtttcttggattttgggtcaatgtgctactgttgatgaagttaagcaagccgtttccgaagttcatatcattaacattgatccaagagcctctactgtccattggagattcactgaaccatctggtagacaaatcgtcttggaaatgattgatggtaagccaaagttctacgaaaacccattgggtgttttgactaactctccatcttttgattggcagttgaccaacttgaacaactacgttaacttgtatccaggtactgctccagctcaatctttaggtggtgctaaattggcttcttttggtgctggttctggttttttgggtattccaggtgatgttactccaccatctagatttgttagagctgctttttatcaagctaccgctccagttcaagatactgctttgaaaactgtgttgcaaggtttccagatcctgaacaatttcgatattccagtcggtattgaattccaagctggtaaaattccagctgatattccatctgctactcaatggacttctgctactgatatggctgatagaatcatctactacaggaccatgtacaactccgccattagatccattgatttgaaaaccatcgacttctccaaggttaagtatagagctgttccattggatgctgaaaagcaacaacctatcgtttccgttaagattgccaacaagtga

SEQ ID NO: 103 - protein, 359 aa, Barnesiella viscericola
MKRLFLLGIAAMLLSTNTFSWACTGITLTAKDGSKIVARTIEWGGSDLNSQYVIVPRGYQQQSFIPGGEQTGLKFTARYGYVGLAVEQKEFIAEGLNEAGLSAGLFYFPGYGQYEAYNPTHQATSIADLQLVAWILGSFSTIDEAVKGLKTVHVVSIDPRASTVHWRMTDVSGRQVVLEIVGGQLNFYENKLGVLTNSPGFEWQITNLNNYINLYPGTTDTKSFGDITLSSFGAGSGFIGLPGDITPPSRFVRAAFYQTTAPEQNDAIGAVTQSFHILNNFDIPIGAEFLAGQTPTDIPSATQWTSSADVTHRVLYYRTMYNSAIRSFDLKSIDFGRVKYQVQPLDVNKQQPIQTISIK

SEQ ID NO: 104 - DNA, 1080 bp, Barnesiella viscericola
atgaagaggttgttcttgttgggtattgctgctatgttgttgtctaccaatactttctcttgggcttgtactggtattactttgactgctaaagacggttctaagatcgttgctagaactattgaatggggtggttctgatttgaactcccaatatgttatagtcccaagaggttaccaacagcaatcttttattccaggtggtgaacaaactggtttgaagtttactgctagatacggttatgttggtttggccgttgaacagaaagaattcattgctgaaggtttgaacgaagctggtttgtctgctggtttattctattttccaggttacggtcaatacgaggcttacaatccaactcatcaagctacttctattgccgacttgcaattggttgcttggattttgggttctttctccactattgatgaagccgttaagggtttgaaaaccgttcatgttgtttccattgatccaagagcttctactgttcattggagaatgactgatgtttccggtagacaagttgttttggaaatagttggtggtcagttgaacttctacgaaaacaaattgggtgtcttgactaactctccaggtttcgaatggcaaattaccaacttgaacaactacatcaacttgtacccaggtactactgataccaagtctttcggtgatattaccttgtcatcttttggtgctggttctggttttattggtttgccaggtgacattactccaccatctagatttgttagagctgctttctatcaaactaccgctccagaacaaaatgatgctattggtgctgttacccaatccttccatatcttgaacaatttcgatattccaatcggtgctgaattcttggctggtcaaactccaactgatattccatctgctactcaatggacttcttctgctgatgttactcatagagtcttgtactacagaaccatgtacaactccgctatcagatctttcgatttgaagtctatcgatttcggtagagtcaagtaccaagttcaaccattggatgttaacaagcaacagccaattcaaaccatctccatcaagtga

SEQ ID NO: 105 - protein, 345 aa, Lactobacillus hominis
MTKNLLLGIAAVCGSTFQAVACTSILYSPKDHYFGRNLDYEIAYGQKVVITPRNYEFEFTDLPAEKSHYAMIGVAAVADNTPLYCDAINEKGLGVAGLSFAGQGKYFPNAADKKNIASFEFISYLLATYETVDQVKESLTNANISNVSFAKNTPASELHWLVGDKTGKSIVVESDEKGLHVYDNPVNALTNAPLFPEQLTNLANYASVVPGEPDNNFLPGVNLKLYSRSLGTHHLPGGMDSESRFVKVCFALNHAPKDSNEVENVTNFFHILESVEQAKGMDQVGPNSFEYTMYTSCMNLEKGILYFNCYDDSRISAVDMNKEDLDSSDLVVYDLFKKQDINFVN

SEQ ID NO: 106 - DNA, 1038 bp, Lactobacillus hominis
atgaccaagaacctgttgttgggtattgctgctgtttgtggttctacttttcaagctgttgcttgtacctctatcttgtactctccaaaggatcattacttcggtagaaacttggattacgaaattgcctacggtcaaaaggttgttattaccccaagaaactacgagttcgaattcactgatttgccagctgaaaaatcccattacgctatgattggtgttgctgcagttgctgataataccccattatattgtgatgccatcaacgagaaaggtttgggtgttgcaggtttgtcttttgctggtcaaggtaaatactttccaaacgctgctgacaagaagaatattgcctcctttgaattcatctcctacttgttggctacttacgaaactgttgaccaggtcaaagaatccttgactaacgctaacatctccaacgtttctttcgctaagaatactccagcttctgaattgcattggttggttggtgataagactggtaagtctatcgttgttgaatctgacgaaaaaggcttgcacgtttacgataatccagttaacgctttgactaatgctcctttgtttccagaacaattgaccaacttggctaactacgcttctgttgttccaggtgaaccagataacaattttttgccaggtgtcaacctgaagttgtactctagatctttgggtactcatcatttgcctggtggtatggattctgaatcaagattcgttaaggtttgcttcgctttgaaccatgctccaaaagattctaacgaagttgaaaacgttaccaacttcttccacatcttggaatctgttgaacaagccaaaggtatggatcaagtaggtccaaattctttcgagtacactatgtacacctcttgcatgaatttggagaaaggcatcttgtatttcaactgctacgatgactctagaatctccgctgttgatatgaacaaagaggatttggattcctccgatttggttgtttacgacttgttcaagaagcaggacattaacttcgtcaactaa

SEQ ID NO: 107 - protein, 356 aa, Lactobacillus tucceti
MTKNLLLGIAAVCGSTFQAVANQKGVVAMCTSISYEALDGAKFLSRTMDFAFELNGRPTFLPRDYEWISSFDKKTYKADYAILGTGAKYGNNYMVADGTNEYGLAAAELYFAHKAEYDHEPTDGAINLVSEEFILWALGNNKSLAELKDNIKNVHIIESDKGVMGKNQPLHWIFSDLTGATYVIEPEGDGLILQEDKTGVMTNTPDYQWHETNLANYLGAQPNNFGSKKFGDLEVQPLGQNGTFRLPGGYTAVDRFVRTAFNREVIEKAADATEAVNTIMHILDSVDVPKGANTKPDGPSYTQYQSIFDMTNRIMYYIPYGNRTVFKVRMTDEMIGAQKTPKEFPVALTQEFNELN

SEQ ID NO: 108 - DNA, 1071 bp, Lactobacillus tucceti
atgaccaagaacctgttgttgggtattgctgctgtttgtggttctacttttcaagctgttgctaatcaaaagggtgttgttgctatgtgcacctccatttcttatgaagctttggatggtgctaagttcttgtctagaactatggatttcgccttcgaattgaatggtagaccaacttttttgccaagagactacgaatggatttccagctttgataagaaaacctacaaggccgattacgccattttaggtactggtgctaaatacggtaacaactacatggttgctgatggcactaatgaatatggtttggctgctgctgaattatactttgctcataaggctgaatacgatcacgaacctactgatggtgcaatcaatttggtttccgaagaattcattttgtgggccttgggtaacaacaaatctttggctgaattgaaggacaacatcaagaacgttcacatcatcgaatctgataagggtgtcatgggtaagaatcaaccattgcattggatcttctctgatttgacaggtgctacctatgttattgaacctgaaggtgatggtttgatcttacaagaggataagactggtgttatgactaacactccagattatcaatggcacgaaactaacttggctaattacttgggtgctcaacctaacaatttcggctctaaaaagttcggtgatttggaagttcaacccttgggtcaaaatggtacttttagattgccaggtggttacactgctgttgatagatttgttagaaccgccttcaacagagaagttattgaaaaagctgctgatgctaccgaagctgttaacaccattatgcatatcttggattccgttgatgttccaaaaggtgctaataccaaaccagatggtccatcttatacccaataccaatccattttcgacatgaccaacaggatcatgtactacattccatacggtaataggaccgttttcaaggttagaatgaccgacgaaatgattggtgctcaaaagactccaaaagaattcccagttgctttgactcaagaattcaacgagttgaactaa

SEQ ID NO: 109 - protein, 366 aa, Turicibacter sanguinis
MTKNLLLGIAAVCGSTFQAVASSKLMFYFNRKMGLKNMCTGLSLVTKDNKHLFGRNLDVPATYGQAVHIVPRNYDWFNIVSDETYTSKYACIGMGIVVDRLPLLFDAVNEKGLAGAGLNFTHFAKFNEKAVDGKTNISGSTFLYWALSNFSNLDELKEALKQLIITNIPVKEGLPVAGLHWMFTDLSGKSIVIEYMEDGMHVYDNPVGALTNDPTFPWHLTNLCQYVTLDTKTPAPKQFGEYVAKPFGHGLNMCGIPGDASPAARFVRTVYFRDTVVEADDEVSGVSAFFQVLTGVTVIKGSEIDQNGDMNYTVYKSCMCQESGTYYYTDYKNRRISAVCLSKADLDGKEIISFEYQGKQDILFQN

SEQ ID NO: 110 - DNA, 1101 bp, Turicibacter sanguinis
atgaccaagaacctgttgttgggtattgctgctgtttgtggttctacttttcaagctgttgcttcctctaagctgatgttctacttcaatagaaagatgggcttgaagaacatgtgtaccggtttgtctttggttacgaaagataacaagcacttgttcggtagaaacttggatgttccagctacttatggtcaagcagttcatatcgttccaagaaactacgattggttcaacatcgtttctgacgaaacttacacttctaagtacgcttgtattggtatgggtatcgttgttgatagattgcctttgttgttcgatgccgttaacgaaaaaggtttggctggtgctggtttgaattttactcatttcgctaagttcaacgaaaaggccgttgatggtaagactaacatttctggttctacgttcttgtactgggctttgtctaatttctctaacctggacgaattgaaagaagccttgaagcaattgatcatcaccaacattccagtcaaagaaggtttgccagttgcaggtttacattggatgtttactgatttgtccggtaagtccatcgttatcgagtatatggaagatggtatgcacgtttacgataatccagttggtgctttgactaacgatccaacttttccatggcatttgaccaacttgtgtcaatacgttactttggataccaaaactccagctccaaagcaatttggtgaatacgttgctaaaccattcggtcatggtttaaacatgtgtggtattccaggtgatgcttctccagctgctagatttgttagaactgtttacttcagagataccgttgttgaagccgatgatgaagtttctggtgtttctgcatttttccaagttttgactggtgttaccgttatcaagggttccgaaattgatcaaaacggtgacatgaactacaccgtttacaagtcttgtatgtgccaagaatctggcacttactactacactgattacaagaacagaagaatctccgctgtttgtttgtctaaggctgatttggatggtaaagagatcatctcctttgaataccaaggtaagcaagacatcttgttccagaactga

SEQ ID NO: 111 - protein, 350 aa, Lactobacillus sakei subsp. sakei
MTKNLLLGIAAVCGSTFQAVACTSLTYSTVDGHHFLARTMDFSFELNGNPLFLPRQHQWQPVLEKQAIQNQYALMGAGAKLGDQYLVADGFNEHGLGCAELYFAHAAVYEPQPVPNKLNLVAEEFIVWVLGNHQTLEEVAADLDNVRIIESDQGVMGANQPLHWILSDRSGKTMVIEPRGNGLQLIDDPVGVMTNTPDLDWHIKNLSNYLNLQPQPFMERPFGNYQAGLFSQGTGTQALPGSYTPPDRFVRAAYSRQYMPEANNVAEGVNHILHILDNVTIPKGVNIGSGGSSDYTQYQGISGLNNLAYYMVNYDNRHVYQTNLTTDLIENQKEPLIYHLPQGQQNTILN

SEQ ID NO: 112 - DNA, 1053 bp, Lactobacillus sakei subsp. sakei
atgaccaagaacctgttgttgggtattgctgctgtttgtggttctacttttcaagctgttgcttgtacttctttgacctactctactgttgatggtcatcatttcttggctagaactatggacttctcctttgaattgaatggtaaccctttgttcttgccaagacaacatcaatggcaaccagttttggaaaagcaagccatccaaaatcaatacgctttgatgggtgctggtgctaaattgggtgatcaatatttggttgctgacggttttaacgaacatggtttgggttgtgctgaattatactttgctcatgctgcagtttatgaaccacaaccagttccaaacaagttgaatttggtagccgaagaattcatcgtttgggttttgggtaatcaccaaaccttggaagaagttgctgcagatttggataacgtcagaatcatcgaatctgatcaaggtgttatgggtgctaatcaaccattgcattggatcttgtctgatagatctggtaagaccatggttattgaacctagaggtaacggcttgcaattgattgatgatccagttggtgttatgactaacactcctgatttggattggcacatcaagaacttgtctaactacttgaacttgcaaccacagccattcatggaaagaccatttggtaattatcaagccggcctattttctcaaggtactggtactcaagctttgccaggttcttatactccaccagatagatttgttagagctgcttattctagacagtacatgcctgaagctaacaatgttgctgaaggtgttaatcacatcctgcatattttggacaacgtgactattccaaagggtgttaatattggttctggtggttcttctgattacacccaatatcaaggtatctccggtttgaacaatttggcctactacatggttaactacgataacagacacgtttaccagactaacttgaccaccgatttgatcgaaaatcagaaagagccattgatctaccatttgccacaaggtcaacaaaacaccatcttgaactga